SEQUENCE LISTING (385 sequences)

SEQ ID NO: 1 - DNA, 118630 bp, Homo sapiens
  source 1..118630: mol_type = other DNA
agtagtttgcggcatccggaggagcagcagcagcagtagcggcggcggctggcgcagcgcggagacggacgagcgggcacagacggcagcacagctcccgtgcaccatgcctggcttgcccgggaggcactggcacccgcgtccctagcccggcgctggcccttggcgcgttgcgctccgcagcattccggctgagcgatttcatccgaaatcctaaacctcctaccccaaacccattgcctcctgcttgctcatactccacactcacactccccacaccacacactcactcttcctctctctctcacacattcacacacagcccgagacagacagagaaagcgagacagacctcccgagtgcgcgagcgcccgggaggaagggagcccaacgcagcgcggccagcgcctctccgggaagatgaaatcaagacgctcctgcaagtaaacttcggcggcgacaaaatcgtcgactgtggcagctgctgaggcggaccccagcgacgtgcaactgagctaggagggcttcctcccgcggcggcggcggcggcggcggcggcggcggaccttggagcgacaccccagcccaaacggagagaagagaagcacccggtacccacggcggcggccccgcctctcctggccggtactgttgttgcctccccttccctcttccccattccccgccacccccggccattagaaagaccggggctttgattttcttggacaaattgccatctccccgtttccacaaacttgacgtggagggtcggactgcgttgttaactttgatttcctagacgaaaacgcccgcagaccctcaactgctctgtggggggaactccgctcccacctcggatcctggacagacgcgcccccaagcccttcgaagccagccttagcgcgagggagcgcgctcaagctctggcagcctctctcccctcccccttcctctgctggccccgggaccgccatccgactcaaccctataaccgggtgcccgcggcgcttgccagtaactgactcccgtctgcgacaccccctttctcccgaacaggcctcgaagaggccagtgtggcccccccagaccccggagggcgcattccaagcgggtactctctccgcagtgaactgggagcgctggggggaggtctttgcgcatgccaggtgcacagacagcgagttcgccagcctcagtgccggtgcaccgcggcgggccgggcagtgcctgaaaggctgcgcctcctccgccctctttaccttcgcctcaagtactgtccggactcagcggtgcgtcctccaggccgcgcagacgccgcccgacccgcagccctgggcggctgccgacagtcgcgcccggctaactttgaaggggtaaaactgagtagccgggctggagggagggggctcggggccgcgcggcgctcccttggcccctcggagcccccggggcccccgcgagcccccgccgcgtccgccgcaacctcagcattggagccgccgcgggtgtcccccgcgcgccgccgggccgccctgggcgggacttctcccgcggcctccggggccacggggcgcgcgcaacaggcggcccgagccggcgggaagctggagcgccgacggcgtcggtctcggaggggtgtggagaggcgaggccaggcagagccccgtgcagccatggagtcgctcctgctgccggtgctgctgctgctggccatactgtggacgcaggctgccgccctcattaatctcaagtactcggtagaagaggagcagcgcgccgggacggtgattgccaacgtggccaaagacgcgcgagaggcgggcttcgcgctggacccccggcaggcttcagcctttcgcgtggtgtccaactcggctccacacctagtggacatcaatcccagctctggcctgctggtcaccaagcagaagattgaccgtgatctgctgtgccgccagagccccaagtgcatcatctcgctcgaggtcatgtccagctcaatggaaatctgcgtgataaaggtggagatcaaggacctgaacgacaatgcgcccagtttcccggcagcacagatcgagctggagatctcggaggcagccagccctggcacgcgcatcccgctggacagcgcttacgatccagactcaggaagctttggcgtgcagacttacgagctcacgcccaacgagctgttcggcctggagatcaagacgcgcggcgacggctcccgctttgccgaactcgtggtggaaaagagcctggaccgcgagacgcagtcgcactacagcttccgaatcactgcgctagacggtggcgacccgccgcgcctgggcaccgttggccttagtatcaaggtgaccgactccaatgacaacaacccggtgtttagcgagtccacctacgcggtgagcgtgccagaaaactcgcctcccaacacacccgtcatccgcctcaacgccagcgatccagacgagggcaccaacggccaggtggtctactccttctatggctacgtcaacgaccgcacgcgcgagctctttcagatcgacccgcacagtggcctggtcactgtcactggcgctttagactacgaagaggggcacgtgtacgaactggacgtgcaggctaaggacttggggcccaattccatcccggcacactgcaaggtcaccgtcagcgtgctggacaccaatgacaatccgccggtcatcaacctgctgtcagtcaacagtgagcttgtggaggtcagcgagagcgcccccccgggctacgtgatcgccttggtgcgggtgtctgatcgcgactcaggcctcaatggacgtgtgcagtgccgtttgctgggcaatgtgccctttcgactgcaggaatatgagagcttctccactattctggtggacggacggctggaccgcgagcagcacgaccaatacaacctcacaattcaggcacgcgacggcggcgtgcccatgctgcagagtgccaagtcctttaccgtgctcatcactgacgaaaatgacaaccacccgcacttttccaagccctactaccaggtcattgtgcaggagaacaacacgcctggcgcctatctgctctctgtgtctgctcgcgaccccgacctgggtctcaacggcagtgtctcctaccagatcgtgccgtcgcaggtgcgggacatgcctgtcttcacctatgtctccatcaatcccaactcaggcgacatctacgcgctgcgatcctttaaccacgagcagaccaaggcgttcgaattcaaggtgctggccaaggacggcggccttccctcactgcaaagcaacgctacggtgcgggtcatcatcctcgacgtcaacgacaacaccccggtcatcacagccccacctctgattaacggcactgccgaggtctacataccccgcaactctggcataggctacctggtgactgttgtcaaggcagaagactacgatgagggcgaaaatggccgagtcacctacgacatgaccgagggcgaccgcggcttctttgaaatagaccaggtcaatggcgaagtcagaaccacccgcaccttcggggagagctccaagtcctcctatgagcttatcgtggtggctcacgaccacggcaagacatctctctctgcctctgctctcgtcctaatctacttgtcccctgctctcgatgcccaagagtcaatgggctctgtgaacttgtccttgattttcattattgccctgggctccattgcgggcatcctctttgtaactatgatcttcgtggcaatcaagtgcaagcgagacaacaaagagatccggacctacaactgcaggtaaagccaagttttctttctttgtcttggatgaataaggtgtgtttatctgtgtctctgtgtgaaaatcctggtgcttttgtttcctacttaaatgcatgcagcatacttgcagtgcttctttttgtagggtgaaaagtttgtgacagtccaagttgatcaattcttttgatctgacaatttgctggaagacaatcatttagtgtttgaatggattctgtcacataatgatggtgagcagaaacgcaattatcattttaaaatttgcatagctgtctccatgaagttatggtaggaaaaacagagagagagagagggagggggagagagagagagatcatatttttaggaaaaccaccaactaccacaacttggtgtctggggcacagtgtttttgggacaggtactgaatgaaaggagagggagtgttggcagctaccctgtcttgttgcagtgatgaactctggagagagactgggagtgggagtgtaagatgggaaagggagatgagggggaggagagaacaggagtgtgtggagatgcaggtgattagtggtttttagtttattcttggcttgttttttatctagtccccaaacatctggctatataaagtgaaattctatcaggcatctgtatgttttcaaatatctggttggttggtagtgtggagaccaaacatcccaatgaggaacaagactttgcatccagatgtttcagtaaaaatagcaatcttctagagttgatgatgctttcctctaatgtgagactgtgttgtggcattaagatctggttcctaccagtggatgtcactgtgggctattgtgttacagtttctcttgaagtgtgtgaaggatgtgtgtgtgtctgtgtgtgtatgggggggggagggagagagagagggatagaagggggaggaaaagggagaaaagaagaaacacagtattttttaaaaagatttcctgataagcacattagaaggtcttactgaagccctttcttacatttggatagagcacataagtctaagcatttctagaatcctagaatccaatttaggataatacatgacattttttttccagcagtgtctcatcgaaacagttgaaaaaagttgtttctttagtttgctgaaaatgaatgatgctgctatttacaaccaaggtgtgtgtgtgctttcaacacacctacacatcagtgtgtcttttgtgccatttgtgtctgcatgttaatcagtcaaaatatgttcctgtaggttcagtttgtatttgacaccgtctgaaatgcacacttctgtgaatcagagccccctgaatatgtaacagccatgcaattattgtttcccttttactattctctgcgttacacaagttagtagacaaatgctaaccacacatgaatatcaatcaggtgccataaaagagcagctgcaatgcctcataaatgctttaatcctgttactagtgaaatggccatgacttttctttccttcagcaccaagtcatttcagatgggagaaatgtaaattaatattgctcttagctttcaaacctgtgactgctagtggcgatttttataggagaaaaatgtggggtcaataaaattttatttatatggagataattatggagaggactctttttaagccatcttctcagcataaagtaaatgcataagatcctttccttgatgaaaaagaaatcagcttgaaataaagatattgaaggctttgccacctcttggtgatgtatgactagaagtgagagccctttctgagaacttgccaaggagatgattaaacaggaggagagcatctgaatattgaaaaaaaaaaaaaagagtaaatctcagtgctctccaacttcacccaaatgtgtattttgtgaagcgttctgtttcctgtgatggtttgggattaatgaagtacagtataattaaataattggttaatacagaaagggctgcacaatgaatgcttcagctgaataatttgatggggcattgccaaagttactcatcttgggtaaacaaaatgattaaaatttcccattgattctgtaaacagattagaatagaaaatcaaccttagtactagggttttggggattttatctgctcccaaaccccagtagtaccagtttgccatcactgcaaatcttcctgagcacagagcgaaggggtgaggggaactcaattgcttaactctaggtagatcctggggacagagcaagaatttgcctctgtagatgtgtagtctccctatctgctcagactttttagaaacctaaagttcaggagcgtgtctttccatgtgattaggcagaggtttctttttctaagcctccgcagaacctcttcctgaaatgcattcatttatgagacttgcgagatgatctgcaattgctgcttgaattgggctccctttttgtattcagacaattagctttgtcgagtgagaataaggcacagtctttcccttgtaaagatcaacctgtattacttattaaccctattgttttgctgattattgcccagaaatcgaatttccaaaaaagaaactgaatcattatgtgcaaagtaataaatgactttgagaatctggctacatttaaatgtatttcttttgacaagtctcttaatgcctggtgtctttggtgcacgttgctgggtctgtgtcagtgttaattaaattgagctctttatgttccagttgcagcacagctgcttgtaggtacacctgaatggagaaggatgctgaatgcactaacctttaaattcctttgcagtaattgtttaaccatcacttgtctcctcggctgttttataaaaggacaaaacagcaagtgtctgcattgcatctcggtttctcccattagcgaggagcaagacaaaaagacagaggagaaagtgagcctaaggggaaagaggtaaaaaggcatttcccctcatttaaatgggtttggctattagatttttctaggagaggagggggtcggggggtgagtaaaagggaaccgggctagggcagggaggagggtgtggaggagacgcgaaagaatggggtctgatctcctaccccagtccaaaactgatcgagtgagtcatagaggaagctgattgagggcgtgtgccacatggcaaatgcctggaggaactggcaagggggtgggggcgggtattttttattttcctctatctcctctttggtgacttgctttgtatttcacctaggagttccttttggttccagggaagctccagatttgagcattttcatgctttctctcccataggctcaactttccctgtccccccacccgtgctcacaggccactcacaatgaaaaattccaaatccaaagtatagaaacttttttagggggggttggtgggaggttactatgtttaatatgaaaagtttatggccctcagcctgttttcccttgtaaaaagcacagggagaaacccgatgatgtaatgcaccagaataaggagctggagcggagatggggtgcaaccctctggggtgcttaatgtttattaatcagtgcatgatttgggagggtgttagtgattcatgctgccttttctgtcacatagaattgctgagtactcctatgggcatcaaaagaaatcaagcaagaagaaaaaaatcagtaagaatgacatccgcctggtaccccgggatgtggaggagacagacaagatgaacgttgtcagttgctcttccctgacctcctccctcaactattttgactaccaccagcagacgctgcccctgggctgccgccgctctgagagcactttcctgaatgtggagaaccagaatacccgcaacaccagtgctaaccacatctaccatcactctttcaacagccaggggccccagcagcctgacctgattatcaacggtgtgcctctgcctgaggtgagtgcagctaagtggctctgtgaggttctcccaggtctccttctttagctgctcgctggataccataagctgctgtgctggcacatcccctggcaggggtggggagaatttctctgcttatgcccattttcatactaagggtttgctatcagtgcttcttggagcaaaaggtaacccaacatactgggcctttgtgtaggtatgggtattggaattggtaactcaaagagtctgagcccttgaaatcattatcctggcataactcaagtttgctccacctgttgcaaatatagcatgcttgacagccagcagtaccagtatgccagtaaagtggcttagaaagagtcatcaagtagtgttgaaaatggatgaaaccctctaatgccctgggaccaattttccagtgtgagagtgattcagcctcctaaagaccccatttgctttttctttcgtcacatcactgttctctagcattttggtgagagaatatggataggctggttaaagggaggcaaagcaaaaaaattaaaaagccactttgccatttcttagcagttctagtggtaacatttgatggttgaggagattgaaactgtcattattgctgaaaatttaagaattagttggctggttgcagtggctcacacctgtaatcccagtactttgagaggccgaggcaggagaatcggttgagcctggaattccagaccagcttgccagcctgggcaatatagtgagacttcgtctctaacaacgaaaaaaaaaaagagtaagaataagaataagaattatctatgcatttgagtgagctctgtttctttctcttcactgggaatgaatgtatttaagtagaattttttttttttttttgagatggagtttctcttgttgtccaggctagagtgcaatggcacaatgttggctcaccgcaacctccacctcccggcttcaagcaattctcctgctccagcctccagagtagctgggattacaggcatgcaccaccacgcccagctaattttgtatttttagtagagacggggtttcaccatgttggtcaggctggtctcaaactcctgactgcaggtgatcctcccacctcagcctcccaaagtgctgggattacaggcatgagccactgcaccgggcttaagtagaaatattttttaaaatgtgatgtggtcagatgtttgctggctattgaaatattttagggcagtagagtggtatatggatgatggctggtgggtggctgggaggaaggctataagtaacatttcttaagttgatttcatttttccatgaattgcttaagtccctctttggttagaaatctgatgcactgggaggcatatgtcctcttccaagctaccagcgaaaagaatactgcatacttagagagcactttcacatctttcaaagcaccttcccaaacattgtttcgtttgatcctcacaatcaggtatcttgataggttgcccttcagtagatgatgaaacagaggtccagagatgctaagggacttactcagactcacaaagctggtcatcaagagagggaggacgagactgaaggactctatactgcctggcaaatgtgatttcagtgacaccttcctgccttaaaagtaattcagcctgacatacccaggctatgatgcatacataaaatggcctcatagtatttgtgtcaattttgtaggataaggggtcttaggaccacttacattgattgcttcttctagttcctgaacaataattcccatcagaattaatggggccaaacagcagtgcttttgatgagtaactgatggtattctgccagcaacaaaagcaccaatattttcaatttataactctataaataaggattttttgaaagtccagtattgtgctaggtaaactcagttataattattaatgtcagtgaatactactaaggcctaggccgtagactacttacaagactacttgtggatcaccaatagctcagcaatagaaagtgagaggtagtggtgtgtagtggaatcagattgatcatgattttgaatccaggaatcactgcttgctgtggaacataagactctgtttctttatctacaaaatgggcacacctgtcatgcaaagttgttccaaggaatatgacttgtgtagtgtagtaggttcaactcattggtgtccccaaaatagtagacctcttcactatacagttggagaatcagtgttacaatggcttgcactactgattgtgtctccctaggcacttgtaaccaaagaggaatttaggctgcttctatatagatacatcctacaaagtattatggccatactactcatattgatcatttaaacaatagaggtgaaagatgccaaagttgtggggttcagttgttcacaagtaacggcactgttacttgtggagatggacaaaagggttgaatcctgaggacttggtgatcacttaggcaatcaattcaaggcactagagaaatgtgagttctggtgtctatgaaatagatggagaggacagaaaacatatgacgacatctgattggtttacctaagacttagtatcatttctgggattcttccatgggctgaatgaaacaaaatattgcttcaattatttagaaaagccgtttaaaatctcagttctgatcctgtctctgactcacaaagccagatgcttttccttggttaacaaaattacacagttttggtgtgtacagctggtcatggccaagacagtgtgtactagacatagtgaggttgtgtgtatttctaatattcagtccctcctggagagttcaactggcaaacccattcagaaaggagagaggcccacttacagctgaatacagaccaggccatttaacctgaatcagtcacaatgccctgcgtagggaaaatgacacttcttttcttttataccaaaggccactggactcggtggaatacttaggtccaatgaaatctatagaagagcagggatgattgatttgttcattcaactgatatttgttgagcctactctttgtaaggcactgctctaggctctggcgttagagagatgaaaaagacaagaaatgcacattctaaagtgtgagacaatcaagtaaataggtgatttgcaatatagagtaataaatgctaatatagggtgcaatgggacattctccattgctaggataataacattttgatatatttcctacattatctttatattttcctaattaattaattaatccattcaagaaagcaacatttattaagcaattatagcatagaatgataagtgctatgatgtggcaaatataagattctatagaaatttctctatattatgttacttacattgttctttatattatttcattaaaccattctttcatttgttcattaatttatcccattagcagttattatacatttagaatgtgccagataatgtactacttcccggggatcaaaaataataaaacacttgtttcttgaacccagggagcttacagaaatacagacaagtaaacaggtaatttcaattcagaataacatcctattcctgagcacgttacattcttactctccaggttgggacaaaagggtactacatagtaggttcctctgttgggttacctgtgtcaaacccagtccctttatgagctgtagatccagttacagggcttactctcaaagggattatagacgagtccactcagacctgttgcgtgctagctgtactcactctatcacactttgaaatttatcaccatggcattagatcaagtcagcaagcattgttggagtatccactatgtgctcagctgtgtggcagggaccatggagattccaaataagtttaagacgtaacttctgccctcagagagttcacagtctagttggggagctaagatcacccacaatgatttcacgggcaagcaatacaagatagtttctgatcacctgtcgctttgtagactcctaagaactcctttctcctcctttaatggcagccttacattttatgtagatttgccattgaaccctcaaaatttagtgctgaatggaagatagctcttgtatcagtgtccaaacctagccacatctgaacattgttggctctagaagctaacagaaatatgactttgaatgcagttaggtaggcaaggtaaagggaatggaacactaatcaagaattggaactcagtttacagtattgcagagataatcttgtgtagaattgtatatcattatttacttgtcctctgatgttggctgggaaatcagctatgtggattttgtggtttcgtacacaaatttgaggggcaaagtggaggatgcctttgctgtttacagaagacttttgtagaattattgatgtcaggtatcttttacatattttaaactgaaaggtgaggtttgcattccttggtatcctggtttattttactcatagacttagtgtatatgtgaatgaattagagggcgtgtgtatgtgtgtgtgtatgtgtgtgtgtgtgtgtatgagggcgtagaaaagagtgaagagagaggaaaggaggttgaaaaagaaggaaaatggagaagaaagatagtaggtttgtcagtttgctaagatctttctaaaaatagtccaaattttttgagaggctccttagtaaggcaaatgatttttcttccggtcttatggaatggagacatgcctactggtgtctctggatgctggaaagtgttattcctttgtacagcagcattgctgcattaccatttttgtcacctggctgcagtcattattggaggaagaaaatgcaactcattgcttctactggaaatggagacttgggtgaggcttcttcttgcctccaggaggggtgaaatggcccaaacacccagtgtctttgtgcagtatttttaaattttagttttctgatgttgaatgtttctgagctgacagctgtaagggagtgatagtaggcaaaaacttagagaattccagtcataggtccgtagggatttaaattttggtttcagtctcaatcttgaattctattttctacctttcatcagcaaactgtagtttctttgatatgaggcatctaaggggtgtgtgtgcatgctctgctatagccactcatgcaacagtcttcataagtagtgacgtagccgtggaagagcttgcagaacaagaatgtgtgtggcagccaaattgaatgcttccaattatcctacaaaaagtctgaatgcatggtcatcttagtggaaataatgaaaataaaacatcagttaggctttgaaaaaaaatagcccagtacactttagttcctaaaacagagttttaaagttagagatttgcagtgacttgaatgaaattctgggctaaattagacagccacaaacctatcaggtagtagattcagaaggcactaaaaaataataggtatgataaggctggtttggatagcatggaaatgacacaaagcatcacctctaattaaaagaaaccaacaaaccctttagtgcgcgtcttgtttgtttgtctccttattacagtagggtcttggaatcatttaagtcctatccctaagggtcaaaagtaaccctgaaggagaaagcaatgagaaacaatgcagcccacatcttttcgagcattctcatttcagttttcttcaaaaatcaagggctcaatttctaccagcagtcattctagggccacccattgcctacattgtatcttattggcatgtttcacctctagctgagacagccggtttcccagtggggatgggcttgcaagatgtgttccagaataaactgttcctctgttgtctccatgcaactgccacagaactgcagaactgagttagtttacattagtatctgctaatttattattatcattccttatcggcaattaactggttaccttagagatcacctctctccccgtacaccttcactgaagttgagctcagccagagctcttgctaatcatcctgagatttttctccccaaggggctggaggcaggacattttccacagagggctctaaggctttggaattcctcttatcagaggaccacttgttttgggtccaggtggctccacctggttttttctctgattgtcagtcagacttacaaggtaggatgagctgcagacctctctctccctaaccttgccccagcttgtgctgtggcttgggcttattgtgcccagcgcagaagctaagggcttaggggattgcttgttaggaattaggggactgaaaaaggatgaagtcacctcctggcaaagctggagtctttcaattgtggcagtgctgtggctcttaggcgtctgcctcgagtttcagatgttggcccctctgcatgtgtacctcagcttccaggatttaggggaggccaagcttggttgaatcgagtgtctcagatttgggtaaataaagccgtttttacaagaggcctgtgtgtgagaggtgggaggatggctctggagcagccattttcccccacggagaagaggcttgatttcttgatgcgtgactggctggcctttgatgtgagcctcatttacttcgaacaattgagaggcttgaagtgaaaatcccagaggatcagttgtttgcagagggaaatgctcttcaagttacatttcctatttggctgttgtgtatttagagtagtttttgtgtgggctgcatagtggggtcactcagcttctagtgggtgtacccaggctgctagcacctccctgtgccccccaggggaatgatgaggaacattcctgcagctgtggggacagcaactctgccaactccgggagcttgagctcctgtgcagaaaccttacttgttatcattaggcatattggttaggaccacacagctccacagcatttctagaagaggtgtcagaatggaccagtgtttccactctacagtaatgaaaatggaaaaggcaaatgacttacccaggcacacaacatttgcggccaaacaagcctccattttgcttcacagtagttcctctggtgcagcttccaagcgttttctgtcacataatgaaaaacctgacaggactctctaggaggacctgcgggtatactttcgatggtagttgtgttcctgaaaagctggtcagccaaagggtgtttgttttgtaaattagaccccatgctaatcacagcagtggaattctgtatttaaagaaagcctgtgatggatcctttcataaaggagagtccttatataatgtgaataatcaactcctaactgatttcttttgtaagtctggattttcacatatcttgtttgcttaaaattagattcctcaggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtggtggcgggcgcctgtggtcccagctacttgggaggctgaggcaggagaatggcgtgaacccgggaggcggagcttgcagtgagccaagatcccgccactgcactccagcctgggcgacagagcgagactccgtctcaaaaaaaaaaaaaaaaaaaaaattagattcctcaaattgacaagatttacaaaacctttgaaaggtgggccaattcatttttcccttgattgaaatctggcccttgaatcctcactggttagccttcctgtttgatttttacttaattaaacaacttgcaactatgtaatatttaaaatatagtgtccagaaaagatgggtacgtgtcattgaaatttaacaagtattagcatttaatatttgcttcagattctaaaaaaatgaaataaaatatgacagctgccatcttcccatccttttctctcctttgtaaataaacccttggctcaaagaacagaagcccatcacgaatttttttttgtcttcacctgtagagctcgttcctgaagagatggcttcccctttgctgtctcagattatgaaagctccccaagtcagtaaggatttcctggattgggtagagcctgggaattactttggaaagtgaaagagctctcttagacttgcctttaaattcagtgtttggggagggagaaaaatcacttttcagaatggggaaaaaatgagaatgagagtgagacagacaaattgacagacacatgttaggtccagctacctacagaattttgagcagaccctttgggactgaagcaggcaggaagcttggtgtcaagtctgtgttctccttgagctcagtgttcatggactagcaccgcggcttctttttgttgttgttgttgagacggagtctcacttcgtcacctaggctgtagtgcagtggtatgatctcggctcactgcaacctctgcctcccgggttcaagcgattctgctgcctcagcctcctgaatagctgggattacaggcatgcaccactatgcccggctaactttttgtatttttagtagagacagggtttcaccgtgttggccagggtggtctcgaactcctgacctcaggtcatccacccgcctctgcctcccaaagtgctgggattacaggcatgagccactacgcctggctgcatcttggcttcttaatgttagatgcttaagcactgtcttgatgtgcatagtcttctgatgctccattaagtctctgagtgccagggtggccagttttcagtttctcccactgggccccatctggccgctgttctgtggcaagctgctctccctcactgccaccagcactggccttggcggtctctgccttctggattattttcgctcccaaatctgatttccagagcatgaagggcacctcttctcagattgtgtagtttgctcttctgagcaaaatggattccctgtgtaagccgacaggaagaaaagttcaatccaaaggccagatccctctttaggggagtttgtgtgtgtgtgtgtgtgtgtgtgtgtgtgtgtgtgtgtatgtatgtatgtgtatgtgtgggagagggagagggggaaaggagagagagaaagaagcaggagggaataaaatgaacattatctgcttctctcactcactttttttgcctctgtgatgatggagcctttgccggctgggctgaccgtacacatgtctatgatattaggtcctcagagcccccttcccaactctttgacattctgctggttgtctccctgaggaaatgctcataagcttagttacagcagggtgtgatcccccaaatgtgaggccatgacaccccaatttgatatagctcaaggatagatctctcagagactctttttaattgagatctcggtgagtaggaagcagccaaataatcattgctgtgcatacaactcagcttaagaataccttatagtgcatattttcaatttattagaaattattttctgaggcttgctttctctttttgtttcttttgggcacaattctgtatcaagctttcctcttctttaattctcttggcttgagggagtctgctatggactgcctatgtgtacaggtcgtttgctataaaaccatatcttttctaagtgagaagaatggacagggagaccctgtcaaactgcattcagagggattggcttaacctttaagaggcttttttataactaaaaatgtccgtaatctagcattcatatttgaaacttgacatgcttgagggaggcagggctctggagatacagggacgaacagaggcatatggctacaggtaatggtgaacggcttgttggaagactgatttcctcctctgttctgccaggcacagaggccatggtaaggacacagacacactgcactcataagactggcattgactatgagcttgctgttctgatcatttctttaattagtgcatttccattttttcaagtatagacttttgagtgatggagtatgctaatggtaacatttgttcctaaaagcttaggggagagaacacagtggcattcgtactaaataatggttttttcgtagtgaagattttgcctgattttaaaattgcaaaataattttatggtagttatggctttttggaaacgaatctcaaatactacatatgaaacaccaattttctgtgtggcttgtgcacttactaatggcatgtgtgtgcttacacttctaggtcattctattttgcctttactggtaattctttagaaatagaaaaagaagtgtgctttgttgcagtaaaaaatataataattttctgaaatggaaagaaagctaacctgcctttgcaaaaactatgcattatgtagtggcctgatggagcaaaaccttataaaactggtaagctataaataccatggcactgggcgttgttggaatgacatccggggcttggtctttgcacatctgatcaccaggtaccctcaagaggagatcatggaaggtggagaaaaatccgtacctgtaaaggtttgtggctatgcatggtgtcatatttggaaatatgggatcaaataaatgtgtgtgatgtttccaacactttgctgtcctcactctttgagcctagcaaactctatcaggacagttttctctagtgtgtgagtgagcccaagtgactgtatcatcaaagataaccatctagaaatctgcagttctgaggtaggggagatcactaccaaaacagatgaaacaattaaaaacctcataaaagagaagctttagtttattgaagcctgttattactagggaaatcagaatccaagacatagagaattctgaaaattccagacaaatgccaaactacaccaaacctgcaacaatatgtgaagagattgggttgggggatctccaagttatctttcagctctgggtgtaaatgtgtgaatgtttgcagcattggtttgaagggcttcatatagctgcctgcattgaagtttgagtccatgtgatgacaatggttgctctgtaggtggtattgaaggcagaggacttagctatggcaggtgaaacaataggtacagcacattagcttttaatgagggcagcatattctgtgtataatctgagttgtgagtgtgcatgccaaaactcagagcctctctgttcaaatatgtcttcctgtccaacgcgagttctgccataaaggcagcaatgggtattctaaccttgcttcatttggtttgtacttgttctagggatgaataggagatgcacgcgtcttgcctggcaaactagacctttcaggagagcacaaattcacttaaacataaattatgttgattatcactctacctaaaaatttatagagctggaagtcaccgagatctccttttaagttgggtattcttgaaaactgttttgtatatcatgataaattatctaaccttttatacagttgaaggccaacattatggaaataagcagctttttattccattctaaggtcagtctaaccaaattatgcaacttctaatgcagttgcttgaagcttccactttgaaagttttttcattttaataccttgcaacagagataaatattttagctactattcatgcttaacaatatatttcactgattgtcattctctttgtgggatttgttggcgtgagaggcagtccaccatgcctctgctcattttgatgtttgttataggttatcagaaaagagccagaattcaataaaaaatgtatttttgttcttgctgttatttttaatttcaggccataggaggctgactgagccctacagaatcaatatatacttggcacttggggtttgacttaagagccaaaaatgtggtagattttcttttaaactagatttgattccagaattattaaacccaatgcatgttggaggctgatggactgcatccagcaggatttggggcactttctgtaacaaagcaatttatttctttcagggagtcactgctacttcaggggaaggtgcagtcggtggggacgcccttggaagttctgttctatcctaggctccctaaacaataatttgtaggagctccttgatggtaggatcaactctatgcagtttcttttggatagtgtcaggtgtttattaaatgtattttgttactgatgactagctctatagtggttatagtgccctgctgttagagagtattcatttattctgggtcaaaccttgccctctatgatgatttgcctccagactttagtgaatggacttactgtttagtagttaggctctttaaaaaaatattttggcttgaaaatatccactcattcatccatccaccaaatatttatagatcatccttcactgaccaggcctgtattcagcccagagagacatagagaaagcataagtagtacctcaccttgtagtttatattgccactgaggagacaagatagaagttaagcacattttctttaagtcaatgagaaaagcagagatcatttactaaatggtgccatgtaattggttaacaattggaaaaaatatgatttatttcccactatatgacaaaaataaatttcacatttattaaagagttaatatgaaaactcaaataataaaatcattagaagaaaatataaataaatatccaattgattttgaagctgaagaggacttcaagagcataaaaacaatgaaaatgagcacaaagaaaaataccagcagctctgactatataaaatttaggaatgtctatatatctacaagtatcataaactaaattaaaaaataagccataagctgggaaaatattttaaatttatagcgcagagaacctatatttattaatttgtttttcagctttattgggagtgtaattgacaagtaggaattgtgtatatttaagatgtacaacttgatgttttgactcatgtatacattgtgaaatgattgccaccatcaaactaattaacatagcctttacctcatatagttaccattttttaaaatggtgagaacacttaagatctactctcctagaaaatttcaggaatacaatacagtattgttaattatagtcaccatgctagacattaaatctccagaacttatcttgtataactgaaactttgtatcctttggccaatatcttcctatttcccccacccacaacccctggcaaccaccgttctgttctccttctatgaagaacctgtgcttattaattcattaattaatgcttcaattatttatagagtgcttaccacatgttagtcactattcaaggtgctagggatacaacagtgaacaaaaatctctgtctccattgagtttacatttttatggacagatggtgatacgaagaaaaataaggaagtatgaagcatacatgttttgtcgagaatgaggattgctctaggaaggtcagggaagacatctcaaataagaggacttttaaacaatacccttatatataaagagctcttacaaaacaatgaaaagacaaacaccccaatataaaaaatgtaaaatgaacacaaatgggtaattaataaagaagccattcaaatggccaatagatatataaaaatatttcaaccatagtaatcaaataatagcaatgaactgctattaatttttaattgcaaagatgatgcctacttactactaaaaagtcaaggaacacaggaatgtgtaaaacaaaagttaaagtctccacaactatttcctcaccaatctgttacctcaggggaatcacagacaacgatttgatatgtaagatgtcattttgtacttatcaaataagcaaagattaaaaaaaaaacagtgatggcaagagtgtggttatgatacactgcaacatggagtataatttaaaaaattgatataatctttcctgggggcaatttggcaatacacattaaaagccctaagatatctaatatttctacttctggaatttatcctaaggaaataattttagatcagcgtaaaaattcatgtacagggttgttaatctcagtataatttatgaggataaaacatcggaaatatcctaaatatcatcaatatgttaatagctaaatatattatgatatatacatatgctagaatactgtgaagccagtagaatgttttgaggaatacttaattacacatgaaaatatggtataatatacattaattgaaaaaatcagaaaataacacacatagattaattccagtttaatgtatgcatatattattccaattgtttaactataatacaatatataaatatatataatatataaacaattccaattgtgtgtatatatatgtgcgtgtatgtgtgtgtgtacatatatatattcatacacacacatacacatgcacatatgcacacacataagactagaaagaaaattaccaaagcattaacagttatctcaggttgtaggaagtcaggcaatttttactttttaaatggtatacttttttaattttccaaattttctgcattgaacatgtgttatttttgacatcagaaaagcaattacttaaattaaccgtaatactatgcagtaggtattaagggccaaaaaggtggcatagacagtttttaatgagttattccgaggaggggcagaccaaaatgttccttattttcattttccaattagatcataccaattttttcttcttaattttcttctcttggaggtctctctcagaaggtaacaagtatgaagttttacattttaaaataactattttatttacagtttaccttttaaatgtgagaaatgttatgaacaatatttgtttcctttttcattctttttgtccttgtgtatactcttgtaaaagtagatttgatttgtctcaccagtgttagacatctagttcacatactttatttttagaatgagaggttttaaaatagtaaaatttaactgcaacttatacatatatttaatactatagaaatgatataggctttatctaagggccacttttggcaaaatatatttttttcagtgcccttttcagtaccaaatggccaatttggagggaagtgcttttttagtattctccaatacttgatttcatgttcttgcttctcggctctgaagcatcctgtttcaacatgatagccccaaccatgtggaacacaaaaaagaaggagtcaatgatttggtggtcattgagatgctaaaaattttaaattcaaccttgactaaatgcactatttgtgttcttttttcaacaaattagaatgggttcaatccttttctgaaaggcagatttatttgattgcttcctcatctagtaacctagttttggacagcataaatacaaaagccaacatcctttggaacacctctttaattgatggctgtataaacttctcacatcctaagcagcacagttaagggcaaaatatttaattcagtggtcctgtgtagatagacatcagaactacttagatattgtaaagaaaattctcatggaaaaaaaactcacacagatgcttcactctccaccccatagccaatgcatgagaaccctgtacaacatcactctgattgtgaatgaaatctttgctacaggaaagtgtctgtggcctaattctgtagtctgtcccttggataaatggagactatgactttcagcaggactatatatttcttatatgtgcaaccatttaactagatgggtttaaatttgaattataaggttttcatgttacagcatggagcatttgatagaaagaaatatcttgattttttcatgtgagtgggcatgaaagaagggtctaaaaattgagggacctcccaccccagcctcctttgtagttcagttccttaaagtaaaagtcttcatttaaagaagtattgagaatgtttgaaacttgattatggagcaacttaagattcatattttctcttcagatggcttaaagttgagattgtttggattagaaaagaatttccatgaaattaaaatatttaattgcagttaagccaggatttggaaaaagttaatgtgataaagcagagtgttctagctgaaaaaaatttatgtaatattatgtaaattcatatcagcatgaaagttaatataaaaccccagaaggtgataagaagatcgaaagcaaggaattttgtttattgaattcaaattctatttacacttgccaatcagtgagggttttggactggctgtgaagagagccctactaagcacacgatgctttattcgatgtgtgtgaaggcctgactctgaacttgcttatgggccccagacagaccaaaaggccttgaggtacccttagctcccaccctccacttcctatggctgctgttcgctaggtcccaagaacagaactgatgggatgatgtgactgaaatgtttttatttaccagtgccgatgagggagatagaaccatgtagggagatatagatacacaaaagctccctgcaaccaatactaatctcaatgagttggtatttttgaaaacacatatattaatgtcatgactaaataaatgcccaataaatgtttgttgaattaatgagaaaaacatccatggttttaatgttttaaaattttagttgagatcattctatttctcttttctcatctttacattatgttataagattttttctttttttttgagatggagtctcactctgtcacccaggctggagtacagtggctcgatctcggctcactgcaaactctgcctcccaggttcaaaagttcctcctgcctcagcctcccaagtatctgggagtacaggtgcctgctaccatacctggctaatttttgtatttttagtgagacggggtttcgccatgttggccaggttggtctcgaacacctgacctcaagtgatccgcctgcctcggcctcccaaagtgctgggattacaggcatgagccaccatgcccagctaacatttttttctatgtcattagagaatatttataagcaattttttagtggctgaattgtattctgttctacagatagggtttagttttactcaaacaatcgctagcattgaactttgaggttatttaaatttttgttttatataaataacaccatgatggctatccttatatataaattttgttgtgatctctgattgtttgcctgtaataagttctctacaagtgaaattgttttatctgagggtttatatatttttaaaagtggtatttcccccagataagttgtagcaatgtgtactcccactgacagtgtatgaaagcccatttcactgtactggcaaaacattattatagttttaaacgtcaccaccaccaatctggtgaagctgtgggaaaacaggcagtcccatgcattattggtgggagtataattttttttttttttttttttggagacagagtctcactctgtcgcccaggtctgagtgcagtggtgtcatctccattcactgcaacctccaccttccaggttcaagcgattctcctgcctcagcctcccaagtagctgggattacaggtgctcaccacaacgcctggctaatttttgtatttttagtagagacaggttttcaccctgttggccaggctggtcatgaactcctgacctcaggtaacctgcctatctccaccttccgaagagctgggattacaggcgtgagccaccgggcccggcctgggagtataaattagtaccagtcccatagagggcaatttgacaatatctctcaaaattacaaatgcatgtacactttgacccagcaattccacttttagggatttaatcttatagatatgcttgaccacatgaaaaatgatgtatatacaaggttcttcattgatgtattacatgaaatagcaacaatggaaacaatgtgtatgtctgtcagtggaaagttttttttaaaggagtgttttatatattgatatggaaaatctctaaatatattgttaactaaataaaaggaagattcaaaaaaaagcgtagggcacactattatttctgtgaaaaagggagggaaaaagagaatatatatcttattggtttatatatacatgaaattgtatggaagaataaataggaaattaattagaatggagacctgttgggagaatggccaataagaggataggggacaggggtgggaaggagaattattagtttcatagatggaaaatgagtatctaattttagtggaaatttgtatttctttgattactaatatatatattaaggtaaatccctggtttaatttattggaaatacttttcttcagtttgttgaatgacttttaatttgttatgcttatttttgacatagtgttttggaatcatatgaaaaaacaatttgtcagtgctttaatctgcgatttcttctgttgcttttaagctattcaactttctaccaacaaagagactatatatataatatttcttccagtatttttatgaattcacttttacattacattttaaattcattaggaatttattttgacatatagttggaagtatgatgtaaaaaacattttcctagatggttaattggttgagtttgtaccatttattgtttatttcttcttcttcaccgtaactttctggtgcttctgaaaaacatgttgagtttttatagagactaggccctttcctgggctattcatttattttccactgatctgtttgtctactataccaataccacactgatttgattattgatactttataatagattttaatagatagtaggtttagtttctccttattctttgtctttaaaaagagtttcaatttttctgtttattgtttcagatgaactttggagtcatgttaagtctcaaaaatgattatgttgggattttgattgaaaatgtaccaaactgataaatttaggaacaattgacatgtatataatatttagtctttttatctagaaatatgacatctttccatttagtcatgtttcattctaatgtcatcagtatatttacatagttctccttacattttttgttaagattacttctggctatttaatggtttcactgttattttgagtgggctctttattccattacattttctaatcaggcaaagctggcagagtacatacgagagctcttgacttttgcatctctatttcgtatggagcgctccatgttttcttagatacggtttgtcacttgagtgttcatgtaatagttattttgatttattgcatctttgcttttagaagttagagtcaggagccaagtggctttagggaaattacttcctctctgtgcctcagctttcttatctgtaaaataagagtaatgggacctacctcgtagattcttgtgaggattcaatgtgagtgttcaagcacctggcacataataagccatcagtaaatgttaagtagctattactattatgatctctttttgactaggaaaatatctaatttgtgcctcttaatctggaaggaggcaatcagattcttactgcctctagcaatgtactgtagagggcaacacaattgaattttgaaattaaagaaaactttcatttgcctctattctgttactgcaaacaaattccacctcagtgtgcttttctgcttttagttacacaaattcctttttacatcacagtattcttttccttagtggctccttcagagtttctcattgttccttactgtgacacccatctctagttcaaatgtgaagtgggaggaggtcctacgtgaatcagagttagctagatgtgctggccagagagccagaagccctcttctgatgttatcccttagtattttctttccagagtcagtggatatcaatttaggccttaatctggcatttttcaacctttctcctgcatgactctgtgctgggtatctccattagctaggaagctctggagatggcaatttcatggaccactggcttggggttgtgacctgcactttaataaatactaccccaagcctttcttatcctgtgacctgctgcttccgcagacctgggctctgtttcagggccactgccttttcttctttctttcatgcaagtgacttacaaatgaattttaaatagccagttcatggtgaggaacttttgagatcatgttcccaggactatgctctgtctcagacatgttttacttctacccaggaaaccagcttcttgtatctcttatccaagaatattttatactcttttctcaattttggggtgaagtgtagtggcctagggcagggactccaatttcactttggcaacttgtggaacaatttatgcttgtggggggttttaaggaagtgctaggtggcttggggagggggagaacaaaagcttggttatctgttataatatttaacctgaatgtgaaatgtgtgaactctgccaaacagagtctcactgtaagtgaagcattgccaagaaaataataggtaggtggttgtgtctatttctttttttttttttaaattactggatattgttcccaatagtcagcagttgagtactaaccattttctgttcatttcagtacaggctgattattttccttctctagcttatagagttgtaatccatgcacgtataattaaaatgatatccaaatgcagttttcctttgaatttgtagtggctgtgtgtgtagggaaggttgttgctttgctataaaccacagacccagagtaaacctcagctgtgctgcagctattttggacgacagctttctagttgcttgtgtgtaattgcccatattacatcatcccaaccattgtgaaatgtgcaaaatacaaggcttatctaatgattcatttcaggtggacagtttcttttaaattaattttaggagtggttctctgcactaaacagatctccctactttgttcttgcttttttgttcaggcctacggtaatttaatggttgataattgatgtagcagagagaagtgtatcatattcattgtgccagtaacctttacagtctacctaaaatgttccatgggatgtgctttctcttggcgtaagtaaagaaccaaggggagaaaaattccctccacagcatatcaagtaagcattaacctcaaatgaaattaatttccctgtcattgagtagcagagaaaaaagctatcttcaacggctacaagaggcttggtcttactggggtttcagtttgtcatattatactgtctttcattatcacagacatatactatagggaagactttcaatgtctctgttggtggattgcactaactcatctttaagtcaggtatgtgaaattcaagtaagccaagaccttccagtttttttcttatcaatgttgaacattcataggctaggtaatattacatgtttagtcacctactagtaagatgaggtgtttacaactaatatttatctttggccttggaccttctacgttgctcacagagtacagttttgtaaaatgctttataggtaatatttcaaagtggacaaaaaaagaagattgcttagaaccagaaaggtcagaaatagatcaaataatcctgggactttttattgctgaataatggactttgtaaatatcacatgtgactattagaccaaatgagactacatagaattcaatattaattggttcagtatccgctgatggcctgtttgcaggattcaatgaaattatattatcaatacaatgttttattgattccttggtttgcaaaattaaataaacatagcgtaagcacttgcagtggtaagttgtagcagggaaacgggttttgatttggaatttgaaaacaaaattaaaggtgattgaagcagaaagcaggctgtggcataacatttctgccagcacttttgaagtaactgaatatatctgggtgtttgggaggaataagaatcagagaacagagactttggaggaagtgtttcaaaatgctttcttaagggttatttcaatactagcaactcattttgttgaatagtttagtttgctttactatggatatattaatttatttcattctatgctatttttttccagtatctatgccattctttgtgttgttctagataatggctaaatagtggtcagcaaaactgacatagtcccctgccttcatggggtggggggacaaaacaccaacaatataaaattataatgctatgaaagaaaagagcatattattatgaaagagtaaaatggggaaactcaacctagtctgggggaagggaagtaatatttgagctgacccttgaagggtgagcaggagctgtgaggcaaaaaggtgggggacaggaagatggttgacggctggctgttgacattaaactcttggcctaatctcagggaaccatttcccacccctcccccaccaaggtggtctgaatttgagtgccaaagtcttgaatccaacacacagccattcgttaaaaaagtggaaagaggctgaggcaggaggtggtggttgtggtaacacacagagtctaccatgaattctgaaaaaatcaaattcatttcagaaaagcagccatgaaaagaatctgtttcaaagtaaagaaagaagcagcctgcagccagcttcagatggatggtttgtggctcaaatccattcctccagttactgctcgatggcccttttgagtccatgccacctcccacccccaaccaatgtcaatgggtggtgtgcagatgcagctcagtaagatagtgttttagggcatgcaaggctcatctagtcacatttataacatagaatcagagtactgtcaaggcaatatttatcattttatattctttcttggctactcttgataaataggaggtcaagggcctcttcctctttatattccctctggataaacagacagcagtctccagagaagaaaggcgtctttccttccctattttctgcttccaatctgtttctttttccctctttctgctccaactttttttctgtgacgttctgtctgacagtcagcagagggcgtatagacgcatcaaaagccagcctttattcaacttggccaccgtaatttcattgtctttatctcttcatctttacattcctcaaaggcagggatacatcctggcgttttctctttgtaatagtaccatcaagggcaaccattccttacctattttataaaggtgggcaaaagctactgtctagataaagggaaagaagttgtgttcctgagagctgggaggaaatgtgtgtgtgtgtgtgtgtgtgtgtgtgtgtgtgtgtgtgtgtgtatgtgacagatctcagagttgttggtgccctacgtgactcatttgggaaggactcgttttgccaatagcaaagcagacagtctgggttcctcttcttctttgctttcccggggctgttgtcctgatagcagctgccctgcttcctgacatgtcctaataatgaagatgatgtggccagggggagattcatcttcttccctaatctgacttcttcattgaattttccccttactgaagtccaaagaggattgtcccctcttaactgtgtgccctttttactctgcccaaggtcagagaatccagctgtggcctgtctgcctcccacatctctggctgcagagagtgatccatgtttgtctgtgacagctgtgtgaatggtacgtgagaggcagaggagagctgtagtttaagctgctccaaagatgtcttggtatggcagtgctgacgaaaggggagacttgtttgtgtcgatgacataatcagagaagacatccatggcccagctgcagggagtgaagaagtgtgcagcttagtatagtcttctgacaaaagcccacacacttcaggtttatctgcccgtagtgctaatctccttctgatgtcttaacctgacttttgttgtagtttgttcagacggttccagtgacagaaagaattgcttggcttgtggtggttatactggctagcacgttaatacaagtgcagggtgttttataaccaactagtctcataataactacacatgaagaacaggcaaggtttagtttagcatattctgcctattggaaagcatcagacacggttatagctcctcataaataatactgatggctcctaaaggccatttttcatgtaaatttccttactgttagtgagcttgaaaaagaaccgtattgacgaacaggttattttaaaagtagaatgattactcaataaaatacttttcagctttttcctttggggctctggggttcccccttatctcaaggatccacgtgggcagattgcatgttagctggagaacccaatagacacattcttatttccatgccagcaaaacactaataatctaaacacaaaggacatttaacacagtaactgtttctgatggagcaagtggtactgccagctcgctttctcttggatggcttctgtacggtgagagcaggattgggagttgagcccaggctactccttagtttgtgtggtataaatgaacaacttaagaatagtgtttgtatagtcagggtcatttctgccctttctcaatggacttgaaatcctgggctgaatatttatccagcttggttttgtgtttttggaacaaaggaaatttccagtgaccagtctggttaatcaccaacgcttattccctcttcaccactatttcgtattggatttccttctataccagtacattggatttccaatcacatcgaaccctatttggctgccttccattcacagaagattagattcccttacacctcactgcctctcaggatttttctctccacaacgtgtagagtattaggttcccttacatgagttgctgcttgttaagttgtgttcagtgctgcgtagtatcgaattctctttctcatcagcagtcactgggttgttaattacacacttgcagattcacttctcctgcacatgctgtgacacacagctctccaaagctctgaactcacacttcaatgccttagccatatctgcccagattcagtttacttcagctgtgtggtattgaacacctatgcatcagtctgggctagatactgcggagtttacaaaaatgagtgctgaataatccttgccctcaaagagtttacaatctactggggtggatccagggcagggcagtgcataagatgaagaattatgtgcgtgaaggttaagtattggttttactcttttcctagaaacagtttagtatctggcgtatttaaagcaccttagaaggcaacatgttcatcataatgccattattgccaccattctgttccataatgggaagaaaacattaaaaacacaattgtgtgaaaaaaaacccttttaccaggttccaatgcagttattggtagagttggtagaaaactcttaggtctcttaacaaactcttttaacagccctgccttcatacagacttaagacagtattagaaatcatataattctaagagacataaattaggcatagagaaataaacattgctatgtagactgtgctctaacatgccttgacattacttgcaaactaggatctatctctgttgtatttcttttggtaccaaaatactgtgcaattttattcacccctggaaagagagggaaccaaaagtaatacaaagtggagcaagtcatggtttcccacaagcaacagaaagtggagaaggcattaccagttgtgattttattgctcagcattctcaacttcattgcaaattcacaatctagtgtctcacaatggtgtaaatattgcctaataccttcttcatgtagcttctcattccttgaatgcttaacatgtgccaggcactgttccagatgctttactgtagaatcccagttattgctcatcataaatgaatcttctctgcatttgctgtagataaagaaaaaaatctaagttactggcaacaaagggatgtgtttttatccctttctgccaattcaaccagagcacttcattaatgccaaatttctaatttgatgtgaacagcactcctagcatagtctgaagcttgaaatgacagtgatttaaaaagaaaatagtttagaaaaagacctagctaataagaaaagctatttgctataggaattcagtaaaaacccaagctttggtttaagttttttctttaacaataaaatattgtgtcattttaaaaaaaatccctggaaggaagggaaccaaaagtaagtcaaagtggaaagaggtcacaggaccaatgcttttgctgtctacccaatcttttctaactactcatatgtattgtgtgtctaaaattcagagtatgatatctgtcttttcttttttcttttcttttctttttttttttttttttttgagacggagtttcactcttgttgcccaggctggagtgcaatggcgtgatctcggctcactgcaacctccacctcctgggttcaagcgattcacctgcctcagccgcccaagtagctgggattacaggcatgtaccaccatgcccagctaattttgtaatttagtagagacggggtttccccatgttggtcaggctggtctcagactcctgacctcaggtgatccacccaccttagcctcccaaagggctgggattacaggcatgagtcactgcacctggccgatatctgtattttctaacctaattgtgtagcataaagagactttattagtatggaaaagtgacagcaacatttagaactcatttttcaaaggtgtgttactattcaaagcctttggaccatagcaccatttcattagaataatagcatgttgatcctcaattgaaatgccctactttcttgtccatggttatcaccagtagtcagggaggttttcttttccgaggcttgtctttaatcatatagggcttgggactgtactagttcaaccactgtggaagacattgtggcgattcctcaaggatctagaactagaaatatcatttgacctagccatcccattactgggtatatacccaaaggattataaatcatgctgctataaagacacatgtacaggtatgtttattgcggcacgattcacaatagcaaagacttggaaccaacccaaatgtccatcaatgatagactggataaagaaaatgtggcacatatacacaatggaatactatgcagccataaaaaggatgagttaatgtcctttgtagggacatggatgaagctggaaaccatcattctcagcaaactatcgcaaggacacaaaaccaaacaccacatgttctcactcaggtgggaattgaacaatgagaacacttggacacaggaaggggaacatcacacacctgggcctgtcttggggtggagggagggggtatatctaatgtaaatgacgagttaataggtgcagcacaccaacatggcacgtgtatacatatgtaacctgcacgttgtgcacatgtaccctagaacttaaagtataataaaaaaataagaaaaaaatcatatagggcttaatatgaacatctagacacagactttattcttccctaatgctgtccccaaacctttctggagctcctgctatgtgtaatgtagcatgttcctaccttcacaaacattcctgccagggcatgtacttccatagctcaaatatttcatttttttttctttttttttgagatggaatcttgctctgtcgcctgggctggagtgcaaaggcatgatctcggctcactgcaagctccgcctcccaggtgtatgccattctcctgcctcagcctcccgggtagctgggactacaggcgcccaccaccatgcccggctatttttttgtatttttagtagagacggggtttcactgtgttagccaggatggtctcgatctcctgaccttgtgatccgccagcctcggcctcccaaattgctgggattacaggcgtgagccactgcgcttggccaaatatttcatatttcatcttcttttgaatccaagacttgtaagcataatttatggatgatggcttcagagaaacaatgagttacaatgtatcttatgattccttggacatgccttatttaatggtaaagtttccattttccccctgttgctcatttttagctataaagaccataatgccagtcactgcctgaaatctagccaccccctttcacattaatattaatagcataatggcaaacattctgtacattgcctgtgactcacataatcaccatgcaaaatgcatcattcataaattgtttagtgcgacctttggcacagtagcactaattattattatcacagaagcccagtaaactagactggtaattatcagggatttatttgaatggacagctaagctattcttggcaagttagtcaaagcagttgtcatgacagccatacaaatatgtcttatgaattgggttaattagggtaattaataactgttatttgttccccttttcccaagatattgcctcagcattatcaaaagggtgaggggtgtaaactttaaaataatgcttgaatccttctgctaagaaagctactgacgaaatcaatgaaagctacctaccttccaaaggtgctcccttagagaaaccctcatttttatgggaccttctcctacttttggatgtttcagagtcagcaagcacaagccatactgaaaaaaagtagttaaaaagagaagctaaatagtcagttccccgaggctgctgctttccttttagtcacctgtgagaactcccaagagaccatccctctggcttctacttttagcagagccctgctttgttttttgctcatctagaactttcttgccttgctctgcagcctgttcattgtataatttttttttcttatttttccctcacaaggaaattctgagtttttctcacccacatttctgcccctctgaggttttccaagttttaattttgttggaggccttttttaatttaatgccttagggaccatctaacattgcctgatggtggttttgctcttttttcgaagaaaggaaaataggctagctctgaagagctttctctttctcttccttaaaaaagtaaaggataaaagtccaagtttcttctctctgcatcacagcatattaagttgtttttcttattctagtgcaaagaagagagaaagggagaggttaaaaactgaagctgattcttttccccaatgagatgattctttctttcagagtaaatgttgttcacaaaaggactttaagactttttgtgatggttttgaacagcaaatcaatttctctccatttcctgcataaaactaggagaaggaggctgggggaggtggctcacgcctataatcccgagggaggctgaggggggcagatcacaaggtcaagagatcgagaccatcctggccaacatggtgaaaccccgtctctactaaaaatacaaaaattagctgggcgtggtggcacgcgcctgtagtcccagttacttgggaggctgaggcaggagaattgcttgaaccccggaggcggaggttgcagtgagctgaggttgtgccactgcactccagcctggcgacagagcaagactccatcaaaacaaaaacaaaaacagaagagaagggacacaaggcagaaagagaactgaggatgttaattaaacatctaatctatgtcagtttctgggctcagtacttttcatatattaactaatcctcccaacaactctttgagactggaaacattattatcctagctttacaggtaaataaactaaattatatgaaagttaagtatttttctgagatcacacataaacaggacttgaacctcaagtctctgatccagagtctcctttcttctacaccagctagaacagagtttccccaaaaaagcaagaggcttttagtacttcatgaattccagatgccttgctcacgccacaaatttctattgaatataaatgcccatttattttgtttgtttcaatgcctgttcagcaattgagatttctaggtcagtgccagcattctggctgacccataaaattcaagtttgggaggaattctggtaaaatgcttttagcagagggtgttagattaataggttagaaaaagtgccaactttcttcccatgtcatactggaatctcatgatcagaaggtagcttcctctattttactttcagttgccctgttagggaagaaaggctttctattgcagaagtggcttggtggtgcttggaagctgcacaggatatatggaagatggagtggtttagggttctccaggtgtccctgtcctactaagtcagagatcaggatcaggttctgacatcttaggaagctgaaaactgacacacagagacccatttcttttttctttttttctgaggtggagtcttgctctgtcacccaggttggagtgcagtggtgtgatcttggctcactgagcctccacctcctgggttcaagggattctcccatctcagcctctcaagcagctgggattacaggcgtgcgccaccactcctggctaatttttgtgtttttagtagagatgaggtttcaccatactggccgggctggtcttgaactcctgacctcaagtgatccatccgccttggcatcccagtgtgctgggattacaggtgtgagccactgtgcctggcctccatttcttgatatgttattattaaatgggccatggtatcatatatttagtatttttaagttttgtgtgggtgtgtgtgtgtgtgtgtgtgtgtgtgtgtgtgtatagtcatttgtctcgtccatgaaggaccttcattcacttgataaacactttgtgccaggagctgttttcagggttccaggaacagcagtaaactaaacagacaaaaaattcctaacatcatggagttaacattctagcaggggaagagagataataaacaaaataaatatgtgaaatgtatggtattttagaagataagtgatatggagaaaaataaggcagaaaaggaagataatgagttttggagaaaatttgcaaatttaaatagggtggttaggtaaggcttcaatgaggcctaattttatttcttaccacttcacacacaaactttcttctccagcaaagcagatggttcattcattcattaatgcctactctacctacctttatatcctaccaggtatagaatacacatttaaggacaaaacacacttgcccctacccttttggagcccaaagtacaggagatagacgttgaaaaaaataattacacagataattacttgcaattgtgataagtgtgatggaagtagtagaaagtactaacacatactacatatggaggccaatacacagctactaattgtattaagctaaaagccacagatggaccaccaaacatttggtatccctggattctagtaggaaggaccacgatgacatgattacaaagccacaagtctcatttggtttaacctgtgtctagggatctgtgaatttgtgatttcaaatgtttgattgccataattttattagaaaccttcagcctccaacctaggcaattactggcatttgagtttaaacacatgcatacacacacacccacacacacacacacacacacacacacacacatgcacacacactgttgtattctttggaaaacaaatgaccttttgatttggtgtattgtggaactgctagaaaactggcttagaaggagggaaaatgttaatgaaggtaatggctgatttttagagcagagtacctatttgttttttcttgtcctttcattgtgcctccaaagcttcaaaacctcctaaaaatgaaagattataggaaaaaatgcatcttttactttttaaatggtagacagcaaagtattttaatgaggaaatatattatattattctcctacaaaccaagggctaatagtctggaactggtactttggctgcataaaaagtccagcaagacaagccaattatggactctgaaacatttttatttttagttccttgtaatagcgattcattctctgctatggtacattaactgggaaacaagttgaaatgagagttttggcaactttagaataattgactatatttaccaaattcttaatcgtgcagagaggttagcaggggaggggcatagaagaacaaaagattaggtcagggaaaaatacgtctaggagagtctgactttcaataaaggctaaatagacagctttagaatacagttttagagaactcttttttcctttcagtttattcttcaagtagacaacagtttggttatttgtgctgatttctgcctttatgtatgatgtggtgttacttttgtgagcagaagtaattagttgtttacaaatgaagaatgcagtaggcacagctgtgatattcttttccctaccacgagatctggtagtccccaaatttccctctacatctgtgtgcattggtaacactgttaactgctctgttccctttcttttccagtagaatttcttttgtttgggatgactgttacttttccttttggtcctcttttccttcagtggctctgcttgcctatctggtggtcgttttggaagttccaacccagcaccaatccagagtggagctggtgatgccgaaatggaaggagtgtttgggaagggtagatatgttatgagggaatcacgttgaagttaaatgaaccgctaaaacaaatgtaatcttaggcctcattaaggaggtatagttttctgattataagaggtattagtccctctgaattctttgctgttaagactacatctggataatgatgtctgattctggatgccttattttatgtgaaacactgacacactagaatgagcttcaagggaggttaagagactgataaacggcctggagaccacatcacttgtggaacaatgttcatactgcttaattatgagagaggcaaaaagttgtatgaagccaaccttaacattgatctgcagtaggcaaatgtgttgaaatgaagagactaaaactaaactttcacaaaagcatgacacattcttcttagtgtttggcttgttaattagttacgttgagtacaaatatttatagtatttgagtctaggaccagctaactgaccagcacaacagctttcacatactcagcaagggcaattcgttcttaggtataaggccaaagactgacccttgggtgatcatggagatttcccttccctttgtagagtgctctgtatttaatcttctaatcattaaaaaacagatgctcaccactgacagaggtggggttcctagaaaagatggactgatggctgttatgctgtcttccatcttcttgagacccttagcagctttgtagtggtatgctttagtggggttaggaaggagttaatatcttggaaaaaccttggatgtggcttgatttcctcttaggaatattgagaagatgaacacatagtgggaaatttttaaatttactggtgctacaggcttttttccaaaaggcatttgtagtagtaataataatagtaataataatatgcccaaatgacagaagaaatattaacttctgaaaataaaatttcaaaaggcaaattttataatatggttgaccaccttattctttttagatcacctagggagaggtcattcatacctttatttattaattctctatcttctcatgccttattgatttacttttccaaagcacagcagaggaaactttggggggaagagataaaagtaatccctgtttgcacagcaacacaatgtgtacattgttggcttaaggcctgtggtggacagaaagactctggccttagagtccttcaggccctcttatccaggaagagaagacacagaaacatcgttctcagagtagagacagtgtttctgagcgacccaagatttctcgtttcttcttgtgaccttcttcctcttttgggcactgaatggggtcaggcctctagggatggcacacagttagttggtgagagacaggaagggactcagggtccagcagctcctggaagggagaaaggtgacccagtaacttttcagcttcttttggagaaaaggcacagtgtcaagcaatcaggagaggaagaaaacaagcataaaatccacatccttaaagtccataaatggtgaaaggaaagctctctctgctccccaccaagtgtggaagttttatggtgtattctctattcatagggctgacagagagtgggactgacctgctcaggaaggtgaaggtctccacctacaggctgctgcaggaaagagctcagtctgcctttcctatccttgacaaggggagtagagccagttttcagaggctatccaagctagggttgacttgtctcaggactcatctttatgcaaagtctgcaaaaagaatttgaaattcatgaacatctgaccactggagaaaacctttactggctcaaaaaataagtctctgtggtttaattagctaccccttcggatattgatttttctagcacttgtacagaatgtaactcatcttccaaactttacggaagagccaacccaaatccagctggatttgtggctgtagagaagttgcttgattttattaagcatggcttacgttctgtctttctctctcccctgacatttatatgtaacttgcaaaatccaaaatcacagctagagagaagcaaactcttcccccaaaccaaatattttccctaggtctatcccccttagatcatatagtctgagggcacttctgatggcatgtgttacccatggaggctttcttctctgttgctctctcctccgtgctcccatggcacactgtatataccacttgctatggcctaaatgttggtgttccccacaaatttatatgttggggacctaagacccaatgtgatagtattaaaaggtggagcctttaagaggtgattaagtcatgagggttccacccttatgaatgggattattggccctgcaaaatggctccagggaaccaaccagcacctttttgtccttccatcttctgtcttgtgaggacacagtgttcctcccgtccagagaaagatacagcaacaaggtaacatcttggaagcagagagcagctctcatcagacaccaaatctattgataccttgatctgggacttcccagcctccagaactgtgataaataaatttcctttctttataaatttcctagtctcaggtattttgttatagtagcacaaatagactgagataccactatggtagcacttacaaccctgcattataattgtctctccatttaactgtccccagcactagaaatgtgaactccttgagggcaggagtggaatcgggttaatctccagatccctagcgtctcccatggggcctgacacatagtcttaacagtatttgttgaattgaattaaattatttcagccctgagctgtacaagctgtcatttgttctgcaacagtcagtcgaggacagctagggagcatgaggcaaatcttttcaattgtggggaagcctctgaatgatctgtttttgcctttttcttctacccttctcatcgattccatctatcccttttcattactggttgcctgcaccttgtgcaaacagaagtcttaaacagtgcaggatcttcagtgacttttttgtcttttgatccggggctgatggcttttcattttactcatgctttatatggcatagtcaagtctgattgagacttatttggtggtagggcagtcagaattactggtagaaaagaaatgcaattttattactttcaagttcatagaatgagaagaagtagactaataaagtctgtgccaggaagttttgccaagtagaagtccttggacttgttttaatgaataaataagaatgatgtgcaattaacatacttattgtttgtaaattgcttctaaagtgcctaaaaacaatttattcttgttagtttaaacattttccttgtaaccttgtttataccacaatttgcccagcaaacctttataaaaatagaaacagcagcctagctctatccgaatgctcaccaaatatgaattactgaaatctgaactgcaaaggtatgactccaacacgggaaagcaagtaagcctgagatggatgcttttaggaagtgagcagaacagattttcccttttctacttaggtgttagttgcaaaggcagaaggttatagttagttgctcagtactatttagagaggaaaaatgctcccttcaccagggagcaatacttgtcatcattccagctccccttggtggtctaatgccagagagtttctttccactgaggcagtaaaaatgagaaaatttaccttttagataatttcactacgatcaaacaatccccagtgggcaaaactaatagaagatactgtattttgattgcagtttatctctagcaattataaatgagcaatttaagtggcaacaaagcaagttgtttgctagaatgaggccgtatttcacagagctttatcattcaatattctctggatcactcagctaaaggcagtctatttccttgggtggatcatacatcctctttctacagtatccctaggaacaattacttttcttaatgatagggtattgagaagaagtgatctacaaacataatttacactaattttcacttttttcctcccatgagaaatttagttgacaagactttgagtattaggttttattcttccaggatttcttaagagaactaacaagaaggagctgggcaagtagagagaatgtcagaagagctcagttctgaatccatgcctgagtttccaataaccagctgcgttgctttgggggtggttgtaagtttctctgggcccatttccttatcttcaaaatattgtgtgtgtgcacgcacacgtttgtgtgtgcgcgtgtgcaagagagataatagattagatgaactctaaggtcatattgaattttaatgctttacgagtatatattctgtgattgttcatgagcccaaaggggaatttttcagtttttcaaagctttcagtggacagcatagcattgtcaggttgcagtaatgatttcgagaacctttgcaaatgactacatttggcaataaagctactgtaggctctaccatggcctgaagaaataattaatattgcataaaaatatatattttttaatttatgaaggatagatctcaaaagggactatagattgaaagcagaaacagtaagtgagccaattgggcttccattttctactgtttataaatttagactgggtggaaggtcccctggtttggaaagaaggagtcttaggttcttgctctgactctgccagtaattggctatgatcatggattcagaaacttttgggcctcagtttttccatctgttaaaaggggaaaggagtattttgcctatatacatctgcagactgttgtggttattagaagggagcataggtcaaggctttgtaaaaaaaaacaattgctatattaatgcaaggtgttatttgagaaaataatttttaaaattgggacattgagatacacagttctacaggtgtttctcattgtgctagattgtgaaacattgtctttgaaggtgtggattatatcatgttcaatattatgtatattacacattggcatttcaatggaatgggaatagagggatggggaaacaacattagtttggttttgacttaaagccagttttttctactaagttcttgggaaatgttgaaagaaatacaagtctaggaagcacatcagcctttcttaaaagagctttgcaaactttgcatttctgattcaatctatgaaagagacaggtgagaggttcctatcagctgttaatcagttaaccaacagtgggtgagcatttagccagtgccagggacagtgacaggcactgaagataccaatactaaaggtgtggttcttgcccttaaggagttctccaacctagtgggggtgagatataactaagcaacacaatgtgttaggtgatgtgatagaggtaagtatattccttatatttccttaggtaacatttcagaaccggcgaatcctgggtgacaagcctccggtttctctctaaggtactgctgtccagtagaactttctgtgatgatggaaatgttctatgtctgctctgtccaggacatggtagccacatgtggttactgagcacttggaatgtggctactatgattgaaaaactaaaattttaatttaatttaaattttgtgaatttaaatttaactagctacatgtggctaatgtctatgctattggacagcacccccctgaggaatggtagtgtgagcccacagtcaggctccaaggaggtcctgagttggatcatagaaagctctggtcaatttgcccctgtcctttaaccctgttttcctatccaaatactgcactagcccttgaatggctcctttccagccgacaactctgaattatgcatgctaatgtgtgttactcaaccatggcatgaggttccctcactcccctcccccaaccctgccgtaaagaaagggtactctgtgacagttcaatcatgatgaaggaagaaaatgggtcgaagaaaactagtttataaaagcttttaaaatgtctgaaatcttgcttttcacataatttcagatggtgtgaggtaatttcaactctctttaagctgagttttagctctctttctctctccctctccctctctctcacacacacacacacacacacacacacacacgcacgcacattctacctaaagtgtctgctgtggtagatggggctccttgtattcctattgaatcagcaattagagaagttgggtatataatatatttattttatatatatattatttatatatataaaataaatatattatatataaaataaatatattatatataaaatatgttttatatatatttatcatgtacagaatatatattatatgcccatatatatatatatatatatatataaaacttcccatgccaaaaacacaactcttcatcacattattctttttctgatagcgtagataaagggagactgagataaaatatcctagtaggaaacctcagaaagaaaactattcacaatattcaatgggagtttagttggacatagaaataaatattcagaagaaatcaagtttacgacctggtaagaagacttgccatttgtctggtgattcacagttttggaaagtgcttgcacatttggtcctaaaaaaaaaaatcccctagaaggtatttgggacagatattatttccattttttttttttttttgagatggagtttcgctcttgttgtccaggctggagtgcagtggcgtgatcttggcttactgcaacctctgcctcccgggttcaagcgattctcctgcctcagcctcccgcgtagctgggattacaggcatgcgccactacgcctggctaattttttgtatttttagtagagacagggttttatgttgggcaggctggtctcaaactcctgacgtcaggtgatccgtccaccttggcctcccaaagtgctaggattacaggcgtgagccaccgcactcggctgatattatttccattttatagattaggaaatcaagagtcagagaggctccatgatgtctgcaagggcacacagctagtaagaactttttctattttcatcttgaaggctctgtgattggaggctttgtaggatttttcttttacatttttttcttatgtgcttttttttttgaagcttataaccatctgcaagaaacaatcaccagagtaactttttttttttttttgaatcggagtctcgctctgtctcccaggctggagtgcagtggcgcgatcttggctcactgcaagccctgcctcctgggttcacgccattctcctgcctcagcctcccgagtagctgggaccacagcacccaccaccacgcccggctaatttttttttttgtatttttagtagagacggggtttcaccgtattagccaggatggtctcaatcagctgaccttgtgatctgctcacctcggcctcccaaagtgctgggattacaggcgtgagccactgcaccgggcccagagtaacttttgaccctaaatgaagctgggtctaccaggaaccaacatttaaaaatacctatacacaaaggtacatcattgtttccttcacaaaaattatatataatatacgtatatatagacacacacacacagttaagaaatgactgcccacacctgtttctactggtttagccccgataaaagtgtttctttgctatcaaatagacacaggtattgagccgaccaggatgaggaaagggtgtaccagcttccttcgagagaaaggtgtgtacataagagtgaaagaagaaaggggtctctaaacatatttgaaaaattgaccaagagagagctgtggagggaggaagctgagaacaatgtggggaaaggaaaggacacatcaaccacactggttgaggagtatgctgtctgacacagtacagcgttatgtttctgacagtgttaacttttgttttttgaaagttaagtttaaaattggtcctgatttttgctgctgggagtctgatggtagaggtagagtgaggtgttcccctcattatttcatttcttaatcttcctgcaggaggaaattgctctggttgtgttcactgcctttgaacctgtcgtgtttaagcaggctagtgtattttttttctcccaaatagtatacctcgtcagccagtcctatttactttgatttcactgcattttccagaatcctaatgaggtgtggcctagtggtgtgagcgcaggacctggcaggaagactctcaaattctagttgcagatgctagatgcttctatgtagtagagggcaagtctcttgacctcttctgtaatgccgttaacagggttaataatatttatttagctcataaaggcagcagggggattaaatagttaatgttgtttaaaaggctttgagaaaaagaaaaggcaagtttttttgtattgtgtgcattttcaaatgctaattcttattaacaggaagcatctgctccattacggtcattcagcacctcatgaaagtcttgctggctggatgggaaggaaaattcttcaattttgtgccatgagatctgagaaaatgtagctaccttcagccccagttatgaacttggatgtctgttttctccctatctaaaaacattcttcttctttatgtggtttttctaaggtttagtagtttttctctcttttgcaatcttttatgcttaccattactggtgcaaaatgactattgtgtttcagctgtggggggaatgaaccctttttataataggcagttagacattctggaagtctttgtgataaaagatttcatagaaatacaaggtgcttcagtaattgttctgttggtgccttgcataactgcattacagatttctaatactctggcagtttcaagatgctgatgatattagccactttgtgaagggtaattatggctttctatgcatggttgccagcactctcatgtcagattatagtagccttttggttaatctctattttaaatcagatcaaattctaacccaccctcatccctcctcatcccccttcccaaataaaatgagaagcactgtgaggatttgggagaaggacaggtggggtattttagagaatgagaaaacatttctattattattttttgagtcatcttcttattgcttgtcagttatggtttatgaagtgatgccaaagtataaggtctatcttgcctccaacgctgatttgctgcaatgcttgtttaagttgctagaaactggaggctcctgtgggtttccttctgtgcctaataatttcaacagagccaaatgccctttatttactacagtcataattagagggctgccagtgttaagcaaccatggaatctgagtttgagaaatgtttattgtgcctcattaaatctgaaatgagtacttaaaggtcagaaataaataatctgtttatattttttgtgacagtaaatataagtgaatgctgtgtgtacatatccttgactctactcatttcagtgtactgagacacccctctgtttgccaaggcagtaatcacaatatctgcgacaaaagagaactatggcaggtttcattgatcacaaacaagaaaatgatctattgtattgtatctaattagaaaacatcaatattagaagctaatgcattcactgtttccaagatggtctatcacttgattgttttttgtcagcatgatcacattttacgggcttgagcaacaagctggtacaagggaaatgaatttagaatgatatgccaaaggctgttgctccttcagtcttaaataagcttgtagaggcaactgggtttttagtggcacataatataaataatgggattggggttggaacagaatttgggggtgttgttttggcccaaaggcacaaggccacccaccgctttctctctaaatattttgaaagtcttttcagctgacaaagcttactcaatagaaattgtactctgcacactccgcaggcagtcagggctctgtagctggtaagtggcagagaactgaggcactgatataatagaatttagcagaaaggaatggaccagctcaggttctgagggtgctagtccttctgtgagccagaagggctcaaaggtaggctggatccaggagagctcatactgtgggtttctggatcccattgcttgtgggagattacacagacacacacacacacacatacacacacacacacacactcttatatgtgtgtacatatatgtatatatattatgtatatgtatatatgtgtatatttatttctttattagagatgggctctcgctatgttgcccaggctggtcttgaactcctggccttaagcgatcctcccacctcagtcttccaagtcattggggttacaggcatgagccacaatgcccaaccctcttgtgggagatcttatatgggtttcttattgtcagtttgaccagctttaccaaatcatagcttctaaaattgaaaccaccatcatgactattaatcttacatcttaggtcaccttctaacaatggttgccattgcctctaaagggcaaccaaaatggtcaccaaggtttcagggaacaaagtggctcatttctggatcccttgggctctgtgagtaggccctccagctaggtctggagaatgatttccagttagtgtgtgccaagtttttcctttgtctaactgctggactatagtcccctgactctttcattggaattagccaaggaaatcactcagagaaacataggtaggaagcaggctaatcacttccctgatgttactgttttctgattgcttcctatcccagtagcagaaaatgccaaaaacggtgctcctggggtttctcttattgtccttgttgcggcatgctctagctgagttagtacttaatgtggctgtagcacttaacgtgtgtgtttgttttcatggctctttaaaggggaaataacagttggcacttgcttgtaaagtgctttgatatttggggatgaaaggcactctgtgcctgcaaaatagctttaatgtgttgtagttacatgtttggtagggcgattggactccagaatttgttccactggcaaatgtggttgagagctatttggttctccattaagttctgtttacatgggattaaatagtttgacttacatgactgaatttttcattttatttgctcttgaggtactcttactcatactgagatattgttgattctgcatttgtacatataataggcattaactggttccagagagctttctataagcagatactatttcagagtgcactatgccccacaaaggacttagaaaagggtcgagggccttattcagaagcctgtatttgttttatggcatggtgtttaagctcgtctttcagcaggctttgaattgtatggtttactgcagacagctgctaggtttctcaaggaggcctctgtagtgaaattcacaactgggttgctcttctaaaggcataactttgacccaaagatgagagcaaagaaaagtgctttccctaagccatctttgccacctctaggtagggtgaccacatcaatggtgccagtgaaacaataggaatgacgatgagaggctgctaggaggggaattctttcaatttaatttgttgaatgttaaccacatggcctgtgtttctcattgatagtcctgtcctgacagtcactgggatgctagagttgtttaccacctgtagaaaagtagctgccacactgtccgtctgtgactcagaggtctacattctggtgaaccctcgtcttctcacagaaacgctatcaaattgggttcaggaacactatcatatgttgtcaccaccattcccagagatgtggcaggactgatggagttagatgggcagaaggcacctaactgtgtcatcttatccctttcagttatatataggctattgtgtatttataaggtagtagttgggagtgataacagtgaacaaattgtaactacagaaggcagtgtgcagagtggatgctttctgaagaagtagaaccaccttggcctaataaacttcgtcacctgccttaattgtgataagtagcctcattcttaatcccttccatctcttctctgatcattcattgatgcattatatgaacgcatgctaaatgtttagcgttgtggcagatggcacagggacctcaaaagagagcttccagtcccgttgagggatgaactgacacactacacatgaaataatcatagaacctgaggaaatgcataattacattgtacagtctgtatacaaagtgctgagaaaatgctaaggaacgtgggatgatcagtgatggagatatattagaaatgagggaaattagatggcttgtgttggatgactgcttagctaaggacttcatgtagatttaatgttaattgcaacaaggagctactgtaaggtcttgaaaagagaagtgatgtgatgaaaatagtgggtgaggaagattttccaaacagcattttgcagaatgaggtagaacaaatgcaggtgagaaaaaaatgacacaaggtgatgaaggctgtggaaatggagccaaaaggacagataatatgggctatttctaagaacgttatgaattgtataacctatttgataggatagggagatttcttgccttttatgatgggggttatgtacatatgacacatatgagggccaaattctcccatgtgagtcacctacagccattgagccattagggtgttatttagttctacaaagtagcagtgtaattctccaagttgcagtccagtagcctgcgtacaagtatatgcctgtatctgcaggtacataggtgccagtctgtgtatgtgtatttacctgaatgtctgacattttgatcatctctggtaaaagatccattagtttgtgagctccttgaaggtgcaggctatttttttttaattcccagggtttggttcatgcttactggatagataaatgaataaattaagaatcactttgttttagaagcctagaattgtggagcttgaaggcaacatggagtccttttggttattctcctcattttaaagaaagataaattaaggcctcaagagggttaatgtgattcatccaaggtcatctgtaagttaatgacaatcctagggagagaatccagctgtccccactataccacattgctttctgtagtcaaactgtgtgtgtttgtgtgtgtgcgtgtgagtgtgtgtgtgtgagagagagagagagaatgaattagggctggggtgtgtggggagcatacaactgacaataagtggttagtaggagtttacctagcacgttttctagttcagggcaaatatgaactttgtaatgttggcagctggggatggtgccttttattccaccttcactaattcctgcttcagttgctgctgtctggatggatgagtctttgaatatacattctagctggaggcacgtcagctttatttttatacagaatttgcatattcatttttatgtgttgggggtaggaagtaggcaaaaactatttattatctcatttccaaaagccatctttgtgaggagatgaccatttgaacatctaaaacagcaaccagctttaggaaaggttatgtgtcttttcattatggatcaataggcacaattatcattattggaagtcagcagttaaaaaaagaagcagcgttttataatcactttgttgtgtatctgatacgggattccagttttcctgttttgattgctaaaatgacttaagagttaaaaactttaaaaaattaggtagcaaattatagttataatgcttttcagctcgatgggactttaaaagtgtaacttaaaagttcgttcattttcaaatgaaaatctaaagatttgagtaacctcagttttaatcccaatgtctgcaaccttttttttttttgcttggtatagtgacctgggatttgtgccactggacaaagagtctagtcctggatatctgcatttattgggcttccttctccttaggtttgaagtctctctgctgataaatgtggtgctgagagagctgcagtcactcttctagagactagggcacgattcagtgaactgtaaaagtaatgcaaaatgacaattaaagtagggcattctctggtaccttgggggcctgacaaagtggaagtatttgacttctgatatgccagacttactaacgtagaaatgtaataacatatcccccacccaaacgtcttactagaatcctcctatatccagaagggacctgagaacagcaagtagtaagcaaagaaagctcccaagcagctaacaaacaaacacagaaacaaacaacgaaaatcaaaacattgcacaaaggtccctatattaaagatcatgtgatttcccccttaggagcgcaacatctcagctgtctatgcagaacgttacaactcttttttttctagacacaaatataattgtatagtttccaagaacacagtagagtaaaagcatcaaacttgaaatggaagaaaagctacttttgatataccaacagcactgaaaagttaaaaacgctgtgtttgatagaaggttggtgacgatcctatgttaaaaattaaagacaagaaaaaaaccacagcagtttggggtccttgagtatacaggcaaaatctgcatggggtaataatttacatttataaagatgctcctaagtaaccaataagtggttaaattatgttttttataccctattttaaggagggcaactttgtgatatgtttcagaaacaaggcagatggttaggattttaaatcaaagttttggttcttaaaggataacaccctaagctatctggaaaactgagctgtctagaattatttattgtttgagaatttttaaaaatcaacattttattaaacctaggtcatgtgagacacctgctttcagtgttatacccaaggataaagaattcgaattagaaaaagaatttgtgataaatatactaaataataagaaagtgactcttatttaccactgagatgaaataaaaaagtttgctctagacccatttttttttaatgtgacaattaggtttttccaaagcaaatgttgttcttatctctttgctgtgttcttgtgttaacctggctgtctgattagaaatggggacatttcctagtgaaattaatgtgacatttagagaaaatttcctctctcaaatgtttcatgttgtttgccatttagcatacttagagaaggtattggaataatgtcaagcaaattttgattaaataaggctaactaggagccaatacattcatgcaaaaaaaaaagctcttgtattattttaaagaatgtatttcttctgtggttctttatcccatattgagatgaccttggcaggtaatctgacaaaagaaatgacatcttctaaacagctgggcttgctttagaaaatgcagcacattaggtcagagcctgtggccagtcctttgctcaagacagttacttgccaccccggaggtacactaaaatttcctggaaagctttttaaaaaaatcacccagtgcctgggtccaattccaggctaattacctcagaatctcttggagtgggtccttggtgacagtatttttatttttatgttttacaagaagaagattctgagatgattctcatgtgtagttagggctgagaaccactggcttaagacatacaaactattcattaagaaaatatttccttcttcaggatgatctgatgatctgagtctcatcctttgctccaccagaagtgtgaaaaaatatactctctcagtgcacaggagttgggtttgggcatacaccttgaaccagtatgagctagaacacttgatgtcagtatacatagacccataatataatagcccatctatggccagttaactcagttgtttggaatgtagtactaatgaggccagaatagtgtattctatccaattagaataattctgctgcatagatttttcccataactgtaacccaactgcctcacaaagagacctgagacggattacaaaggcacaggacaatgggtgaatcagcatagccccatccattctgggaaaatcggttctaggaacctgccacattgactagcgctactaattgcattaggatgcagcatgagacatctgcccatgatggtttgtgctgatttgggggaatgatatattttcatttgaaggtcaggacccacaagaatgggtggtttattccttttggagttaaaaaaggaggataaaagataggaaggttacaagcttctatgatattcttctctttacagtagcagatcatgcctggcacatctgcctggggttaactgacctgtatttgggggaagaattgttaaggtaattactaacaagccctcgagacttgtctcttctcaactcagtgcctgggagtgggaatcctcatgatgagatatattatggcttaggaccaagaagtagtattttcattagtgatacaatgtgcatggtattgcttctttgcctgaacacgtggctctggcaccatgcctggaggtcaggtctccgcggagcttcatatttgatgcagagtgcacacaggttccatgccttcatttgcaaagatatggaaaattttctacttgagaactgtcttctttaaaggcagtaaagacagattctttaaatacagtagcttgggttgctgcttatccttttcctgcaaagccagtatcctctagatttcctttttgtatttttgtctctttaccaatcttcttgaaaaaatgatttagtttgaaagatgagatggaacatgcatgtgccactattttattgcctgaatgaggtcaaattcctgatcctacttggagttagttgcaggttctgtataaagcaggggatcaggattcccaccattcctgtcatgggtctgataaatgcagctgaaattctcctttggattcatgttctattatccctattttacagagagggaaactgaggcatgcaaggattaagtaatttgcccaagatcgcagagtgaaatgaatatttgaactgctgagtgcttaatcaattccgggttttaacagatgtattagtccattcttacattgccctaaagaaatacctaagactgggtaatttcataaagaaaagtggtttaattggcccgtggtcctgcagactgcacaggaggcatgatgctgacatctgcttagcttctggggaggcctaaggaaacttacaatcatggtggaaggttaagggggagtaggcacatcacatggccggagcagcaagagagagagagatggggaggaaggactacacacttttaaataaccagatgtgataagaactcattcactatcgagagaacagtaccaagagggatggtgctaaaccattcatgagaaaccgccaccatgatcaaatcacttcccaccaagccctacctccaacattggggattataattcaacatgagatttgggtggggacacaggtccaaaccacatcaacagggtagcataatttttgtggggtagtgaacttcccacttgttaaattatatattgtactgaatatcgtagatgaacacatttaaccaacatcatttttacctgttatgttggtacaacgaattaactcaaagagtgtttgttggttgactgactgttggataaacttcttccagcaaatacttggattgtattcctaacacagcttccaagtttgcctcctattcctgtttgtgacaatttcagcattcactctctatgaagaaaccagaaatgaactgacccttttgatttttaatcccccaacccatgcttccaatagcattaaaaattgcttgagtttacattaaaaaggagatataaacttctagataaaagcagtgtgagtagatattacacagtgcatcttcaaatgaggggcatgggtgggcacatgaacatacctgagtaagtgcagggcaatgttggggtttagtttccttttaagtactgtctcctcctctctctcacacattcaccgtcccccttcctacctattgtaggcacttggggtctgtattctgactgctggctgtttggtgcccatgtggtagctggatctgctattgcctgttctgttttcttctccatcaaactgtgagctccttggaagcaagggctgcatctaggagcctagtacagttagtatctgacaggtagaggtctgagaggaatctgctcttttgtttgctagacagaaattgtgtttccttttccaaacactcccagttgaagtaatttcagaaggctcctaaggaatggtgggaacagtggagatttctagtgggatggcaagagggtgggggagatgtgtttgggtagtttctagatcatacaatggttcaagaagagtggtactttagtgctgggggcgagatccactttactaggacattcagttactttagtgtcgacttttctctggatttcacccttctaaggcttttcccctctagttctcccaacctctgggtgatcattagctgtttttccatctctttcatctggcaggtattctgcatttgcttttataaagccattgctccagcagtctgctgtaaccagattaaacaggggtgctctaatgataggagctgagacaggggtttgcttgcaaacttcatgtttcacatgaaaggctttggcaccccctgtggactgcccttcaccctgttttaggcatagtatttgtttgcttttgtatctgtgataaaacaaaggccttcctgcataaccagtgctgagcaaatgtcttgaaacaaagggaagtgcagagtttaaatccacagtggtgtattaccttgcctgcatttccatatctttggaataaatattttaacatgttaattatctactactaataaaaatacctttgaacttccagtcaatttgatgggccatagaattgtctccactggggaatgggaagtaattagtttctgtaaggattgtaggtcacgttgtctcttctcagtaagtggttgcatggtcagtagccagtcatattctagggaatgtttgtgccaaggccgccctggtctgcagcggggagctgctgcctccacggagcccctagggcctgaaggaggcagaggctgctgctgcactgtggccttgggttccaattcactcactgcttcttgacccagaagaaaaattccttctaagtagggcagttcgaaatctacttatgaatcatctgtgtaacctttttaattttttcttctcattgcagactgaaaactattcttttgactccaactacgtgaatagccgagcccatttaatcaagaggtatgttggtttgcttgcttattgctgcaactaagcttgatttaacattaaaaagcaaaacagattaaaatttttaaaacgtatttttttgggggattttaaaatgaatgtccaacctactatttgtagaaagtttggaaaattgagaaaagcataaaaaagtaaataaacatcactggtaatttcatcaaccaaaggtaatcactgttgacattttacatttcttggtatgaaaatgtacgtataaacactcaacaataaaattggaattatattaaaatatagttttatctagtgacagaaatctgatattacaaatattcactatttctaatatttctaatttctaatattatttctaatatttctaatgtgatattagaaatatacaatagaaaactttcacaatcatgttgataccaaaattcttgttgctcttcactttaaataaaggaaagccttaaaaatgtttttcatgtagaacttcccatgagttttgagattgatgctatttcaacaactttgaaatcattttaaagatattttaatgacagagaagccaatcctttcctttctagatgagagagaaaaagaaatttgcctgattcttcttaaaatttttagaagtgtttcagaggaaacattcaaagttgagatttatgtgattggttgatcataatttagtatccaaggagccataatggatgtgagtacagagctgaatcctgccaagaaagatcccatctggtgcagtgggctgcacccaatgggagaaggacaaggggtttgggaagccctctaaaaggtggaaactgagcaaagaagagaacctcttgcacagaaagaccagtcctaacctttcaaatgatgcctgtattgctgtttcatgtaaatatcacttaaatggtccagtggttattctgcagctaaaattgaactctgagtatgtttgttaaaataatagaaatgaaacagagaggccagcagggtatgttagagctgtagcagagaaggcttgcaaattgtattattttgctggctctaatcccataaatttacatcaccagaagagtagaattcaaggaataaacaagcttcaagtaacttgcattcatgtctgatttaattcatgaggcttcagtaacccctaaaacatgctaagtttatctgtattggtctggaattgcttgtgaccttaagtgatgatcattagttgtctgaaaatgggtagcccttgtattatcagtgaaagcacagttgagaattttttttgaggggttgggaggaatccatttagaatattttcctgggtcacagaataaaagcaagaggtgaggaattcaaaattatctgacgaatttgttttagaaagaaaaaaataaccctcttatgtgcactgattagagtgcctttgtagcagaaatctgctttgtttctgcttccgaattgttattttcaaagctccattttaaaactttgataaaaacagtgacttgccttaccctcacaggtccctcgggctaactcagggtacctgggaattctatagccaggttaaacagataacatgatgctgggcgcagtggctcatgcctgtaatcctggtgctttgggaagctgatggtggagaactgcttgaggccaggagttggagaccagcctgggcaaaatagtgcaacccctcccccccaccccccaccatctctaccaaaaagaaaaataaataaataaaataaaaaaacctgatagtatgagtctgattgtcacagcctggaagaaagctaagactcccagggacccttgcatcttacatcagtcctgactatccaatttacaaggtagactgatagataaaaacttatgtttgaacatttatctttggggatacatgtatttccacagcagcgttgagacttactgggaacctctgattttggttaattactttatatgttttttgtttatagacacatacacacaaaaggcacaacctagacttaatgggctcttggtgaaaaagtctcctgattttgcatgaaatcaaagatagctttttcttcttttttttaaataaaaccagggaccaccttgatgaagaggaaacatctgaaaactcagggacacatgcatttttgccagggttctgtttacagagccatgagagctttgggaaactctgaaggccttgaatttgtgtggtcaattgagtttagatgaagcataatacctagaaaaatacagggaatcttgtgtgctatgctgttctggggaccaactgtgttaaatgtaaaatgtgtggaaaatgttgagagtggtggctaacaccaggtgataaagtcatgcgaagcctgttcaaatagctcacaacagtgtttgaaaagggttttgctgtatgtggtttcttgaagatccatttacatgcatggtaaacgctcttggccaggaaattctgaatctgattatcagttttaattttactatcaagtccctgtttctttgaatgccaatacaatacaatttcaggctaactttgagaacttctttttctttctttctttctttttttttttttttttttttgagacggaatcttgctctgtcacctaggctggagtgcagtggtgtgatcttggctcactgcaacctccacctcccgggttcaagtgattttcctgccttagcctcctgagtagctgggattacaggcatgcgccaccgtgcctggctaattttttttttatttttagtagagatggggtttcgccatgttagccaggctggtcttgaactcctgacctcaaatgatccacccaccttggcctcccaaagtgctgagattacaggtgtgagccaccgtgcctggctgagaacttatttttctatccatgttctcttcttctattcactttctacttatccaaggatgtttcttggacccttcttttcggcattctctagcagttgttagcattcttagccagactgtttacatacaggcatcttttcctcttttacatctatctaccttgagtagttgaacagcagctaccactgtccacctctggttctttccttcatcagtcagggttcctacatgaaatagttggcacgcctaaattggaatggcttgaggaaagtttatttacaaagaggaccatttacaaaggcttgggcacggttagtgaacctcaagggatactgcagtattatggcactggaggagctaaaaatacttcagacaggaccagggaagagagcatgtagggaacattgacttgagagtaacagtgacttctggttggccaagggatccagtcatcatgaagtgaccctggaggaagggagcagagacaatcaatatcctgatatcactgtcctcccctgctctgatctcccgctgatgtcttcctttggctaaacccaacagaaagccagagggcaagggggttctcataggcacaaatcaaggttgagaaggatctcaactgtgggaaaaggaagtgatatggcaggctccccataattttatgcaccaaggaatctagcattatatttctgtagttttttttttttccagactgtcacttgcaacccgcaggtgtacccaaggaggcttatgtgagggccccagattaacaaatcatggcagtgcagagtttcttctgaatggacttccttcctgctctgtttgtttcttgctgtcttctcagagcaaggaaagccagcagtggtactttgtcgctgctgcagcactctttcctcatgcctgttttgtgggctgctgtctgatatccactcctacagtgctttgggaacaatatcaagcagggcataaggctgtggcccaaagaaaatgaaataggaaatgaacaaagcactgactcagcgtggaaaggattaggagctttggctccctaaatcatgcacaatgctttctttttccacccaaattgtcctggggtttttccagcacttcgtatggtgccttgtatataataaatgcttggtacatgtttattgaatgaatgcatgagagaaatggaagaaaatcttagcatttttctattaaagactagcactagatttctctttaagccagtgatttctggattttaatgtgcattggaaacacctggaaggcttgttaaacgcaggtatgctagctccatccctagagaacctccagttgtaacaggttcccaaggtgattgtgatacaggtggtccagaaaccactctaggattttccttgtattacttaaatacagttataataatcttcattctcactccttgctcattcttagcctcagatatcattccctgggtatgtagtatcacctactagtaagatataacctgtttccattctgagtcttatgtcttttggaaaagagttctaatatttcagatggtaaatatgggtcagaatttgtgtgatgttcattcgtcatgcatctgctgagtaactcttctgtgcagaaccctgttctaggtgttttcaaacatatctgtctttttgttggccaccagagcagtaatagaaacatgggcatttacaataccattggaaacaggaacccctaatttattgtcccacaaaagttgttttacaagtccttcatctcccaagaggtatgcaggtaataaagcaagtgaaatcagcatttttatgcctggcataaattgttatttgttttgggtgagtagccatttaccaggctattttacaatatgtggcatcttgctcactgggtcttaaactgccttacacaaatccattcattaaaccactaaatgctcttacaagatcagtgtaagtggtgcttagtttgtcagatgggcaaactaatctttaaatgagttaactattccaaggtttgggggagttgttataccttgctttttgttctgagaatacacaacttctcgtgctcacactaccagtcccttacatcctagtagctaatccttcactgttttattatttccataatttctattcaaaaagcaaaatctctgaggttgtttgtaacattcagatgcctatgaaaaagatgatcaattttttaaaaggcagatccaaaaccaattaggagaagtaaggtagacatccctggcatccagggaggtaactgactgcaattaaacattacatttatgttgaagtttttgagaagtgaagacaaagcagtaagtacaccaagtgatatagttttaattattcattaaaggaaggatggccatttgacaggagaactattattttctgttctttaatgttagaagaaattaacacacacacacataatagtgtgtgactattgtttacagaagatatagaagtgttctttaaatataaattttctgtaccaatcctataaaaactatggacataatatttaattgtaactcagtgaaaacattactacacagaataatgatggtattaaacaataaccatttatggagcatctatgtgtatctggtacttcatgtacagagtctctttgagtcttctaaatggccttgtgaagggggttttagcaaacacattttacagataaagaaactgagattcaaagacatcagatatcaggtccagggtcacacagatgaagtggcagattatagattattacttgtcctaatgactccaagttcagtgctctttactacagtctgctatagaaataaagaacctagagcagttgttctcaactgtggggtgatttggcaatgtctctttggagacacttttggtttttttcctttaagtgggggtcactactggcatccagtagataaagggcagggatgttgctaaacaccttataatgcacaggacggccacaacaaaggattttgtagccccaaatgtcaatattgccaaggttgggaagccctgctccaagggctggttaattacattgcccccatactagatccctcaccatcatatgtttcaagcaagtttcaacagctgctgaattcagagttgaattctctggcttaaagtgacaactttgtgttgtggattaaggacattttcagataccagaaaatatacaggttggaaagttaagtatttgttataaaaatggacctgaatatcatccaggcaggtcatttcctttctccagaggtacaggggtgcttgtaggcaggcactcacctcacgtgacagagaataatttctaaaaaacaaactttacatctatttaaactgtgggattataatagtatttacagaaaactccgtaatatgtgccataaagctagaaatggtgaaacactttaaatcatgtgtttccaattggccatcatggccataaaagaactgtaagataatttgcttgtttgtttgttttttaaagaacggcatcctggtgggtccataaggctgagttagctgtgtgacataaattgaaggagctgagtgagctggcctattgagtaattttaattttagtgcagttgatgctcagttattctttctgaaaataattagctaacaggtcaatgcttcactctctcctgctcttcattttcttataattatggctttccagttactggcctcctggtctttcctttttatattcaggtcagcaagcattctacatctaggttggtcatatgttaaaaatctgctgtgattcttcttgactaagtgtggagggtgacaatgtatccattgtgtccatggcatagctcttcattatatgtgctgaatcttcttatcagtctcagagagccaggcctgtgaatgtccaaggttgagcagatcaagtgagtttgcggtgtcatttccacagggctcttttctcagaggcataggtctattagggtttgtgttaattcacaaacatgccattcttccttgttgaggcaacaacacattttcttgagtgtgcctttgctgcttcctcaccgactagggaatggattcaccatctactaggtatttcagatgcataatttatggttcatggtgtagcaaagctggggattctgtcaggacatacaaagtattagaaatagtaggtgggtgccctatagctgtaccttatgcattattctccatttcatgagaaatggtgtgaagttcttggattgcatgcatagtgcattgtatggtagattgatttaataaatgcaagaatcattttacctatcaacacttgagctgaaagttttcatgttcttagtataactatactcagagttgcattagtgctcactctctggctctcacttttgtcttgccttctatattagtcagggttctgcagagaaacagaaccaataggcatatctaaatctataaatatgtagaaagagttttattgtaaggtatgggcttatgtgattatgggggctgaggaatctcataatctgccatctgcaaggtggagaaccagggaagatggaagtgtagtttgaaggcctgagaaccagagagccaatagtgtagattcaagtctgggtttgaagtcttgagaaccaggagcgctgaaggcgggagatgattaatgtcccagttcaagcagtaaggcagagaacaaactcaacttctttcttaatttttgttttattcagtccttcaatggattggatgatgcccacccacattggagagaattgtctgttttactgagtctgccagttcaaatgctaatttcttctggaaacaccttcatagacacacccagaaataatgtttaaccagctatctgggcattccatggtgcagtcaagttgactcacaaaattaaacatcacaccttccacgctcagctactgactctcctctttttctttgaatatgttcatctatttttctcagataaaactgacttactgggttctctgatcctgataatccttcctattttagttgcactacttcaatttctgctacctttgctttttacagttggttagtagacatcttttaagaatcagtgtacatagtggttaaatttctaggcaggctctggagctagactacctgttttcagtcttaggtctgctatattttttaccagctgtgtgaccttgggcgagttatctcacttccatgtacctctgttttttttcatctgcaaatgagaatattagtagcacctttctcttacagagttgtgatgatttcaacagttaattaatgtcaaacacttaaaacagtgcctcacgaatagtaattgctcagtaaacactagctattattattttactttgattttataggaagagaaaagtcttctgaaatgatgacattctttagaggcagttttagaattgtagcttcttccaccttgtctgtagtgatgcattgaagataagtgaacgaaaatattagctgtatcagcatccaatagtgggaacctctgtgcaatgttaagttttattatctttaagcacaaatttctcttccccttattcctaccctattggacagtctagccactgtttgcctcattacagagccgacatcatcctggctcagaaagatttttaaaatcagatctgtcacggtcgtaaattatcattctgtcttcaacccagcagctccaccttcaaggacttagagggcaacagcctgaaggatagtggacatgaggagagtgaccaaactgacagtgagcatgatgtccagcggagcctgtattgtgatactgctgtcaacgatgtgctgaacaccagtgtgacctccatgggatctcagatgcctgatcatggtaaggactggttggatgtaagttgcagcaatcgactccaaagaacccaaaatgtttgtaggcacactatactactttgcttaatgagtgtattatcaaggtgtctacagagctcacacctcagcagcatctacaggtttttgagatgtcctgacacctagccagattcttctttgccctccctaagtggatcccctaggtctgtataaagttcaggcactttagaagctgactacatgtgcatattacatacattttcttcaataaaactgcactgagctcataggagatgaacagatgccttctctccaggaataaatgcctatttttgctttagtacatgaattcagctgatcaatcgaattgcatagaacacagcactaatgagaccaaaggggtgggttttatttccctatcagactggttagctttacccagagtgagactctgttctctcattacagattgtgtactatttactgaggatagaaaataccaaacttcccattttgtctatggggctgctatgccaaatccctccctctagcttccacacaaaaccatgccattagtcataggggagaccagggtaagagaacgtggctgagtcaatgtaaaatattccgttaatgtgaaaggcacctcaaagcacatgtcaggagtggcatcctcatgtatgaagaagagtctatttctgtgaactcggaactcagccacacatgctttatcgagaggcactatttaaaactttgctaggcagaagccagggtataaaaacattagcagagttcagctaaggcagaatgtttagatggacttgggcacaaatgtcattttcaatattttactgatgcaaacatgctatgcaaacagcctgtctttttcaattaagaaatatgaattcaacttggtggttaggctcagtgaagcttcagaatgcttggctatgtcttgagaaaagctagactcaagctcagctggaagccctactgctaaaagacttaaaaatgagaaaacaaaatatggtgattatgtgctggtcttcagctgtttcatttcactgtaaacatcattaaatgcttttgttttggtttttggaaataagtccaaaggaaaaaatgtactatccacaaaacaaaatgtttttcatggaaaaaaattagtcactgtagattctattaaattgtcttaatccattccctccccagcagacccaagtgtcaaagtctattatatagagaggtcactatgaattgtgcactgctgtcttccctaattggtgagtggtgacatctgcagagataatacactttctgggaaaccttactatgactaaagatgaaaaaaattaaacagttatataactgaaacagagacaatttaatactcagaggattagcagtatgtcacaggatgcaaactgatatatatcatttactagttgtgtgtttcaaagaaatgctttttcatttatatcaagaaagggttttagttagatgctctctaaaatgaaatgactccaggaaaggaaatagtcataattgattctcactttatgcctggctagaacaacatctgttttatttttttgtcacatttctctaggaaaacagtatcagggaactgcagctagttttttctgagagagtgtttcttacaaattggatgtattacatatatatgtgtgtgtgtatatatctatatatgtgtactgtgtagattggataataagacgagtattcttttttgtgaatgcatacctaaattttatttcagcttagtcatttgggttgaaggtagagaccaccagtctccatataggtagagtgaccacataacccagtttgccttgtatacctcactttacatctgtgtccttggcataatcaataatgagaatattctaaaggaaatgtgtggcaatactccaagattcccctacatataagatgatgattttacacacacacacatgcatacacacacatacacactttctgctgggccattttggcaacactttcacattttgtttcttactatttgatttgcatgtgtgtatcttcattttttctattattccggcagtggttaaaccttgagccaacccttgagtgagttcttttcaacttgtgtgattgcctaaagtttaggtacaaaaagaatttgggccttgtttatacctcagtctctcccttgtgatatcccactacttctgaagctttctgtaataatacttgtgtgtcccatagcaacttcaggcttgccacagattccatcaccagttcacttggactgatactgcaacctcaaaaaagagcaacaattccaacaattaacatttcatagcattttaatatctcaagtctgcatttgcataataagtatatcacattttatcttaaggtacagtggttgaaaggcagtcactgggtgatccaccatgcatgcctgatcttatagccattttagttagattgtcccagggtaacatggtattgttgcttcaaaggattaccaatttagaaaggtgagaagggaaaatagaatctgcactggactgagacctgtactgaaaatctcaagaattccttgaggagaaaaaaactagtccccaatccccttggttcattgacagtgtgatgggaatcttaaacccaagtgttctgaaattcttggagttccagagcaactgttcttgaggtttgggtaggtgtcactggagcagaatgaagaatgtagtgaatttgtcaggctaactctgatcctggtatttcttttagtcctgccagaaccactccaaagttgggccaaatgtatccaacaagagcgtaccttacacctttgttttggggagtttggctcaccttgtattccatgtctgtagttctctctgtgacttttctaatttggatattagtactgcatatttccttgcacgtatgtggacacatgattgaaggggaatctcagaatttcaaaataaagataattttgtgcatctgttagtttttcctttgagcaggaatggtttgttcaaggagctggtattgtgttgcctgtgtttggggtaaaggtataaactatatttgctcctctttttaagagatacctgggccctgtgcttttccacactgccaaaatttctatgggatttgtccttaagattttattgtagcagaagaatgatcggtgtgaatgacaagggtgctttgtgactctgagtaagggcccaaattccttaattttcagtaacccttctctgagaacattcctagaggtgggaacccagaatcactatatttaatgaaggaaggagaatgttatattcaataaaggaaggagaatatctagaagacttctttttttttttttttttttgagacagagtctcactctgtcacccaggctggagtgcactggtgcgatctctactcactgcaacttctgcctcctgggttcaagcgattctcctgccttagcttcccaagtagctgggactacagatgcgcactaccactcctggctaatttttgtatttttttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgccccgctaattttttgtatttttagtagagacggggtttcaccatgttgggcaggctggtctcgaactcctgacctaaagtgatccacctgccttggcctcccaaagtgctgggattacaggcgtgagccaccgcgcctggcctagaagacttttgaactcaaagggcaaaagtcaggtatttagtcactaggaagtttggattatgtgtttccttttcaatttttaaaaatgtctaaatttattttttattttgtttataattgacacatcataattttacatgtttgtggggtacaatgtgatgcttcaatacatgtatatgttatataataattaaatcggggtggttagtatatccagcaacctcaaacatttatcatttctttgcagtaatagcttttaagatcctcttttttagctatcttgaaatatacaatgcattgttataagctatagtcaccctactatgtatagacaccagaaggattgtgtttgattgtccaaccagacacattcgtgagctcagtgagggtggaaatgagagagaatgacaggaatctctgctttctggattactgtcaatcttggtatttctcttgacatgcctaagaccaggtcagttggttgatgaactggctaagctgaatttcctggttgccagctgactgaattacaaattgatttgttatccagtgcccatgaattaatacgtttcagtttgtccttagtattcagctcttgttatggcttcccagaaaagaagatgatagagaattctttatcctgtgctttctactggtgtattgtagaatggcaatttggaatgctgtctcttaatagctctgagtggcttgagaccaagtaagtgttgtcattggcgggttttaccacttgatttggttcttgaggcaatttggtgctgagcttgggagtcaacactcaaattttttaactgatgccttctgtccctgagggaattacttggcttgagggagtctgagttacctggtaaaattaccaccctttggtggggataagaattttgcctttatgcggttcctgcagagactctctaaaaagagtgattgacaagtagagtagtgagaggtgccagaggctcatgggtttgttgcgtgattaggggtatcatggggcttgaatttcttcctatccgggttatctctgagctaagtagtgggggctggggtctggctccttcttgcttctttgcataacaatttttgatgatgtggatatcccattattcttacttaaatatgtaatcctgatatgagattggggtttatagggatcatgccctagaagtgtacactagtcccaagctcagctattttgtttcaaattacactcagtccctctttccttatggttttaattattgattataaattcatcagatgaaaagatgactctaattgctctaatatgcacccaatttggagttcgctacaacactgaattggtttccctccatgctaactgataccaaatggaagcaacttacagaatttttttgtaagatttagctgcttttcatatccaaatgcgtccaaatgcaatataatgattgctttatttctggaagtatagattcttctgttttagtagacttgtttatttaccagtttcatcacctgaatagcatttaacatgtttgctgtgtataacatggatttgaagaacttgatctgtacctaatgcaactgctgttaacaaatttgccatgaaaatgttctgtaggctgcgtactatgcttattctagtgtcaaccacaggtatattaagatgtttacactttcaacactctaccaacaatatgtaattttgtttcctctagccatttaaagaaattatgattccatgactgaacatccttcttttatcttcttatctgtcccccgactcacctctctcctcactgaattttgaaacactcatttcaattagtctcttggcaaaatagggagttctcaatggagactattttgtgtcattgcaaagcagtgtgctgctgggctccagatatggtagcatgggccagatgttcatagagtaattgaaaattccattttatcagaatagcatttgctgtcaaaaagttttatctttacgtgaacaaagcggttgattagatccccctggacctgaattagcatctttgaggctagatacactgttttgcaattcatgtggttatttctttcccagttttgctcatatagagactttaaaaaatgtcttcatcctcttgcaaatatacctagtgaaatttaattacttgaacccaggaatgtgtatacagcgtttctatatatgtgatttccagctgctttcatcctgcctctggtatactaggcaggaataaaccatgaacagatatatatcactcattactcagtaatatttttgagtgcccactatgtgcctggctctgggaattgaatgtgtcaactactgctgttattgactgtaaaagttgttatctttttacttttattaatctggggtttactgaggtgaccaaggccagctcaatgatgaaatgaataattctattttaaaaaattataagaatagattttggggccattaaaaaccttgtgaatattgtgccagtaaagcaagagaagcaatatgaaaagactgttacaatcaagatcttattttcaatgggaatgtcatgaaatattcctcctttgatgttctaatttggacagtatcacattggtaaacatttttttttaaaatccacctgttgtgaggttgaatttcctactcatctttggttaatgtcattgtaggatgctaagtttaatctctgacatgtccaattaccttgctggtttagaggcagtcagtagcactcaatgcctagtacccctttttttctgatgcccagaaactttatcctttctctcagtctcagcagtttttgggcaggtaggaagagaggtgacaggtggagctttttcttttttttagttcaaaaccaattatctgctgtaccacataattggattttttccaaggcaacatttacattttattggaaaaataatcactaaaataaaaagttgttcttcactaatggaaacctccctgttgttagaatttctttgctagtattcttttggcatgggccctttggtcaaataattgttttaggacagttcaccattggtttcttcatgcaggacattaagaataatatcaaccgtttgtctaattttaaatagatacgctattgtgttatagcatatctatttaacatattgtgttatagcatattgtgtttcccaacatatatatatatatatacagacatatatatgtatatgtgtatatatatgtgtatatctatatgtatatatatcgggaaaacacatgcacacacacactatacacacacacacacacacacacgttggttatatatatgttatgcggttcctgcagagagtctctaaaaagagtgattgacaagtagagtggtcagaggtgccagaggctcatggatttgttgcatgattaggggtatcatggggcttgaatttcttcctgtctgggttatctctgagctgagtagtgggggccagggtctggctcctccttgcttctttgcataaccatttttgatgatgtggatatcccattattcttacttaaatatgtaatcctgatatgagattggggtttatgggaatcatgccctggaaatgtacactagtcccacattcctggattcaagtaattaaacttatatttgcaagagtatgaagacatttgtaaaagtctctatatgagcaaaactggggaagacataaccatatgaattgcaaaacagtatatctaacctcaaacatttatcatttctttgtggtaatagcttttaagatcctcttttttagctatcttgaaatatacaatacattgtttctttccatatgtatatttatgcttgctttcttactctctgtatatgtagtgtgtgtgtgtgcatgtgtgttttcccaatctacttggaacttttagttgtctgatattcaaggttattttaatatttattattactaataattacttgtggtgggctgggcatggtggctcatgcctgtaatcccagcactttgggatgctggagcaggtggatcatttgaggccaggagttcaagaccagccttgccaacatggtgaaaccctgtctctgctaaaaatacaaaaattagctgggcatggtggcacatgcctgtaagtccagctactcgggaggctgaagcacaagaatcacttgaacccatgaggcagaggttgcagtgagctgagattgtactactgcactccagcctgggtgacacagtgagactctgtcttaaaaaaaaaaaaaaaaaatgaagaggaaaaaaacaaacaaaaaacgaaaacaaaaacaacagaaaaaaaactacctgtggtaaggaattcattactcatcaaaaaataaatgccaaagcttttcttgattagttgacattaagaatttccatatttttaagcattttgaaagcattagaggatgcaggatatgtaaaaggagcacaatgaacaattaagaatttcattctccagtagtttatggcccaacagcaaaaatggtccagtatggaatagactacataggatcggtgaagaaagtaattaagatttttgagctggagaccaaaatgcatttaaaaggacactttggtgtaatgtgatctctttggaggaatggtggaagccaggccccaacatagtatgctagataaaattatgctctatttgggtgatttactaggtgaatgttttccagtagcttcatgaaaaatcaacttctataggccatgaaaagtagggacactgccaataagttaatagatgagagacaaatcaagttgttttgtgggactttccctttctttctttctttctttctttctttctttctttctttctttctttctttctttctttctttctctctctctcccttccttccttccttccttccttccttccttccttccttccttccttccttccttccttccctccctccttccttccttctttccttttttctctttctctctctcttttccccttcccttctcctccctcctttcttttcttttcttttttctttctttcttttctttttttgagatgggtctcactctgtcacccaggctggactgcagtggtgcgatcacagctcactgcagccttgacctcctgggctcaggcgaccctcccaccttaaccttctgagtagctgggaccataggtgcatgcttggctaatttattttggtagagacgggattttgctatgtttcccaggctggtctttagcccctaggctcaagcagcagtcctcctgctttggcctcccaaagtgctaagattacaggcatgagccaccgctcccagcctaacattatttctgtcctgtcataagatgattaagagccataaactcttatcaggatgggaaatgataaatctaaacatggattttaaatatttgtagagtatttttttctgtttattttcctcttaatccttttgttatgaaataaatatggggaattaagtccattcatagccagtttaagggattgataccaagcattttttttttttttttgagacggggttttgctccaggctggagtacaatggcacgatcttggctcactgcaacctccgcctcccgggttcaagctattctcctgcctcagtctcctgagtagctgggattacaggcctgtgccaccaagcccagctaatttttgtacttttagtagagacggggtttcaccacgttggccaggctggtctcgaactcctgacctcaggtgatccacccgccttggcctcccaaagttctgggattacaggtatgaaccaccgcccctggccccatagttttaaaagcaatttcaacaataccaaaattatgtatcatttatttattctgtggacaaaatagtcgtgagatggctttcctcttttatgatagatgctttttgtcatccagggtcaggacttgcccaaggtatttttggtctcttgtttttatcagatggctcagtgccctgtatactttgcatcaaggaaagcttaattgatacatccactttggaaacgtggctttttgacaagaaagcctttttaatttctctttctttatataaggcttatccccagtaaaactccagcctagaaaaatcattgtgttactccattctcacactgctataaagaaatgcccgagactgggtaatttataaaggaaagaggtttaattgactcacagttctgcatggctggggaggcctcaggaaacttacagtcgtggcagaaggggaagcaggtactttcttcacaaagtgacaggagagagagagaatgagggaatgagaaagtgctatactttaaaaccatcagctcctgtgagaactcactcaccatcatcagaacagcataggggaaaccgtccccatgatccaatcacctccctcccttaacaggtggggattacaggtccctccctcaacacatggggattacagtttgagatgagatttgggtggggtcacagagctaaaccatatcaatcatcaagcaccaaattagaaaaggtttgagctgctctttttaggaacatcagaaaagttgctttttaggggatcacacacaggttcttcttctgggcatattttcaaagatctgacataatctaggcttaggtcttagatggacttgtaaagctcacagtggatagataaaacagactcttttgcaactggacaaagtggaaccccatagaccaatcattctgccaggtagagagaatataaagcagttcctcatagtttgcgatgaagagattattaatgaaatatcagtctccagagctgttttggagcaaatcctttgggactgtaagtgtgtgtgtgcaggtgtcaatgtcagaagggtgctgtaggattttaggttttgtggtaaacactggacttcacttaacaatgtgccattcagagatctaatatgtggttggaagtatgtgtaggatggtgtgtaaaataggtaattcgaaaggcagtgaatctggaaggggccaaaagaattgatcagaaaatgaactctttgtgactgaagttactcaaagttttgtcacattgaggcggcctaatgacaaatacttttttcttttgagggtctctggtcatcagaaacaatagttgtacttcattatccttcatgacagtttagacaacaaaaatgcaagctattaaatttgaaataatagacaaccctgttttatcattagtttagttctttttaaccttagtaatactcattttaatggcaaataaggctctttaggttactgccttagtttaacaggaaaattttcaatctggctaaagaactctagagttttagaactctaaaagcaagtttatctccctctgcccccagtaagctaagccttagggcctttctaaattatggtctagtccctagactctaagtaatgcaggactgcaactgtaatctgctttgaataaaaatacaacttataatgttgctttctttttaagtagaaatctgtaaacatacagtactctaataatttggagaaacttgatactcttgcagcaaggcagcttccaagtcatgggtttgcaaatccaagtccaatgccagagatatattttccaatttctgtggtcgcttttccacctctaagctcatatattctctccttttctatccttaacttgcataataataaatgtttgacaacttgctttctttcctcctctttcagtctctatggagtttccaacctgtgtgaacaatagaatatttcccttttaatacatggaaaagaggaaaaagaggaatgatgtatgtttcgagatatttaggctggtcaaactgttccaaggcatcaccacctgctgtttttaatcattaatgaggctgtcagttgctctaatctgggaggtgaaccaaaagaattgttttgactttaatttaatttaaaaatcagcttattgaaaaaaaatgcttccagcttctatgagtcaggttgcacactgttgataaaacaaagcaagtcaaatgttgatgctgaaccatcattcatatgcaaacgtgaaccgttttaaatttgcagaactcttattattaccatcactagccagaaattttagttctttttttgtttgtttgttttaaacagcgtctccctgtgtcgcccaggctggagtgcagtggcgcgatctcagctcactgcaacctccgcctcctgggttcaagagattctcttccctcagcctcccaagtagctgggactacaggcgcctgccaccacgcccggctaatttttttttgtatttttagtagagacggggtttcgctatgttggccaggctggtcttgacctcctgacctcgtgatctgcccacctcggcctcccaaagtgctggaattacaggcgtgagccaccgcgcccggccaaattttagttcttttaattcaagtagtcattgtattctggagacatgctgcgtgtaaatatctgttccattttttagaagaaaaactgatgcaccaaaattttaaacactctattaatgtcagaacacaaatctatagcaaagtccagagtgaagcctaaagtgcaaattcttttgcttgactcaatgctcatcacactgtttgtatgttttagaagtttagtgactatctgtgagtactctattaagttggagttttcttcactggatggttttgcttagtgacaaaatgagaactcagaaatacagtgtctgtgcacaagtggattttaacgacctagagccagacctttgtttgtgccaactgtcaatttggaaggtttcattatgcctctctcagcttcatcaaacactttcagatttataattgatgtcaactgaagttgaagtcaaagaaaaccttctttttttgactgtagaaaatatcaccttattgttgggccctggttagccggagggtaggtgtatcaaatcacactagccttctgctaaaacagcaagtgagcacttgctgaattcagctgataatcaatctgcagctaatgagtctgcagacagactgagcggctggagctggcaccaggaaaccaccattccaactaaccatctcttattaaccttttggtgtttcctttgtgacgctaattagagacccataaaactatctgctctagatcttccgtttaaaatacaattttcaaagaaacacaattcactttaaggctcctgttagtatggccttttcaaacctcatgaaggaaaatgactgacttgccatttaagtaaagtttggataattaatctgtaatgttttggtaattaaaatgccaggaaaagttggttctgtagttggtataatttataatactctccagattccggtggtactgaagtaggcggcagatgtttctaccaccgttatgttttgactgtctgaaagtacccattcccacgctgtagccctgtctgccagactgcctgtctgcctcctcctttggtgtgaatttgttttgattgcctgagggggttagatgtgtgctttgcttcctttctgggggatgatgagccaaggaatgagctttgtactcttgggagagggaaaagtcttcgaagttgacatgctgtttagctcaaaatgagttatcaaatgggcccactcaacgttcaaactgttacatttgcatgatgaagatggaggagcaccatgccttcctgctctagtcccccatctcatttcctctttaatattttttacacccagaacaagagcattatgagataagtgttctaagcgtgttggacccatgtattcatcctgtgctttcgtttacctgtctacagcaccttttagtgcctcatttctctcatttgtttaaaataaaaaattacctagcataaatgacttacatgatcctaggggacagaatctctagatgaggaattgactgactattaaataagtgagatctgcaaagctggacattgttttatttgatttagcaaagaggtactcttcgagaagcccaatattaatcatctaggatcctttagagattaaaatatccataccagaaatgcaggcacagaacccaatattcttgcagcattacctggagcccctttggtatcttaagttttcattgtcatcaatatggaatgttcaaagtcctaaaaagtccctgaaatcattgtgatatggaactcatgttataatatatcatttatcccttacagcaacactgaaagggagagacaagcatagaattccaaatagtcaacagaggaatgacagccatctatagtgaggatcaaaatcagaatcttggctgatggggaaaggttcagaaaattagaatgaggtgataagagagaatgaaataagataaaaatatctatggttgaaaccgagattggcagggacaaaaggggtataatccagatttccttttatatgtactatctagctaatactcattgataaacctaagaattctaattctcattggagtcttccagacagatagtgtccaatcacttcaggctccaactaatatgagccagtcctaatgaatcagtacaactatttccactctactccattatattcaagattgacatcattagtctgcctgtaaatacaaaggcaggcagaacatcatagtggctaagagtacagactttgaaataatacagatttaagtttcatgcttatctttgccacttaatagctattgaccttatttgttcgaatcctcaattttcacaactgcaaaatgggagtaatgatagaaattacctatcagaatgttgtgagaagatccagataaatacttcctggaatatagcaagtgtccaaaacatagcaagtgttattgtggggactgagtcagtggaatagccactttataggatttcaggtaccctaccactcaaagcccctcatctgtttgcttctatctctgatccctttaacagaaaatgaggcatttcattttacagttactgttcacactttaagcagtccatggggagagaaaatgcatttatttaaattcacactaagttgtaaatagttgctattagattgtttctaagcttattatttgagaggagtggtggggggaagaggatcgtttctagaatattgaactttaaaagtctcagaggggagaatcagaggggagaaatgcatagaaatggggtgacaacctcatgagtttgccagagctattggtgtccaaagaaagtaatttgatttactcactgtgaaatgtctggatgctggaaagctgcctttactatcaaatcatttttctgaatcagcccagactttgagaaattataacatgtctgccaaaggtttgaaaagaggttgtgttcatttacattgcaaaatatacatattcttgagcacttactgtgaggtaagagaagccctggttcctctctggaggcattgactccactttgccctccctgtatcaaaagctgcataccccagccccaggatccctgctgctcttccctgctctccctctcttgcttcacggttcttaaatggctaatgggtggtagacgtcattttgattcctttattttctctcccactgcatgcccagactttcattaaaaaataaatgaggggatgcatttttcatttttagtttattttctaaaaaagccacactgcaaagaatcccttctattctttttttctaacaatggactgaaaaatgctcagctcctacctgaaaggtcatatctccttgccatatgaatgagcttccagcaggtccatagggagagtgtcatctgagaaatgatgggaatttatgtgcctttggattttggcctcattttgtagtggaaaaataaatttgttaatgatagaaaaaaaatctcaaccaaagccctttataaaattctctctctataactcccaggagggagcctctcagacacacagttgataaatgaaaaaatattttttacccttgccttgcttagagctgtttctcttatattaatgtaagccaggtggcccaccttcgctgataatatggtgttgaatatagcaacctgcagttctattgcataaagtattttggcttgtaagggaccttgtgacatgtttgtgtttgtcaacatcctattggaaagtagggcttggggagacatgtacacacatagaactgtgtggaattagggatatgaaaaaaacaacttataacacagaggcaagcactgtgtttttgaaaaaggcagattcattgtgaaaataaactttcttttatagtttgaacattccatgaacaatgtaactggaagtgcatactttttgccttccccaaaatctaaatccacgtgctatatacaaaatgctaagatcattatagtcctggccgggcgcggtggctcacgcctgtaatcccagcactttgggaggccaaggcaggtggatcacaaggtcaggagttcaagaccagcctggccaacatggtgaaaccccatctctactaaaaatacaaaaattagctgggattggtggcgcatgcctgtaatcccagctacttgggaggctgaggcaggagaattgcttgaacccaggaggtggaggttgcagtgagccgagacctcgccactgcactccagcctggcaacagagtgagactccgtctcaaaaaaaaaaaaaaaaaaagatcattatagtccctaacatactttaatgccatgacctactgggaaatgagaattgtagttttcattctattgtttgttcattgtagagccttggataagtcactaacctctcaaagtctcaattcgtatatttacaggtgggatgatgagcaatgttgcattaatgttgataagaccaataatcaaagtgtctatctgtctatctatctatctatctatctatctatctatctatctatctagtattttttcttagtacaatataattgtgaatcggatggctaggtgttttgccggtttttggtttttgtttttgtttttttgttcccttctcttccttggcctgttgacctgacctgagaattgggaactttatgtgctcatcatattccacagattcatagcctataaacaagttacttgtccagtctggtctttggatttctcatctgcagaatggaggatactggtagtacctgccttgtggggtggttgtaaggattaaatgagatgacacatgtaaaacacttagtgcagtgtctgttccacagtaggcacttaaaaagagtagcggcaagatgatgctgatgaaacattctccctgggttcctgtctcccctcttcacttatctacgtctctttctctctttaaaattacaagcaaaatttaagtcccagaaagcagcctcttttgactaatggtttgtcctgatctctcctttacttggcgttctttaatatccatgtacagagtttatatcatgttaatttgcttttttcctgttttttaaaaatgtaaaatgagagagttgataattgttaagacatttcaacttctgagcttcttttattctgattttgggttctgggtacaaccttttcagattcctgagcacacagccatttgccaatggatctgtaatgagtgccacactcaaattaacagatggtgtggtgcgcttgctttgaggggtaattactctttcatcttaatttcctccctttggcattccttctttggatactgttcaatggcaatgccacgaagcataggatgaaaatcaaacaagtagcaggtcacttgtctatctcagacaaaattattgttaaaactaagaaccagtgaaggtctaccaagtgaggggcttgtaacttctagtattagggaccaagtaactcttaagggtacttaattgtctgatcgttcctcctttcctaaacaaggtggctatttctagttgataactgtctccatttttcttaaagtttaagtagattcctcctctatggccatcatgtcacacctctcacagttggtttgttcttaaactcttaacactgtaagccaaatctgatgtcctttcatcgttggagccagttgtaaactgctaactttactatataaactcctttcccctatcttcttcctcctctctcgtcacacacactttttcccctgatgcttcttatttagttctttctccaaccctccctgctttcacctcagcctaattctatccttttcccctctaggcaatttctcattcatcactgtgtttttacagagaatagaaataatttttaatgacattgagaattttttttttgggttttagtggttcagtcttcccttgtgaagatttttatggcttatttttatttggtaaagttaccttctttactattttttctctccttccacacaattcacagtatactaccatgagtgaggaaaaccctcagtgaataagcgctgatcaatttatttctctgaacattaccaaacaacagatgccatccaccaatgtttgatccccagggcagttcttatctaccttattttacagattccagaggtgaaatgatctattgtaaagcagtgcaaggatcagaagagagtttgtgtttcctggatatattatgtgacttcagggcattgttctgctttccatttgaaatctcaaaggctattggactgacaggaagttttcattcctgttatgaaagggttactgcctccagtttggaagagatgttgcttagggaaacagcagcagattatagcagtaaagccagaaagatcagggagtcagaagtattactatgagatggaaggaaatagctggtgtgcctaactagctcaggggcatgtttgatagtacccaggtatgtctacagaactggcacctgtatagatattgacctctcagtgtgtgttttactttttactctttgagttggactgctgtgttttctgtgtcactggtaattccattgaccaaaacacttcagttgaatcacagcttgagtcttcaagtttccttccctctaaccttgctggtttcttgctcatgttgatatttaagaacgttttctttcaattcagcaaagtttccggatacaagattaatttacacaaatcagtagttcttctataccacaacagcgaccaagttgagaatcaaatcaagaactcaaccccttttacaatagctgcaaaaatatatatatatatatatatatatatatatatacttaggaatatacctaaccaaggaggcaaaacacctctacgaggaaaactacaaaacactgctgaaagatatcatagacgcaaacaaatggaaatacatcccatgctcatgaatgagtagaatcaatattgtgaaaatgaccataatgccaaaagcaatctacaaattcaatgcaatccccatcaaaatactaccatcattctttgcaggattagaaaaaacaatactaaaattcatatgaaaccaaaaaagagcctgcgtagccaaagcaagactaagcaaaaacaaaacaaaacaaaacaaaaaacaaaaaaacaaatctggaggaatcacactacctgatttcaagctatactataaggccatagtcaccaaaacatcatggtactggtataaaaataggcacatagaccaatggaacagaatagagaacccagaaataaacccaaatacttaacagccaacttatcttcaacaaagcaaacaaaaacataaagttggggaaggacacccttttcaaaaaatggtgctgggataattggctagccacatgtaggagaattaaactggatcctcatctctcaccttatacaaaaatcaactcaaaatagatcaaggtcttaaatctaagacctgaaactataaaaattctagaagataacattggaaaaaccttctagacattggcttaggcaaggatttcattaccaagaacccaaaagcaaatgcaataaaaacaaagataggccaggcgcggtggctcacacctgtaatcccagcaatttgggaggccgaggcaggtggatcatgaggtcaggagatcgagaccatcctggctgacacggtgaaaccccgtctctactaaaaatacaaaaaattagccgggtgtggtggcgggcgcctgtagtcccagctactcaggaggctgaggcaggagaatggcgtgaacccgggaggcggagcttgcagtgagccgagatcgcgccactgtactccagcctgagcgacagagcgagactctgtctcagaaaaaaaaaaaaaaaaaaaaaaaagaataggtgggacttaattaaactaaagagcttttgcactgcaaaagaaacagtcagcagagtagacaggcaactcatagagtgggagaaaatcttcacaatgtatgcagactaatatccagaatctacaacgaactcaaacaaatcaatgagaaacaaacaaacaatcccatcaaaaaatgggctaaagacatgaacagataattctcaaaagaagatatacaaatggccaacaaacatatgaaaaaatgctcaacatcactaatgatcagggaaatgcaaatgaaaaccacaatgcggtaccaccttactcctgcaagaatggccataatcaaaaagtcaaaaaacagtagatgttagcatggatgcggtgaacaggtataacttccacactgctggtggaaatgtaaactagtacaaccactatggaaaacagtgtggaaattccttaaagatctaaaggtagaactaccatttgatccagcagtcccactactgggtagctatctagaggaaaagaagtcgtcatacgaaaaagatacttgcacacacatgtttatagcagcacagttcacaattgcaaaatcgtggaaccaacccaaatgcccatcaaagaagctgtggtatatttatatgatggaatactaccagtgataaaaaggaataaattaacggcatttgcagcaacctggatgagattagagactattattctaagtgaagtaactcaggaatggaaaaccaaacatcacatgttctcactgatatgtagaagctaagctatgaggacgcaaaggtgtaagaatgatacagtggactttggggacttggggggatgggtggaaggggggcgattgataaaagactacaaatagggtgcagtatatactgcttgggtgatgggtgcaccaaaatctcacaaatcaccactaaagaacttactcatgtaaccaataccacctgtaccccaatcacttatgggaaaaaaaaaggttaaaaaaaaaaaaaagaactttttctttcaaaaaaatgctgatgaaatgcaagtttttagcttctttttttcttgtcatttccactgatctgggctgttttagataggatgttttcaaaagttttatcatatggctctattctttttggcttattggactgcaagtgtgctaaggcccagtcattcaagattgattagttctttgtggtactatatgggcattttggtgattgtcatttctcaaaaatgatccaaaaacgattttccacattgtttaaacacactgttattactcttaacatcccaatagttattgaggtgtttattattgcctttttcttttaattggggtttttattgcataggaaaggggaaaagccctgattcaagcatttggattcaggtcctggaactgccactaactctgtgtgatctcaagcaacttgcttcctcgctttgggttccagtttttttcatctgtaaatgaagagtttggattggaatgacttataggtgcttattattgctaatactctattactgtttattgtatatttaatatggaaagattgtttggaaagtggatgacattttggctggattttcctgtgtatttctaaagaccagggcaaggtgggaaatctgactcttactcctggggctttgggctgtaataatccttccgttgtggcaagaggaatgagggtgagacattggtgaagttggcttttgttcaacattctgagcttattccttcttttaggcttttgtttgcatatgtttagcttaactctgctttcaagtgtgtgtattttcgtggaaatggtcctcttttccttttacaccaaattcatgttctcatcttccttctcccttactgaatcatccatttctgaatagttagcaattttcaacttaatgaggtctttcccctaagtgaagaacacagctgttcctcctctgatactttggcagctgcctctgcctttcccaaaatggaatgattagctctttcctttgattttttagctggccattagttcaggactactagcccatttcctgtgtgatggaactaccaaatcccatggccagaattgttccaatttgcctttagtgtcttttagtcttctggggacttaattcatatgaagtggaggatttatcaatctttcagaggtccatttgcctcattatctccagtgatttcattgttcagccttttgagatgctgatctacgaggagaacctagcctggtgtccctgatgtggttttgctggagaaatagcagataagaagatcctcaatgtattttgttcctgaatcaaatagccctcatttgtcacaggatctccacttcagtttctgaaaagcccttttgcctattccattttagtcttatgtacctgtatttaagaaagtgattactgtttttcctcatgtactttgacttcatctgcccctccagcctttaattttatgatttcacatcccttgttaattgttgggatttccttttatcatatgtgtcccttacacttaagatatcaagtcaacttctccactccactccaattcccctctcacattatgctccctgttaatcaatcaaaagcattgtgggtttagtagtgctgttccaaatgtgtgtggtgcagcccgtttttgcttccccaccccaaacgcctcattcaatttaattccatatacttagcatattaggcatcttgtcaatatttcctttctgaggtctatttttaaaatttgaatgcattctgcatgtgtaggtttttttttttcttaaatgatgagatctactgcaacagagagccagaattagctctgtgcctaggagaaaaggaaagcacagagggaaagaatatttctcacattctcagaaggtgtatgcaagagccagcccttggcacaattatttatacagtgccactatatctatctgcgtgcaccttcccctccatttgggagcataggcatccagtattctccttgggttttcttagtttctgtcaccattaacccttctactcagggccctgcattcccagtctttcctcgtttcctactttccccataaaaaacttacctggtccatgttccatttccccaaaatcctaattatttcaagggacctgttaaaatgacctgttctttctttccaaatagtatttgtatctcaatgtgatgataatccaaaagaatacctaatggtggcatttgggtcaccaatgcagcctgatggcgtgatgtcttcaggatatggccttttccccagcatcagttcctttatatcccaccaatagttggctacatggtcaaatgataaggctagcttggactccatttatcccagagaattagagctgcttgatgaataataatacctacctaacatatattaaattcttccttagtgcgaggcactgtgtagctcagcttccatgcatttcctcttttagtcattccaacaattccttatgaaggtcagtgctattagtcctgacttatggaagggaaaaaaggcacacagagtaactttaagtaacttctataggatagggctgttaagtcttagatctgggactctaaggtagtctcgctccagagcccatagtcttagactctgctgcactgcctctgggaaaaggatggtacctttttcacatgtacacctcactcagaagagctaacttcagatgtgctgctgggtagtaagcttccctttggcaaggaacaaatgaagtcttgagttgctccagtagatctagcttatatctcatcctttctggagagaagtttgcctttggacacaatgctacagcagaaaggtttgcctttgcttttctgcaccccaaaacttagcagtattaaaaactttcttcaccagtccctgtagttgtaagtcatcagtagggatatttgccaaatgcttaggaccaagcttacactctatcttttctctaccttcagagctgaaggagctatgtatgtgtgtgtgggaaggaggctctctttggtgttggtgagcatgaatatgacagactcggggtagggggatacatgaaggggagagcagactcatccacactctcaaggcaagtatggttagaaaccctgcttcactcaggtagcgctagtctcttcactgtaagtaggttttttttttttttttaaatatggtgatacaatgaattgttttctgaggtggatggttgtgttttctgttattactgcttttccttctggaccgaacttttattggctgctgcttacttactagtagaatgcttcacacctgtgggctgctgcagtcatgacaatgaacattctgttcagaaggggagcattcattctctttcccagcagctgttaccccaacattgggaatttaatgcattcatttgttctttagataaataggattaaattcagaatgaagcaaggagtagtcttagtctgtgctttgcatgattttcagggtctgtacagttccagctgtgtaaacatgattgcagaacttaagtatcgtctaatgggggaggatttcaatttaggagtggcacaccatagctgggctggagccagacattcttccacccacttcagtccacaccctgcggagtcggcttgctcagctcctagagtcagaagtgaagatggggcagctgtagttaattatactgtattatagcttcttatcagtaggggatgagaactaggcaagggctaggaaaatctaaaattatatcctcaaatatgctccagttatggcagtaggaaacaaagaaatggaagacattggacaaagtagggcagccccaaatattaaatatgacaagaaaaaatccaataaaatatctattgaaattgtagctacatttatattccaattgattcttttttgtggtcactgaaagcaagagtagagaaatattgtattgattgtaaaatattgatttgttctaatatttggactcagttttgttttcttgagatttgtttaaataatgaatagctaagtaatgctaaagacttctaataaaggagcactattattatggaaggaaatgcaatttcttcataacaaatacaaatattatggatcatccaattcctttgtccttgttcctccacccacaattatgtacagtgttggccctgctcagttcaatagagtcttagatccagtaattattctgtctctctaaagaggaacatgggcatacgtattcctgagccctaaaggatgggatttctgatgatatagcaggttttgtttgatgccatagattctccaggttattcttgggggagtatcattgatagctaacacaaaggtacttggcttcctactggatatgaatgtcaagacatggtcctgatcaaggaagacaacaaagcattgccttttgaatgtgctggggattgtaatgcatttaggaataacccaatcaatgatattcgttcctattacaactggctattgaaaaactatttttctgagaagctcacttcaaaatgtctctgagacagaatattcttttataggatggggagctctggattgaaaaagagaaatgctaggctgataaatcactgagtttgaatcatatggaaagtgttcaagaacagaaagagaggaaaattcaataaattactgttgatgcaatattggatggtcagggtgataagacactcactagagatttccagtctatgatgtacacacatgcattattttgtttactagttgtggtagcctgaagaggcagaaacatgcctccagaataccagcagggcataggggaacctctccatcatccatcatattcaactgtagtcggggggtgatcctgtcaatattgcctatgtggtatctctggaacttgggactgggatgcctattagtgcttcactgaattctgtatttgtagcatgtgcattgtagcatgtacataccatagcacttagctcgtggatgcttgtctgaatcattaggtatgaacattatgggtgccataatagcacaggaattactgcaactaaaaaggaaacacatggcttgtacttcatccacaatcacttacacttaagctattgatagttgattgggatatgtgaactgctgggcttgtgccatgggacttctaagcatatttggtgggcggaacagacaggaaggctgtagattttggcaagctgtggttcccattagtcaggctaactgtatctgacagtagagatcccaattcaccaagatgaagcttacttctctaggttttggctgatggcccaacaatattgacgtgaaggacaggagaggaccaatgttacgttaggctgtgagtttctaatgagcaaatagttgaatggtttagtgaataccctgttggttttttggtggggtaaacacaaaattcataatttgagagacatgtaagactcgaaattttcttttatagtacatgttaaccattcattctcctaatagggaaatgtgggctcttttatagggtgtgcatattatgtagttgtacagttaatactcttctctgtgtttttcttgtaaggggtgctgataacataccctactaaggaaattccaggcacagctgtttctaataataggtgccccaccccacagaattgtcaggcttactctcaaggttctttggtgggaaggaggattttctggcagagtcttggccactgccattgtctcagattgcctccctgtagcttccttggaggtatgagtttctaagcagaaagatttgctgtgatgagggaaggccacaaaactgaaggaagagagatgaggtcttatagaactaagtgagaggctgttttggtcatggcagagatgagttgaacactatggaatggagacaagatgagaagaggagtgggaatcaattcatgaaagcacttattatttataagttggaataaaaggttcattgaactccaggggacccttcttatatgaaatgcttgtgaatatgttgaaattttgtaacggcaattccatcttaaatacactgggggagctgacaacctgcagttcatggattattctatctagtgtacaatcactcttctattctaccctggacattcttccaggtgtggtcaagagaaagcggatagaacagatggaatatttcctctcaggagagttcaagagcttccttaaacaaaggcaagatgtgagcataaccttttactttctaaacaactttttctgagagctacctggaaaaatttctagcttcctcacagatccaagtaatttctgaaaatatttagagactagtaatcgtttaacttctaaaaatcaaagttggtttgtgctaaaaatatcatccggacagtctgtgtctagttgagtggggaagaaaaaaaggcaagctcttggtatcaatctttattccctctgtaaacacacatgaccagaagagaaggctgttattaatggtaatgcagaccagaaaaaagggtattattttcagtctgttgaacagtacaaaaatgcttctcaagcactgtgttcagatgtttctttttattagccatcacacaaggaattagactcatttcaaacagttcgtttccctactcttgtctccgtaaaatcgttactcatgagtggtttctttgttgaaatactgctgacctcccatattggaatgcacaacagggtgttaaaaatataaactttttagtttgcataaactctggaagaagggggacaaagattccccaggagaataaaaccattcgccaatgtccttgtttattagtgtcctataagcatcttattcaggaagctatgtcagcattacaattctgtatagactttgtgtgtgtgtgtgtgtgtgtgtgtgtgtgtgtgtgtgtgtgtgtgtgtgattaacagcagagggctctttgaattccagcaggaggttttaaaatataattctgcatctgcccctctttcttgtcctttccaacccttttctacctcccttctcatcaccgacccatccagggccattaggatagataaaggagttgtgccatgtcttaatatatgagcaaatgatgatccttagctctacagctaggcgctgtttgcattttgccaaaggtgattattttatctgcagttttaatagcaaaccagattatttgctgttcactaactaataaaagcaaataaaaacagattgcaaagtggaattaaggggataagaaagtactagagccaaagaaggctttttctttaattaataatttccatttactcttagaatatttttatgctagagttcaacatcttatattaaacatgtgtgcacccttcctcttataactgccgaattggaatctcagaatggaaatcatgcaggcttgaacggagcttcaaattctcccagtgttgtcttattgtctgtacacagaactagactcctcagggcttcccccaccacagcatggcattcctccattagccccatgtttcccagacccagtcatgaagaactggcattttaaccaacaagtagaaaacacaataaaattggagttcaggagatgcaatgttttggagaaacagattattccaagagtgtaaagatacatgatctaaagatggacagaacatttaaatttaccagcctggttttcagtgattttttgatttgttcaattaatttgtgaatgaaaatgcatcttaaggatggtgaggactggtggtagtggtggtgaatgtgcgtgtgtgtgtgtgtgattttgtggccatgatccaggcttactatgcatcagctcctaaagagatttttacagagctgtgtctgagtgtcttaattcaggtcatctcaaccagagagccttctgctactcctacgtgcaaaggctcgagttaactcagaaaaagttctcacagctgtggcccagatagattccaagacttctctgaaacttcagatggaagagcctttctctttaatagagagccattctctctattaaagaagagttgggcaagctggaggccagaggattatattgtaatcttcatgtgctgaaaggtgtaaagaatcttttcttcttaataagagcactgggacaagcatagggtgccttcaaattatcagacttcctcaaacaatagatcaattagcctttcttctgaatttgtgtaattgtttttgaggcaacaccctgaaaaaatatagcttactttttccgactagtataattaagcggacaaatggaaaaagaatcattcaaagtaaattttaaagaaaagagcaaaagagtaaaatttagcattgtccatgtaagatggataatgacaagtaaacacagaaggtggtgtttgatcccttcgattttggtattcattattcatagttattattcataaattccaggttaggcttgtgccccagtgacacatttgcattttactttaagttgatttctacctggttttgcaaacaaggatttaatgatttgggctgcctctcactcttcctctgttgcttgtcttttcatatacagtgctggcttctagccccttttacaccctttgtctccatcatcagtcttgctctcccatccaatgttttactctcactctctttctctcttctctcttctctctctctctctctcaatgttttaggaaaaaagatttctaacgtggaaaactgatatttgaatagagtcccctaaggctatcaatttacttttaatttgtctggagctaagcttttctttgttgcaatgaggaaggattaaaactgctgtaaaatttattagcttccagaaagatatttgaaatgcatttttttaatgattgtgctgtttgctgtacccagaagcagtgtcatcacaactatagtattccagatggttcccgaagttacactgaaacatcaagataatgctagacttttttatgacgcctctattggtaatgaatagtccatcccaggtagctaatatatattaattaatatatatttgtttccaatgaccatttctagtgatacccagctgatgacaaaacacacacactatccctttgaatcattacaggagacagtgattggtttactgagctctttattttccaaatgtggaagaaatgattggagatttactaatgctgtttcttagcttccccacccagtcctaggaacatattggtagtcaagaacctttaaggaatttagaagttacagttgcaacatttttcatggactcatgtatagtatggcctattctacaaaactgaatgtcctgggacttgactgaaggaagtgtcagttcctgttaaggggaacaccagctgctgggtgccctttttgttccattcaacagtaagttttcattagaaaagggaagatttttctcttggccaacgtgctctgtctactcttgctttctgtgtcagtcttatgtgtgccatcttcactatgtattctcctagaagctcatgaccatcatgaaagtggcttcttcacaagctgatgatctgctagcagtttccttattttccacaaaagagtgaaaccaagttggagataagtttaggttcagaactattttttaagtgtgataggctctgtgactgggcgcggtagctctctcctgtaaaccctgctactcctgaggctgaagtgggaggatcctttgagtccaggattttgagaccagcctgagcaacatagtgagattctgcctctaatttttttttttttttaattagccaggcacctgcctgcagtcccagctacttggaagaccgagccaggagaattcttggagtccaggagttggaagctgcaatgagctatgattgcactattgcactccagcttgggcaagagtgagatcttgtctctaaagaagtttttttttaatttttttattatttatttatcatttttttgtaaaggctctgtcatcctagatgtataactgcatgtagaatgcatctgacttttaaatctacaccaagtcaaataactgatttgaactcttcagagaagtgacaagatgttgtaggtatcccactaatataggtttgagggacttaattctgtgggcgtctttatatgaaactttcaaaaacggtcctatgtttttcagagttgtagtatctcagtgttctatcttttggggaatagctcattggccttctgtgtcacctcatatttactagtaaagttgctttttcaaatggtttataagcacctagatgaagtccagcactttgtcagattattttctccatcacagaagccaaaatacaaagatttcagtctagtcaatctctgcttatctcagtcactgtcagcacctcttatttcttagtctcaattttccaggcagccttttgcttgatttccttgctccccatgcatcagcacaaatgatttatacttatgtacatcttgaaatctcttctttatggattgaattgaatcgagccccagtacaaaaatgaaaactccaggaactgttgtgattgcccaaattacaacttggattgtatgactgtacacgccacactctcatctatcataaatattaaattctcaagcacttatacttcaatatgttttaactttgtctgtttgaccagctcagcactaaagattccattgaagataattcaattgctttgaattctgaaaggggcatctttttaatctattttcatgttgcactgggagttaagtgaatttcaggtgcatagcctgtgaatgatgggagaggcctatgtgccatttggcatcacagaggccaaatattgggtgacatttgctaactaatgctgatcagtgaaatggtctaggacaagattggggttcatcagtcttttcagccattctccctatctcaaactaaaactaaactttgcagaatttctgctgttttgtcagccatactgaaaatgccaagttatgctaaccagggctggccttttttcttctgtcttgtcttttggcaacatccctttgccagtctcaggaactttgttcccacaccccatccccatccttcatccttcctgaggcatctccacatccagacatttctagtgtttttaaccccatcaaatctctcttatcctggatacccttattcagggctctgttatgtgacccaaaaaaatctgataatctcagttcgataatggtgcctcttctaaacatttttttaaatttattttttattttaatagtttttgtggaacaaatggtgtttggttccatgaataagttctttagtggtgatttctgagattttggtgcacccatcacccaagatgtgtacactctacccaatgtgcagtcttttatctctcacactcctcccaccctttcccctgagtctccaaagtccattgtattattcttatgcctttgcatcctcatagcttagctcccacttattagcgagaacacataatgtttggttttccattcctgagttgcttcacttaaaataatagtctccaattctatccaggttgctgcatatgccattattttgttcctttttatggctgagtactatttccatggtgtgtgtgtgtgtgtgtgtgtgtgtgcatgcatcacattttctgtatcctctcattgattgatgggcatttgggctggttccatatttttgcaattgcaaattgtgctgctataaacatgagtatacaagtgccttttccatataaggacttcttttagatacctagtagtgggattgctggatcaaatagatacacttttagttatttaaggaatgtccacactgttttccatagtggttgtactagtttacatactcaccaacagtgtaaaagtattcccttttcaccacatccatgccaacatttattatttttttattatggccattcttgcaggagtaaggtggtatgacattgtggttttgatttgcatttccctgataattagtgatgttgagcattttttcatatgcttgttggccatttgtacatcttcttttgagaattgtctattcatgtccttagcccactttttgatgggattgttggttttcttcttgctgatttgtctgagttccttgtagattctggatattagtcctttgtcaaatgtatagattgtgaagattttctccctctctgtgggttgtctgtttattctgctgattatttcttttgctgtgcagaagctttaattaagtcccatctatttatgtttgtttttgctgtgtttgcttttgggttcttggtcatgaagtctttgcctaagccaatgtctgtaagagtttttccgatgttacctttagaatttttatggtttcaggtcttagaattaagtccttgatgcaccttgagttgatttttgtataagatgagagatgaagatccagtttcattcttctaagtgtgacttgtcaatgatcccagcaccatttgttgaatagagtgtctttcccccactttgtttttgtttgctttgttgaagatcagttggctgtatttacctttattcctgggttctctattctgttccattggcctacgtacttatttttataccagtatcatgctgttttggtgactatgcccttatagtatagttctagtcgggtaatctgaagtttccagatttgttcttttgtttagtcttgtttttgctatgtgggctcttttttggttccatatgaattttaggattgttttttctagttctgtgaagaataatggtggtgttttgatgggaattgcattgaacttgtagattgcttttggctgtatggtcgttttcacaatattgattctacccctccatgagcatgggatatggttccattggtttgtgtcatctataatttttttcagcagtgttttatagttttctttgtaggggttctttcacctctttgcttaagtatattccttattattattattttgcatctgttgtaaaagaggttgagttcttgttgattctcagtgtggttgctgttggtgtatagctgtgctactgatttgtgtacatttgattttgtatcctgaaactttgctgaattcatttaccagatctaggagctttttggatgactcgttagggttttctagatatacgatcatatcatcagcaaacagtgacagtttgacttcctctttaccaatttagatgccctttatttctttctcttgttggattgctttggctaggaattccagtaatttgttgaatagaagtggtgaaagtgggcatccttgttttgtttcagttctcagggggaatgctttcaacttttcgccattcagtataatgttggctgtgggtttgacatagacagctcttgttaccttgaggtatgtcccttctgtgctgattttgctgagggttttaatcataaagggatactggattttgtcaaatgctttgtctgaatccattgagatgatcatgtgatttttgttttaaattttgtgtgatgtgtcacatttattgacttgcatatgttaaaccatccctgcatgcctggtatgaaacccacttgatcatggtggatgatctttttgatatgctgttggattcggtcggctagtattttgttgaggatttctgtatctgtgttcatcagggatattggtctgtagctttctttttttgttatgtccttccctggttttggtattaggatgacactggcttcatagaatgatttagggaggattccctctttctctgttctgtggaatagtgttaataggattggtaccaattcttctttgaatatctgatgaaattcagctatgaatctgtctgactcttgttgacaattgttttattaccatttcaatctcactgctcattattggtctgttacgagtttctatatcttcctggtttaatctaggggagttgtatatttccaggaatttatccatttcctctaggttttctagtttatgcacataaaggtgttcatagtagccttgaataatcttctgtttctctgtggtatcagtagtaatatatcccgttttgtttctgattgagcttatttggatcttctctcttcttttcttggttaatcttggttaatcatttttattcatttttttcaaagaaccaggtttttgtttcatttatcttttgtattatttcttttgtttcaatttcatttagttctgctctgatcttggttatttcttttcttgtgctgggtttgcatttggattgttcttgtttctccagtttaatgaggtgtgaccttagattgtctatttttgccttttcagactttttaatgtaggcatttaatgctatgaactttcctcttagcaccgcttttgctgtatcccagaggtttggataggttgtgtcactattatcattcagttcaaagaatttttcagtttccatcttggtttcattgttgacccaatgataattcagaagcaggttatttaacttccatgtatttgcatggttttgaaggctccttttagagttgatttccagtttttccactgtggtctgagagtacttgatatcatttcgattttcttaaatttactgagacttgttttgtggcctatcatatggtctgtcttggagaatgttccatgtgctggtgaatagaatgtatgttctgcagttgttgggtataatgttctgtaaatacctgttaagtccatttgctgtaggttatagtttaagtccattgtttctttgttggctttctgtcttgatgacctgtctagtgctgtcagtggactattaaagtcccccactattattgtgttgccatctatcttatttcttaggtctagtagtaattattttataaatttgggagctccagcattaagtgcatatatatttaggattgtgatattttcctattggactagtccttttatcgttatgtaatgtccttttttgtctttttaaactgctgttgctttaaaatttgtctgatgtaagaatagctattcctgctcgcttttggtgtccatttgcatggaatatctttttctaacccctttaccttaagtttatgtgagttcctatgtgttagttgagtctcctgaagacagcagaaacgtggttggtgaactcttattcattctgccattctgtatcttttaagtggagcatttaggccatttacattcaacattagtattgagatgtgaggtgctattctattcactgtgcaaattattgcctgaatacctttttttcattgtgttactgttgtatagttcctgtgagatttatgctttaaggaggttctattttcatgtatttcaaggacttgtttcaagatttagagctctttttagcagttcatatagtgctgtcttggtagtggcgaattctgtcagcatttgtctggaaaagactgtatctttccttcatttattaagcttagtttcactggatacaaaattcttggctgataattgtttcatttaaggaggctaaaaataggaccccaatcccttctagcttgtagggtttctgctgagaaatctgctattattctgataggttttcctttgtaggttacttgatgcttttgactgacagctcttaagattcttttcttcgtcttgatttcagataacctgatgactatgtgcctaggtgatgatctttttgcaatgaatttcccaggtgttctttgagctccttgtatttggatgtctagatctctagcaaggctggggaagttttcctcgattattccctcagatgtgttttccaaccttttagatttctcttcttccttgggaacacccattattctttggtttggatgcttaacatagtcccaaacttcttggaggttttgttcattttttaaaattctttttctttttctttgacagattgggttaattcaaaaatccttgtctttgagctctgaagttctttcttcttttgttcaattctattgctgagactttccagtgcattttgcatttctctaagtgtgtccttgatttccagaagttgtgattgttttttgtttatgctatctatttccctgaagacttttcctttcatatcttgtgtcatggttttgatttctttaagttggacttcacctttttctggagccgctttgattagcttagtaattgaccttctgaattttttttctggcaattcagaaatttctccttggtttggatccattgctagtgagctagtgtaatcttttgggggtgttaaagaaccttgttttgttatattaccagaatactttttctgcttccttctcatttgtgtagactgtgtcagagagaagatctaggactcaagggctgctgttcatattcttttgtcccatggggtgctcccttgatgtggtgttcttccccttcccctggggatggggattcctgagagccgaactgcagtgattgttatttctcttctgggtgtagccacccagtggagctactgggctatgggctggtactggggagtgtctgcaaagagtcctgtgacatgatctgtcttcaggtctttcagccgtggatatcggcacctgctccagtggagttagcaggagagtgaagtggactctgtgagggtccttggctgtatttttgtttagtgcactggctttgtgttggttggcctccagccaggaggtggtgctttcaagagtgtatcagctgcagtactatagggtggatgctggatcatcttattttaacaggcattttttaatgcatgtatccagatgaatgaaaaatgtggaaatttttctccatgctgcaagggctggttcttggatagcacttctttggcatctgtcaggtaacaccacataggtgttttgaatgccaatcagtgctggcatcctttcccttctgcctgatgtctgtctgtcattccatagtgttgtaggggagggctgctaacaaggacgaggctagggaacgggagaacaaaaagaaagttttagggagcaaccaaaagtggagaaaattgctgaaaaaaagggaagaagggagtgagaaagagacaagtagttgtttgtagtgtaatttgtgatgtgcttcttgtcacatagagtgactacaggatagcttggtatcattggttcatgagtgtctacagatatctcagccttcactggtgttcccttgaatgttttacggatggaattccatcttttttttgctaaaattttactttagaatacgactgtaaaccccctgcaacattacagcctgggagggctctaggaagttgtcattacgctcatattgaatttttgctagatacataacagatattttccatatgatatctaaacacccaaataagcccaatcctcaagatatcaaacatatcaaaaccaaaataaaatgcctaaagccttggtaatattttccttccttccttccttccttttttccttccttcctttcttccttccttccttcttccatttccttcctttcttttccattgatcccgttgctgctttacttgccttgttaagttacagagatgggattgataatagtgcaggagagagcactattaactattgttagtatagaaaacttgcaggtctatggcaaatgtattttcaacgggatgttagagggtactagatctctctaacacctgtatatagtgtgcaggaaaccacacagaggctaagaggacacttaatgtttctcaagttctaaatttactttacgcctctgacaaagctacatagtttactttgtaggtttctgtagtatttatgataaaatagttatttccactttaggagttgcacagtatctggggtgggacagtatctggggcgttgaccaatattcttccttctttctgtgcatttttcattcaattaaattaaactattattaacagtttaattgacagctctgtgctggacgctttgctaggttgagatagatgtaatctttgctcatccagagcttaaaaggtagcagaaaatatagacatgaaacaagtacagttgtctcttggcatccatggaggattggttccaggactcctgagaatacgaaaatctgtacatgctcaagtcctgcagtttgccctggggaatctggtggaacctgtggataaatgaaaaggtgaccctacaaatctgagggttttgcatcccacagatgttgtattttctattcacattttgtggcagatgcagaacctgcagatacagagggctgaccatatttatttttaaaaacctacatgtaagtggacccatgcagtttcaaacccatgttgttcaagggtcaatggtaattacgcgtgtgatgaatgttataaaagggaagtgctgtgagatctaccttagtctaaaggatcacagaaggactccctggggaagtgaaatttaaatggagacttcagggataatgtattaatcatgaattgtgtttaaccgctagtaataaagatgcccctctcctactaaagaaaataaatcaatgacttaaataagagatatttatttttcttcttattaaagaagttcaagtttggtgagggtagctctacagtatctttaataacccaaggtccttatatctttctattctgccacccacaggcacatggcttctatcttcagggttgcctcatggtagtcagacagatgttgcagcatcagtcattatgtctccattccagacaggacaaagcaggaaggaataaagacaaaatagtctcttacctaagtcagctctccccttttaaggaattttcttaacatcttgctttataatgttggtccaatcttatgttacatgtctgcaagggagcctgtgaaatgtagtttcttttagctgagcacactgccattcctaacagaaatggggctttgtttctatttaacaaaaacagataaagatatttggcaggcaattagttatctctgcttcatagagtaggagttagataggtcaagggtgggtggtgtacttctcaacaattaggtttcctcgattaggcacatgtgaccaattttaacagcactaaacttcttcataatcattgtcataatctcctccattatatctcacaaaaaatggccctgctttggttacagattgtcttgttcatgaggtgatgcagaataaactaagtagatgtataagagtacaattcaggaagcttgtctgatatacacatcacttgcttgttggttctactactgctgccacttagaagtagccaactacttgagctctcaggaactggaactcactttaacttttttctttctgattcattgattgcagattatttctgaagaggaaacatattgctcttgtaactgtaaccttgaactattttagtgctggattttcgtctgtgatcattaggatctttgagttaatacacccaaactctgaatcacagtaaagaactgccagcattatcaaacacatccattgtgactggatatctatacaaacccagcagagcctcttagctcttctattccaccaggacttggaaagttttagcaaagcctgaaatgatgttttcatcattttcttttattactttggtggggaccatgggtaggctctttctagtgtggaagtacattgtttggccacatctgaggagatggcagtttcatgtcctgaggagagtagtaattgctgcataaagttttgctattatctgcacaaaatagcatattctggtctttttctgtttgaattacttccttaatctatcatgacttagtcattactgccttactgcctcttttgcctcttgtaccctcgcaagactgatgctttaattttttttttttttttggccaggggttttggaattctctcaccaagtttcctgatgacaaattgtgaccaccaaatacttactgctatgtagattgaagaaactttcacttttttctgtattttttatggtctaaaataatcagatttgttttgaactaaggagttctctggtctcaagatttgtgttcctcttccaaatttatgcactctgttcttgtttcttttgcaaatcctagggacaacttcagaagcagggaaaaccaatttacaaattcatcagtcaaccctcatcatgagtatgtttaaattaaagacacctgccttcattcatctatacgtgaatttccacaactatatgtggattttagaatgccacattttgctttgcctaggttcaggtcagaactttcagataaaaatgtagcaaagtatcaggtcacagataggttcataagaagaatctcagcaggaacttagtgaatcgtgagcccatctctctatataagctggagccagcaccttgacagtttgtgatgctacaaaatggttcttctctagtctcatagctccattcccagccactatactacatgaggactggcacataatctttacaagtcctatacaattacagaagtagtctttttaatttttatttttttacatctggtctgtcttcattgctatttttaaggggctttgtattgtgactttctcacatttactacacaatttctttactaaactgaaattttgatttttgaaaggtaattcatctgcagtatatttatgaatgtaaattaccctgtgctgggcagagatgttcttcctgcctgtctatattgacaatttttatttcatatataaagtggctataaacatatttaagctaagttagacttcattgtaactgtatagacaaaaatgtcacaaaatatctgaaacacaaaaataagaaacagtaactcagtgcccttggtaaggacttaaaaaaaatataggtatcagatccaagccaaggatgagacactgtgagtaaaagagtctcttagattcaagtcttcacttgcgtgaggaagggcagtgcatataataagtttacataagtaatctgagaaaactgcatatgattccagctcatatggagctgaggtaggctcagtgagcttggtggggaggcttagactgattgcattgttgggtgtgtcctgagttagtctccgtttctcttttcccaacagtgattgtttcatttagacagtgggtcagctgaaggcaagtactatctatacagagaaacctcagagaagattgaattccctgctctggtcttccaagatttataagatcttggaggtgtctgtctctgggctctctattctccctccccagatattctggggatccaagtctcctcatgctgttcatttgaggcagcctagtatagggaaggagcattgtacttggattcagggtgacacatgggtggtattctcctattgaatgctgttgaaaaaaactaaatctcaaaattagaggacagtcacttcatattaaaaaactcaaagaaataagagcttcgtgttgaagacattgggcagatggtactcaattgtacttcactactgctctgctctagaagtgtgagcaagtggcctgaaatatcagtcttaatcttggctgggattttcttttcttagttggtcatatgtccatggtttcagggctcaagggttaggtctttatgcttttatgcctttcaggtagggacaaaaagggcagagatcttatagccttctggacatttttttgctggcttccagggatttttactctgatttgtagaaatggaaataatttattgaacttttcccccacctgcaactgcttactctctctaccctgaaacactctcactaattttttagaggtattatttggaaagttaccagataggccttggaagagatgaagaggcaaaaacagcttattgggaagccctaaagatgtgacttctgttctactgaagggatcttgggacctcctgtggaaagcactatatttggaggaaactgattaaaagtagatcaactaactcttccactcatactgtttagtctacacaaagtgcttctcaagtatatcccaaaatgcttcccttactttcatattttctgcctcccaggtacatgcatgttgaatgataacagggtatcagatggcaacacagagagtttgcagggaaggggcggagaaagggccttgacagacctttcattttgcagaagtggccatgcagcctgatgtggaactgagctggagtttgcagaaacattgtcctaactaggtggaaggcactttccctgttcatgatgttgttcatttgttatttggttccactgtacaaaattctgagtgtttcattgcccagcactgaaagggggatgaaagtgtccatcttaaagataaattggcaaattatgtcaaggtacactgaagaggtcttgtctttagtttgcttttaagtagcttaaatgaggtcaagggccatgcccagaattgtgttgaaatctggatttagcttggattaggtccttgatatttaacaggcttctaatgtctttcctccaaataatgaagtgtacagccaattcaaccattattccttgagccctctgccttgtgccccttcaccaaagaacctttgatgaatgaggcatagcagtaccttgctttgctatgccatttactctttaaaaacaatgaagaagcaaacaaaaaacaccaaaaatatagaaatacaagtatacaaggaagatgtgttatatatagactgatgccaagtaagactctgaattgcagacatcttccccctgcaaaataagtcacaacataaatatatatattttatatacatatgtatatataagatatatatatgtattttctgtaaatgttaagctgctcatattttgatgcattgcgcaatgttttcattaggtcatcctcttgtaagaatgtaacataaatataaatagatctggaaaactagggctataaaacaaacattgagattaacagttttccagtaacaggcagaagttaaccctgggtccatgccttggtaaagcctgtgcagtaagcctctctgagagccagattgaagctttaacccttagtgaacctgacctggaaagataaatctatgtctgcccaataattatgaattgtaactgctgagatcttccaggatggagttaaaataatccatgaaagatttctttaagaaatatttctctaacaaaactgcagataagaaagataaggagggatggctagttgggtctaattaacaatgaccaacatgtcactgatgtttgcgtagggccaattgaagcttatgggtcagctggatagaagggctctcagtttattccttctgatgggaaatcaagtcatggaagtggcatagtgaggagattggctggtgggactggtggtcaaaaagaaggggtgtgattgggctttcacagagagtaggatatcaatctaaaattaaattaagcactgtgtatcagtgatgggtagggtggaagggtgtttggcagtgctgttgttcaaaaatatggccaaggcttcttaaatacactgactgttggattcccttccctgcctccactccctcatctgctgaatccagtttcaaacacaggtgatttttagcaggagttgttgattaaaatgctgactctcagcaccttgacactccagatgaactttagaatatatatgtattttcatcataacgtgtttccctttaggttttttgttttgtcagtagtgggaaccttaaaaaggatatacatagagggagatcttcagccttttaaaaaaatccagttcttcacacttgtcccaaaaggcaataaaggactacctaaaagtgccatttcccacaagggcctgtggatatctaagaaaccagaacactggggagtaacagtgaatcctgggaaaagctgatgaaattgatattttcttttttccaaacagatcagaatgaaggatttcattgccgggaagaatgccggattcttggccactctgacaggtgctggatgccccggaaccccatgcccatccgttccaagtcccctgagcatgtgaggaacatcatcgcgctgtctattgaagctactgctgctgatgtcgaggcttatgacgactgcggccccaccaaacggactttcgcaacctttgggaaagatgtcagcgaccacccggctgaggagaggcctaccctgaaaggcaagaggactgtcgatgtgaccatctgcagccccaaggtcaacagcgttatccgggaggcaggcaatggctgtgaggcgattagccctgtcacctcccccctccacctcaagagctctctgcccaccaagccttccgtgtcttacaccattgccctggctcccccagcccgtgatctggagcagtatgtcaacaatgtcaacaatggccctactcgtccctctgaagctgagccccgtggagctgatagcgagaaagtcatgcatgaggtcagccccattctgaaggaaggtcgcaacaaagagtcccctggtgtgaagcgtctgaaggatatcgttctctaaaccagtctccaggaagaagagaaagaaaccacactggctagtgaagaagcaggagcttcttgttttaattgctcaccaatggttggttcttgagtggctatatttcagagcttttcctaaatgtattgtttataggtgattatcattctgtgacagtcccttgtttcaacaggcagcaggggtgttcagttggagcaaattagctttggcttgagttgttcatggggccttgatgttggggaaacagagacaaattcagttgtgaaaagtattatgtattaagtgtttgaatttatatatttttctatgtcaaaattataatataaattaccattgtttgtggaggattacatttaaaaaagcaaaaagtgaaaaaaaaaagctctggacacctttaataagctcgccactgttttttgtagtgtagacaagttaatggtcatttattgtgtactattcattgattcagtgatgtgaaattgagcccccaaaaggttgtttctgaagctcgagtacttcccaatgccgctactttgctgtggacacccctgctttaaaaacccttttgctagctgtgctattgttgtatttgatattgcaaattgctatgtgtgtggtgatggcaaaaggcttttaaaagttggtgttttctttttcttaaaaaaataataaaactacagacaaaaacaaagtgcaaacaactgagacagcaaatgaatgagcaacaccatgcatatagattgagtacttggagaatactcacgttttttaactcatgtagtgattgctcaccactttgcaaagtatttttcctgcctttcagtggtctgcccaggtccatgatagatcattgcagaaagtcatttttggataggctgctatctaatttgcagttgtcagaaatactgtgctgaaagttttatgacatccatcttttaaattttcaggccctgaacaggaaagttgctcctgaagttatgtttccaggcttagaaattccctctctccaatcatctaaaattgaagatgactgaatctattttaagatctaaattagcatctcttcagacacacacttcctgattcagtgtttcccaatcatgattagaattagactgcaaagcacgtcatgggctgggattgagaactccatgttgctctgtatcttaggcttatatacgtagggatagagtaagcagtacaaagtgtatattttggaaaagacaggtaacaggtaacagcatcgctaatccaattactgtgccttctaaacggagacactcagacacttgaactcatctttttatgactaatagttttttgattaaaaatgtgaacaagaaaatactaaaataaaaatcctttcgatttagccaactctttttgctgtaggctaggaatacaccctttttaaattaacacactgtagatcctttttttctgttttaacattcctctaactcccccatttatcttttgtctattaatattcactagccaacatagtatttttgcagcctaagagttcattatttaaaaataaactaaagaaatatgtcacctttgttctgccttggtcttaagagagttgtttctagagaaacaagttttatggttctgttttcatttgtttcattttttgaaattcaggaatacacagagagaaggcctagaggttagagcactagaatgcaaaagaggatttattttcttaagtttaggtagataagtcagctcttttgaatgttttacttatttttgcctgagttcctagctttacgcattactaggaatattttcttttacaggagggagaagggttttgagggagggggtgggtaaggcagtagggatggggttaggtaggggagaacatttctgaaaaagaacttataatgaagctacaaatccatccttatttcttattcagtgctgaatactacctcatgcaaaatattggtgtggctgcaaatttccctctgaagctgacacaattaaggatgaattaccataacatcttcatttttcctcatttcattgcctctcatacagttttctccctctgatttatttcattttggtagtggattttgaattaagtatttatttctctttgcaagtgactatttgattaacacattaaaaattttttaaaaatttcccctttaagttatgatggctgcctatagaatttagactgtttctcaccctgatccatcctgatattatgttattagctacgatttcaaggtcactttgaagtcagacttcacagttttctacaggtgtatttcttgctatgctcagtgggaaccagggagcagaaaagattgtggaaagtgggaacattaattgtcacgtggctgttgagccagagaggccactggctgccatgcctcttaccagccaagttttaaatggttagagtggcattggcattgattttgcttttctcttgggttttcttcatttcttttcataattcatcttcaagctatagatccatccttcttccctatccaaatactttgttctcagtgggcagtcagatttttttggcttccaaacctcactgagcatgttcagagaattcctctcattaaggaaatatttttccccacaattgtctgatgtatgagtcctctcagaagcacaagtgcatactaggaaggctttgtaaactggttttgtaggcaggctgtacctgcaaataggcattctgagcaaggatcctaaatttccctttggctcatgtcccccccgccccaccccaccttctcctttttggcatgtctggtgaaaagctacaaggttcacagggccaaatattctcccttttgttctctaaacatgttgcacacataaactcaagggagtgatttcggttcctaagtcctagggagttctattccatttattctgatgtcccttttaaggaggagttggtgttgaagtgtgacatgctttaatgataactgctttggcaattccccagacaggtatacttcttcatccaaaattgcacctaaggtgttcatttagacaaaattgttcgcttagtgatccactgatctctaatcagaaatatttttaaaaaatacaatttagatctctgaaaattggaaccactcaagagtatcatcttgatagtgttacttcagcatatgactgactgattttatgaagggagtttagctgacttttccctgtagccctatcatattaaaaacaaaaagtggtccacttgaagcagcaaggacaaagggcagtttagggagaggcagggtgaaggggatagctcgattgggcatccagagtggaaaaggattattattgtacataatagcagttctggctggaattccaatccactttctggggatcattttgatgttgaatgttcccccaaagcattctgacaagtttggtccacttggctctttaccccgggtaataagacccttattgactttgaaaaactggcataaatgagcttaagaaatccattgtatacagtcctagtgtttgccttcttggtaaaattacatttaaggatgtcatttcatgagtcaaattgtatctgctgacactgaagtgtattgagatgttgttctttagtttaagtatgtaacaaagtaatattcaaaggggatgtttgggggtggggctgggaccgggtggctttatattgataatgaaatattccagagcaaaacacattgtcctaatgaagttgttgcagctctcttcgttttcttgtgccgctggaattggcttgtgggtcacttgttgtgcaaaagtgaattaatttcaaataggaatcatattgcttttgtgaaacaaagaaaatttaccaccttgtctgatacatagattggaaggcgctcttggtaaagaataacccacaaacacctgatcttgattggaaagcattgtaggtcattagggaaaactgatgatgctgactgttgctccagctgtgttggcaattcaagatgaccttgttactaactgaaagttgctgtcaaccttagcaaacaaaccagctaacaccattacagcactggttaagtagatttttactggcactggctggcgttcagctgaggaaggctaggatacagactttaatgaccaagagccagactagctcgagctagaaatcagcaacaaatacctccttgtgctatttgaatgctaaggtattagttgcaataggcttacgccttcgctcctctagtgaggggaatgagcctgatgacactattactgtttacctgtcaaaccagggtggaaatttagagggatgcaagtacagtatgtctcattaagcagtctctgttgctgattatatgaagataaaattgttgtatatgctgatctgtatgttatgtacattttcacagtgattgaatcattgtaaaaagaaaaaagacaaaaaagaatcaccactgtctattttatgaagatgataaagcagctttattaaattattacagttatgtttcaaatggtgttatctgccacattgtttccttttatctggtggttctctgtttccccctttaaatgtgttttctctgcttcaaaaatttaatgcattttagtattaaaggctattatggaaaaagta

SEQ ID NO: 2 - DNA, 15 bp, synthetic construct
  source 1..15: mol_type = other DNA
atcaccgtcccggcg

SEQ ID NO: 3 - DNA, 16 bp, synthetic construct
  source 1..16: mol_type = other DNA
aatcaccgtcccggcg

SEQ ID NO: 4 - DNA, 17 bp, synthetic construct
  source 1..17: mol_type = other DNA
caatcaccgtcccggcg

SEQ ID NO: 5 - DNA, 20 bp, synthetic construct
  source 1..20: mol_type = other DNA
cgttggcaatcaccgtcccg

SEQ ID NO: 6 - DNA, 20 bp, synthetic construct
  source 1..20: mol_type = other DNA
ttggacaccacgcgaaaggc

SEQ ID NO: 7 - DNA, 20 bp, synthetic construct
  source 1..20: mol_type = other DNA
agttggacaccacgcgaaag

SEQ ID NO: 8 - DNA, 20 bp, synthetic construct
  source 1..20: mol_type = other DNA
gagttggacaccacgcgaaa

SEQ ID NO: 9 - DNA, 20 bp, synthetic construct
  source 1..20: mol_type = other DNA
cgagttggacaccacgcgaa

SEQ ID NO: 10 - DNA, 20 bp, synthetic construct
  source 1..20: mol_type = other DNA
cctcgagcgagatgatgcac

SEQ ID NO: 11 - DNA, 20 bp, synthetic construct
  source 1..20: mol_type = other DNA
ggacatgacctcgagcgaga

SEQ ID NO: 12 - DNA, 17 bp, synthetic construct
  source 1..17: mol_type = other DNA
actgggcgcattgtcgt

SEQ ID NO: 13 - DNA, 16 bp, synthetic construct
  source 1..16: mol_type = other DNA
actgggcgcattgtcg

SEQ ID NO: 14 - DNA, 15 bp, synthetic construct
  source 1..15: mol_type = other DNA
ggatgcgcgtgccag

SEQ ID NO: 15 - DNA, 20 bp, synthetic construct
  source 1..20: mol_type = other DNA
taagcgctgtccagcgggat

SEQ ID NO: 16 - DNA, 20 bp, synthetic construct
  source 1..20: mol_type = other DNA
ggatcgtaagcgctgtccag

SEQ ID NO: 17 - DNA, 20 bp, synthetic construct
  source 1..20: mol_type = other DNA
ctggatcgtaagcgctgtcc

SEQ ID NO: 18 - DNA, 20 bp, synthetic construct
  source 1..20: mol_type = other DNA
tctggatcgtaagcgctgtc

SEQ ID NO: 19 - DNA, 15 bp, synthetic construct
  source 1..15: mol_type = other DNA
ggatcgtaagcgctg

SEQ ID NO: 20 - DNA, 16 bp, synthetic construct
  source 1..16: mol_type = other DNA
tggatcgtaagcgctg

SEQ ID NO: 21 - DNA, 17 bp, synthetic construct
  source 1..17: mol_type = other DNA
tctggatcgtaagcgct

SEQ ID NO: 22 - DNA, 20 bp, synthetic construct
  source 1..20: mol_type = other DNA
gagtctggatcgtaagcgct

SEQ ID NO: 23 - DNA, 16 bp, synthetic construct
  source 1..16: mol_type = other DNA
tctggatcgtaagcgc

SEQ ID NO: 24 - DNA, 17 bp, synthetic construct
  source 1..17: mol_type = other DNA
gtctggatcgtaagcgc

SEQ ID NO: 25 - DNA, 20 bp, synthetic construct
  source 1..20: mol_type = other DNA
tgagtctggatcgtaagcgc

SEQ ID NO: 26 - DNA, 17 bp, synthetic construct
  source 1..17: mol_type = other DNA
agtctggatcgtaagcg

SEQ ID NO: 27 - DNA, 20 bp, synthetic construct
  source 1..20: mol_type = other DNA
gctcgtaagtctgcacgcca

SEQ ID NO: 28 - DNA, 20 bp, synthetic construct
  source 1..20: mol_type = other DNA
ccgaacagctcgttgggcgt

SEQ ID NO: 29 - DNA, 20 bp, synthetic construct
  source 1..20: mol_type = other DNA
gccgaacagctcgttgggcg

SEQ ID NO: 30 - DNA, 20 bp, synthetic construct
  source 1..20: mol_type = other DNA
ggccgaacagctcgttgggc

SEQ ID NO: 31 - DNA, 15 bp, synthetic construct
  source 1..15: mol_type = other DNA
actgcgtctcgcggt

SEQ ID NO: 32 - DNA, 20 bp, synthetic construct
  source 1..20: mol_type = other DNA
gtgcgactgcgtctcgcggt

SEQ ID NO: 33 - DNA, 20 bp, synthetic construct
  source 1..20: mol_type = other DNA
gaagctgtagtgcgactgcg

SEQ ID NO: 34 - DNA, 16 bp, synthetic construct
  source 1..16: mol_type = other DNA
tagcgcagtgattcgg

SEQ ID NO: 35 - DNA, 20 bp, synthetic construct
  source 1..20: mol_type = other DNA
cgtctagcgcagtgattcgg

SEQ ID NO: 36 - DNA, 17 bp, synthetic construct
  source 1..17: mol_type = other DNA
cgccaccgtctagcgca

SEQ ID NO: 37 - DNA, 16 bp, synthetic construct
  source 1..16: mol_type = other DNA
tcgccaccgtctagcg

SEQ ID NO: 38 - DNA, 17 bp, synthetic construct
  source 1..17: mol_type = other DNA
gtcgccaccgtctagcg

SEQ ID NO: 39 - DNA, 17 bp, synthetic construct
  source 1..17: mol_type = other DNA
ggtcgccaccgtctagc

SEQ ID NO: 40 - DNA, 15 bp, synthetic construct
  source 1..15: mol_type = other DNA
gtcgccaccgtctag

SEQ ID NO: 41 - DNA, 20 bp, synthetic construct
  source 1..20: mol_type = other DNA
cggcgggtcgccaccgtcta

SEQ ID NO: 42 - DNA, 20 bp, synthetic construct
  source 1..20: mol_type = other DNA
cgctaaacaccgggttgttg

SEQ ID NO: 43 - DNA, 15 bp, synthetic construct
  source 1..15: mol_type = other DNA
ctcgctaaacaccgg

SEQ ID NO: 44 - DNA, 20 bp, synthetic construct
  source 1..20: mol_type = other DNA
gtggactcgctaaacaccgg

SEQ ID NO: 45 - DNA, 20 bp, synthetic construct
  source 1..20: mol_type = other DNA
ggtggactcgctaaacaccg

SEQ ID NO: 46 - DNA, 20 bp, synthetic construct
  source 1..20: mol_type = other DNA
cgttgaggcggatgacgggt

SEQ ID NO: 47 - DNA, 20 bp, synthetic construct
  source 1..20: mol_type = other DNA
ggtcgttgacgtagccatag

SEQ ID NO: 48 - DNA, 20 bp, synthetic construct
  source 1..20: mol_type = other DNA
cggtcgttgacgtagccata

SEQ ID NO: 49 - DNA, 17 bp, synthetic construct
  source 1..17: mol_type = other DNA
gtcgttgacgtagccat

SEQ ID NO: 50 - DNA, 20 bp, synthetic construct
  source 1..20: mol_type = other DNA
gcggtcgttgacgtagccat

SEQ ID NO: 51 - DNA, 17 bp, synthetic construct
  source 1..17: mol_type = other DNA
ggtcgttgacgtagcca

SEQ ID NO: 52 - DNA, 20 bp, synthetic construct
  source 1..20: mol_type = other DNA
tgcggtcgttgacgtagcca

SEQ ID NO: 53 - DNA, 16 bp, synthetic construct
  source 1..16: mol_type = other DNA
ggtcgttgacgtagcc

SEQ ID NO: 54 - DNA, 17 bp, synthetic construct
  source 1..17: mol_type = other DNA
cggtcgttgacgtagcc

SEQ ID NO: 55 - DNA, 20 bp, synthetic construct
  source 1..20: mol_type = other DNA
gtgcggtcgttgacgtagcc

SEQ ID NO: 56 - DNA, 16 bp, synthetic construct
  source 1..16: mol_type = other DNA
cggtcgttgacgtagc

SEQ ID NO: 57 - DNA, 20 bp, synthetic construct
  source 1..20: mol_type = other DNA
cgtgcggtcgttgacgtagc

SEQ ID NO: 58 - DNA, 20 bp, synthetic construct
  source 1..20: mol_type = other DNA
gcgtgcggtcgttgacgtag

SEQ ID NO: 59 - DNA, 17 bp, synthetic construct
  source 1..17: mol_type = other DNA
gtgcggtcgttgacgta

SEQ ID NO: 60 - DNA, 20 bp, synthetic construct
  source 1..20: mol_type = other DNA
cgcgtgcggtcgttgacgta

SEQ ID NO: 61 - DNA, 16 bp, synthetic construct
  source 1..16: mol_type = other DNA
gtgcggtcgttgacgt

SEQ ID NO: 62 - DNA, 17 bp, synthetic construct
  source 1..17: mol_type = other DNA
cgtgcggtcgttgacgt

SEQ ID NO: 63 - DNA, 16 bp, synthetic construct
  source 1..16: mol_type = other DNA
cgtgcggtcgttgacg

SEQ ID NO: 64 - DNA, 17 bp, synthetic construct
  source 1..17: mol_type = other DNA
gcgtgcggtcgttgacg

SEQ ID NO: 65 - DNA, 15 bp, synthetic construct
  source 1..15: mol_type = other DNA
cgtgcggtcgttgac

SEQ ID NO: 66 - DNA, 17 bp, synthetic construct
  source 1..17: mol_type = other DNA
cgcgtgcggtcgttgac

SEQ ID NO: 67 - DNA, 15 bp, synthetic construct
  source 1..15: mol_type = other DNA
gcgtgcggtcgttga

SEQ ID NO: 68 - DNA, 16 bp, synthetic construct
  source 1..16: mol_type = other DNA
cgcgtgcggtcgttga

SEQ ID NO: 69 - DNA, 20 bp, synthetic construct
  source 1..20: mol_type = other DNA
tgtgcgggtcgatctgaaag

SEQ ID NO: 70 - DNA, 16 bp, synthetic construct
  source 1..16: mol_type = other DNA
gtgcgggtcgatctga

SEQ ID NO: 71 - DNA, 20 bp, synthetic construct
  source 1..20: mol_type = other DNA
tcgtagtctaaagcgccagt

SEQ ID NO: 72 - DNA, 20 bp, synthetic construct
  source 1..20: mol_type = other DNA
ttcgtagtctaaagcgccag

SEQ ID NO: 73 - DNA, 17 bp, synthetic construct
  source 1..17: mol_type = other DNA
cgtagtctaaagcgcca

SEQ ID NO: 74 - DNA, 20 bp, synthetic construct
  source 1..20: mol_type = other DNA
cttcgtagtctaaagcgcca

SEQ ID NO: 75 - DNA, 15 bp, synthetic construct
  source 1..15: mol_type = other DNA
gtagtctaaagcgcc

SEQ ID NO: 76 - DNA, 17 bp, synthetic construct
  source 1..17: mol_type = other DNA
tcgtagtctaaagcgcc

SEQ ID NO: 77 - DNA, 20 bp, synthetic construct
  source 1..20: mol_type = other DNA
tcttcgtagtctaaagcgcc

SEQ ID NO: 78 - DNA, 15 bp, synthetic construct
  source 1..15: mol_type = other DNA
cgtagtctaaagcgc

SEQ ID NO: 79 - DNA, 16 bp, synthetic construct
  source 1..16: mol_type = other DNA
tcgtagtctaaagcgc

SEQ ID NO: 80 - DNA, 17 bp, synthetic construct
  source 1..17: mol_type = other DNA
ttcgtagtctaaagcgc

SEQ ID NO: 81 - DNA, 20 bp, synthetic construct
  source 1..20: mol_type = other DNA
ctcttcgtagtctaaagcgc

SEQ ID NO: 82 - DNA, 17 bp, synthetic construct
  source 1..17: mol_type = other DNA
cttcgtagtctaaagcg

SEQ ID NO: 83 - DNA, 20 bp, synthetic construct
  source 1..20: mol_type = other DNA
gtgcccctcttcgtagtcta

SEQ ID NO: 84 - DNA, 15 bp, synthetic construct
  source 1..15: mol_type = other DNA
accggcggattgtca

SEQ ID NO: 85 - DNA, 16 bp, synthetic construct
  source 1..16: mol_type = other DNA
gaccggcggattgtca

SEQ ID NO: 86 - DNA, 17 bp, synthetic construct
  source 1..17: mol_type = other DNA
tgaccggcggattgtca

SEQ ID NO: 87 - DNA, 17 bp, synthetic construct
  source 1..17: mol_type = other DNA
ttgatgaccggcggatt

SEQ ID NO: 88 - DNA, 20 bp, synthetic construct
  source 1..20: mol_type = other DNA
aggttgatgaccggcggatt

SEQ ID NO: 89 - DNA, 15 bp, synthetic construct
  source 1..15: mol_type = other DNA
tgatgaccggcggat

SEQ ID NO: 90 - DNA, 16 bp, synthetic construct
  source 1..16: mol_type = other DNA
ttgatgaccggcggat

SEQ ID NO: 91 - DNA, 20 bp, synthetic construct
  source 1..20: mol_type = other DNA
caaggcgatcacgtagcccg

SEQ ID NO: 92 - DNA, 20 bp, synthetic construct
  source 1..20: mol_type = other DNA
cgcaccaaggcgatcacgta

SEQ ID NO: 93 - DNA, 20 bp, synthetic construct
  source 1..20: mol_type = other DNA
ccgcaccaaggcgatcacgt

SEQ ID NO: 94 - DNA, 17 bp, synthetic construct
  source 1..17: mol_type = other DNA
tcgcgatcagacacccg

SEQ ID NO: 95 - DNA, 20 bp, synthetic construct
  source 1..20: mol_type = other DNA
gagtcgcgatcagacacccg

SEQ ID NO: 96 - DNA, 20 bp, synthetic construct
  source 1..20: mol_type = other DNA
ttggtcgtgctgctcgcggt

SEQ ID NO: 97 - DNA, 20 bp, synthetic construct
  source 1..20: mol_type = other DNA
attggtcgtgctgctcgcgg

SEQ ID NO: 98 - DNA, 17 bp, synthetic construct
  source 1..17: mol_type = other DNA
attggtcgtgctgctcg

SEQ ID NO: 99 - DNA, 20 bp, synthetic construct
  source 1..20: mol_type = other DNA
tgaggttgtattggtcgtgc

SEQ ID NO: 100 - DNA, 16 bp, synthetic construct
  source 1..16: mol_type = other DNA
tcgcgtgcctgaattg

SEQ ID NO: 101 - DNA, 17 bp, synthetic construct
  source 1..17: mol_type = other DNA
gtcgcgtgcctgaattg

SEQ ID NO: 102 - DNA, 20 bp, synthetic construct
  source 1..20: mol_type = other DNA
gccgtcgcgtgcctgaattg

SEQ ID NO: 103 - DNA, 15 bp, synthetic construct
  source 1..15: mol_type = other DNA
tcgcgtgcctgaatt

SEQ ID NO: 104 - DNA, 20 bp, synthetic construct
  source 1..20: mol_type = other DNA
cgccgtcgcgtgcctgaatt

SEQ ID NO: 105 - DNA, 17 bp, synthetic construct
  source 1..17: mol_type = other DNA
ccgtcgcgtgcctgaat

SEQ ID NO: 106 - DNA, 20 bp, synthetic construct
  source 1..20: mol_type = other DNA
ccgccgtcgcgtgcctgaat

SEQ ID NO: 107 - DNA, 17 bp, synthetic construct
  source 1..17: mol_type = other DNA
gccgtcgcgtgcctgaa

SEQ ID NO: 108 - DNA, 16 bp, synthetic construct
  source 1..16: mol_type = other DNA
gccgtcgcgtgcctga

SEQ ID NO: 109 - DNA, 17 bp, synthetic construct
  source 1..17: mol_type = other DNA
cgccgtcgcgtgcctga

SEQ ID NO: 110 - DNA, 15 bp, synthetic construct
  source 1..15: mol_type = other DNA
cgccgtcgcgtgcct

SEQ ID NO: 111 - DNA, 17 bp, synthetic construct
  source 1..17: mol_type = other DNA
cgccaggcgtgttgttc

SEQ ID NO: 112 - DNA, 17 bp, synthetic construct
  source 1..17: mol_type = other DNA
gcgccaggcgtgttgtt

SEQ ID NO: 113 - DNA, 16 bp, synthetic construct
  source 1..16: mol_type = other DNA
gcgccaggcgtgttgt

SEQ ID NO: 114 - DNA, 20 bp, synthetic construct
  source 1..20: mol_type = other DNA
ggtcggggtcgcgagcagac

SEQ ID NO: 115 - DNA, 20 bp, synthetic construct
  source 1..20: mol_type = other DNA
gacccaggtcggggtcgcga

SEQ ID NO: 116 - DNA, 20 bp, synthetic construct
  source 1..20: mol_type = other DNA
cgacggcacgatctggtagg

SEQ ID NO: 117 - DNA, 20 bp, synthetic construct
  source 1..20: mol_type = other DNA
gcacctgcgacggcacgatc

SEQ ID NO: 118 - DNA, 15 bp, synthetic construct
  source 1..15: mol_type = other DNA
ctgcgacggcacgat

SEQ ID NO: 119 - DNA, 16 bp, synthetic construct
  source 1..16: mol_type = other DNA
cctgcgacggcacgat

SEQ ID NO: 120 - DNA, 17 bp, synthetic construct
  source 1..17: mol_type = other DNA
acctgcgacggcacgat

SEQ ID NO: 121 - DNA, 20 bp, synthetic construct
  source 1..20: mol_type = other DNA
cgcacctgcgacggcacgat

SEQ ID NO: 122 - DNA, 15 bp, synthetic construct
  source 1..15: mol_type = other DNA
cctgcgacggcacga

SEQ ID NO: 123 - DNA, 16 bp, synthetic construct
  source 1..16: mol_type = other DNA
acctgcgacggcacga

SEQ ID NO: 124 - DNA, 20 bp, synthetic construct
  source 1..20: mol_type = other DNA
catgtcccgcacctgcgacg

SEQ ID NO: 125 - DNA, 16 bp, synthetic construct
  source 1..16: mol_type = other DNA
cgcgtagatgtcgcct

SEQ ID NO: 126 - DNA, 16 bp, synthetic construct
  source 1..16: mol_type = other DNA
gcgcgtagatgtcgcc

SEQ ID NO: 127 - DNA, 20 bp, synthetic construct
  source 1..20: mol_type = other DNA
atcgcagcgcgtagatgtcg

SEQ ID NO: 128 - DNA, 20 bp, synthetic construct
  source 1..20: mol_type = other DNA
gatcgcagcgcgtagatgtc

SEQ ID NO: 129 - DNA, 17 bp, synthetic construct
  source 1..17: mol_type = other DNA
tcgcagcgcgtagatgt

SEQ ID NO: 130 - DNA, 20 bp, synthetic construct
  source 1..20: mol_type = other DNA
ggatcgcagcgcgtagatgt

SEQ ID NO: 131 - DNA, 17 bp, synthetic construct
  source 1..17: mol_type = other DNA
atcgcagcgcgtagatg

SEQ ID NO: 132 - DNA, 16 bp, synthetic construct
  source 1..16: mol_type = other DNA
atcgcagcgcgtagat

SEQ ID NO: 133 - DNA, 17 bp, synthetic construct
  source 1..17: mol_type = other DNA
gatcgcagcgcgtagat

SEQ ID NO: 134 - DNA, 15 bp, synthetic construct
  source 1..15: mol_type = other DNA
atcgcagcgcgtaga

SEQ ID NO: 135 - DNA, 16 bp, synthetic construct
  source 1..16: mol_type = other DNA
gatcgcagcgcgtaga

SEQ ID NO: 136 - DNA, 20 bp, synthetic construct
  source 1..20: mol_type = other DNA
ggttaaaggatcgcagcgcg

SEQ ID NO: 137 - DNA, 20 bp, synthetic construct
  source 1..20: mol_type = other DNA
tggttaaaggatcgcagcgc

SEQ ID NO: 138 - DNA, 20 bp, synthetic construct
  source 1..20: mol_type = other DNA
gtggttaaaggatcgcagcg

SEQ ID NO: 139 - DNA, 17 bp, synthetic construct
  source 1..17: mol_type = other DNA
aattcgaacgccttggt

SEQ ID NO: 140 - DNA, 15 bp, synthetic construct
  source 1..15: mol_type = other DNA
attcgaacgccttgg

SEQ ID NO: 141 - DNA, 16 bp, synthetic construct
  source 1..16: mol_type = other DNA
aattcgaacgccttgg

SEQ ID NO: 142 - DNA, 15 bp, synthetic construct
  source 1..15: mol_type = other DNA
gaattcgaacgcctt

SEQ ID NO: 143 - DNA, 15 bp, synthetic construct
  source 1..15: mol_type = other DNA
ttgaattcgaacgcc

SEQ ID NO: 144 - DNA, 16 bp, synthetic construct
  source 1..16: mol_type = other DNA
cgtagcgttgctttgc

SEQ ID NO: 145 - DNA, 17 bp, synthetic construct
  source 1..17: mol_type = other DNA
ccgtagcgttgctttgc

SEQ ID NO: 146 - DNA, 15 bp, synthetic construct
  source 1..15: mol_type = other DNA
cgtagcgttgctttg

SEQ ID NO: 147 - DNA, 16 bp, synthetic construct
  source 1..16: mol_type = other DNA
ccgtagcgttgctttg

SEQ ID NO: 148 - DNA, 16 bp, synthetic construct
  source 1..16: mol_type = other DNA
gtcgttgacgtcgagg

SEQ ID NO: 149 - DNA, 20 bp, synthetic construct
  source 1..20: mol_type = other DNA
cggcagtgccgttaatcaga

SEQ ID NO: 150 - DNA, 20 bp, synthetic construct
  source 1..20: mol_type = other DNA
tcggcagtgccgttaatcag

SEQ ID NO: 151 - DNA, 20 bp, synthetic construct
  source 1..20: mol_type = other DNA
ttttcgccctcatcgtagtc

SEQ ID NO: 152 - DNA, 16 bp, synthetic construct
  source 1..16: mol_type = other DNA
tcgccctcatcgtagt

SEQ ID NO: 153 - DNA, 16 bp, synthetic construct
  source 1..16: mol_type = other DNA
ttcgccctcatcgtag

SEQ ID NO: 154 - DNA, 20 bp, synthetic construct
  source 1..20: mol_type = other DNA
ggtgactcggccattttcgc

SEQ ID NO: 155 - DNA, 20 bp, synthetic construct
  source 1..20: mol_type = other DNA
gtcgtaggtgactcggccat

SEQ ID NO: 156 - DNA, 20 bp, synthetic construct
  source 1..20: mol_type = other DNA
aaagaagccgcggtcgccct

SEQ ID NO: 157 - DNA, 20 bp, synthetic construct
  source 1..20: mol_type = other DNA
tcaaagaagccgcggtcgcc

SEQ ID NO: 158 - DNA, 17 bp, synthetic construct
  source 1..17: mol_type = other DNA
aaagaagccgcggtcgc

SEQ ID NO: 159 - DNA, 20 bp, synthetic construct
  source 1..20: mol_type = other DNA
ttcaaagaagccgcggtcgc

SEQ ID NO: 160 - DNA, 20 bp, synthetic construct
  source 1..20: mol_type = other DNA
tttcaaagaagccgcggtcg

SEQ ID NO: 161 - DNA, 20 bp, synthetic construct
  source 1..20: mol_type = other DNA
atttcaaagaagccgcggtc

SEQ ID NO: 162 - DNA, 17 bp, synthetic construct
  source 1..17: mol_type = other DNA
ttcaaagaagccgcggt

SEQ ID NO: 163 - DNA, 16 bp, synthetic construct
  source 1..16: mol_type = other DNA
ttcaaagaagccgcgg

SEQ ID NO: 164 - DNA, 20 bp, synthetic construct
  source 1..20: mol_type = other DNA
ctatttcaaagaagccgcgg

SEQ ID NO: 165 - DNA, 17 bp, synthetic construct
  source 1..17: mol_type = other DNA
atttcaaagaagccgcg

SEQ ID NO: 166 - DNA, 20 bp, synthetic construct
  source 1..20: mol_type = other DNA
gtcttgccgtggtcgtgagc

SEQ ID NO: 167 - DNA, 20 bp, synthetic construct
  source 1..20: mol_type = other DNA
tgtcttgccgtggtcgtgag

SEQ ID NO: 168 - DNA, 20 bp, synthetic construct
  source 1..20: mol_type = other DNA
atgtcttgccgtggtcgtga

SEQ ID NO: 169 - DNA, 17 bp, synthetic construct
  source 1..17: mol_type = other DNA
tgtcttgccgtggtcgt

SEQ ID NO: 170 - DNA, 17 bp, synthetic construct
  source 1..17: mol_type = other DNA
ttgccacgaagatcata

SEQ ID NO: 171 - DNA, 16 bp, synthetic construct
  source 1..16: mol_type = other DNA
cgcttgcacttgattg

SEQ ID NO: 172 - DNA, 17 bp, synthetic construct
  source 1..17: mol_type = other DNA
agttgtaggtccggatc

SEQ ID NO: 173 - DNA, 16 bp, synthetic construct
  source 1..16: mol_type = other DNA
agttgtaggtccggat

SEQ ID NO: 174 - DNA, 20 bp, synthetic construct
  source 1..20: mol_type = other DNA
accctagtactaaggttgat

SEQ ID NO: 175 - DNA, 20 bp, synthetic construct
  source 1..20: mol_type = other DNA
gatcatctcgcaagtctcat

SEQ ID NO: 176 - DNA, 16 bp, synthetic construct
  source 1..16: mol_type = other DNA
gatcatctcgcaagtc

SEQ ID NO: 177 - DNA, 20 bp, synthetic construct
  source 1..20: mol_type = other DNA
aggaatttaaaggttagtgc

SEQ ID NO: 178 - DNA, 20 bp, synthetic construct
  source 1..20: mol_type = other DNA
ccgaggagacaagtgatggt

SEQ ID NO: 179 - DNA, 20 bp, synthetic construct
  source 1..20: mol_type = other DNA
cttgctcctcgctaatggga

SEQ ID NO: 180 - DNA, 20 bp, synthetic construct
  source 1..20: mol_type = other DNA
ctgacaacgttcatcttgtc

SEQ ID NO: 181 - DNA, 20 bp, synthetic construct
  source 1..20: mol_type = other DNA
aagtgctctcagagcggcgg

SEQ ID NO: 182 - DNA, 20 bp, synthetic construct
  source 1..20: mol_type = other DNA
aaagtgctctcagagcggcg

SEQ ID NO: 183 - DNA, 20 bp, synthetic construct
  source 1..20: mol_type = other DNA
tgcgggtattctggttctcc

SEQ ID NO: 184 - DNA, 15 bp, synthetic construct
  source 1..15: mol_type = other DNA
cgttgttagagacga

SEQ ID NO: 185 - DNA, 17 bp, synthetic construct
  source 1..17: mol_type = other DNA
ttcgttgttagagacga

SEQ ID NO: 186 - DNA, 20 bp, synthetic construct
  source 1..20: mol_type = other DNA
gagctattggtgatccacaa

SEQ ID NO: 187 - DNA, 20 bp, synthetic construct
  source 1..20: mol_type = other DNA
gagtggactcgtctataatc

SEQ ID NO: 188 - DNA, 16 bp, synthetic construct
  source 1..16: mol_type = other DNA
agtggactcgtctata

SEQ ID NO: 189 - DNA, 16 bp, synthetic construct
  source 1..16: mol_type = other DNA
gagtggactcgtctat

SEQ ID NO: 190 - DNA, 16 bp, synthetic construct
  source 1..16: mol_type = other DNA
tgagtggactcgtcta

SEQ ID NO: 191 - DNA, 17 bp, synthetic construct
  source 1..17: mol_type = other DNA
gtctgagtggactcgtc

SEQ ID NO: 192 - DNA, 20 bp, synthetic construct
  source 1..20: mol_type = other DNA
agccatatgcctctgttcgt

SEQ ID NO: 193 - DNA, 20 bp, synthetic construct
  source 1..20: mol_type = other DNA
tagccatatgcctctgttcg

SEQ ID NO: 194 - DNA, 20 bp, synthetic construct
  source 1..20: mol_type = other DNA
gcctttatggcagaactcgc

SEQ ID NO: 195 - DNA, 20 bp, synthetic construct
  source 1..20: mol_type = other DNA
acttaaaaggagatctcggt

SEQ ID NO: 196 - DNA, 20 bp, synthetic construct
  source 1..20: mol_type = other DNA
gagcctaactactaaacagt

SEQ ID NO: 197 - DNA, 20 bp, synthetic construct
  source 1..20: mol_type = other DNA
tcgtgtgcttagtagggctc

SEQ ID NO: 198 - DNA, 20 bp, synthetic construct
  source 1..20: mol_type = other DNA
catcgtgtgcttagtagggc

SEQ ID NO: 199 - DNA, 20 bp, synthetic construct
  source 1..20: mol_type = other DNA
agcatcgtgtgcttagtagg

SEQ ID NO: 200 - DNA, 20 bp, synthetic construct
  source 1..20: mol_type = other DNA
ccctcatcggcactggtaaa

SEQ ID NO: 201 - DNA, 20 bp, synthetic construct
  source 1..20: mol_type = other DNA
ctccctcatcggcactggta

SEQ ID NO: 202 - DNA, 15 bp, synthetic construct
  source 1..15: mol_type = other DNA
gccgttgaagatagc

SEQ ID NO: 203 - DNA, 20 bp, synthetic construct
  source 1..20: mol_type = other DNA
acgtgctagccagtataacc

SEQ ID NO: 204 - DNA, 16 bp, synthetic construct
  source 1..16: mol_type = other DNA
gtattaacgtgctagc

SEQ ID NO: 205 - DNA, 16 bp, synthetic construct
  source 1..16: mol_type = other DNA
acctgttcgtcaatac

SEQ ID NO: 206 - DNA, 20 bp, synthetic construct
  source 1..20: mol_type = other DNA
cgttggtgattaaccagact

SEQ ID NO: 207 - DNA, 16 bp, synthetic construct
  source 1..16: mol_type = other DNA
ggttcgatgtgattgg

SEQ ID NO: 208 - DNA, 20 bp, synthetic construct
  source 1..20: mol_type = other DNA
atagggttcgatgtgattgg

SEQ ID NO: 209 - DNA, 20 bp, synthetic construct
  source 1..20: mol_type = other DNA
aggtcgcactaaacaattta

SEQ ID NO: 210 - DNA, 20 bp, synthetic construct
  source 1..20: mol_type = other DNA
cttgccaagaatagcttagc

SEQ ID NO: 211 - DNA, 20 bp, synthetic construct
  source 1..20: mol_type = other DNA
aaggtccttcatggacgaga

SEQ ID NO: 212 - DNA, 20 bp, synthetic construct
  source 1..20: mol_type = other DNA
tcgtggtccttcctactaga

SEQ ID NO: 213 - DNA, 20 bp, synthetic construct
  source 1..20: mol_type = other DNA
cgaccaccagataggcaagc

SEQ ID NO: 214 - DNA, 20 bp, synthetic construct
  source 1..20: mol_type = other DNA
ggccttatacctaagaacga

SEQ ID NO: 215 - DNA, 20 bp, synthetic construct
  source 1..20: mol_type = other DNA
tggccttatacctaagaacg

SEQ ID NO: 216 - DNA, 15 bp, synthetic construct
  source 1..15: mol_type = other DNA
ccgaaggggtagcta

SEQ ID NO: 217 - DNA, 20 bp, synthetic construct
  source 1..20: mol_type = other DNA
tggaatcgatgagaagggta

SEQ ID NO: 218 - DNA, 20 bp, synthetic construct
  source 1..20: mol_type = other DNA
cccgtgttggagtcatacct

SEQ ID NO: 219 - DNA, 20 bp, synthetic construct
  source 1..20: mol_type = other DNA
gacaagtattgctccctggt

SEQ ID NO: 220 - DNA, 20 bp, synthetic construct
  source 1..20: mol_type = other DNA
gcctacagtagctttattgc

SEQ ID NO: 221 - DNA, 20 bp, synthetic construct
  source 1..20: mol_type = other DNA
aatggaagcccaattggctc

SEQ ID NO: 222 - DNA, 20 bp, synthetic construct
  source 1..20: mol_type = other DNA
taggtagaatgtgcgtgcgt

SEQ ID NO: 223 - DNA, 17 bp, synthetic construct
  source 1..17: mol_type = other DNA
ggtagaatgtgcgtgcg

SEQ ID NO: 224 - DNA, 20 bp, synthetic construct
  source 1..20: mol_type = other DNA
ctgcttaaacacgacaggtt

SEQ ID NO: 225 - DNA, 16 bp, synthetic construct
  source 1..16: mol_type = other DNA
gcctgcttaaacacga

SEQ ID NO: 226 - DNA, 20 bp, synthetic construct
  source 1..20: mol_type = other DNA
acttgtacgcaggctactgg

SEQ ID NO: 227 - DNA, 20 bp, synthetic construct
  source 1..20: mol_type = other DNA
tacttgtacgcaggctactg

SEQ ID NO: 228 - DNA, 20 bp, synthetic construct
  source 1..20: mol_type = other DNA
atacttgtacgcaggctact

SEQ ID NO: 229 - DNA, 15 bp, synthetic construct
  source 1..15: mol_type = other DNA
ttgtacgcaggctac

SEQ ID NO: 230 - DNA, 20 bp, synthetic construct
  source 1..20: mol_type = other DNA
tatacttgtacgcaggctac

SEQ ID NO: 231 - DNA, 15 bp, synthetic construct
  source 1..15: mol_type = other DNA
cttgtacgcaggcta

SEQ ID NO: 232 - DNA, 20 bp, synthetic construct
  source 1..20: mol_type = other DNA
cgtgccctagtctctagaag

SEQ ID NO: 233 - DNA, 20 bp, synthetic construct
  source 1..20: mol_type = other DNA
gaatcgtgccctagtctcta

SEQ ID NO: 234 - DNA, 20 bp, synthetic construct
  source 1..20: mol_type = other DNA
actgaatcgtgccctagtct

SEQ ID NO: 235 - DNA, 20 bp, synthetic construct
  source 1..20: mol_type = other DNA
gattctagtaagacgtttgg

SEQ ID NO: 236 - DNA, 20 bp, synthetic construct
  source 1..20: mol_type = other DNA
gatataggaggattctagta

SEQ ID NO: 237 - DNA, 20 bp, synthetic construct
  source 1..20: mol_type = other DNA
taggatcgtcaccaaccttc

SEQ ID NO: 238 - DNA, 17 bp, synthetic construct
  source 1..17: mol_type = other DNA
taggatcgtcaccaacc

SEQ ID NO: 239 - DNA, 20 bp, synthetic construct
  source 1..20: mol_type = other DNA
acataggatcgtcaccaacc

SEQ ID NO: 240 - DNA, 15 bp, synthetic construct
  source 1..15: mol_type = other DNA
taacataggatcgtc

SEQ ID NO: 241 - DNA, 16 bp, synthetic construct
  source 1..16: mol_type = other DNA
ttaacataggatcgtc

SEQ ID NO: 242 - DNA, 16 bp, synthetic construct
  source 1..16: mol_type = other DNA
taatccgtctcaggtc

SEQ ID NO: 243 - DNA, 17 bp, synthetic construct
  source 1..17: mol_type = other DNA
gtaatccgtctcaggtc

SEQ ID NO: 244 - DNA, 16 bp, synthetic construct
  source 1..16: mol_type = other DNA
gtaatccgtctcaggt

SEQ ID NO: 245 - DNA, 20 bp, synthetic construct
  source 1..20: mol_type = other DNA
tagcgctagtcaatgtggca

SEQ ID NO: 246 - DNA, 20 bp, synthetic construct
  source 1..20: mol_type = other DNA
gtagcgctagtcaatgtggc

SEQ ID NO: 247 - DNA, 20 bp, synthetic construct
  source 1..20: mol_type = other DNA
agtagcgctagtcaatgtgg

SEQ ID NO: 248 - DNA, 20 bp, synthetic construct
  source 1..20: mol_type = other DNA
tagtagcgctagtcaatgtg

SEQ ID NO: 249 - DNA, 17 bp, synthetic construct
  source 1..17: mol_type = other DNA
gtagcgctagtcaatgt

SEQ ID NO: 250 - DNA, 20 bp, synthetic construct
  source 1..20: mol_type = other DNA
ttagtagcgctagtcaatgt

SEQ ID NO: 251 - DNA, 15 bp, synthetic construct
  source 1..15: mol_type = other DNA
gtagcgctagtcaat

SEQ ID NO: 252 - DNA, 16 bp, synthetic construct
  source 1..16: mol_type = other DNA
agtagcgctagtcaat

SEQ ID NO: 253 - DNA, 15 bp, synthetic construct
  source 1..15: mol_type = other DNA
agtagcgctagtcaa

SEQ ID NO: 254 - DNA, 16 bp, synthetic construct
  source 1..16: mol_type = other DNA
tagtagcgctagtcaa

SEQ ID NO: 255 - DNA, 17 bp, synthetic construct
  source 1..17: mol_type = other DNA
ttagtagcgctagtcaa

SEQ ID NO: 256 - DNA, 16 bp, synthetic construct
  source 1..16: mol_type = other DNA
ttagtagcgctagtca

SEQ ID NO: 257 - DNA, 15 bp, synthetic construct
  source 1..15: mol_type = other DNA
ttagtagcgctagtc

SEQ ID NO: 258 - DNA, 16 bp, synthetic construct
  source 1..16: mol_type = other DNA
attagtagcgctagtc

SEQ ID NO: 259 - DNA, 17 bp, synthetic construct
  source 1..17: mol_type = other DNA
aattagtagcgctagtc

SEQ ID NO: 260 - DNA, 20 bp, synthetic construct
  source 1..20: mol_type = other DNA
tgcaattagtagcgctagtc

SEQ ID NO: 261 - DNA, 15 bp, synthetic construct
  source 1..15: mol_type = other DNA
attagtagcgctagt

SEQ ID NO: 262 - DNA, 16 bp, synthetic construct
  source 1..16: mol_type = other DNA
aattagtagcgctagt

SEQ ID NO: 263 - DNA, 17 bp, synthetic construct
  source 1..17: mol_type = other DNA
caattagtagcgctagt

SEQ ID NO: 264 - DNA, 20 bp, synthetic construct
  source 1..20: mol_type = other DNA
atgcaattagtagcgctagt

SEQ ID NO: 265 - DNA, 16 bp, synthetic construct
  source 1..16: mol_type = other DNA
caattagtagcgctag

SEQ ID NO: 266 - DNA, 17 bp, synthetic construct
  source 1..17: mol_type = other DNA
gcaattagtagcgctag

SEQ ID NO: 267 - DNA, 15 bp, synthetic construct
  source 1..15: mol_type = other DNA
caattagtagcgcta

SEQ ID NO: 268 - DNA, 15 bp, synthetic construct
  source 1..15: mol_type = other DNA
tgcaattagtagcgc

SEQ ID NO: 269 - DNA, 20 bp, synthetic construct
  source 1..20: mol_type = other DNA
acccggaattgattaagcac

SEQ ID NO: 270 - DNA, 20 bp, synthetic construct
  source 1..20: mol_type = other DNA
cggctattcacgtagttgga

SEQ ID NO: 271 - DNA, 20 bp, synthetic construct
  source 1..20: mol_type = other DNA
tcggctattcacgtagttgg

SEQ ID NO: 272 - DNA, 20 bp, synthetic construct
  source 1..20: mol_type = other DNA
gctcggctattcacgtagtt

SEQ ID NO: 273 - DNA, 16 bp, synthetic construct
  source 1..16: mol_type = other DNA
cggctattcacgtagt

SEQ ID NO: 274 - DNA, 15 bp, synthetic construct
  source 1..15: mol_type = other DNA
cggctattcacgtag

SEQ ID NO: 275 - DNA, 16 bp, synthetic construct
  source 1..16: mol_type = other DNA
tcggctattcacgtag

SEQ ID NO: 276 - DNA, 20 bp, synthetic construct
  source 1..20: mol_type = other DNA
gggctcggctattcacgtag

SEQ ID NO: 277 - DNA, 15 bp, synthetic construct
  source 1..15: mol_type = other DNA
tcggctattcacgta

SEQ ID NO: 278 - DNA, 15 bp, synthetic construct
  source 1..15: mol_type = other DNA
ctcggctattcacgt

SEQ ID NO: 279 - DNA, 15 bp, synthetic construct
  source 1..15: mol_type = other DNA
gctcggctattcacg

SEQ ID NO: 280 - DNA, 17 bp, synthetic construct
  source 1..17: mol_type = other DNA
gggctcggctattcacg

SEQ ID NO: 281 - DNA, 20 bp, synthetic construct
  source 1..20: mol_type = other DNA
ttgattaaatgggctcggct

SEQ ID NO: 282 - DNA, 17 bp, synthetic construct
  source 1..17: mol_type = other DNA
tgattaaatgggctcgg

SEQ ID NO: 283 - DNA, 20 bp, synthetic construct
  source 1..20: mol_type = other DNA
ctgataatacaagggctacc

SEQ ID NO: 284 - DNA, 20 bp, synthetic construct
  source 1..20: mol_type = other DNA
ccgaaaagaagggtccaaga

SEQ ID NO: 285 - DNA, 20 bp, synthetic construct
  source 1..20: mol_type = other DNA
gcatgacgaatgaacatcac

SEQ ID NO: 286 - DNA, 20 bp, synthetic construct
  source 1..20: mol_type = other DNA
tgaatccattccctagtcgg

SEQ ID NO: 287 - DNA, 20 bp, synthetic construct
  source 1..20: mol_type = other DNA
cgaccgtgacagatctgatt

SEQ ID NO: 288 - DNA, 20 bp, synthetic construct
  source 1..20: mol_type = other DNA
acgaccgtgacagatctgat

SEQ ID NO: 289 - DNA, 17 bp, synthetic construct
  source 1..17: mol_type = other DNA
cgaccgtgacagatctg

SEQ ID NO: 290 - DNA, 17 bp, synthetic construct
  source 1..17: mol_type = other DNA
acgaccgtgacagatct

SEQ ID NO: 291 - DNA, 17 bp, synthetic construct
  source 1..17: mol_type = other DNA
tacgaccgtgacagatc

SEQ ID NO: 292 - DNA, 20 bp, synthetic construct
  source 1..20: mol_type = other DNA
atttacgaccgtgacagatc

SEQ ID NO: 293 - DNA, 15 bp, synthetic construct
  source 1..15: mol_type = other DNA
acgaccgtgacagat

SEQ ID NO: 294 - DNA, 16 bp, synthetic construct
  source 1..16: mol_type = other DNA
tacgaccgtgacagat

SEQ ID NO: 295 - DNA, 17 bp, synthetic construct
  source 1..17: mol_type = other DNA
ttacgaccgtgacagat

SEQ ID NO: 296 - DNA, 15 bp, synthetic construct
  source 1..15: mol_type = other DNA
tacgaccgtgacaga

SEQ ID NO: 297 - DNA, 15 bp, synthetic construct
  source 1..15: mol_type = other DNA
ttacgaccgtgacag

SEQ ID NO: 298 - DNA, 16 bp, synthetic construct
  source 1..16: mol_type = other DNA
tttacgaccgtgacag

SEQ ID NO: 299 - DNA, 16 bp, synthetic construct
  source 1..16: mol_type = other DNA
aatttacgaccgtgac

SEQ ID NO: 300 - DNA, 17 bp, synthetic construct
  source 1..17: mol_type = other DNA
taatttacgaccgtgac

SEQ ID NO: 301 - DNA, 15 bp, synthetic construct
  source 1..15: mol_type = other DNA
aatttacgaccgtga

SEQ ID NO: 302 - DNA, 16 bp, synthetic construct
  source 1..16: mol_type = other DNA
taatttacgaccgtga

SEQ ID NO: 303 - DNA, 17 bp, synthetic construct
  source 1..17: mol_type = other DNA
ataatttacgaccgtga

SEQ ID NO: 304 - DNA, 15 bp, synthetic construct
  source 1..15: mol_type = other DNA
taatttacgaccgtg

SEQ ID NO: 305 - DNA, 16 bp, synthetic construct
  source 1..16: mol_type = other DNA
ataatttacgaccgtg

SEQ ID NO: 306 - DNA, 17 bp, synthetic construct
  source 1..17: mol_type = other DNA
gataatttacgaccgtg

SEQ ID NO: 307 - DNA, 15 bp, synthetic construct
  source 1..15: mol_type = other DNA
ataatttacgaccgt

SEQ ID NO: 308 - DNA, 17 bp, synthetic construct
  source 1..17: mol_type = other DNA
tgataatttacgaccgt

SEQ ID NO: 309 - DNA, 15 bp, synthetic construct
  source 1..15: mol_type = other DNA
gataatttacgaccg

SEQ ID NO: 310 - DNA, 16 bp, synthetic construct
  source 1..16: mol_type = other DNA
tgataatttacgaccg

SEQ ID NO: 311 - DNA, 20 bp, synthetic construct
  source 1..20: mol_type = other DNA
tccgctggacatcatgctca

SEQ ID NO: 312 - DNA, 20 bp, synthetic construct
  source 1..20: mol_type = other DNA
gctccgctggacatcatgct

SEQ ID NO: 313 - DNA, 20 bp, synthetic construct
  source 1..20: mol_type = other DNA
ggctccgctggacatcatgc

SEQ ID NO: 314 - DNA, 17 bp, synthetic construct
  source 1..17: mol_type = other DNA
gctccgctggacatcat

SEQ ID NO: 315 - DNA, 17 bp, synthetic construct
  source 1..17: mol_type = other DNA
ggctccgctggacatca

SEQ ID NO: 316 - DNA, 20 bp, synthetic construct
  source 1..20: mol_type = other DNA
caatacaggctccgctggac

SEQ ID NO: 317 - DNA, 20 bp, synthetic construct
  source 1..20: mol_type = other DNA
acaatacaggctccgctgga

SEQ ID NO: 318 - DNA, 16 bp, synthetic construct
  source 1..16: mol_type = other DNA
caatacaggctccgct

SEQ ID NO: 319 - DNA, 20 bp, synthetic construct
  source 1..20: mol_type = other DNA
cgtattaattcatgggcact

SEQ ID NO: 320 - DNA, 20 bp, synthetic construct
  source 1..20: mol_type = other DNA
gcatgatccctataaacccc

SEQ ID NO: 321 - DNA, 20 bp, synthetic construct
  source 1..20: mol_type = other DNA
gatctaatcaaccgctttgt

SEQ ID NO: 322 - DNA, 20 bp, synthetic construct
  source 1..20: mol_type = other DNA
ggatctaatcaaccgctttg

SEQ ID NO: 323 - DNA, 20 bp, synthetic construct
  source 1..20: mol_type = other DNA
ggggatctaatcaaccgctt

SEQ ID NO: 324 - DNA, 16 bp, synthetic construct
  source 1..16: mol_type = other DNA
ggatctaatcaaccgc

SEQ ID NO: 325 - DNA, 20 bp, synthetic construct
  source 1..20: mol_type = other DNA
gcctctaaaccagcaaggta

SEQ ID NO: 326 - DNA, 17 bp, synthetic construct
  source 1..17: mol_type = other DNA
cgatcctatgtagtcta

SEQ ID NO: 327 - DNA, 16 bp, synthetic construct
  source 1..16: mol_type = other DNA
cgatcctatgtagtct

SEQ ID NO: 328 - DNA, 17 bp, synthetic construct
  source 1..17: mol_type = other DNA
ccgatcctatgtagtct

SEQ ID NO: 329 - DNA, 20 bp, synthetic construct
  source 1..20: mol_type = other DNA
tcaccgatcctatgtagtct

SEQ ID NO: 330 - DNA, 15 bp, synthetic construct
  source 1..15: mol_type = other DNA
cgatcctatgtagtc

SEQ ID NO: 331 - DNA, 16 bp, synthetic construct
  source 1..16: mol_type = other DNA
ccgatcctatgtagtc

SEQ ID NO: 332 - DNA, 17 bp, synthetic construct
  source 1..17: mol_type = other DNA
accgatcctatgtagtc

SEQ ID NO: 333 - DNA, 20 bp, synthetic construct
  source 1..20: mol_type = other DNA
ttcaccgatcctatgtagtc

SEQ ID NO: 334 - DNA, 15 bp, synthetic construct
  source 1..15: mol_type = other DNA
ccgatcctatgtagt

SEQ ID NO: 335 - DNA, 16 bp, synthetic construct
  source 1..16: mol_type = other DNA
accgatcctatgtagt

SEQ ID NO: 336 - DNA, 20 bp, synthetic construct
  source 1..20: mol_type = other DNA
cttcaccgatcctatgtagt

SEQ ID NO: 337 - DNA, 15 bp, synthetic construct
  source 1..15: mol_type = other DNA
accgatcctatgtag

SEQ ID NO: 338 - DNA, 17 bp, synthetic construct
  source 1..17: mol_type = other DNA
tcaccgatcctatgtag

SEQ ID NO: 339 - DNA, 16 bp, synthetic construct
  source 1..16: mol_type = other DNA
tcaccgatcctatgta

SEQ ID NO: 340 - DNA, 16 bp, synthetic construct
  source 1..16: mol_type = other DNA
ctctaattagcgtcac

SEQ ID NO: 341 - DNA, 20 bp, synthetic construct
  source 1..20: mol_type = other DNA
tacttcagtaccaccggaat

SEQ ID NO: 342 - DNA, 20 bp, synthetic construct
  source 1..20: mol_type = other DNA
ctagtagtcctgaactaatg

SEQ ID NO: 343 - DNA, 20 bp, synthetic construct
  source 1..20: mol_type = other DNA
ggatttggtagttccatcac

SEQ ID NO: 344 - DNA, 20 bp, synthetic construct
  source 1..20: mol_type = other DNA
gctagccttatcatttgacc

SEQ ID NO: 345 - DNA, 20 bp, synthetic construct
  source 1..20: mol_type = other DNA
gtccaagctagccttatcat

SEQ ID NO: 346 - DNA, 20 bp, synthetic construct
  source 1..20: mol_type = other DNA
taacgattttacggagacaa

SEQ ID NO: 347 - DNA, 16 bp, synthetic construct
  source 1..16: mol_type = other DNA
cgattttacggagaca

SEQ ID NO: 348 - DNA, 17 bp, synthetic construct
  source 1..17: mol_type = other DNA
acgattttacggagaca

SEQ ID NO: 349 - DNA, 20 bp, synthetic construct
  source 1..20: mol_type = other DNA
gtaacgattttacggagaca

SEQ ID NO: 350 - DNA, 15 bp, synthetic construct
  source 1..15: mol_type = other DNA
cgattttacggagac

SEQ ID NO: 351 - DNA, 16 bp, synthetic construct
  source 1..16: mol_type = other DNA
acgattttacggagac

SEQ ID NO: 352 - DNA, 17 bp, synthetic construct
  source 1..17: mol_type = other DNA
aacgattttacggagac

SEQ ID NO: 353 - DNA, 20 bp, synthetic construct
  source 1..20: mol_type = other DNA
agtaacgattttacggagac

SEQ ID NO: 354 - DNA, 16 bp, synthetic construct
  source 1..16: mol_type = other DNA
aacgattttacggaga

SEQ ID NO: 355 - DNA, 15 bp, synthetic construct
  source 1..15: mol_type = other DNA
aacgattttacggag

SEQ ID NO: 356 - DNA, 17 bp, synthetic construct
  source 1..17: mol_type = other DNA
gtaacgattttacggag

SEQ ID NO: 357 - DNA, 16 bp, synthetic construct
  source 1..16: mol_type = other DNA
gtaacgattttacgga

SEQ ID NO: 358 - DNA, 15 bp, synthetic construct
  source 1..15: mol_type = other DNA
gtaacgattttacgg

SEQ ID NO: 359 - DNA, 16 bp, synthetic construct
  source 1..16: mol_type = other DNA
agtaacgattttacgg

SEQ ID NO: 360 - DNA, 20 bp, synthetic construct
  source 1..20: mol_type = other DNA
catgagtaacgattttacgg

SEQ ID NO: 361 - DNA, 16 bp, synthetic construct
  source 1..16: mol_type = other DNA
gagtaacgattttacg

SEQ ID NO: 362 - DNA, 17 bp, synthetic construct
  source 1..17: mol_type = other DNA
tgagtaacgattttacg

SEQ ID NO: 363 - DNA, 20 bp, synthetic construct
  source 1..20: mol_type = other DNA
tcatgagtaacgattttacg

SEQ ID NO: 364 - DNA, 20 bp, synthetic construct
  source 1..20: mol_type = other DNA
tccgttcaagcctgcatgat

SEQ ID NO: 365 - DNA, 16 bp, synthetic construct
  source 1..16: mol_type = other DNA
tgcacgtaggagtagc

SEQ ID NO: 366 - DNA, 17 bp, synthetic construct
  source 1..17: mol_type = other DNA
ttgcacgtaggagtagc

SEQ ID NO: 367 - DNA, 20 bp, synthetic construct
  source 1..20: mol_type = other DNA
cgagcctttgcacgtaggag

SEQ ID NO: 368 - DNA, 20 bp, synthetic construct
  source 1..20: mol_type = other DNA
tcgagcctttgcacgtagga

SEQ ID NO: 369 - DNA, 20 bp, synthetic construct
  source 1..20: mol_type = other DNA
actcgagcctttgcacgtag

SEQ ID NO: 370 - DNA, 20 bp, synthetic construct
  source 1..20: mol_type = other DNA
aatagacagcgcgatgatgt

SEQ ID NO: 371 - DNA, 20 bp, synthetic construct
  source 1..20: mol_type = other DNA
tcaatagacagcgcgatgat

SEQ ID NO: 372 - DNA, 15 bp, synthetic construct
  source 1..15: mol_type = other DNA
tagacagcgcgatga

SEQ ID NO: 373 - DNA, 16 bp, synthetic construct
  source 1..16: mol_type = other DNA
atagacagcgcgatga

SEQ ID NO: 374 - DNA, 15 bp, synthetic construct
  source 1..15: mol_type = other DNA
atagacagcgcgatg

SEQ ID NO: 375 - DNA, 16 bp, synthetic construct
  source 1..16: mol_type = other DNA
aatagacagcgcgatg

SEQ ID NO: 376 - DNA, 20 bp, synthetic construct
  source 1..20: mol_type = other DNA
cttcaatagacagcgcgatg

SEQ ID NO: 377 - DNA, 15 bp, synthetic construct
  source 1..15: mol_type = other DNA
aatagacagcgcgat

SEQ ID NO: 378 - DNA, 16 bp, synthetic construct
  source 1..16: mol_type = other DNA
caatagacagcgcgat

SEQ ID NO: 379 - DNA, 17 bp, synthetic construct
  source 1..17: mol_type = other DNA
tcaatagacagcgcgat

SEQ ID NO: 380 - DNA, 20 bp, synthetic construct
  source 1..20: mol_type = other DNA
gcttcaatagacagcgcgat

SEQ ID NO: 381 - DNA, 15 bp, synthetic construct
  source 1..15: mol_type = other DNA
tcaatagacagcgcg

SEQ ID NO: 382 - DNA, 20 bp, synthetic construct
  source 1..20: mol_type = other DNA
ggttgcgaaagtccgtttgg

SEQ ID NO: 383 - DNA, 20 bp, synthetic construct
  source 1..20: mol_type = other DNA
cgatatccttcagacgcttc

SEQ ID NO: 384 - DNA, 20 bp, synthetic construct
  source 1..20: mol_type = other DNA
gaacgatatccttcagacgc

SEQ ID NO: 385 - DNA, 15 bp, synthetic construct
  source 1..15: mol_type = other DNA
gatatccttcagacg

(19) United States
(12) Patent Application Publication
(10) Pub. No.: US 2025/0002912 A1
(43) Pub. Date: Jan. 2, 2025

(54) COMPOSITIONS AND METHODS FOR THE TREATMENT OF PCDH19 RELATED DISORDERS

(71) Applicant: THE FLOREY INSTITUTE OF NEUROSCIENCE AND MENTAL HEALTH, Parkville (AU)

(72) Inventor: Steven Petrou, Parkville (AU)

(21) Appl. No.: 18/694,295

(22) PCT Filed: Sep. 27, 2022
(86) PCT No.: PCT/AU2022/051153

§ 371 Date: Mar. 21, 2024

Related U.S. Application Data
(60) Provisional application No. 63/333,840, filed on Apr. 22, 2022.
(60) Provisional application No. 63/248,803, filed on Sep. 27, 2021.

(51) Int. Cl. C12N 15/113; A61P 25/08

(52) U.S. Cl. CPC C12N 15/113; A61P 25/08; C12N 2310/11; C12N 2310/315; C12N 2310/321

(57) ABSTRACT

Provided herein are antisense oligonucleotides to a target region of the protocadherin 19 (PCDH19) gene. The antisense oligonucleotides or compositions comprising same may be administered to a subject with a PCDH19 related disorder, such as epilepsy, schizophrenia or autism, in order to treat, reduce the symptoms of, or prevent the PCDH19 related disorder. Accordingly, also provided herein are useful compositions and methods to treat PCDH19 related disorders.

### RELATED APPLICATIONS

This application claims priority to U.S. Provisional Application No. 63/248,803 entitled “Compositions and methods for the treatment of PCDH19 related disorders” filed on 27 Sep. 2021, U.S. Provisional Application No. 63/333,840 entitled “Compositions and methods for the treatment of PCDH19 related disorders” filed on 22 Apr. 2022, the entire contents of which are hereby incorporated herein by reference in their entirety.

### FIELD OF THE INVENTION

The present invention relates to antisense oligonucleotides complementary to a target region of a of a human PCDH19 gene, a pre-mRNA transcript thereof and/or an mRNA transcript thereof. Embodiments of the invention also relate to compositions and methods for treating, reducing at least one symptom of, or preventing, a PCDH19 related disorder, such as epilepsy, schizophrenia or autism.

### BACKGROUND

Protocadherin 19 (PCDH19) is a cell-surface protein expressed in neurons that may play a role in cell-cell adhesion. Mutations of the PCDH19 gene have been associated with neurological disorders, such as epilepsy, autism, and schizophrenia. Interestingly, PCDH19 related early infantile epileptic encephalopathy 9 (EIEE9) is an X-linked disorder in which heterozygous females are affected but hemizygous males are not affected. Although it is rare, mutations in PCDH19 can also affect mosaic male carriers. While people with PCDH19 mutations can be diagnosed by genetic screening, no known cures currently exist to treat PCDH19 related disorders, such as epilepsy. Accordingly, new compositions and methods for treating these diseases are needed.

### SUMMARY OF THE INVENTION

In one aspect, the invention provides a compound including a single-stranded oligonucleotide that is 10-80 nucleosides in length and having a nucleobase sequence including a portion of 10 contiguous nucleobases having at least 80% (e.g., 85%, 90%, 95%, 97%, 99%, or 100%) complementarity to an equal length portion of a target region of a human PCDH19 gene, pre-mRNA transcript thereof and/or mRNA transcript thereof.

In particular embodiments the target region is within the nucleotide sequence set forth in SEQ ID NO:1 or a variant thereof having at least or about 80%, 81%, 82%, 83%, 84%, 85%, 86%, 87%, 88%, 89%, 90%, 91%, 92%, 93%, 94%, 95%, 96%, 97%, 98% or 99% sequence identity thereto. In particular embodiments the single-stranded oligonucleotide hybridizes to the PCDH19 pre-mRNA transcript or mRNA transcript.

In some embodiments, the mRNA transcript or pre-mRNA transcript may include at least one PCDH19 mutation. The mutation may be, for example, a mutation that encodes N340S, E307K, V441E, Q85X, N557K, D594H, S671X, L677fsx717, I119fsX122, and P364fsX375. The mutation may be a mutation selected from Table 1. The oligonucleotide may selectively hybridize to the mRNA transcript or pre-mRNA transcript that includes the at least one PCDH19 mutation over a wild-type mRNA transcript or pre-mRNA transcript. Thus, the oligonucleotide may be an allele-specific oligonucleotide.

In some embodiments, the oligonucleotide consists of 12 to 40 (e.g., 15 to 30, e.g., 16 to 22, e.g., 15, 16, 17, 18, 19, 20, 21, or 22) nucleobases.

In some embodiments, the oligonucleotide includes a gap segment including linked deoxyribonucleosides; a 5′ wing segment including linked nucleosides; and a 3′ wing segment including linked nucleosides. The gap segment may include a region of at least 10 contiguous nucleobases having at least 80% (e.g., 85%, 90%, 95%, 97%, 99%, or 100%) complementarity to an equal length portion of the target region of the pre-mRNA transcript or the mRNA transcript of the human PCDH19 gene positioned between the 5′ wing segment and the 3′ wing segment. The 5′ wing segment and the 3′ wing segment each includes at least two linked nucleosides, and at least one nucleoside of each wing segment includes an alternative nucleoside.

In some embodiments, the oligonucleotide includes at least one alternative internucleoside linkage. The at least one alternative internucleoside linkage may be a phosphorothioate internucleoside linkage. The at least one alternative internucleoside linkage may be a 2′-alkoxy internucleoside linkage. The at least one alternative internucleoside linkage may be an alkyl phosphate internucleoside linkage.

In some embodiments, the oligonucleotide includes at least one alternative nucleobase. The alternative nucleobase may be a 5′-methylcytosine, pseudouridine, or 5-methoxyuridine.

In some embodiments, the oligonucleotide includes at least one alternative sugar moiety. The alternative sugar moiety may be a 2′-OMe modified sugar moiety or a bicyclic sugar moiety.

In some embodiments, the oligonucleotide further includes a ligand conjugated to the 5′ end or the 3′ end of the oligonucleotide through a monovalent or branched bivalent or trivalent linker.

In some embodiments, the oligonucleotide includes a region complementary to at least 15 (e.g., 15, 16, 17 18, 19, 20, or 21) contiguous nucleotides of a PCDH19 gene.

In some embodiments, the oligonucleotide comprises a sequence set forth in any one of SEQ ID NOs: 2 to 385.

In another aspect, the invention provides a pharmaceutical composition including the oligonucleotide of any of the above embodiments and a pharmaceutically acceptable carrier or excipient.

In another aspect, the invention provides a composition including the oligonucleotide of any of the above embodiments and a lipid nanoparticle, a polyplex nanoparticle, a lipoplex nanoparticle, or a liposome.

In another aspect, the invention provides a method of treating, preventing, or delaying the progression of a PCDH19 related disorder in a subject in need thereof by administering to the subject the oligonucleotide, the pharmaceutical composition, or the composition of any of the above embodiments in an amount and for a duration sufficient to treat, prevent, or delay the progression of the PCDH19 related disorder.

Also provided herein is the use of an oligonucleotide of the present invention in the manufacture of a medicament for treating, preventing, or delaying the progression of a PCDH19 related disorder.

In another aspect, the invention provides a method of inhibiting transcription of PCDH19 in a cell of a subject having a PCDH19 related disorder by contacting the cell with the oligonucleotide, the pharmaceutical composition, or the composition of any of the above embodiments in an amount and for a duration sufficient to obtain degradation of an mRNA transcript of the PCDH19 gene, such that the oligonucleotide inhibits expression of the PCDH19 gene in the cell.

The cell may be contacted in vivo or ex vivo.

In another aspect, the invention provides a method of reducing a level and/or activity of PCDH19 in a cell of a subject having a PCDH19 related disorder by contacting the cell with the oligonucleotide, the pharmaceutical composition, or the composition of any of the above embodiments in an amount and for a duration sufficient to reduce the level and/or activity of PCDH19 in the cell.

The cell may be contacted in vivo or ex vivo.

In some embodiments of the above aspects, the subject is a human. The subject may be a male.

The subject may be a female.

In some embodiments of the above aspects, the cell is a cell of the central nervous system.

In some embodiments of the above aspects, the PCDH19 related disorder is selected from the group consisting of epilepsy, schizophrenia, and autism.

In some embodiments of the above aspects, the PCDH19 related disorder is epilepsy. The epilepsy may be epileptic early infantile encephalopathy 9.

In some embodiments of the above aspects, the subject has a mutation in at least one allele of the PCDH19 gene. The mutation may be heterozygous (e.g., if the subject is a female). The mutation may be hemizygous (e.g., if the subject is a male). If the subject is a male, the subject may have a mosaic mutation in which a subset of cells include the mutation. The mutation may be a missense mutation or a nonsense mutation. The mutation may be a frameshift mutation. The mutation may be an insertion or a deletion.

In some embodiments of the above aspects, the oligonucleotide selectively reduces expression of the allele including the mutation relative to an allele not comprising the mutation.

In some embodiments of the above aspects, the oligonucleotide reduces expression of the allele including the mutation and an allele including a wild-type sequence.

In some embodiments of the above aspects, the treatment reduces one or more symptoms of the PCDH19 related disorder. The one or more symptoms of the PCDH19 related disorder may be selected from the group consisting of prolonged seizures, frequent seizures, behavioral and developmental delays, movement and balance issues, orthopedic conditions, delayed language and speech issues, growth and nutrition issues, sleeping difficulties, chronic infection, sensory integration disorder, disruption of the autonomic nervous system, and sweating.

### BRIEF DESCRIPTION OF THE DRAWINGS

Exemplary embodiments of the present disclosure are described herein, by way of non-limiting example only, with reference to the following drawings.

FIG. 1. Relative PCDH19 mRNA expression in HEK293 cells 48 h post-transfection of 0.2 nM (grey bars) and 2.0 nM (black bars) of exemplary antisense oligonucleotides (ASOs). Data for PCDH19 mRNA was normalised to GAPDH mRNA and is expressed relative to the pooled means of mock-transfected cells and cells transfected with Ahsa1 ASOs that do not cross-react with PCDH19. Data is shown as means and standard deviation calculated from n=4 separate experiments. ASO numbering (abscissa values) corresponds to the ASO numbering shown in Table 2.

SEQ ID NO:1 depicts the nucleotide sequence of the human PCDH19 gene of NCBI Reference Sequence NC_000023.11 (region 100291644 . . . 100410273). Exemplary ASOs of the present invention are shown in SEQ ID NOs:2-385 (Table 2).

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

In this application, unless otherwise clear from context, (i) the term “a” may be understood to mean “at least one”; (ii) the term “or” may be understood to mean “and/or”; and (iii) the terms “including” and “comprising” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, an oligonucleotide with “no more than 3 mismatches to a target sequence” has 3, 2, 1, or 0 mismatches to a target sequence. When “no more than” is present before a series of numbers or a range, it is understood that “no more than” can modify each of the numbers in the series or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “administration” refers to the administration of a composition (e.g., a compound or a preparation that includes a compound as described herein) to a subject or system. Administration to an animal subject (e.g., to a human) may be by any appropriate route, such as one described herein.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “PCDH19” and “protocadherin 19” refer to a calcium dependent cell adhesion protein that is primarily expressed in developing brains. PCDH19 may have an amino acid sequence from any vertebrate or mammalian source, including, but not limited to, human, bovine, chicken, rodent, mouse, rat, porcine, ovine, primate, monkey, and guinea pig, unless specified otherwise. The term also refers to fragments and variants of native PCDH19 that maintain at least one in vivo or in vitro activity of a native PCDH19. The term encompasses full-length unprocessed precursor forms of PCDH19 as well as mature forms resulting from post-translational cleavage of the signal peptide.

PCDH19 is encoded by the PCDH19 gene. The nucleic acid sequences of exemplary human PCDH19 genes are set forth in NCBI Reference Nos. NC_000023.11, NG_021319.1, NM_001105243.1, NM_001184880.1, and NM_020766.2. The term “PCDH19” also refers to natural variants of the wild-type PCDH19 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type human PCDH19.

The term “PCDH19” as used herein also refers to a particular polypeptide expressed in a cell by naturally occurring DNA sequence variations of the PCDH19 gene, such as a single nucleotide polymorphism in the PCDH19 gene. Numerous SNPs within the PCDH19 gene have been identified and may be found at, for example, NCBI dbSNP (see, e.g., www.ncbi.nlm.nih.gov/snp).

As used herein, “target sequence” refers to a contiguous portion of the nucleotide sequence of an mRNA molecule formed during the transcription of a PCDH19 gene, including mRNA that is a product of RNA processing of a primary transcription product. In one embodiment, the target portion of the sequence will be at least long enough to serve as a substrate for oligonucleotide-directed (e.g., antisense oligonucleotide (ASO)-directed) cleavage at or near that portion of the nucleotide sequence of an mRNA molecule formed during the transcription of a PCDH19 gene. The target sequence may be, for example, from about 9-36 nucleotides in length, e.g., about 15-30 nucleotides in length, e.g., about 18-22 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

“G,” “C,” “A,” “T,” and “U” each generally stand for a naturally-occurring nucleotide that contains guanine, cytosine, adenine, thymidine, and uracil as a base, respectively. However, it will be understood that the term “nucleotide” can also refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of oligonucleotides featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “nucleobase” and “base” include the purine (e.g., adenine and guanine) and pyrimidine (e.g., uracil, thymine, and cytosine) moiety present in nucleosides and nucleotides which form hydrogen bonds in nucleic acid hybridization. In the context of the present invention, the term nucleobase also encompasses alternative nucleobases which may differ from naturally-occurring nucleobases, but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. Such variants are for example described in Hirao et al (2012) Accounts of Chemical Research vol. 45 page 2055 and Bergstrom (2009) Current Protocols in Nucleic Acid Chemistry Suppl. 37 1.4.1.

The term “nucleoside” refers to a monomeric unit of an oligonucleotide or a polynucleotide having a nucleobase and a sugar moiety. A nucleoside may include those that are naturally-occurring as well as alternative nucleosides, such as those described herein. The nucleobase of a nucleoside may be a naturally-occurring nucleobase or an alternative nucleobase. Similarly, the sugar moiety of a nucleoside may be a naturally-occurring sugar or an alternative sugar.

The term “alternative nucleoside” refers to a nucleoside having an alternative sugar or an alternative nucleobase, such as those described herein.

In a some embodiments the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methyl cytosine, 5-thiozolo-cytosine, 5-propynyl-cytosine, 5-propynyl-uridine, 5-bromouridine 5-thiazolo-uridine, 2-thio-uridine, pseudouridine, 1-methylpseudouridine, 5-methoxyuridine, 2′-thio-thymine, inosine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g., A, T, G, C, or U, wherein each letter may optionally include alternative nucleobases of equivalent function. In some embodiments, e.g., for gapmers, 5-methyl cytosine LNA nucleosides may be used.

A “sugar” or “sugar moiety” includes naturally occurring sugars having a furanose ring. A sugar also includes an “alternative sugar,” defined as a structure that is capable of replacing the furanose ring of a nucleoside. In certain embodiments, alternative sugars are non-furanose (or 4′-substituted furanose) rings or ring systems or open systems. Such structures include simple changes relative to the natural furanose ring, such as a six-membered ring, or may be more complicated as is the case with the non-ring system used in peptide nucleic acid. Alternative sugars may also include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O—(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization.

A “nucleotide,” as used herein, refers to a monomeric unit of an oligonucleotide or polynucleotide that comprises a nucleoside and an internucleosidic linkage. The internucleosidic linkage may or may not include a phosphate linkage. Similarly, “linked nucleosides” may or may not be linked by phosphate linkages. Many “alternative internucleosidic linkages” are known in the art, including, but not limited to, phosphate, phosphorothioate, and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide,” as used herein, refers to a nucleotide having an alternative nucleoside or an alternative sugar, and an internucleoside linkage, which may include alternative nucleoside linkages.

The terms “oligonucleotide” and “polynucleotide” as used herein are defined as it is generally understood by the skilled person as molecules comprising two or more covalently linked nucleosides. Such covalently bound nucleosides may also be referred to as nucleic acid molecules or oligomers. The term “oligonucleotide” refers to a short polynucleotide (typically of 100 or fewer linked nucleosides). Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention may be man-made, is chemically synthesized, and is typically purified or isolated. Oligonucleotide is also intended to include (i) compounds that have one or more furanose moieties that are replaced by furanose derivatives or by any structure, cyclic or acyclic, that may be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that may be used as a point of covalent attachment for the base moiety. The oligonucleotide of the invention may comprise one or more alternative nucleosides or nucleotides (e.g., including those described herein). It is also understood that oligonucleotide includes compositions lacking a sugar moiety or nucleobase but is still capable of forming a pairing with or hybridizing to a target sequence.

In the context of the present disclosure the terms “compound” and “oligonucleotide” may be used interchangeably. However falling within the scope of the term “compound” may be a compound that includes or comprises an oligonucleotide as disclosed herein and further including or comprising one or more additional moieties.

“Chimeric” oligonucleotides or “chimeras,” in the context of this invention, are oligonucleotides which contain two or more chemically distinct regions, each made up of at least one monomer unit, i.e., a nucleotide or nucleoside in the case of an oligonucleotide. Chimeric oligonucleotides also include “gapmers”.

The oligonucleotide may be of any length that permits specific degradation of a desired target RNA through an RNase H-mediated pathway, and may range from about 10-30 base pairs in length, e.g., about 15-30 base pairs in length or about 16-22 (e.g., 18-20) base pairs in length, for example, about 10, 11, 12, 13, 14, 15, 16, 17, 18, 19, 20, 21, 22, 23, 24, 25, 26, 27, 28, 29, or 30 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

As used herein, the term “oligonucleotide comprising a nucleobase sequence” refers to an oligonucleotide comprising a chain of nucleotides or nucleosides that is described by the sequence referred to using the standard nucleotide nomenclature.

The term “contiguous nucleobase region” refers to the region of the oligonucleotide which is complementary to the target nucleic acid. The term may be used interchangeably herein with the term “contiguous nucleotide sequence” or “contiguous nucleobase sequence.” In some embodiments all the nucleotides of the oligonucleotide are present in the contiguous nucleotide or nucleoside region. In some embodiments the oligonucleotide comprises the contiguous nucleotide region and may optionally comprise further nucleotide(s) or nucleoside(s), for example a nucleotide linker region which may be used to attach a functional group to the contiguous nucleotide sequence. The nucleotide linker region may or may not be complementary to the target nucleic acid. In some embodiments the internucleoside linkages present between the nucleotides of the contiguous nucleotide region are all phosphorothioate internucleoside linkages. In some embodiments, the contiguous nucleotide region comprises one or more sugar-modified nucleosides.

The term “gapmer” as used herein refers to an oligonucleotide which comprises a region of RNase H recruiting oligonucleotides (gap) which is flanked 5′ and 3′ by regions which comprise one or more affinity enhancing alternative nucleosides (wings or flanks). Various gapmer designs are described herein. Headmers and tailmers are oligonucleotides capable of recruiting RNase H where one of the wings is missing, i.e. only one of the ends of the oligonucleotide comprises affinity enhancing alternative nucleosides. For headmers the 3′ wing is missing (i.e. the 5′ wing comprises affinity enhancing alternative nucleosides) and for tailmers the 5′ wing is missing (i.e. the 3′ wing comprises affinity enhancing alternative nucleosides). A “mixed wing gapmer” refers to a gapmer wherein the wing regions comprise at least one alternative nucleoside, such as at least one DNA nucleoside or at least one 2′ substituted alternative nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-F-ANA nucleoside(s), or bicyclic nucleosides (e.g., locked nucleosides or constrained ethyl (cEt) nucleosides). In some embodiments the mixed wing gapmer has one wing which comprises alternative nucleosides (e.g., 5′ or 3′) and the other wing (3′ or 5′ respectfully) comprises 2′ substituted alternative nucleoside(s).

The term “linker” or “linking group” is a connection between two atoms that links one chemical group or segment of interest to another chemical group or segment of interest via one or more covalent bonds. Conjugate moieties can be attached to the oligonucleotide directly or through a linking moiety (e.g., linker or tether). Linkers serve to covalently connect a third region, e.g., a conjugate moiety to an oligonucleotide (e.g., the termini of region A or C). In some embodiments of the invention the conjugate or oligonucleotide conjugate of the invention may optionally, comprise a linker region which is positioned between the oligonucleotide and the conjugate moiety. In some embodiments, the linker between the conjugate and oligonucleotide is biocleavable. Phosphodiester containing biocleavable linkers are described in more detail in WO 2014/076195 (herein incorporated by reference).

As used herein, and unless otherwise indicated, the term “complementary,” when used to describe a first nucleotide or nucleoside sequence in relation to a second nucleotide or nucleoside sequence, refers to the ability of an oligonucleotide or polynucleotide comprising the first nucleotide or nucleoside sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides or nucleosides.

“Complementary” sequences, as used herein, can also include, or be formed entirely from, non-Watson-Crick base pairs and/or base pairs formed from non-natural and alternative nucleotides or nucleosides, in so far as the above requirements with respect to their ability to hybridize are fulfilled. Such non-Watson-Crick base pairs include, but are not limited to, G:U Wobble or Hoogstein base pairing. Complementary sequences between an oligonucleotide and a target sequence as described herein, include base-pairing of the oligonucleotide or polynucleotide comprising a first nucleotide or nucleoside sequence to an oligonucleotide or polynucleotide comprising a second nucleotide or nucleoside sequence over the entire length of one or both nucleotide or nucleoside sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via an RNase H-mediated pathway. “Substantially complementary” can also refer to a polynucleotide that is substantially complementary to a contiguous portion of the mRNA of interest (e.g., an mRNA encoding PCDH19). For example, a polynucleotide is complementary to at least a part of a PCDH19 mRNA if the sequence is substantially complementary to a non-interrupted portion of an mRNA encoding PCDH19.

As used herein, the term “region of complementarity” refers to the region on the oligonucleotide that is substantially complementary to all or a portion of a gene, primary transcript, a sequence (e.g., a target sequence, e.g., a PCDH19 nucleotide sequence), or processed mRNA, so as to interfere with expression of the endogenous gene (e.g., PCDH19). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the oligonucleotide.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, “hybridization” means the pairing of substantially complementary strands of nucleic acids, such as between an antisense oligonucleotide of the invention and a PCDH19 nucleotide sequence, e.g. an mRNA or pre-mRNA transcript. One mechanism of pairing involves hydrogen bonding, which may be Watson-Crick, Hoogsteen or reversed Hoogsteen hydrogen bonding, between complementary nucleobases of the strands of nucleic acids. For example, adenine and thymine or uracil are complementary nucleotides which pair through the formation of hydrogen bonds. Hybridization can occur under varying circumstances. Reference to “specifically hybridizes” as used herein means that the antisense oligonucleotide hybridizes to a target region in one PCDH19 allele, such as a mutant PCDH19 allele, and not to the same target region in another PCDH19 allele, such as a wild-type PCDH19 allele.

As used herein, an “agent that reduces the level and/or activity of PCDH19” refers to any polynucleotide agent (e.g., an oligonucleotide, e.g., an ASO) that reduces the level of or inhibits expression of PCDH19 in a cell or subject. The phrase “inhibiting expression of PCDH19,” as used herein, includes inhibition of expression of any PCDH19 gene (such as, e.g., a mouse PCDH19 gene, a rat PCDH19 gene, a monkey PCDH19 gene, or a human PCDH19 gene) as well as variants or mutants of a PCDH19 gene that encode a PCDH19 protein. Thus, the PCDH19 gene may be a wild-type PCDH19 gene, a mutant PCDH19 gene (e.g., insertion, deletion, nonsense mutant, missense mutant, frameshift mutant), or a transgenic PCDH19 gene in the context of a genetically manipulated cell, group of cells, or organism.

By “reducing the activity of PCDH19” is meant decreasing the level of an activity related to PCDH19 (e.g., an ion channel function). The activity level of PCDH19 may be measured using any method known in the art (e.g., using standard biophysical methods).

By “reducing the level of PCDH19” is meant decreasing the amount of PCDH19 in a cell or subject, e.g., by administering an oligonucleotide to the cell or subject. The level of PCDH19 may be measured using any method known in the art (e.g., by measuring the levels of PCDH19 mRNA or levels of PCDH19 protein in a cell or a subject).

As used herein, the term “inhibitor” refers to any agent which reduces the level and/or activity of a protein (e.g., PCDH19). Non-limiting examples of inhibitors include polynucleotides (e.g., oligonucleotide, e.g., ASOs). The term “inhibiting,” as used herein, is used interchangeably with “reducing,” “silencing,” “downregulating,” “suppressing,” and other similar terms, and includes any level of inhibition.

The phrase “contacting a cell with an oligonucleotide,” such as an oligonucleotide, as used herein, includes contacting a cell by any possible means. Contacting a cell with an oligonucleotide includes contacting a cell in vitro with the oligonucleotide or contacting a cell in vivo with the oligonucleotide. The contacting may be done directly or indirectly. Thus, for example, the oligonucleotide may be put into physical contact with the cell by the individual performing the method, or alternatively, the oligonucleotide agent may be put into a situation that will permit or cause it to subsequently come into contact with the cell.

Contacting a cell in vitro or ex vivo may be done, for example, by incubating the cell with the oligonucleotide. Contacting a cell in vivo may be done, for example, by injecting the oligonucleotide into or near the tissue where the cell is located, or by injecting the oligonucleotide agent into another area, e.g., the bloodstream or the subcutaneous space, such that the agent will subsequently reach the tissue where the cell to be contacted is located. For example, the oligonucleotide may contain and/or be coupled to a ligand, e.g., GalNAc3, that directs the oligonucleotide to a site of interest, e.g., the liver.

Combinations of in vitro, ex vivo and in vivo methods of contacting are also possible. For example, a cell may also be contacted in vitro with an oligonucleotide and subsequently transplanted into a subject. In one embodiment, contacting a cell with an oligonucleotide includes “introducing” or “delivering the oligonucleotide into the cell” by facilitating or effecting uptake or absorption into the cell. Absorption or uptake of an ASO can occur through unaided diffusive or active cellular processes, or by auxiliary agents or devices. Introducing an oligonucleotide into a cell may be in vitro, ex vivo and/or in vivo. For example, for in vivo introduction, oligonucleotides can be injected into a tissue site or administered systemically. In vitro introduction into a cell includes methods known in the art such as electroporation and lipofection. Further approaches are described herein below and/or are known in the art.

As used herein, “lipid nanoparticle” or “LNP” is a vesicle comprising a lipid layer encapsulating a pharmaceutically active molecule, such as a nucleic acid molecule, e.g., an oligonucleotide. LNP refers to a stable nucleic acid-lipid particle. LNPs typically contain a cationic lipid, a non-cationic lipid, and a lipid that prevents aggregation of the particle (e.g., a PEG-lipid conjugate). LNPs are described in, for example, U.S. Pat. Nos. 6,858,225; 6,815,432; 8,158,601; and 8,058,069, the entire contents of which are hereby incorporated herein by reference.

As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide composition. The lipophilic material isolates the aqueous interior from an aqueous exterior, which typically does not include the oligonucleotide composition, although in some examples, it may. Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

The term “antisense,” as used herein, refers to a nucleic acid comprising an oligonucleotide or polynucleotide that is sufficiently complementary to all or a portion of a gene, primary transcript, or processed mRNA, so as to interfere with expression of the endogenous gene (e.g., PCDH19). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

As used herein, the terms “effective amount,” “therapeutically effective amount,” and “a “sufficient amount” of an agent that reduces the level and/or activity of PCDH19 (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating a PCDH19 related disorder, it is an amount of the agent that reduces the level and/or activity of PCDH19 sufficient to achieve a treatment response as compared to the response obtained without administration of the agent that reduces the level and/or activity of PCDH19. The amount of a given agent that reduces the level and/or activity of PCDH19 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of PCDH19 of the present disclosure is an amount which results in a beneficial or desired result in a subject as compared to a control. As defined herein, a therapeutically effective amount of an agent that reduces the level and/or activity of PCDH19 of the present disclosure may be readily determined by one of ordinary skill by routine methods known in the art. The dosage regimen may be adjusted to provide the optimum therapeutic response.

“Prophylactically effective amount,” as used herein, is intended to include the amount of an oligonucleotide that, when administered to a subject having or predisposed to having a PCDH19 related disorder, is sufficient to prevent or ameliorate the disease or one or more symptoms of the disease. Ameliorating the disease includes slowing the course of the disease or reducing the severity of later-developing disease. The “prophylactically effective amount” may vary depending on the oligonucleotide, how the agent is administered, the degree of risk of disease, and the history, age, weight, family history, genetic makeup, the types of preceding or concomitant treatments, if any, and other individual characteristics of the subject to be treated. A prophylactically effective amount also refer to, for example, an amount of the agent reduces the level and/or activity of PCDH19 (e.g., in a cell or a subject) described herein refer to a quantity sufficient to, when administered to the subject, including a human, to delay the onset of a PCDH19 related disorder, as described herein, by at least 120 days, for example, at least 6 months, at least 12 months, at least 2 years, at least 3 years, at least 4 years, at least 5 years, at least 10 years or more, when compared with the predicted onset.

A “therapeutically-effective amount” or “prophylactically effective amount” also includes an amount (either administered in a single or in multiple doses) of an oligonucleotide that produces some desired local or systemic effect at a reasonable benefit/risk ratio applicable to any treatment. Oligonucleotides employed in the methods of the present invention may be administered in a sufficient amount to produce a reasonable benefit/risk ratio applicable to such treatment.

As used herein, the term “a subject identified as having a PCDH19 related disorder” refers to a subject identified as having a molecular or pathological state, disease or condition of or associated with a PCDH19 related disorder, such as the identification of a PCDH19 related disorder or symptoms thereof, or to refer to identification of a subject having or suspected of having a PCDH19 related disorder who may benefit from a particular treatment regimen.

As used herein, “PCDH19 related disorder,” refers to a class of genetic diseases or disorders characterized by aberrant function of PCDH19. PCDH19 related disorders include, for example, schizophrenia, autism, and epilepsy. For example, the epilepsy may be an epileptic encephalopathy, including early infantile epileptic encephalopathy, such as early infantile epileptic encephalopathy 9 (EIEE9).

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

By “level” is meant a level or activity of a protein, or mRNA encoding the protein (e.g., PCDH19), optionally as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, and ng/mL) or percentage relative to total protein or mRNA in a sample.

The term “pharmaceutical composition,” as used herein, represents a composition containing a compound described herein formulated with a pharmaceutically acceptable excipient, and preferably manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for intrathecal injection; for intracerebroventricular injections; for intraparenchymal injection; or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a subject. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., a PCDH19 related disorder); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition. The subject may be of any age, such as a newborn, neonate, infant, toddler, adolescent, or adult. The subject may be in utero.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The details of one or more embodiments of the invention are set forth in the description below. Other features, objects, and advantages of the invention will be apparent from the description and from the claims.

### DETAILED DESCRIPTION

According embodiments of the invention, inhibition or depletion of PCDH19 level and/or activity in a cell is effective in the treatment of a PCDH19 related disorder. Accordingly, the invention features useful compositions and methods to treat PCDH19 related disorders, e.g., in a subject in need thereof. The invention features single-stranded oligonucleotides that that target the PCDH19 gene. The oligonucleotides (e.g., chemically modified oligonucleotides) may be administered to a subject with a PCDH19 related disorder (e.g., epilepsy, schizophrenia, and autism) in order to treat, reduce the symptoms of, or prevent the PCDH19 related disorder. The oligonucleotides are antisense (e.g., at least partially complementary) to a target region of PCDH19 (e.g., PCDH19 mRNA, including pre-mRNA and processed mRNA). Following administration, the oligonucleotides reduce the level, expression, and/or activity of PCDH19 (e.g., PCDH19 mRNA and/or protein), thereby providing a therapeutic effect to the subject with a PCDH19 related disorder.

### PCDH19 Related Disorders

PCDH19 is a cadherin family protein that is primarily expressed in the developing brain. The gene encoding PCDH19 is located on the long (q) arm of the X chromosome at position 22.1. While its function has not been fully elucidated, it is believed to play a role in calcium dependent cell-adhesion. Full-length human processed PCDH19 mRNA is 9,765 nucleotides long, with exon 2 alternatively spliced.

Exemplary human PCDH19 genes include those with nucleotide sequences set forth in NCBI Reference Nos. NC_000023.11 (SEQ ID NO:1), NG_021319.1, NM_001105243.1, NM_001184880.1, and NM_020766.2, and variants thereof, including variants containing single nucleotide polymorphisms, such as those described in the various publically available databases (e.g. the Genome Aggregation Database (gnomAD) (http://gnomad.broadinstitute.org/). By way of example only, a PCDH19 variant may have at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO:1.

Mutations in PCDH19 (e.g., missense or nonsense mutations) have been associated with schizophrenia, autism, and particular forms of epilepsy, such as early infantile epileptic encephalopathy 9 (EIEE9). As PCDH19 is located on the X chromosome, EIEE9 is associated with a unique X-linked mode of inheritance. X-linked mutations usually affect males and do not affect carrier females. However, EIEE9 spares transmitting males while only affecting carrier females. EIEE9 is associated only with females, male subjects affected by mosaic PCDH19 expression have also been shown to experience cognitive impairment and/or epilepsy. Mosaicism involves the presence of two or more populations of cells expression different genotypes of PCDH19. In general, the mutations arises (e.g., de novo) in a particular cell and is only passed on to the daughter cells during division. Known mutations associated with PCDH19 related disorders include mutations that encode N340S, E307K, V441 E, Q85X, N557K, D594H, S671X, L677fsx717, 1119fsX122, and P364fsX375. Other known mutations associated with PCDH19 related disorders are listed in Table 1 below.

A subject with one or more of these mutations may be treated with compositions and methods described herein. Any of the PCDH19 related disorders described herein (e.g., epilepsy, autism, and schizophrenia) may be treated by administering the oligonucleotides (e.g., chemically modified oligonucleotides, e.g., ASOs) as described herein to reduce or eliminate the symptoms of the disorder.

TABLE 1
PCDH19 mutations

| | | GRCh37 | GRCh38 | GRCh38 | Variation | AIlele |
|---|---|---|---|---|---|---|
| DNA / Protein mutation | Name | Location | Chromosome | Location | ID | ID(s) |
| 1682C > G (p. Pro561Arg) | NM_001184880.1(PCDH19): c. 1006dup | 99662590 | X | 100407592 | 521360 | 512551 |
| | (p. Val336Glyfs) | | | | | |
| 1019A > C (p. Asn340Thr) | NM_001184880.1 | | | | | |
| 1700C > T (p. Pro567Leu) | NM_001184880.1(PCDH19): c. 1148G > A | 99662448 | X | 100407450 | 566197 | 575310 |
| | (p. Arg383His) | | | | | |
| 361G > A (p. Asp121Asn) | NM_001184880.1(PCDH19): c. 1281C > G | 99662315 | X | 100407317 | 533852 | 534955 |
| | (p. Asp427Glu) | | | | | |
| 424G > C (p. Ala142Pro) | NM_001184880.1(PCDH19): c. 1330A > G | 99662266 | X | 100407268 | 206296 | 203912 |
| | (p. Thr444Ala) | | | | | |
| 437C > G (p. Thr146Arg) | NM_001184880.1(PCDH19): c. 1335C > A | 99662261 | X | 100407263 | 206331 | 203911 |
| | (p. Asp445Glu) | | | | | |
| 593G > T (p. Arg198Leu) | NM_001184880.1(PCDH19): c. 1351C > T | 99662245 | X | 100407247 | 206332 | 203910 |
| | (p. Pro451Ser) | | | | | |
| 701A > G (p. Asn234Ser) | NM_001184880.1(PCDH19): c. 1355A > G | 99662241 | X | 100407243 | 568499 | 574230 |
| | (p. His452Arg) | | | | | |
| 134_135 del (D45G fs) | NM_001184880.1(PCDH19): c. 1382T > C | 99662214 | X | 100407216 | 206333 | 203908 |
| | (p. Ile461Thr) | | | | | |
| 152 dup T (A51R fs) | NM_001184880.1(PCDH19): c. 1384G > A | 99662212 | X | 100407214 | 432615 | 426389 |
| | (p. Val462Met) | | | | | |
| 1018A > T (p. Asn340Tyr) | NM_001184880.1 | | | | | |
| 1019A > G (p. Asn340Ser) | NM_001184880.1(PCDH19): c. 1419_1422delCTCT | 99662174- | X | 100407176- | 426961 | 415727 |
| | (p. Ser474Cysfs) | 99662177 | | 100407179 | | |
| 1031C > G (Asn340Arg) | NM_001184880.1(PCDH19): c. 142G > T | 99663454 | X | 100408456 | 11020 | 26059 |
| | (p. Glu48Ter) | | | | | |
| 1031C > T (p. Pro344Leu) | NM_001184880.1(PCDH19): c. 1434C > T | 99662162 | X | 100407164 | 415290 | 404010 |
| | (p. Arg478=) | | | | | |
| 1036_1040dup(N347K_fs) | NM_001184880.1(PCDH19): c. 1435G > A | 99662161 | X | 100407163 | 420880 | 411061 |
| | (p. Asp479Asn) | | | | | |
| 1049C > G (Ser350Ter) | NM_001184880.1(PCDH19): c. | 99662148- | X | 100407150- | 465297 | 472034 |
| | 1442_1448delACCTGGG (p. Asp481Valfs) | 99662154 | | 100407156 | | |
| 1091delC (p. Pro364Argfs) | NM_001184880.1 | | | | | |
| 1091dupC | NM_001184880.1 | | | | | |
| (p. Tyr366Leufs) | | | | | | |
| 1098C > G (p. Tyr366Ter) | NM_001184880.1(PCDH19): c. 1465T > C | 99662131 | X | 100407133 | 450108 | 446442 |
| | (p. Ser489Pro) | | | | | |
| 1101delG (p. Ile368Serfs) | NM_001184880.1(PCDH19): c. 1469A > C | 99662127 | X | 100407129 | 451251 | 446441 |
| | (p. Tyr490Ser) | | | | | |
| 1123G > T (D375Y) | NM_001184880.1(PCDH19): c. | 99662111- | X | 100407113- | 430517 | 422393 |
| | 1478_1485delTGCCGTCG (p. Val493Alafs) | 99662118 | | 100407120 | | |
| 1129G > A (D377N) | NM_001184880.1(PCDH19): c. 1478T > A | 99662118 | X | 100407120 | 408905 | 404419 |
| | (p. Val493Glu) | | | | | |
| 1129G > C (D377H) | NM_001184880.1(PCDH19): c. 1521delC | 99662075 | X | 100407077 | 429878 | 422392 |
| | (p. Ile508Serfs) | | | | | |
| 1131C > A (D377E) | NM_001184880.1(PCDH19): c. 1521dup | 99662075 | X | 100407077 | 533849 | 534422 |
| | (p. Ile508Hisfs) | | | | | |
| 1143dupT (G381W fs) | NM_001184880.1 | | | | | |
| 1178C > T (P393L) | NM_001184880.1(PCDH19): c. 1555C > T | 99662041 | X | 100407043 | 533844 | 534418 |
| | (p. Arg519Ter) | | | | | |
| 1183C > T (p. Arg395Ter) | NM_001184880.1 | | | | | |
| 1184G > C (R395P) | NM_001184880.1(PCDH19): c. 1625C > G | 99661971 | X | 100406973 | 206335 | 203906 |
| | (p. Ser542Ter) | | | | | |
| 1192G > T (E398Ter) | NM_001184880.1 | | | | | |
| 1240G > C (E414Q) | NM_001184880.1 | | | | | |
| 1240G > A (E414K) | NM_001184880.1(PCDH19): c. 1657_2616 + | 99654184- | X | 100399184- | 565718 | 575309 |
| | 3340del | 99661941 | | 100406941 | | |
| 1265_1266delCA | NM_001184880.1(PCDH19): c. 1671C > G | 99661925 | X | 100406927 | 11022 | 26061 |
| (p. Thr422Asnfs) | (p. Asn557Lys) | | | | | |
| 1298C > T (L433P) | NM_001184880.1(PCDH19): c. 1676delA | 99661919- | X | 100406922 | 561073 | 552236 |
| | (p. Asn559Thrfs) | 99661920 | | | | |
| 1300_1301 del (Q434E fs) | NM_001184880.1 | | | | | |
| 1322T > A (p. Val441Glu) | NM_001105243.1(PCDH19): c. | 99661900- | X | 100406902- | 206356 | 203903 |
| | 1683_1696deIGGTCATCACAGCCC (p. Val562Thrfs) | 99661913 | | 100406915 | | |
| 1347_1348insAAC | NM_001105243.1(PCDH19): c. 1683G > A | 99661913 | X | 100406915 | 93672 | 99575 |
| | (p. Pro561=) | | | | | |
| (N449_H450insN) | | | | | | |
| 1352C > T (P451L) | NM_001184880.1(PCDH19): c. 1697C > A | 99661899 | X | 100406901 | 521852 | 512549 |
| | (p. Pro566GIn) | | | | | |
| 1375C > T (Q459Ter) | NM_001184880.1(PCDH19): c. 169C > T | 99663427 | X | 100408429 | 447917 | 442363 |
| | (p. Gln57Ter) | | | | | |
| 1376delA (p. GIn459Argfs) | NM_001184880.1(PCDH19): c. 1700C > T | 99661896 | X | 100406898 | 449356 | 446440 |
| | (p. Pro567Leu) | | | | | |
| 138_139delGCinsTT | NM_001184880.1(PCDH19): c. 1720G > T | 99661876 | X | 100406878 | 425509 | 413843 |
| | (p. Glu574Ter) | | | | | |
| (p. Arg47Ter) | | | | | | |
| 1408_1417 del (A370S fs) | NM_001184880.1(PCDH19): c. 172G > T | 99663424 | X | 100408426 | 588339 | 580730 |
| | (p. Ala58Ser) | | | | | |
| 142G > T (p. Glu48Ter) | NM_001105243.1(PCDH19): c. | 99661837- | X | 100406839- | 206357 | 203901 |
| | 1752_1759delCTACCTGG (p. Tyr585Aspfs) | 99661844 | | 100406846 | | |
| 1456G > C (G486R) | NM_001184880.1 | | | | | |
| 1464_1466 del CTC (S489 | NM_001184880.1 | | | | | |
| del S) | | | | | | |
| 1521dup (p. Ile508Hisfs) | NM_001184880.1 | | | | | |
| 1522_1528 del (1508P fs) | NM_001184880.1 | | | | | |
| 1537G > C (G513R) | NM_001184880.1(PCDH19): c. 1815C > G | 99661781 | X | 100406783 | 445698 | 438970 |
| | (p. Tyr605Ter) | | | | | |
| 1615G > C (G539R) | NM_001184880.1 | | | | | |
| 1628T > C L543P | NM_001184880.1(PCDH19): c. | 99661739- | X | 100406741- | 570838 | 573497 |
| | 1853_1857delACCAGins CGAAGTCAGAA | 99661743 | | 100406745 | | |
| | (p. Asp618_Gln619delinsAlaLysSerGlu) | | | | | |
| 1649G > A (R550Q) | NM_001184880.1(PCDH19): c. 1854C > G | 99661742 | X | 100406744 | 576915 | 574221 |
| | (p. Asp618Glu) | | | | | |
| 1657_2616+3340del | NM_001184880.1(PCDH19): c. 1855C > T | 99661741 | X | 100406743 | 206338 | 203899 |
| | (p. GIn619Ter) | | | | | |
| 1671C > G (p. Asn557Lys) | NM_001184880.1 | | | | | |
| 1681C > T (P561S) | NM_001184880.1(PCDH19): c. 1864G > A | 99661732 | X | 100406734 | 589840 | 580590 |
| | (p. Gly622Ser) | | | | | |
| 1683_1696deIGGTCATCA | NM_001184880.1(PCDH19): c. 1873A > G | 99661723 | X | 100406725 | 206340 | 203898 |
| | (p. Arg625Gly) | | | | | |
| CAGCCC (p. Val562Thrfs) | | | | | | |
| 1752_1759delCTACCTGG | NM_001184880.1(PCDH19): c. 1952dupC | 99661644 | X | 100406646 | 206358 | 203895 |
| (p. Tyr585Aspfs) | (p. Leu652Serfs) | | | | | |
| 1765_1766deIGT V589C | NM_001184880.1 | | | | | |
| fs) | | | | | | |
| 1780G > C (D594H) | NM_001105243.1(PCDH19): c. 1956_1959deICTCT | 99661637- | X | 100406639- | 206369 | 203894 |
| | (p. Ser653Profs) | 99661640 | | 100406642 | | |
| 1786G > C (D596H) | NM_001105243.1(PCDH19): c. 1958_1959dupCT | 99661637- | X | 100406639- | 206359 | 203893 |
| | (p. Ala654Leufs) | 99661638 | | 100406640 | | |
| 1787A > T (D596V) | NM_001105243.1(PCDH19): c. 1969C > T | 99661627 | X | 100406629 | 159555 | 169842 |
| | (p. Leu657Phe) | | | | | |
| 1802G > A (G601D) | NM_001105243.1(PCDH19): c. | 99663389- | X | 100408391- | 206365 | 203952 |
| | 199_207delTCGGCTCCA (p. Ser67_Pro69del) | 99663397 | | 100408399 | | |
| 1804C > T (R602Ter) | NM_001184880.1(PCDH19): c. 2012C > G | 99661584 | X | 100406586 | 11018 | 26057 |
| | (p. Ser671Ter) | | | | | |
| 1825G > T (E609Ter) | NM_001184880.1 | | | | | |
| 1852G > A (D618N) | NM_001184880.1(PCDH19): c. 202G > A | 99663394 | X | 100408396 | 425512 | 413846 |
| | (p. Ala68Thr) | | | | | |
| 1863 dup T (G622W fs) | NM_001184880.1(PCDH19): c. 209_210del | 99663386- | X | 100408388- | 447918 | 442362 |
| | (p. His70Profs) | 99663387 | | 100408389 | | |
| 1864G > C (G622R) | NM_001184880.1(PCDH19): c. 21_39del19 | 99663557- | X | 100408559- | 422077 | 411067 |
| | (p. Val8Tyrfs) | 99663575 | | 100408577 | | |
| 1924G > A (V642M) | NM_001184880.1(PCDH19): c. 2122A > T | 99661474 | X | 100406476 | 586210 | 577910 |
| | (p. Lys708Ter) | | | | | |
| 1955T > C (L652P) | NM_001184880.1(PCDH19): c. 2147+1G > A | 99661448 | X | 100406450 | 489093 | 482227 |
| 1956_1959delCTCT | NM_001184880.1(PCDH19): c. 2147+2T > C | 99661447 | X | 100406449 | 449522 | 446439 |
| (p. Ser653Profs) | | | | | | |
| 1958_1959dupCT | NM_001105243.1(PCDH19): c. 218_219insG | 99663377- | X | 100408379- | 206367 | 203951 |
| (p. Ala654Leufs) | (p. Asp73Glufs) | 99663378 | | 100408380 | | |
| 2012C > G (p. Ser671Ter) | NM_001184880.1 | | | | | |
| 2019 del C (S674L fs) | NM_001184880.1 | | | | | |
| 2030_2031insT (L677 fs) | NM_001184880.1(PCDH19): c. 2191G > A | 99658619 | X | 100403621 | 533846 | 534450 |
| | (p. Gly731Arg) | | | | | |
| 2147+2T > C | NM_001105243.1(PCDH19): c. 2243delA | 99657754 | X | 100402756 | 206370 | 203888 |
| | (p. Glu748Glyfs) | | | | | |
| 215T > G (V72G) | NM_001184880.1(PCDH19): c. 2255A > G | 99658555 | X | 100403557 | 465298 | 470436 |
| | (p. Lys752Arg) | | | | | |
| 2156T > G (L719Ter) | NM_001105243.1(PCDH19): c. 2258delA | 99657739 | X | 100402741 | 206360 | 203887 |
| | (p. Asn753Thrfs) | | | | | |
| 2243delA (p. Glu748Glyfs) | NM_001184880.1(PCDH19): c. 2263_2288 + 1dup | 99658521- | X | 100403523- | 533850 | 534444 |
| | | 99658547 | | 100403549 | | |
| 2341delA (p. Ile781Serfs) | NM_001184880.1(PCDH19): c. 228C > T | 99663368 | X | 100408370 | 193192 | 190357 |
| | (p. Pro76=) | | | | | |
| 2359C > T (R787C) | NM_001184880.1(PCDH19): c. 2308G > C | 99657830 | X | 100402832 | 573321 | 573492 |
| | (p. Gly770Arg) | | | | | |
| 241dupC (L81C fs) | NM_001184880.1(PCDH19): c. 2319G > T | 99657819 | X | 100402821 | 206345 | 203889 |
| | (p. Lys773Asn) | | | | | |
| 2453delA (p. Gln818Argfs) | NM_001184880.1(PCDH19): c. 2341delA | 99657797 | X | 100402799 | 408902 | 404418 |
| | (p. Ile781Serfs) | | | | | |
| 253C > T (p. Gln85Ter) | NM_001184880.1 | | | | | |
| 2567delAGGGGCC, | NM_001184880.1(PCDH19): c. 2372G > A | 99657766 | X | 100402768 | 281920 | 266157 |
| (Q856P fs) | (p. Arg791Gln) | | | | | |
| 2656C > T (p. Arg886Ter) | NM_001184880.1(PCDH19): c. 2391C > G | 99657747 | X | 100402749 | 533847 | 534410 |
| | (p. Asp797Glu) | | | | | |
| 2675-6A > G p .? (intron | NM_001184880.1(PCDH19): c. 2422A > T | 99657716 | X | 100402718 | 408907 | 404037 |
| | (p. Thr808Ser) | | | | | |
| 4/exon 5) | | | | | | |
| 2675+1G > C p .? | NM_001184880.1 | | | | | |
| (exon/intron 4) | | | | | | |
| 2697dupA (E900R fs) | NM_001184880.1(PCDH19): c. 242T > G | 99663354 | X | 100408356 | 575889 | 573500 |
| | (p. Leu81Arg) | | | | | |
| 269A > T (D90V) | NM_001184880.1(PCDH19): c. 2453delA | 99657685 | X | 100402687 | 465300 | 471657 |
| | (p. Gln818Argfs) | | | | | |
| 2705dupA (D902K fs) | NM_001184880.1(PCDH19): c. 2479C > G | 99657659 | X | 100402661 | 566027 | 573488 |
| | (p. Arg827Gly) | | | | | |
| 2903dupA (D968E fs) | NM_001184880.1(PCDH19): c. 2496C > T | 99657642 | X | 100402644 | 196321 | 193482 |
| | (p. Phe832=) | | | | | |
| 339C > A (C113Ter) | NM_001184880.1(PCDH19): c. 2501dupA | 99657637 | X | 100402639 | 422860 | 411059 |
| | (p. Asn834Lysfs) | | | | | |
| 357delC (K120 fs) | NM_001184880.1(PCDH19): c. 2522G > A | 99657616 | X | 100402618 | 206302 | 203885 |
| | (p. Arg841His) | | | | | |
| 370G > A (D124N) | NM_001184880.1(PCDH19): c. 253C > T | 99663343 | X | 100408345 | 11017 | 26056 |
| | (p. Gln85Ter) | | | | | |
| 415_423dup | NM_001184880.1(PCDH19): c. 2564G > A | 99657574 | X | 100402576 | 206347 | 203884 |
| | (p. Ser855Asn) | | | | | |
| (S319_A141dup) | | | | | | |
| 416C > A (S139Ter) | NM_001184880.1 | | | | | |
| 445C > T (P149S) | NM_001184880.1(PCDH19): c. 2592T > A | 99657546 | X | 100402548 | 500578 | 492002 |
| | (p. Ile864=) | | | | | |
| 457G > A (A153T) | NM_001105243.1(PCDH19): c. 2601A > G | 99597007 | X | 100342009 | 159557 | 169840 |
| | (p. Gln867=) | | | | | |
| 462C > A or G (Y154Ter) | NM_001184880.1(PCDH19): c. 2617-7A > G | 99605709 | X | 100350711 | 287548 | 271785 |
| 469G > A (D157N) | NM_001105243.1(PCDH19): c. 2655C > T | 99596953 | X | 100341955 | 138601 | 142304 |
| | (p. Asn885=) | | | | | |
| 471C > A (D157E) | NM_001184880.1(PCDH19): c. 2656C > T | 99605663 | X | 100350665 | 206341 | 203882 |
| | (p. Arg886Ter) | | | | | |
| 473C > G (p. Ser158Ter) | NM_001184880.1(PCDH19): c. 2657G > A | 99605662 | X | 100350664 | 579743 | 575308 |
| | (p. Arg886GIn) | | | | | |
| 488T > G (V163G) | NM_001184880.1(PCDH19): c. 2668A > G | 99605651 | X | 100350653 | 206343 | 203881 |
| | (p. Ile890Val) | | | | | |
| 497_498 ins A (Y166ter) | NM_001184880.1(PCDH19): c. 2676- | 99597066- | X | 100342068- | 420905 | 411058 |
| | 1_2683delGCAGCTCCA | 99597074 | | 100342076 | | |
| 498C > G (p. Tyr166Ter) | NM_001184880.1(PCDH19): c. 268G > C | 99663328 | X | 100408330 | 465301 | 471210 |
| | (p. Asp90His) | | | | | |
| 506 del C (T169S fs) | NM_001184880.1 | | | | | |
| 514 dup G (E172G fs) | NM_001184880.1 | | | | | |
| 569T > G (L190R) | NM_001184880.1(PCDH19): c. 2721T > C | 99597028 | X | 100342030 | 167420 | 177908 |
| | (p. Ser907=) | | | | | |
| 595G > C (E199Q) | NM_001184880.1(PCDH19): c. 2798A > G | 99596951 | X | 100341953 | 447919 | 442356 |
| | (p. Asp933Gly) | | | | | |
| 605C > A (S202 ter) | NM_001184880.1(PCDH19): c. 2828G > A | 99596921 | X | 100341923 | 465302 | 471655 |
| | (p. Gly943Glu) | | | | | |
| 608A > C & 617T > G | NM_001105243.1(PCDH19): c. 2834C > A | 99551747 | X | 100296749 | 159558 | 169839 |
| | (p. Ala945Glu) | | | | | |
| (H203P & F206C) | | | | | | |
| 619delC (p. Arg207Glufs) | NM_001184880.1(PCDH19): c. 2849-2A > G | 99551875 | X | 100296877 | 265682 | 260268 |
| 695A > G (p. Asn232Ser) | NM_001184880.1 | | | | | |
| 696T > A (p. Asn232Lys) | NM_001184880.1(PCDH19): c. 295T > A | 99663301 | X | 100408303 | 421597 | 411064 |
| | (p. Cys99Ser) | | | | | |
| 697_700delinsTAAC | NM_001184880.1(PCDH19): c. 2965A > C | 99551757 | X | 100296759 | 514057 | 507726 |
| | (p. Asn989His) | | | | | |
| (D233 ter) | | | | | | |
| 706C > T (P236S) | NM_001184880.1(PCDH19): c. 2975C > T | 99551747 | X | 100296749 | 374533 | 361419 |
| | (p. Ala992Val) | | | | | |
| 718G > T (E240Ter) | NM_001184880.1(PCDH19): c. 298A > T | 99663298 | X | 100408300 | 206363 | 203949 |
| | (p. Ile100Phe) | | | | | |
| 729C > A (Y243Ter) | NM_001184880.1(PCDH19): c. 301_303delATC | 99663293- | X | 100408295- | 206348 | 203948 |
| | (p. Ile101del) | 99663295 | | 100408297 | | |
| 730dupG (A244G fs) | NM_001184880.1(PCDH19): c. 3011C > T | 99551711 | X | 100296713 | 408906 | 404007 |
| | (p. Ala1004Val) | | | | | |
| 746A > G (E249G) | NM_001184880.1(PCDH19): c. 301A > G | 99663295 | X | 100408297 | 193194 | 190359 |
| | (p. Ile101Val) | | | | | |
| 747A > T (E249D) | NM_001184880.1(PCDH19): c. 3021C > G | 99551701 | X | 100296703 | 571607 | 575307 |
| | (p. Asp1007Glu) | | | | | |
| 74T > C (L25P) | NM_001184880.1(PCDH19): c. 3057G > A | 99551665 | X | 100296667 | 285391 | 269628 |
| | (p. Gly1019=) | | | | | |
| 772_773 del (1258P fs) | NM_001184880.1(PCDH19): c. 3079G > A | 99551643 | X | 100296645 | 206304 | 203876 |
| | (p. Ala1027Thr) | | | | | |
| 78 del G (K26N fs) | NM_001184880.1(PCDH19): c. 3095C > T | 99551627 | X | 100296629 | 206305 | 203875 |
| | (p. Thr1032Ile) | | | | | |
| 790G > C (D264H) | NM_001184880.1(PCDH19): c. 311dup | 99663285 | X | 100408287 | 503908 | 495607 |
| | (p. Val105Glyfs) | | | | | |
| 799G > T (p. Glu267Ter) | NM_001184880.1(PCDH19): c. 3127A > G | 99551595 | X | 100296597 | 449713 | 446438 |
| | (p. Ile1043Val) | | | | | |
| 823T > A (Y275N) | NM_001184880.1(PCDH19): c. 3248A > G | 99551474 | X | 100296476 | 593456 | 584520 |
| | (p. Tyr1083Cys) | | | | | |
| 824A > C (Y275N) | NM_001184880.1(PCDH19): c. 3262G > A | 99551460 | X | 100296462 | 206306 | 203872 |
| | (p. Ala1088Thr) | | | | | |
| 826T > C (S276P) | NM_001184880.1(PCDH19): c. 3319C > G | 99551403 | X | 100296405 | 93675 | 99578 |
| | (p. Arg1107Gly) | | | | | |
| 83C > A (S28Ter) | NM_001184880.1(PCDH19): c. 3341G > C | 99551381 | X | 100296383 | 581970 | 572130 |
| | (p. Arg1114Pro) | | | | | |
| 83C > A & 90A > G (S28* & | NM_001184880.1(PCDH19): c. 3352A > G | 99551370 | X | 100296372 | 378327 | 377619 |
| E90E) | (p. Ser1118Gly) | | | | | |
| 840C > G (Y280Ter) | NM_001184880.1(PCDH19): c. 3370G > C | 99551352 | X | 100296354 | 575844 | 572128 |
| | (p. Glu1124Gln) | | | | | |
| 859G > T (p. Glu287Ter) | NM_001184880.1(PCDH19): c. 3379C > T | 99551343 | X | 100296345 | 533855 | 534560 |
| | (p. Pro1127Ser) | | | | | |
| 859G > T & 3319C > G | NM_001184880.1(PCDH19): c. 3398G > A | 99551324 | X | 100296326 | 431843 | 426387 |
| (E287* & R1107G) | (p. Arg1133His) | | | | | |
| 918C > G (p. Tyr306Ter) | NM_001184880.1(PCDH19): c. 3405A > G | 99551317 | X | 100296319 | 436167 | 430599 |
| | (p. Lys1135=) | | | | | |
| 937G > A (E307K) | NM_001184880.1(PCDH19): c. 3437T > C | 99551285 | X | 100296287 | 590086 | 580852 |
| | (p. Ile1146Thr) | | | | | |
| 949C > T (Q317Ter) | NM_001184880.1(PCDH19): c. 3439G > A | 99551283 | X | 100296285 | 206288 | 203869 |
| | (p. Val1147Ile) | | | | | |
| 958 dup G (D320G fs) | NM_001184880.1 | | | | | |
| 964G > C (G322R) | NM_001184880.1(PCDH19): c. 361G > A | 99663235 | X | 100408237 | 427126 | 415728 |
| | (p. Asp121Asn) | | | | | |
| 966dupG (p. Pro323Alafs) | NM_001184880.1(PCDH19): c. 361G > C | 99663235 | X | 100408237 | 206309 | 203947 |
| | (p. Asp121His) | | | | | |
| 976_981delATCCCGinsTT | NM_001184880.1(PCDH19): c. 369C > A | 99663227 | X | 100408229 | 206310 | 203946 |
| | (p. Asn123Lys) | | | | | |
| CCC (p. Ile326Phefs) | | | | | | |
| 994delG (p. Val332Serfs) | NM_001184880.1(PCDH19): c. 369C > G | 99663227 | X | 100408229 | 429767 | 422398 |
| | (p. Asn123Lys) | | | | | |
| NC_000023.10: g.9966275 | NM_001184880.1(PCDH19): c. 370G > T | 99663226 | X | 100408228 | 206311 | 203945 |
| | (p. Asp124Tyr) | | | | | |
| 5delC | | | | | | |
| PCDH19, 5-BP DUP, | NM_001184880.1(PCDH19): c. 372_373dup | 99663223- | X | 100408225- | 589933 | 580709 |
| NT1036 | (p. Asn125Thrfs) | 99663224 | | 100408226 | | |
| 242T > G (p. Leu81Arg) | NM_001184880.1(PCDH19): c. 619delC | 99662977 | X | 100407979 | 465307 | 472038 |
| | (p. Arg207Glufs) | | | | | |
| 416C > T (p. Ser139Leu) | NM_001184880.1 | | | | | |
| 571G > C (p. Val191Leu) | NM_001184880.1(PCDH19): c. 896C > T | 99662700 | X | 100407702 | 447922 | 442359 |
| | (p. Thr299Ile) | | | | | |
| 785C > A (p. Ala262Asp) | NM_001184880.1 | | | | | |
| 919G > A (p. Glu307Lys) | NC_000023.10: g.99662755delC | 99662755 | | | 597883 | 588944 |
| 91G > A (p. Glu31Lys) | NM_001184880.1: c. 2030dupT | | | | 11019 | 26058 |
| Whole gene deletions | | | | | | |

### Oligonucleotide Agents

Agents described herein that reduce the level and/or activity (e.g., aberrant activity) of PCDH19 in a cell may be, for example, a polynucleotide, e.g., an oligonucleotide. These agents reduce the level of an activity (e.g., binding activity) related to PCDH19, or a related downstream effect, or reduce the level of PCDH19 in a cell or subject.

In some embodiments, the agent that reduces the level and/or activity of PCDH19 is a polynucleotide. In some embodiments, the polynucleotide is a single-stranded oligonucleotide, e.g., that acts by way of an RNase H-mediated pathway. Oligonucleotides include DNA and DNA/RNA chimeric molecules, typically about 10 to 30 nucleotides in length, which recognize polynucleotide target sequences or sequence portions through hydrogen bonding interactions with the nucleotide bases of the target sequence (e.g., PCDH19). An oligonucleotide molecule can decrease the expression level (e.g., protein level or mRNA level) of PCDH19. For example, an oligonucleotide includes oligonucleotides that targets full-length PCDH19. In some embodiments, the oligonucleotide molecule recruits an RNase H enzyme, leading to target mRNA degradation.

In some embodiments, the oligonucleotide decreases the level and/or activity of a positive regulator of function. In other embodiments, the oligonucleotide increases the level and/or activity of an inhibitor of a positive regulator of function. In some embodiments, the oligonucleotide increases the level and/or activity of a negative regulator of function.

In some embodiments, the oligonucleotide decreases the level and/or activity or function of PCDH19. In some embodiments, the oligonucleotide inhibits expression of PCDH19. In other embodiments, the oligonucleotide increases degradation of PCDH19 and/or decreases the stability (i.e., half-life) of PCDH19 (e.g., mRNA). The oligonucleotide can be chemically synthesized.

The oligonucleotide includes an oligonucleotide having a region of complementarity (e.g., a contiguous nucleobase region) which is complementary to at least a part of an mRNA formed in the expression of a PCDH19 gene. The region of complementarity may be about 30 nucleotides or less in length (e.g., about 30, 29, 28, 27, 26, 25, 24, 23, 22, 21, 20, 19, 18, 17, 16, 15, 14, 13, 12, 11, or 10 nucleotides or less in length). Upon contact with a cell expressing the PCDH19 gene, the oligonucleotide may inhibit the expression of the PCDH19 gene (e.g., a human, a primate, a non-primate, or a bird PCDH19 gene) by at least about 10% (e.g., 20%, 30%, 40%, 50%, 60%, 70%, 80%, 90%, 95%, 97%, 99%, or 100%) as assayed by, for example, a PCR or branched DNA (bDNA)-based method, or by a protein-based method, such as by immunofluorescence analysis, using, for example, Western Blotting or flowcytometric techniques.

Similarly, the region of complementarity to the target sequence may be between 10 and 30 linked nucleosides in length, e.g., between 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 linked nucleosides in length. Ranges and lengths intermediate to the above recited ranges and lengths are also contemplated to be part of the invention.

The oligonucleotide may include a region that is at least 80% (e.g. 80%, 81%, 82%, 83%, 84%, 85%, 86%, 87%, 88%, 89%, 90%, 91%, 92%, 93%, 94%, 95%, 96%, 97%, 98%, 99%, or 100%) complementary to an equal length portion of a target region of the PCDH19 gene, for example a pre-mRNA transcript or mRNA transcript of PCDH19. The mRNA transcript or pre-mRNA transcript may contain a mutation (e.g., the subject is heterozygous (e.g., female) or hemizygous (e.g., male) for the mutation). The oligonucleotide may selectively target the mRNA or pre-mRNA containing the mutation (e.g., an allele specific ASO). The oligonucleotide may target both the mutant allele and the wild-type allele.

An oligonucleotide can be synthesized by standard methods known in the art as further discussed below, e.g., by use of an automated DNA synthesizer, such as are commercially available from, for example, Biosearch, Applied Biosystems, Inc.

The oligonucleotide compound can be prepared using solution-phase or solid-phase organic synthesis or both. Organic synthesis offers the advantage that the oligonucleotide comprising unnatural or alternative nucleotides can be easily prepared. Single-stranded oligonucleotides of the invention can be prepared using solution-phase or solid-phase organic synthesis or both.

In one aspect, an oligonucleotide of the invention includes a region of at least 10 (e.g., 11, 12, 13, 14, 15, 16, 17, 18, 19, 20, or more) contiguous nucleobases having at least 80% (e.g., at least 85%, at least 90%, at least 95%, or at least 99%) complementary to at least 10 contiguous nucleotides of a PCDH19 gene. In some embodiments, the oligonucleotide comprises a sequence complementary to at least 17 contiguous nucleotides, 19-23 contiguous nucleotides, 19 contiguous nucleotides, or 20 contiguous nucleotides of a PCDH19 gene. In this aspect, the sequence is substantially complementary to a sequence of an mRNA generated in the expression of a PCDH19 gene.

In some embodiments the oligonucleotide of the invention, or contiguous nucleotide region thereof, has a gapmer design or structure also referred herein merely as “gapmer.” In a gapmer structure the oligonucleotide comprises at least three distinct structural regions a 5′-wing, a gap and a 3′-wing, in ‘5->3′ orientation. In this design, the 5′ and 3′ wing regions (also termed flanking regions) comprise at least one alternative nucleoside which is adjacent to a gap region, and may in some embodiments comprise a contiguous stretch of 2-7 alternative nucleosides, or a contiguous stretch of alternative and DNA nucleosides (mixed wings comprising both alternative and DNA nucleosides). The length of the 5′-wing region may be at least two nucleosides in length (e.g., at least at least 2, at least 3, at least 4, at least 5, or more nucleosides in length). The length of the 3′-wing region may be at least two nucleosides in length (e.g., at least 2, at least 3, at least at least 4, at least 5, or more nucleosides in length). The 5′ and 3′ wing regions may be symmetrical or asymmetrical with respect to the number of nucleosides they comprise. In some embodiments, the gap region comprises about 10 nucleosides flanked by a 5′ and a 3′ wing region each comprising about 5 nucleosides, also referred to as a 5-10-5 gapmer.

Consequently, the nucleosides of the 5′ wing region and the 3′ wing region which are adjacent to the gap region are alternative nucleosides, such as 2′ alternative nucleosides. The gap region comprises a contiguous stretch of nucleotides which are capable of recruiting RNase H, when the oligonucleotide is in duplex with the PCDH19 target nucleic acid. In some embodiments, the gap region comprises a contiguous stretch of 5-16 DNA nucleosides. In other embodiments, the gap region comprises a region of at least 10 contiguous nucleobases having at least 80% (e.g., at least 85%, at least 90%, at least 95%, or at least 99%) complementarity to a PCDH19 gene. In some embodiments, the gapmer comprises a region complementary to at least 17 contiguous nucleotides, 19-23 contiguous nucleotides, or 19 contiguous nucleotides of a PCDH19 gene. The gapmer is complementary to the PCDH19 target nucleic acid, and may therefore be the contiguous nucleoside region of the oligonucleotide.

The 5′ and 3′ wing regions, flanking the 5′ and 3′ ends of the gap region, may comprise one or more affinity enhancing alternative nucleosides. In some embodiments, the 5′ and/or 3′ wing comprises at least one 2′-O-methoxyethyl (MOE) nucleoside, preferably at least two MOE nucleosides. In some embodiments, the 5′ wing comprises at least one MOE nucleoside. In some embodiments both the 5′ and 3′ wing regions comprise a MOE nucleoside. In some embodiments all the nucleosides in the wing regions are MOE nucleosides. In other embodiments, the wing regions may comprise both MOE nucleosides and other nucleosides (mixed wings), such as DNA nucleosides and/or non-MOE alternative nucleosides, such as bicyclic nucleosides (BNAs) (e.g., LNA nucleosides or cET nucleosides), or other 2′ substituted nucleosides. In this case the gap is defined as a contiguous sequence of at least 5 RNase H recruiting nucleosides (such as 5-16 DNA nucleosides) flanked at the 5′ and 3′ end by an affinity enhancing alternative nucleoside, such as an MOE nucleoside.

In other embodiments, the 5′ and/or 3′ wing comprises at least one BNA (e.g., at least one LNA nucleoside or cET nucleoside), preferably at least 2 bicyclic nucleosides. In some embodiments, the 5′ wing comprises at least one BNA. In some embodiments both the 5′ and 3′ wing regions comprise a BNA. In some embodiments all the nucleosides in the wing regions are BNAs. In other embodiments, the wing regions may comprise both BNAs and other nucleosides (mixed wings), such as DNA nucleosides and/or non-BNA alternative nucleosides, such as 2′ substituted nucleosides. In this case the gap is defined as a contiguous sequence of at least five RNase H recruiting nucleosides (such as 5-16 DNA nucleosides) flanked at the 5′ and 3′ end by an affinity enhancing alternative nucleoside, such as a BNA, such as an LNA, such as beta-D-oxy-LNA.

The 5′ flank or 5′ wing attached to the 5′ end of the gap region comprises, contains, or consists of at least one alternative sugar moiety (e.g., at least three, at least four, at least five, at least six, at least seven, or more alternative sugar moieties). In some embodiments the wing region comprises or consists of from 1 to 7 alternative nucleobases, such as from 2 to 6 alternative nucleobases, such as from 2 to 5 alternative nucleobases, such as from 2 to 4 alternative nucleobases, such as from 1 to 3 alternative nucleobases, such as one, two, three or four alternative nucleobases. In some embodiments, the wing region comprises or consists of at least one alternative internucleoside linkage (e.g., at least three, at least four, at least five, at least six, at least seven, or more alternative internucleoside linkages).

The 3′ flank or 3′ wing attached to the 3′ end of the gap region comprises, contains, or consists of at least one alternative sugar moiety (e.g., at least three, at least four, at least five, at least six, at least seven, or more alternative sugar moieties). In some embodiments the wing region comprises or consists of from 1 to 7 alternative nucleobases, such as from 2 to 6 alternative nucleobases, such as from 2 to 5 alternative nucleobases, such as from 2 to 4 alternative nucleobases, such as from 1 to 3 alternative nucleobases, such as one, two, three, or four alternative nucleobases. In some embodiments, the wing region comprises or consists of at least one alternative internucleoside linkage (e.g., at least three, at least four, at least five, at least six, at least seven, or more alternative internucleoside linkages).

In an embodiment, one or more or all of the alternative sugar moieties in the wing regions are 2′ alternative sugar moieties.

In a further embodiment, one or more of the 2′ alternative sugar moieties in the wing regions are selected from 2′-O-alkyl-sugar moieties, 2′-O-methyl-sugar moieties, 2′-amino-sugar moieties, 2′-fluoro-sugar moieties, 2′-alkoxy-sugar moieties, MOE sugar moieties, LNA sugar moieties, arabino nucleic acid (ANA) sugar moieties, and 2′-fluoro-ANA sugar moieties.

In one embodiment of the invention all the alternative nucleosides in the wing regions are bicyclic nucleosides. In a further embodiment the bicyclic nucleosides in the wing regions are independently selected from the group consisting of oxy-LNA, thio-LNA, amino-LNA, cET, and/or ENA, in either the beta-D or alpha-L configurations or combinations thereof.

In some embodiments, the one or more alternative internucleoside linkages in the wing regions are phosphorothioate internucleoside linkages. In some embodiments, the phosphorothioate linkages are stereochemically pure phosphorothioate linkages. In some embodiments the phosphorothioate linkages are Sp phosphorothioate linkages. In other embodiments, the phosphorothioate linkages are Rp phosphorothioate linkages. In some embodiments, the alternative internucleoside linkages are 2′-alkoxy internucleoside linkages. In other embodiments, the alternative internucleoside linkages are alkyl phosphate internucleoside linkages.

The gap region may comprise, contain, or consist of at least 5-16 consecutive DNA nucleosides capable of recruiting RNase H. In some embodiments, all of the nucleosides of the gap region are DNA units. In further embodiments the gap region may consist of a mixture of DNA and other nucleosides capable of mediating RNase H cleavage. In some embodiments, at least 50% of the nucleosides of the gap region are DNA, such as at least 60%, at least 70% or at least 80%, or at least 90% DNA.

The oligonucleotide of the invention comprises a contiguous region which is complementary to the target nucleic acid. In some embodiments, the oligonucleotide may further comprise additional linked nucleosides positioned 5′ and/or 3′ to either the 5′ and 3′ wing regions. These additional linked nucleosides can be attached to the 5′ end of the 5′ wing region or the 3′ end of the 3′ wing region, respectively. The additional nucleosides may, in some embodiments, form part of the contiguous sequence which is complementary to the target nucleic acid, or in other embodiments, may be non-complementary to the target nucleic acid.

The inclusion of the additional nucleosides at either, or both of the 5′ and 3′ wing regions may independently comprise one, two, three, four, or five additional nucleotides, which may be complementary or non-complementary to the target nucleic acid. In this respect the oligonucleotide of the invention, may in some embodiments comprise a contiguous sequence capable of modulating the target which is flanked at the 5′ and/or 3′ end by additional nucleotides. Such additional nucleosides may serve as a nuclease susceptible biocleavable linker, and may therefore be used to attach a functional group such as a conjugate moiety to the oligonucleotide of the invention. In some embodiments the additional 5′ and/or 3′ end nucleosides are linked with phosphodiester linkages, and may be DNA or RNA. In another embodiment, the additional 5′ and/or 3′ end nucleosides are alternative nucleosides which may for example be included to enhance nuclease stability or for ease of synthesis.

In other embodiments, the oligonucleotides of the invention utilize “altimer” design and comprise alternating 2′-fluoro-ANA and DNA regions that are alternated every three nucleosides. Altimer oligonucleotides are discussed in more detail in Min, et al., Bioorganic & Medicinal Chemistry Letters, 2002, 12(18): 2651-2654 and Kalota, et al., Nuc. Acid Res. 2006, 34(2): 451-61 (herein incorporated by reference).

In other embodiments, the oligonucleotides of the invention utilize “hemimer” design and comprise a single 2′-modified wing segment adjacent to (on either side of the 5′ or the 3′ side of) a gap region. Hemimer oligonucleotides are discussed in more detail in Geary et al., 2001, J. Pharm. Exp. Therap., 296: 898-904 (herein incorporated by reference).

In some embodiments, an oligonucleotide has a nucleic acid sequence with at least 50% (e.g., at least 50%, at least 60%, at least 70%, at least 80%, at least 85%, at least 90%, at least 95%, at least 96%, at least 97%, at least 98%, at least 99%, or 100%) sequence identity to an equal length target sequence of PCDH19. In some embodiments, an oligonucleotide has a nucleic acid sequence with at least 85% sequence identity to an equal length target sequence of PCDH19.

It will be understood that, although the ASO sequences are described as unmodified and/or un-conjugated sequences, the nucleosides of the oligonucleotide of the invention e.g., an oligonucleotide of the invention, may comprise any sequence that is an alternative nucleoside and/or conjugated as described in detail below.

The skilled person is well aware that oligonucleotides having a structure of between about 18-20 base pairs may be particularly effective in inducing RNase H-mediated degradation. However, one can appreciate that shorter or longer oligonucleotides can also be effective. In the embodiments described above, by virtue of the nature of the oligonucleotide sequences provided herein, oligonucleotides described herein can include. It can be reasonably expected that shorter oligonucleotides minus only a few linked nucleosides on one or both ends can be similarly effective as compared to the oligonucleotides described above. Hence, oligonucleotides having a sequence of at least 8, 9, 10, 11, 12, 13, 14, 15, 16, 17, 18, 19, 20, or more contiguous linked nucleosides derived from one of the sequences provided herein, and differing in their ability to inhibit the expression of a PCDH19 gene by not more than about 5, 10, 15, 20, 25, or 30% inhibition from an oligonucleotide comprising the full sequence, are contemplated to be within the scope of the present invention.

The oligonucleotides described herein may function via nuclease mediated degradation of the target nucleic acid, where the oligonucleotides of the invention are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing alternative nucleosides, for example gapmers, headmers, and tailmers.

The RNase H activity of an oligonucleotide refers to its ability to recruit RNase H when in a duplex with a complementary RNA molecule. WO01/23613 provides in vitro methods for determining RNase H activity, which may be used to determine the ability to recruit RNase H. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using an oligonucleotide having the same base sequence as the modified oligonucleotide being tested, but containing only DNA monomers, with phosphorothioate linkages between all monomers in the oligonucleotide, and using the methodology provided by Example 91-95 of WO01/23613 (hereby incorporated by reference).

Furthermore, the oligonucleotides described herein identify a site(s) in a PCDH19 transcript that is susceptible to RNase H-mediated cleavage. As such, the present invention further features oligonucleotides that target within this site(s). As used herein, an oligonucleotide is said to target within a particular site of an RNA transcript if the oligonucleotide promotes cleavage of the transcript anywhere within that particular site. Such an oligonucleotide will generally include at least about 5-10 contiguous linked nucleosides from one of the sequences provided herein coupled to additional linked nucleoside sequences taken from the region contiguous to the selected sequence in a PCDH19 gene.

Inhibitory oligonucleotides can be designed by methods well known in the art. While a target sequence is generally about 10-30 linked nucleosides in length, there is wide variation in the suitability of particular sequences in this range for directing cleavage of any given target RNA.

Oligonucleotides with homology sufficient to provide sequence specificity required to uniquely degrade any RNA can be designed using programs known in the art

Systematic testing of several designed species for optimization of the inhibitory oligonucleotide sequence can also be undertaken in accordance with the teachings provided herein. Considerations when designing interfering oligonucleotides include, but are not limited to, biophysical, thermodynamic, and structural considerations, base preferences at specific positions, and homology. The making and use of inhibitory therapeutic agents based on non-coding oligonucleotides are also known in the art.

Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an oligonucleotide agent, mediate the best inhibition of target gene expression. Thus, while the sequences identified herein represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified herein, further optimization could be achieved by systematically either adding or removing linked nucleosides to generate longer or shorter sequences and testing those sequences generated by walking a window of the longer or shorter size up or down the target RNA from that point. Again, coupling this approach to generating new candidate targets with testing for effectiveness of oligonucleotides based on those target sequences in an inhibition assay as known in the art and/or as described herein can lead to further improvements in the efficiency of inhibition.

Further still, such optimized sequences can be adjusted by, e.g., the introduction of alternative nucleosides, alternative sugar moieties, and/or alternative internucleosidic linkages as described herein or as known in the art, including alternative nucleosides, alternative sugar moieties, and/or alternative internucleosidic linkages as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes) as an expression inhibitor. An oligonucleotide agent as described herein can contain one or more mismatches to the target sequence. In one embodiment, an oligonucleotide as described herein contains no more than 3 mismatches. If the oligonucleotide contains mismatches to a target sequence, it is preferable that the area of mismatch is not located in the center of the region of complementarity. If the oligonucleotide contains mismatches to the target sequence, it is preferable that the mismatch be restricted to be within the last 5 nucleotides from either the 5′- or 3′-end of the region of complementarity. For example, for a 30-linked nucleoside oligonucleotide agent, the contiguous nucleobase region which is complementary to a region of a PCDH19 gene, generally does not contain any mismatch within the central 5-10 linked nucleosides. The methods described herein or methods known in the art can be used to determine whether an oligonucleotide containing a mismatch to a target sequence is effective in inhibiting the expression of a PCDH19 gene. Consideration of the efficacy of oligonucleotides with mismatches in inhibiting expression of a PCDH19 gene is important, especially if the particular region of complementarity in a PCDH19 gene is known to have polymorphic sequence variation within the population.

Construction of vectors for expression of polynucleotides for use in the invention may be accomplished using conventional techniques which do not require detailed explanation to one of ordinary skill in the art. For generation of efficient expression vectors, it is necessary to have regulatory sequences that control the expression of the polynucleotide. These regulatory sequences include promoter and enhancer sequences and are influenced by specific cellular factors that interact with these sequences, and are well known in the art.

### Alternative Oligonucleotides

In one embodiment, one or more of the linked nucleosides or internucleosidic linkages of the oligonucleotide of the invention, is naturally occurring, and does not comprise, e.g., chemical modifications and/or conjugations known in the art and described herein. In another embodiment, one or more of the linked nucleosides or internucleosidic linkages of an oligonucleotide of the invention, is chemically modified to enhance stability or other beneficial characteristics. Without being bound by theory, it is believed that certain modifications can increase nuclease resistance and/or serum stability, or decrease immunogenicity. For example, oligonucleotides of the invention may contain nucleotides found to occur naturally in DNA or RNA (e.g., adenine, thymidine, guanosine, cytidine, uridine, or inosine) or may contain alternative nucleosides or internucleosidic linkages which have one or more chemical modifications to one or more components of the nucleotide (e.g., the nucleobase, sugar, or phospho-linker moiety). Oligonucleotides of the invention may be linked to one another through naturally occurring phosphodiester bonds, or may contain alternative linkages (e.g., covalently linked through phosphorothioate (e.g., Sp phosphorothioate or Rp phosphorothioate), 3′-methylenephosphonate, 5′-methylenephosphonate, 3′-phosphoamidate, 2′-5′ phosphodiester, guanidinium, S-methylthiourea, 2′-alkoxy, alkyl phosphate, or peptide bonds).

In certain embodiments of the invention, substantially all of the nucleosides or internucleosidic linkages of an oligonucleotide of the invention are alternative nucleosides. In other embodiments of the invention, all of the nucleosides or internucleosidic linkages of an oligonucleotide of the invention are alternative nucleosides. Oligonucleotides of the invention in which “substantially all of the nucleosides are alternative nucleosides” are largely but not wholly modified and can include not more than five, four, three, two, or one naturally-occurring nucleosides. In still other embodiments of the invention, oligonucleotides of the invention can include not more than five, four, three, two, or one alternative nucleosides.

The nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Alternative nucleotides and nucleosides include those with modifications including, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. The nucleobase may also be an isonucleoside in which the nucleobase is moved from the C1 position of the sugar moiety to a different position (e.g., C2, C3, C4, or C5). Specific examples of oligonucleotide compounds useful in the embodiments described herein include, but are not limited to alternative nucleosides containing modified backbones or no natural internucleoside linkages. Nucleotides and nucleosides having modified backbones include, among others, those that do not have a phosphorus atom in the backbone. For the purposes of this specification, and as sometimes referenced in the art, alternative RNAs that do not have a phosphorus atom in their internucleoside backbone can also be considered to be oligonucleosides. In some embodiments, an oligonucleotide will have a phosphorus atom in its internucleoside backbone.

Alternative internucleoside linkages include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boronophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts, and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Alternative internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable oligonucleotides include those in which both the sugar and the internucleoside linkage, i.e., the backbone, of the nucleotide units are replaced. The base units are maintained for hybridization with an appropriate nucleic acid target compound. One such oligomeric compound, a mimetic that has been shown to have excellent hybridization properties, is referred to as a peptide nucleic acid (PNA). In PNA compounds, the sugar of a nucleoside is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the oligonucleotides of the invention are described in, for example, in Nielsen et al., Science, 1991, 254, 1497-1500.

Some embodiments featured in the invention include oligonucleotides with phosphorothioate backbones and oligonucleotides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester backbone is represented as —O—P—O—CH₂-] of the above-referenced U.S. Pat. No. 5,489,677, and the amide backbones of the above-referenced U.S. Pat. No. 5,602,240. In some embodiments, the oligonucleotides featured herein have morpholino backbone structures of the above-referenced U.S. Pat. No. 5,034,506. In other embodiments, the oligonucleotides described herein include phosphorodiamidate morpholino oligomers (PMO), in which the deoxyribose moiety is replaced by a morpholine ring, and the charged phosphodiester inter-subunit linkage is replaced by an uncharged phophorodiamidate linkage, as described in Summerton, et al., Antisense Nucleic Acid Drug Dev. 1997, 7:63-70.

Alternative nucleosides and nucleotides can also contain one or more substituted sugar moieties. The oligonucleotides, e.g., oligonucleotides, featured herein can include one of the following at the 2′-position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include —O[(CH₂)ₙO]ₘCH₃, —O(CH₂)ₙOCH₃, —O(CH₂)ₙ—NH₂,

—O(CH₂)ₙCH₃, —O(CH₂)ₙ—ONH₂, and —O(CH₂)ₙ—ON[(CH₂)ₙCH₃]2, where n and m are from 1 to about 10. In other embodiments, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chin. Acta, 1995, 78:486-504) i.e., an alkoxy-alkoxy group. MOE nucleosides confer several beneficial properties to oligonucleotides including, but not limited to, increased nuclease resistance, improved pharmacokinetics properties, reduced non-specific protein binding, reduced toxicity, reduced immunostimulatory properties, and enhanced target affinity as compared to unmodified oligonucleotides.

Another exemplary alternative contains 2′-dimethylaminooxyethoxy, i.e., a —O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—(CH₂)₂—O—(CH₂)₂—N(CH₃)₂. Further exemplary alternatives include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other alternatives include 2′-methoxy (2′-OCH₃), 2′-aminopropoxy (2′-OCH₂CH₂CH₂NH₂) and 2′-fluoro (2′-F). Similar modifications can also be made at other positions on the nucleosides and nucleotides of an oligonucleotide, particularly the 3′ position of the sugar on the 3′ terminal nucleotide or in 2′-5′ linked oligonucleotides and the 5′ position of 5′ terminal nucleotide. Oligonucleotides can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An oligonucleotide of the invention can also include nucleobase (often referred to in the art simply as “base”) alternatives (e.g., modifications or substitutions). Unmodified or natural nucleobases include the purine bases adenine (A) and guanine (G), and the pyrimidine bases thymine (T), cytosine (C) and uracil (U). Alternative nucleobases include other synthetic and natural nucleobases such as 5-methylcytidine, 5-hydroxymethylcytidine, 5-formylcytidine, 5-carboxycytidine, pyrrolocytidine, dideoxycytidine, uridine, 5-methoxyuridine, 5-hydroxydeoxyuridine, dihydrouridine, 4-thiourdine, pseudouridine, 1-methyl-pseudouridine, deoxyuridine, 5-hydroxybutynl-2′-deoxyuridine, xanthine, hypoxanthine, 7-deaza-xanthine, thienoguanine, 8-aza-7-deazaguanosine, 7-methylguanosine, 7-deazaguanosine, 6-aminomethyl-7-deazaguanosine, 8-aminoguanine, 2,2,7-trimethylguanosine, 8-methyladenine, 8-azidoadenine, 7-methyladenine, 7-deazaadenine, 3-deazaadenine, 2,6-diaminopurine, 2-aminopurine, 7-deaza-8-aza-adenine, 8-amino-adenine, thymine, dideoxythymine, 5-nitroindole, 2-aminoadenine, 6-methyl and other alkyl derivatives of adenine and guanine, 2-propyl and other alkyl derivatives of adenine and guanine, 2-thiouridine, 2-thiothymine and 2-thiocytosine, 5-halouracil and cytosine, 5-propynyl uridine and cytidine, 6-azo uridine, cytidine and thymine, 4-thiouridine, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uridines and cytidines, 8-azaguanine and 8-azaadenine, and 3-deazaguanine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, Antisense Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines, and N-2, N-6 and O-6 substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., Antisense Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted alternative nucleobases as well as other alternative nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In other embodiments, the sugar moiety in the nucleotide may be a ribose molecule, optionally having a 2′-O-methyl, 2′-O-MOE, 2′-F, 2′-amino, 2′-O-propyl, 2′-aminopropyl, or 2′-OH modification.

An oligonucleotide of the invention can include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include one or more locked nucleosides. A locked nucleoside is a nucleoside having a modified ribose moiety in which the ribose moiety comprises an extra bridge connecting the 2′ and 4′ carbons. In other words, a locked nucleoside is a nucleoside comprising a bicyclic sugar moiety comprising a 4′-CH₂—O-2′ bridge. This structure effectively “locks” the ribose in the 3′-endo structural conformation. The addition of locked nucleosides to oligonucleotides has been shown to increase oligonucleotide stability in serum, and to reduce off-target effects (Grunweller, A. et al., (2003) Nucleic Acids Research 31(12):3185-3193).

Examples of bicyclic nucleosides for use in the polynucleotides of the invention include without limitation nucleosides comprising a bridge between the 4′ and the 2′ ribosyl ring atoms. In certain embodiments, the polynucleotide agents of the invention include one or more bicyclic nucleosides comprising a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

An oligonucleotide of the invention can also be modified to include one or more constrained ethyl nucleosides. As used herein, a “constrained ethyl nucleoside” or “cEt” is a locked nucleoside comprising a bicyclic sugar moiety comprising a 4′-CH(CH₃)—O-2′ bridge. In one embodiment, a constrained ethyl nucleoside is in the S conformation referred to herein as “S-cEt.”

An oligonucleotide of the invention may also include one or more “conformationally restricted nucleosides” (“CRN”). CRN are nucleoside analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an oligonucleotide of the invention comprises one or more monomers that are UNA (unlocked nucleoside) nucleosides. UNA is unlocked acyclic nucleoside, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule may also be modified with a cyclopropane ring to produce a tricyclodeoxynucleic acid (tricyclo DNA). The ribose moiety may be substituted for another sugar such as 1,5,-anhydrohexitol, threose to produce a threose nucleoside (TNA), or arabinose to produce an arabino nucleoside. The ribose molecule can also be replaced with non-sugars such as cyclohexene to produce cyclohexene nucleoside or glycol to produce glycol nucleosides.

Potentially stabilizing modifications to the ends of nucleoside molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other alternatives chemistries of an oligonucleotide of the invention include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Exemplary oligonucleotides of the invention comprise nucleosides with alternative sugar moieties and may also comprise DNA or RNA nucleosides. In some embodiments, the oligonucleotide comprises nucleosides comprising alternative sugar moieties and DNA nucleosides. Incorporation of alternative nucleosides into the oligonucleotide of the invention may enhance the affinity of the oligonucleotide for the target nucleic acid. In that case, the alternative nucleosides can be referred to as affinity enhancing alternative nucleotides.

In some embodiments, the oligonucleotide comprises at least 1 alternative nucleoside, such as at least 2, at least 3, at least 4, at least 5, at least 6, at least 7, at least 8, at least 9, at least 10, at least 11, at least 12, at least 13, at least 14, at least 15 or at least 16 alternative nucleosides. In other embodiments, the oligonucleotides comprises from 1 to 10 alternative nucleosides, such as from 2 to 9 alternative nucleosides, such as from 3 to 8 alternative nucleosides, such as from 4 to 7 alternative nucleosides, such as 6 or 7 alternative nucleosides. In an embodiment, the oligonucleotide of the invention may comprise alternatives, which are independently selected from these three types of alternative (alternative sugar moiety, alternative nucleobase, and alternative internucleoside linkage), or a combination thereof. Preferably the oligonucleotide comprises one or more nucleosides comprising alternative sugar moieties, e.g., 2′ sugar alternative nucleosides. In some embodiments, the oligonucleotide of the invention comprise the one or more 2′ sugar alternative nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA, and BNA (e.g., LNA) nucleosides. In some embodiments, the one or more alternative nucleoside is a BNA.

In some embodiments, at least 1 of the alternative nucleosides is a BNA (e.g., an LNA), such as at least 2, such as at least 3, at least 4, at least 5, at least 6, at least 7, or at least 8 of the alternative nucleosides are BNAs. In a still further embodiment, all the alternative nucleosides are BNAs.

In a further embodiment the oligonucleotide comprises at least one alternative internucleoside linkage. In some embodiments, the internucleoside linkages within the contiguous nucleotide sequence are phosphorothioate or boronophosphate internucleoside linkages. In some embodiments, all the internucleotide linkages in the contiguous sequence of the oligonucleotide are phosphorothioate linkages. In some embodiments the phosphorothioate linkages are stereochemically pure phosphorothioate linkages. IN some embodiments, the phosphorothioate linkages are Sp phosphorothioate linkages. In other embodiments, the phosphorothioate linkages are Rp phosphorothioate linkages.

In some embodiments, the oligonucleotide of the invention comprises at least one alternative nucleoside which is a 2′-MOE-RNA, such as 2, 3, 4, 5, 6, 7, 8, 9, or 10 2′-MOE-RNA nucleoside units. In some embodiments, the 2′-MOE-RNA nucleoside units are connected by phosphorothioate linkages. In some embodiments, at least one of said alternative nucleoside is 2′-fluoro DNA, such as 2, 3, 4, 5, 6, 7, 8, 9, or 10 2′-fluoro-DNA nucleoside units. In some embodiments, the oligonucleotide of the invention comprises at least one BNA unit and at least one 2′ substituted modified nucleoside. In some embodiments of the invention, the oligonucleotide comprises both 2′ sugar modified nucleosides and DNA units. In some embodiments, the oligonucleotide of the invention or contiguous nucleotide region thereof is a gapmer oligonucleotide.

### Oligonucleotides Conjugated to Ligands

Oligonucleotides of the invention may be chemically linked to one or more ligands, moieties, or conjugates that enhance the activity, cellular distribution, or cellular uptake of the oligonucleotide. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In one embodiment, a ligand alters the distribution, targeting, or lifetime of an oligonucleotide agent into which it is incorporated. In some embodiments, a ligand provides an enhanced affinity for a selected target, e.g., molecule, cell or cell type, compartment, e.g., a cellular or organ compartment, tissue, organ, or region of the body, as, e.g., compared to a species absent such a ligand.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g., acridines), cross-linkers (e.g., psoralen, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the oligonucleotide agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an oligonucleotide as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the present invention as ligands (e.g., as PK modulating ligands). In addition, aptamers that bind serum components (e.g., serum proteins) are also suitable for use as PK modulating ligands in the embodiments described herein.

Ligand-conjugated oligonucleotides of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides of the present invention, such as the ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### Lipid Conjugates

In one embodiment, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. A lipid or lipid-based ligand can (a) increase resistance to degradation of the conjugate, (b) increase targeting or transport into a target cell or cell membrane, and/or (c) can be used to adjust binding to a serum protein, e.g., HSA.

In another aspect, the ligand is a moiety, e.g., a vitamin, which is taken up by a target cell, e.g., a proliferating cell. Exemplary vitamins include vitamin A, E, and K.

### Cell Permeation Agents

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ and the Drosophila Antennapedia protein (RQIKIWFQNRRMKWKK have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to an oligonucleotide agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand. Some conjugates of this ligand target PECAM-1 or VEGF.

A cell permeation peptide is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin, or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### Carbohydrate Conjugates

In some embodiments of the compositions and methods of the invention, an oligonucleotide further comprises a carbohydrate. The carbohydrate conjugated oligonucleotides are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one embodiment, a carbohydrate conjugate for use in the compositions and methods of the invention is a monosaccharide.

In some embodiments, the carbohydrate conjugate further comprises one or more additional ligands as described above, such as, but not limited to, a PK modulator and/or a cell permeation peptide.

Additional carbohydrate conjugates (and linkers) suitable for use in the present invention include those described in PCT Publication Nos. WO 2014/179620 and WO 2014/179627, the entire contents of each of which are incorporated herein by reference.

### Linkers

In some embodiments, the conjugate or ligand described herein can be attached to an oligonucleotide with various linkers that can be cleavable or non-cleavable.

Linkers typically comprise a direct bond or an atom such as oxygen or sulfur, a unit such as NR⁸, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times, or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

### Redox Cleavable Linking Groups

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular oligonucleotide moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one embodiment, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions).

The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

In another embodiment, a cleavable linker comprises a phosphate-based cleavable linking group.

A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(OR)—O—, —O—P(S)(OR^{k})—O—, —O—P(S)(SR^{k})—O—, —S—P(O)(OR^{k})—O—, —O—P(O)(OR^{k})—S—, —S—P(O)(OR^{k})—S—, —O—P(S)(OR^{k})—S—, —S—P(S)(OR^{k})—O—, —O—P(O)(R^{k})—O—, —O—P(S)(R^{k})—O—, —S—P(O)(R^{k})—O—, —S—P(S)(R^{k})—O—, —S—P(O)(R)—S—, —O—P(S)(R)—S—. These candidates can be evaluated using methods analogous to those described above.

### Acid Cleavable Linking Groups

In another embodiment, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

In another embodiment, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### Peptide-Based Cleaving Groups

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula

—NHCHR^{A}C(O)NHCHR^{B}C(O)—, where R^{A} and R^{B} are the R groups of the two adjacent amino acids. These candidates can be evaluated using methods analogous to those described above.

In one embodiment, an oligonucleotide of the invention is conjugated to a carbohydrate through a linker. Linkers include bivalent and trivalent branched linker groups. Linkers for oligonucleotide carbohydrate conjugates include, but are not limited to, those described in formulas 24-35 of PCT Publication No. WO 2018/195165.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an oligonucleotide. The present invention also includes oligonucleotide compounds that are chimeric compounds. Chimeric oligonucleotides typically contain at least one region wherein the RNA is modified so as to confer upon the oligonucleotide increased resistance to nuclease degradation, increased cellular uptake, and/or increased binding affinity for the target nucleic acid. An additional region of the oligonucleotide can serve as a substrate for enzymes capable of cleaving RNA:DNA. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxy oligonucleotides hybridizing to the same target region. Cleavage of the RNA target can be routinely detected by gel electrophoresis and, if necessary, associated nucleic acid hybridization techniques known in the art.

In certain instances, the nucleotides of an oligonucleotide can be modified by a non-ligand group. A number of non-ligand molecules have been conjugated to oligonucleotides in order to enhance the activity, cellular distribution, or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an aminolinker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### Pharmaceutical Uses

The oligonucleotide compositions described herein are useful in the methods of the invention and, while not bound by theory, are believed to exert their desirable effects through their ability to modulate the level, status, and/or activity of PCDH19, e.g., by inhibiting the activity or level of the PCDH19 protein in a cell in a mammal.

An aspect of the present invention relates to methods of treating disorders (e.g., epilepsy, szhizophrenia, and autism) related to PCDH19 in a subject in need thereof. Another aspect of the invention includes reducing (e.g., by 10%, 20%, 30%, 40%, 50%, 60%, 70%, 80%, 90%, 95%, 97%, 99%, or 100%) the level of PCDH19 in a cell of a subject identified as having a PCDH19 related disorder. Still another aspect includes a method of inhibiting expression of PCDH19 in a cell in a subject. The methods may include contacting a cell with an oligonucleotide, in an amount effective to inhibit expression of PCDH19 in the cell, thereby inhibiting expression of PCDH19 in the cell.

Based on the above methods, further aspects of the present invention include an oligonucleotide of the invention, or a composition comprising such an oligonucleotide, for use in therapy, or for use as a medicament, or for use in treating PCDH19 related disorders in a subject in need thereof, or for use in reducing the level of PCDH19 in a cell of a subject identified as having a PCDH19 related disorder, or for use in inhibiting expression of PCDH19 in a cell in a subject. The uses include the contacting of a cell with the oligonucleotide, in an amount effective to inhibit expression of PCDH19 in the cell, thereby inhibiting expression of PCDH19 in the cell. Embodiments described below in relation to the methods of the invention are also applicable to these further aspects.

Contacting of a cell with an oligonucleotide may be done in vitro or in vivo. Contacting a cell in vivo with the oligonucleotide includes contacting a cell or group of cells within a subject, e.g., a human subject, with the oligonucleotide. Combinations of in vitro and in vivo methods of contacting a cell are also possible. Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc₃ ligand, or any other ligand that directs the oligonucleotide to a site of interest. Cells can include those of the central nervous system, or muscle cells.

Inhibiting expression of a PCDH19 gene includes any level of inhibition of a PCDH19 gene, e.g., at least partial suppression of the expression of a PCDH19 gene, such as an inhibition by at least about 20%. In certain embodiments, inhibition is by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

Inhibiting expression of a PCDH19 gene includes inhibiting expression of a WT allele, a mutant allele, or a combination thereof. For example, the subject may have a mutation (e.g., nonsense, missense, and/or frameshift mutation) in one allele of the PCDH19 gene. As the gene is X-linked, a female subject is heterozygous, and a male subject is hemizygous (the other copy is inactivated). The oligonucleotide may selectively target the mutant allele. The oligonucleotide may selectively target the WT allele. The oligonucleotide may target both alleles.

The expression of a PCDH19 gene may be assessed based on the level of any variable associated with PCDH19 gene expression, e.g., PCDH19 mRNA level or PCDH19 protein level.

Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In certain embodiments, surrogate markers can be used to detect inhibition of PCDH19. For example, effective treatment of a PCDH19 related disorder, as demonstrated by acceptable diagnostic and monitoring criteria with an agent to reduce PCDH19 expression can be understood to demonstrate a clinically relevant reduction in PCDH19.

In some embodiments of the methods of the invention, expression of a PCDH19 gene is inhibited by at least 20%, a 25%, 30%, 35%, 40%, 45%, 50%, 55%, 60%, 65%, 70%, 75%, 80%, 85%, 90%, or 95%, or to below the level of detection of the assay. In certain embodiments, the methods include a clinically relevant inhibition of expression of PCDH19, e.g., as demonstrated by a clinically relevant outcome after treatment of a subject with an agent to reduce the expression of PCDH19.

Inhibition of the expression of a PCDH19 gene may be manifested by a reduction of the amount of mRNA expressed by a first cell or group of cells (such cells may be present, for example, in a sample derived from a subject) in which a PCDH19 gene is transcribed and which has or have been treated (e.g., by contacting the cell or cells with an oligonucleotide of the invention, or by administering an oligonucleotide of the invention to a subject in which the cells are or were present) such that the expression of a PCDH19 gene is inhibited, as compared to a second cell or group of cells substantially identical to the first cell or group of cells but which has not or have not been so treated (control cell(s) not treated with an oligonucleotide or not treated with an oligonucleotide targeted to the gene of interest). The degree of inhibition may be expressed in terms of:

$$\frac{( mRNA in control cells ) - ( mRNA in treated cells )}{( mRNA in control cells )} \times 100 \%$$

In other embodiments, inhibition of the expression of a PCDH19 gene may be assessed in terms of a reduction of a parameter that is functionally linked to PCDH19 gene expression, e.g., PCDH19 protein expression or PCDH19 activity. PCDH19 gene silencing may be determined in any cell expressing PCDH19, either endogenous or heterologous from an expression construct, and by any assay known in the art.

Inhibition of the expression of a PCDH19 protein may be manifested by a reduction in the level of the PCDH19 protein that is expressed by a cell or group of cells (e.g., the level of protein expressed in a sample derived from a subject). As explained above, for the assessment of mRNA suppression, the inhibition of protein expression levels in a treated cell or group of cells may similarly be expressed as a percentage of the level of protein in a control cell or group of cells.

A control cell or group of cells that may be used to assess the inhibition of the expression of a PCDH19 gene includes a cell or group of cells that has not yet been contacted with an oligonucleotide of the invention. For example, the control cell or group of cells may be derived from an individual subject (e.g., a human or animal subject) prior to treatment of the subject with an oligonucleotide.

The level of PCDH19 mRNA that is expressed by a cell or group of cells may be determined using any method known in the art for assessing mRNA expression. In one embodiment, the level of expression of PCDH19 in a sample is determined by detecting a transcribed polynucleotide, or portion thereof, e.g., mRNA of the PCDH19 gene. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating PCDH19 mRNA may be detected using methods the described in PCT Publication WO2012/177906, the entire contents of which are hereby incorporated herein by reference. In some embodiments, the level of expression of PCDH19 is determined using a nucleic acid probe. The term “probe,” as used herein, refers to any molecule that is capable of selectively binding to a specific PCDH19 sequence, e.g., to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to PCDH19 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of PCDH19 mRNA.

An alternative method for determining the level of expression of PCDH19 in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of PCDH19 is determined by quantitative fluorogenic RT-PCR (i.e., the TAQMAN™ System) or the DUAL-GLO® Luciferase assay.

The expression levels of PCDH19 mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722; 5,874,219; 5,744,305; 5,677,195; and 5,445,934, which are incorporated herein by reference. The determination of PCDH19 expression level may also comprise using nucleic acid probes in solution.

In some embodiments, the level of mRNA expression is assessed using branched DNA (bDNA) assays or real time PCR (qPCR). The use of this PCR method is described and exemplified in the Examples presented herein. Such methods can also be used for the detection of PCDH19 nucleic acids.

The level of PCDH19 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of PCDH19 proteins.

In some embodiments of the methods of the invention, the oligonucleotide is administered to a subject such that the oligonucleotide is delivered to a specific site within the subject. The inhibition of expression of PCDH19 may be assessed using measurements of the level or change in the level of PCDH19 mRNA or PCDH19 protein in a sample derived from a specific site within the subject. In certain embodiments, the methods include a clinically relevant inhibition of expression of PCDH19, e.g., as demonstrated by a clinically relevant outcome after treatment of a subject with an agent to reduce the expression of PCDH19.

In other embodiments, the oligonucleotide is administered in an amount and for a time effective to result in reduction (e.g., by 10%, 20%, 30%, 40%, 50%, 60%, 70%, 80%, 90%, or 100%) of one or more symptoms of a PCDH19 related disorder. Such symptoms include, but are not limited to, prolonged seizures, frequent seizures, behavioral and developmental delays, movement and balance issues, orthopedic conditions, delayed language and speech issues, growth and nutrition issues, sleeping difficulties, chronic infection, sensory integration disorder, disruption of the autonomic nervous system, and sweating.

Treating PCDH19 related disorders can result in an increase in average survival time of an individual or a population of subjects treated according to the present invention in comparison to a population of untreated subjects. For example, the survival time is of an individual or average survival time a of population is increased by more than 30 days (more than 60 days, 90 days, or 120 days). An increase in survival time of an individual or in average survival time of a population may be measured by any reproducible means. An increase in survival time of an individual may be measured, for example, by calculating for an individual the length of survival time following the initiation of treatment with the compound described herein. An increase in average survival time of a population may be measured, for example, by calculating for the average length of survival time following initiation of treatment with the compound described herein. An increase in survival time of an individual may also be measured, for example, by calculating for an individual length of survival time following completion of a first round of treatment with a compound or pharmaceutically acceptable salt of a compound described herein. An increase in average survival time of a population may also be measured, for example, by calculating for a population the average length of survival time following completion of a first round of treatment with a compound or pharmaceutically acceptable salt of a compound described herein.

Treating PCDH19 related disorders can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a compound or pharmaceutically acceptable salt of a compound described herein. A decrease in the mortality rate of a population may also be measured, for example, by calculating for a population the average number of disease-related deaths per unit time following completion of a first round of treatment with a compound or pharmaceutically acceptable salt of a compound described herein.

### Delivery of Oligonucleotides

The delivery of an oligonucleotide of the invention to a cell e.g., a cell within a subject, such as a human subject e.g., a subject in need thereof, such as a subject having a PCDH19 related disorder can be achieved in a number of different ways. For example, delivery may be performed by contacting a cell with an oligonucleotide of the invention either in vitro or in vivo. In vivo delivery may also be performed directly by administering a composition comprising an oligonucleotide to a subject. These alternatives are discussed further below.

In general, any method of delivering a nucleic acid molecule (in vitro or in vivo) can be adapted for use with an oligonucleotide of the invention (see e.g., Akhtar S. and Julian R L., (1992) Trends Cell. Biol. 2(5):139-144 and WO94/02595, which are incorporated herein by reference in their entireties). For in vivo delivery, factors to consider in order to deliver an oligonucleotide molecule include, for example, biological stability of the delivered molecule, prevention of non-specific effects, and accumulation of the delivered molecule in the target tissue. The non-specific effects of an oligonucleotide can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering an oligonucleotide systemically for the treatment of a disease, the oligonucleotide can include alternative nucleobases, alternative sugar moieties, and/or alternative internucleoside linkages, or alternatively delivered using a drug delivery system; both methods act to prevent the rapid degradation of the oligonucleotide by endo- and exo-nucleases in vivo. Modification of the oligonucleotide or the pharmaceutical carrier can also permit targeting of the oligonucleotide composition to the target tissue and avoid undesirable off-target effects. Oligonucleotide molecules can be modified by chemical conjugation to lipophilic groups such as cholesterol to enhance cellular uptake and prevent degradation. In an alternative embodiment, the oligonucleotide can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of an oligonucleotide molecule (negatively charged) and also enhance interactions at the negatively charged cell membrane to permit efficient uptake of an oligonucleotide by the cell. Cationic lipids, dendrimers, or polymers can either be bound to an oligonucleotide, or induced to form a vesicle or micelle that encases an oligonucleotide. The formation of vesicles or micelles further prevents degradation of the oligonucleotide when administered systemically. In general, any methods of delivery of nucleic acids known in the art may be adaptable to the delivery of the oligonucleotides of the invention. Methods for making and administering cationic oligonucleotide complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of oligonucleotides include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an oligonucleotide forms a complex with cyclodextrin for systemic administration. Methods for administration and pharmaceutical compositions of oligonucleotides and cyclodextrins can be found in U.S. Pat. No. 7,427,605, which is herein incorporated by reference in its entirety. In some embodiments the oligonucleotides of the invention are delivered by polyplex or lipoplex nanoparticles. Methods for administration and pharmaceutical compositions of oligonucleotides and polyplex nanoparticles and lipoplex nanoparticles can be found in U.S. Patent

Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

### Membranous Molecular Assembly Delivery Methods

Oligonucleotides of the invention can also be delivered using a variety of membranous molecular assembly delivery methods including polymeric, biodegradable microparticle, or microcapsule delivery devices known in the art. For example, a colloidal dispersion system may be used for targeted delivery of an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA and can mediate RNase H-mediated gene silencing. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169). These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising NOVASOME™ I (glyceryl dilaurate/cholesterol/polyoxyethylene-10-stearyl ether) and NOVASOME™ ∥ (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4(6):466).

Liposomes may also be sterically stabilized liposomes, comprising one or more specialized lipids that result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglio side G^{M1} galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotide (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotide into the skin. In some implementations, liposomes are used for delivering oligonucleotide to epidermal cells and also to enhance the penetration of oligonucleotide into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2, 405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising NOVASOME I (glyceryl dilaurate/cholesterol/polyoxyethylene-10-stearyl ether) and NOVASOME II (glyceryl distearate/cholesterol/polyoxyethylene-10-stearyl ether) were used to deliver a drug into the dermis of mouse skin. Such formulations with oligonucleotides are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application No. PCT/US2007/080331, filed Oct. 3, 2007 also describes formulations that are amenable to the present invention.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotides for use in the methods of the invention can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

### Lipid Nanoparticle-Based Delivery Methods

Oligonucleotides of in the invention may be fully encapsulated in a lipid formulation, e.g., a lipid nanoparticle (LNP), or other nucleic acid-lipid particle. LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to oligonucleotide ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

Non-limiting examples of cationic lipids include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can comprise, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C₁₈). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

### Combination Therapies

A method of the invention can be used alone or in combination with an additional therapeutic agent, e.g., other agents that treat PCDH19 related disorders or symptoms associated therewith, or in combination with other types of therapies to treat PCDH19 related disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the compounds when combined should provide a therapeutic effect.

In some embodiments, the oligonucleotide agents described herein may be used in combination with an additional therapeutic agent to treat a PCDH19 related disorder. In some embodiments, the additional therapeutic agent may be an oligonucleotide (e.g., an ASO) that hybridizes with the mRNA of gene associated with a PCDH19 related disorder.

In some embodiments, the second therapeutic agent is a chemotherapeutic agent (e.g., a cytotoxic agent or other chemical compound useful in the treatment of a PCDH19 related disorder).

In some embodiments, the second therapeutic agent is an anti-seizure agent.

The second agent may be a therapeutic agent which is a non-drug treatment. For example, the second therapeutic agent is physical therapy.

In any of the combination embodiments described herein, the first and second therapeutic agents may be administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The oligonucleotides described herein are preferably formulated into pharmaceutical compositions for administration to human subjects in a biologically compatible form suitable for administration in vivo.

The compounds described herein may be used in the form of the free base, in the form of salts, solvates, and as prodrugs. All forms are within the methods described herein. In accordance with the methods of the invention, the described compounds or salts, solvates, or prodrugs thereof may be administered to a subject in a variety of forms depending on the selected route of administration, as will be understood by those skilled in the art. The compounds described herein may be administered, for example, by oral, parenteral, intrathecal, intracerebroventricular, intraparenchymal, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, intracerebroventricular, intraparenchymal, rectal, and topical modes of administration. Parenteral administration may be by continuous infusion over a selected period of time.

The compounds described herein may be administered during any period of life. The compound may be administered to a newborn, neonate, infant, toddler, adolescent, or adult. The compound may be administered in utero. The compound may be administered immediately after birth.

A compound described herein may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound described herein may be incorporated with an excipient and used in the form of ingestible tablets, buccal tablets, troches, capsules, elixirs, suspensions, syrups, and wafers. A compound described herein may also be administered parenterally. Solutions of a compound described herein can be prepared in water suitably mixed with a surfactant, such as hydroxypropylcellulose.

Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter

The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compositions (e.g., a composition including an oligonucleotide) described herein, can vary depending on many factors, such as the pharmacodynamic properties of the compound, the mode of administration, the age, health, and weight of the recipient, the nature and extent of the symptoms, the frequency of the treatment, the type of concurrent treatment, if any, and the clearance rate of the compound in the animal to be treated. The compositions described herein may be administered initially in a suitable dosage that may be adjusted as required, depending on the clinical response. In some embodiments, the dosage of a composition (e.g., a composition including an oligonucleotide) is a prophylactically or a therapeutically effective amount.

### Kits

The invention also features kits including (a) a pharmaceutical composition including an oligonucleotide agent that reduces the level and/or activity of PCDH19 in a cell or subject described herein, and (b) a package insert with instructions to perform any of the methods described herein. In some embodiments, the kit includes (a) a pharmaceutical composition including an oligonucleotide agent that reduces the level and/or activity of PCDH19 in a cell or subject described herein, (b) an additional therapeutic agent, and (c) a package insert with instructions to perform any of the methods described herein.

### Methods of Selecting ASOs

Oligonucleotides suitable for use in ASO treatment may be selected using bionformatic methods. Oligonucleotides may be from 15-30 (e.g., 18-22, e.g., 19, 20, or 21) nucleotides in length. The oligonucleotides may have a GC content of from about 40% to about 70% (e.g., 45%, 50%, 55%, 60%, 65%, or 70%). The oligonucleotides may include 3 or fewer (e.g., 2, 1, or 0) mismatches to human PCDH19. The oligonucleotide may include at least 80% (e.g., 85%, 90%, 95%, 97%, 99%, and 100%) sequence identity to an equal length target of human PCDH19. The oligonucleotides may include at least 3 (e.g., 4, 5, 6, 7, 8, 9, 10, or more) mismatches to non PCDH19 transcripts. The oligonucleotides may not form dimers. The oligonucleotides may not form hairpins. The oligonucleotides may lack polyG runs, such as GGGG.

### Assessment of ASOs

The activity of the antisense oligonucleotides of the present disclosure can be assessed and confirmed using various techniques known in the art. For example, the ability of the antisense oligonucleotides to inhibit PCDH19 expression can be assessed in in vitro assays to confirm that the antisense oligonucleotides are suitable for use in treating a disease or condition associated with a mutation in PCDH19. Mouse models can be used to not only assess the ability of the antisense oligonucleotides to inhibit PCDH19 expression, but to also ameliorate symptoms associated with PCDH19 mutations.

In one example, cells such as mammalian cells (e.g., CHO cells) that are transfected with PCDH19 and express this gene are also transfected with an antisense oligonucleotide of the present disclosure. Typically, the PCDH19 contains a mutation, such as a missense, nonsense, or frameshift (e.g., insertion or deletion) mutation. In another example, a human neuronal cell line (e.g., SH-SY5Y) that naturally expresses native wild type PCDH19 is used. Optionally, the genome of this cell is edited so as to contain a mutation, such that the resulting PCDH19 is a disease causing variant. The levels of PCDH19 mRNA can be assessed using qRT-PCR or Northern blot as is well known in the art. The level of expression of protein from PCDH19 can be assessed by Western blot on total cell lysates or fractions as described in Rizzo et al. (Mol Cell Neurosci. 72:54-63, 2016).

In a particular examples, the activity of the antisense oligonucleotides of the present disclosure are assessed and confirmed using stem cell modelling (for review, see e.g., Tidball and Parent Stem Cells 34:27-33, 2016; Parent and Anderson Nature Neuroscience 18:360-366, 2015). For example, human induced pluripotent stem cells (iPSCs) can be produced from somatic cells (e.g., dermal fibroblasts or blood-derived hematopoietic cells) derived from a patient with a PCDH19 mutation and presenting with an associated disease or condition (e.g., EIEE9). Optionally, genome editing can be used to revert the mutation to wild-type to produce an isogenic control cell line (Gaj et al. Trends Biotechnol 31, 397-405, 2013), which can also be used to determine desirable wild-type levels of activity for subsequent assessment and comparison of oligonucleotides. Alternatively, genome editing can be used to introduce a mutation into the PCDH19 gene of wild-type, control iPSCs (e.g., a reference iPSC line). The iPSCs containing the mutation, and optionally the isogenic control, can then be differentiated into neurons, including excitatory neurons, using known techniques (see e.g., Kim et al. Front Cell Neurosci 8:109, 2014; Zhang et al. 2013, Chambers et al. Nat Biotechnol 27, 275-280, 2009). The effect of the antisense oligonucleotides of the present invention on PCDH19 expression (as assessed by PCDH19 mRNA or protein levels) and/or activity (e.g., as assessed by calcium binding) can then be assessed following exposure of the iPSCs to the antisense oligonucleotides of the present invention.

The levels of PCDH19 expression (mRNA or protein) observed when cells expressing PCDH19 are exposed to an antisense oligonucleotide of the present disclosure are compared to the respective levels observed when cells expressing PCDH19 are exposed with a negative control antisense oligonucleotide, so as to determine the level of inhibition resulting from the antisense oligonucleotide of the present disclosure. Typically, expression levels of PCDH19 are reduced by at least or about 20%, 25%, 30%, 35%, 40%, 45%, 50%, 55%, 60%, 65%, 70%, 75%, 80%, 85%, 90% or more. Accordingly, the antisense oligonucleotides of the present disclosure can be used for treating a disease or condition associated with a mutation in PCDH19.

The functional effect of administration of an antisense oligonucleotide may also be assessed by using mixing experiments, such as those described in Pederick et al. Neuron 97: 59-66, 2018, which is hereby incorporated by reference in its entirety. K562 cells can be used as they do not aggregate during culture due to a lack of endogenous PCDHs and classical cadherins. The cells may be engineered to express PCDH19 (either WT or a mutant) in combination with another nonclustering PCDH, such as PCDH17 and/or PCDH10. In some particular examples, the cells may express both a WT and mutant PCDH19. Two populations of fluorescently labeled K562 cells expressing identical or different PCDH combinations can be mixed in a 1:1 ratio. The resulting cell aggregates can be assessed for the contribution of each population. Aggregates dominated by one population may indicate cell sorting, due to different adhesion specificity, whereas heterogeneous aggregates may indicate cell mixing, due to identical adhesion specificity. If a disease causing variant causes sorting as opposed to mixing, the cells containing the PCDH19 mutant can then be treated with an ASO (e.g., allele specific ASO) and the outcome of the cell sorting experiment can be reassessed. If the results subsequently indicate mixing instead of sorting, then the ASO may be deemed successful as targeting and reducing the expression of the mutant PCDH19.

Mouse models can also be used to assess and confirm the activity of the antisense oligonucleotides of the present disclosure. For example, knock-in or transgenic mouse models can be generated using PCDH19 genes containing a mutation in a similar manner to that previously described for SCN1A and SCN2A knock-in and transgenic mouse models (see e.g., Kearney et al. Neuroscience 102, 307-317, 2001; Ogiwara et al. J Neurosci 27:5903-5914, 2007; Yu et al. Nat Neurosci 9:1142-1149, 2006). In particular examples, a PCDH19 gene that matches the particular antisense oligonucleotide is used to produce the knock-in or transgenic mouse. The mutant PCDH19 knock-in or transgenic mice may present with a phenotype similar to EIEE9, including, for example, increased neuronal activity, spontaneous seizures, and heterogeneous focal seizure activity on electroencephalogram (EEG). The ability of the antisense oligonucleotides of the present invention to inhibit expression of PCDH19 in these mice and to ameliorate any symptoms associated with the PCDH19 mutations can then be assessed.

In another example, the levels of PCDH19 mRNA and/or protein can be assessed following administration of an antisense oligonucleotide of the present disclosure or a negative control antisense oligonucleotide to the mice. In a particular example, PCDH19 mRNA and/or protein levels in the brain, and in particular the neurons, are assessed. The levels of PCDH19 expression following administration of an antisense oligonucleotide of the present disclosure are compared to the respective levels observed when a negative control antisense oligonucleotide is administered, so as to determine the level of inhibition resulting from the antisense oligonucleotide of the present disclosure. Typically, expression levels of PCDH19 in the mice (e.g., in the brains of the mice) are reduced by at least or about 20%, 25%, 30%, 35%, 40%, 45%, 50%, 55%, 60%, 65%, 70%, 75%, 80%, 85%, 90% or more.

In yet another example, the functional effect of administration of an antisense oligonucleotide of the present disclosure is assessed. For example, the number, severity and/or type of seizures can be assessed visually and/or by EEG. Neuronal excitability can also be assessed, such as by excising brain slices from mice administered an antisense oligonucleotide of the present disclosure or a negative control antisense oligonucleotide and assessing whole cell current (e.g., using the whole cell patch clamp technique). Similar neuronal excitability analyses can be performed using neurons isolated from the mice and then cultured. Additionally, mouse behavior, including gait characteristics, can be assessed to determine the functional effect of administration of an antisense oligonucleotide of the present disclosure.

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claimed.

### EXAMPLES

### Example 1. ASO Design and Production

Exemplary 15-, 16-, 17- and 20-mer antisense oligonucleotides (ASOs) were produced to the human PCDH19 gene set forth in NCBI Reference Sequence: NC_000023.11 (SEQ ID NO:1), located on the complementary strand within the region 100291644 . . . 100410273) (SEQ ID NO:1). The 5′ ends of ASO target sites were assigned to positions within the gene as listed in Table 2 below. Nucleotide composition was determined, and ASOs with <20% or >80% GC content were excluded from further analysis. Cross reactivity of ASOs were calculated for all known PCDH19 mRNA and pre-mRNA transcripts from NCBI RefSeqDB and EnsemblDB to identify ASOs that target as many of the different protein-coding transcripts as possible. Off target ASOs (with up to 2 mismatches) were excluded from further analysis. Single nucleotide polymorphisms (SNPs) located in ASO target sites were identified based on the human PCDH19 gene (NC_000023.11; region: complement (100291644 . . . 100410273)) and ASOs targeting sites harboring SNPs with >1% minor allele frequency were excluded from further analysis. The remaining ASOs were further screened for cross-reactivity with non-human primates and dogs. From this analysis 384 ASO candidates (SEQ ID NO:2 to SEQ ID NO:385) were selected for synthesis and in vitro screening, as shown in Table 2.

### Example 2. Screening of ASOs by Assessing Inhibitory Activity on PCDH19

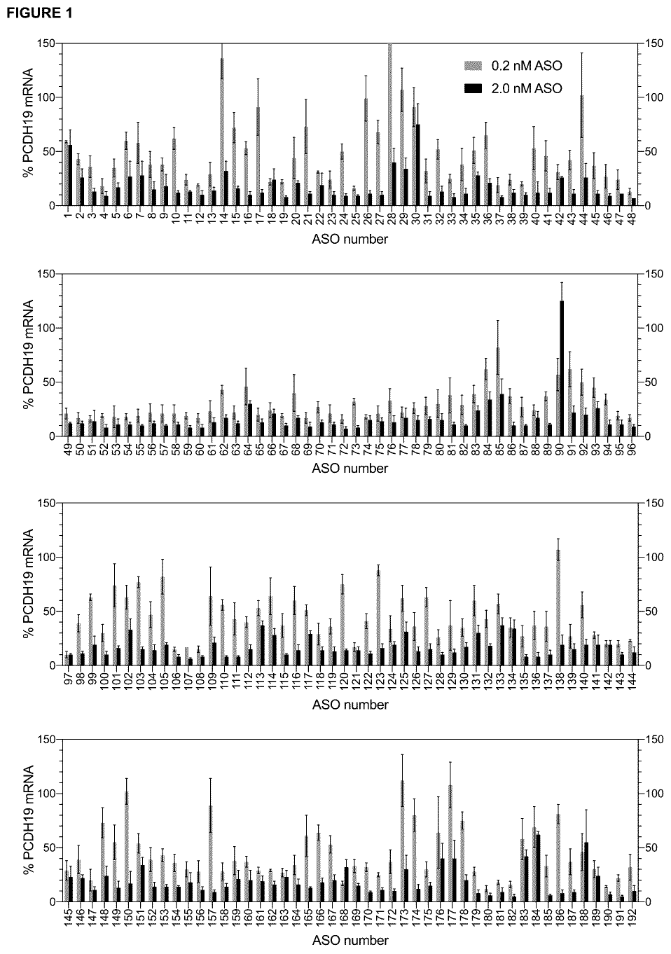

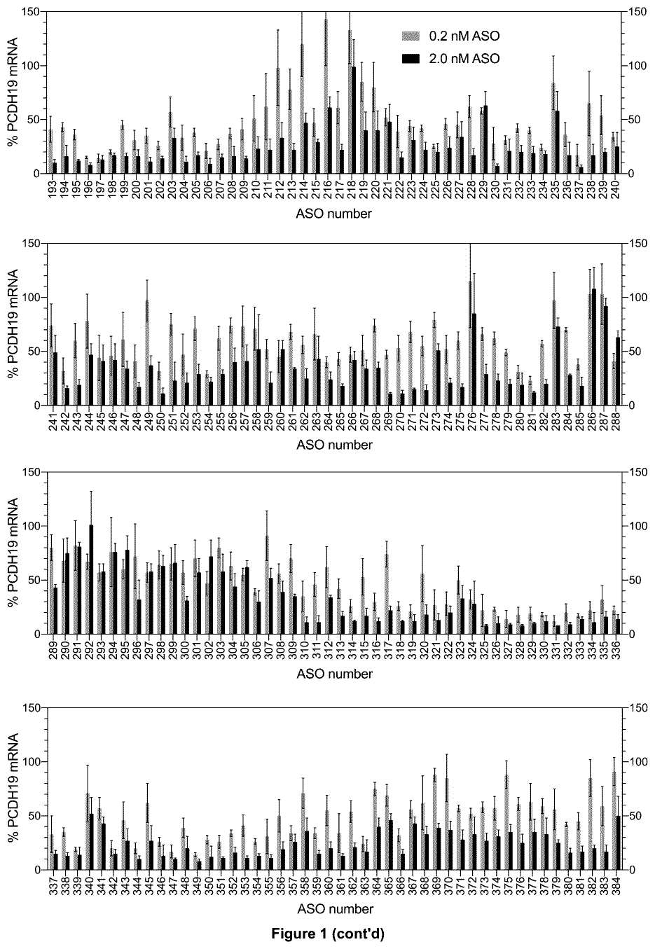

Exemplary ASOs (SEQ ID NOs: 2 to 385) as set out in Table 2 were screened using HEK293 cells (ATCC) seeded at a density of 30,000 cells per well in 96 well plates. Cells were transfected with 0.2 nM or 2.0 nM of each ASO using 0.4 μl per-well of Lipofectamine 2000 transfection reagent (Invitrogen). At 48 h post-transfection PCDH19 mRNA expression was quantified from cell lysates using Quantigene singleplex branched DNA assay (ThermoFisher). PCDH19 expression was normalised to GAPDH and expressed relative to the pooled means of mock-transfected cells and cells transfected with Ahsa1 ASOs that do not cross-react with PCDH19. Data, calculated from four separate experiments, is shown in Table 2 (as mean and standard deviation) and in FIG. 1.

### Example 3. Treatment of EIEE9 by Administration of an ASO

A female patient with EIEE9 is selected for ASO treatment. A 16mer ASO targeting PCDH19 mRNA is synthesized with phosphorothioate linkages throughout and 2MOE modifications on all sugar moieties. The ASO is dissolved in a suitable excipient compatible with administration to a human. A solution containing the dissolved ASO is injected into the brain of the patient such that the ASO solution interacts with targeted neurons in the brain. The ASO transfects the neurons and alters the translation of PCDH19 in the target cells, leading to a decrease in PCDH19 protein. A quantitative assay is performed to measure the decrease in PCDH19 protein. The patient undergoes extensive regular testing to measure a reduction of seizures and other symptoms associated with EIEE9 following administration of the ASO treatment.

TABLE 2
Inhibitory activity of Antisense oligonucleotides

| | Position | | | | | |
|---|---|---|---|---|---|---|
| | in | Localization | | | | |
| | gene | of | | | | |
| | NC_ | target site | | | | |
| | 000023.11, | in exon | Expression of PCDH19 in | | | |
| | region: | (E) or | ASO transfected cells | | | |
| | complement | intron (I) | 0.2 nM ASO | 2 nM ASO | | |
| ASO | (100291644 | within pre- | PCDH19/ | PCDH19/ | ASO | SEQ |
| Num- | . . . | mRNA of | GAPDH2 | GAPDH | sequence | ID |
| ber | 100410273) | NM_020766.3¹ | (% mean ± SD) | (% mean ± SD) | 5′-3′ | NO |
| 1 | 1778 | E | 59 ± 1 | 56 ± 14 | ATCACCGTC | 2 |
| | | | | | CCGGCG | |
| 2 | 1778 | E | 43 ± 5 | 26 ± 8 | AATCACCGT | 3 |
| | | | | | CCCGGCG | |
| 3 | 1778 | E | 36 ± 10 | 13 ± 3 | CAATCACCG | 4 |
| | | | | | TCCCGGCG | |
| 4 | 1781 | E | 18 ± 7 | 9 ± 4 | CGTTGGCAA | 5 |
| | | | | | TCACCGTCC | |
| | | | | | CG | |
| 5 | 1854 | E | 35 ± 8 | 17 ± 4 | TTGGACACC | 6 |
| | | | | | ACGCGAAA | |
| | | | | | GGC | |
| 6 | 1856 | E | 60 ± 8 | 27 ± 14 | AGTTGGAC | 7 |
| | | | | | ACCACGCG | |
| | | | | | AAAG | |
| 7 | 1857 | E | 58 ± 19 | 28 ± 13 | GAGTTGGA | 8 |
| | | | | | CACCACGCG | |
| | | | | | AAA | |
| 8 | 1858 | E | 38 ± 12 | 15 ± 7 | CGAGTTGG | 9 |
| | | | | | ACACCACGC | |
| | | | | | GAA | |
| 9 | 1970 | E | 38 ± 6 | 18 ± 11 | CCTCGAGCG | 10 |
| | | | | | AGATGATG | |
| | | | | | CAC | |
| 10 | 1978 | E | 62 ± 10 | 12 ± 2 | GGACATGA | 11 |
| | | | | | CCTCGAGCG | |
| | | | | | AGA | |
| 11 | 2044 | E | 24 ± 5 | 13 ± 1 | ACTGGGCG | 12 |
| | | | | | CATTGTCGT | |
| 12 | 2045 | E | 19 ± 1 | 10 ± 4 | ACTGGGCG | 13 |
| | | | | | CATTGTCG | |
| 13 | 2107 | E | 29 ± 11 | 14 ± 3 | GGATGCGC | 14 |
| | | | | | GTGCCAG | |
| 14 | 2118 | E | 136 ± 19 | 32 ± 9 | TAAGCGCTG | 15 |
| | | | | | TCCAGCGG | |
| | | | | | GAT | |
| 15 | 2124 | E | 72 ± 14 | 16 ± 2 | GGATCGTA | 16 |
| | | | | | AGCGCTGTC | |
| | | | | | CAG | |
| 16 | 2126 | E | 53 ± 6 | 10 ± 3 | CTGGATCGT | 17 |
| | | | | | AAGCGCTGT | |
| | | | | | CC | |
| 17 | 2127 | E | 91 ± 26 | 12 ± 3 | TCTGGATCG | 18 |
| | | | | | TAAGCGCTG | |
| | | | | | TC | |
| 18 | 2129 | E | 22 ± 3 | 24 ± 10 | GGATCGTA | 19 |
| | | | | | AGCGCTG | |
| 19 | 2129 | E | 22 ± 2 | 8 ± 1 | TGGATCGTA | 20 |
| | | | | | AGCGCTG | |
| 20 | 2130 | E | 44 ± 19 | 21 ± 2 | TCTGGATCG | 21 |
| | | | | | TAAGCGCT | |
| 21 | 2130 | E | 73 ± 25 | 11 ± 2 | GAGTCTGG | 22 |
| | | | | | ATCGTAAGC | |
| | | | | | GCT | |
| 22 | 2131 | E | 31 ± 1 | 19 ± 11 | TCTGGATCG | 23 |
| | | | | | TAAGCGC | |
| 23 | 2131 | E | 24 ± 8 | 10 ± 3 | GTCTGGATC | 24 |
| | | | | | GTAAGCGC | |
| 24 | 2131 | E | 50 ± 7 | 9 ± 2 | TGAGTCTGG | 25 |
| | | | | | ATCGTAAGC | |
| | | | | | GC | |
| 25 | 2132 | E | 16 ± 2 | 9 ± 1 | AGTCTGGAT | 26 |
| | | | | | CGTAAGCG | |
| 26 | 2159 | E | 99 ± 21 | 11 ± 3 | GCTCGTAAG | 27 |
| | | | | | TCTGCACGC | |
| | | | | | CA | |
| 27 | 2181 | E | 68 ± 11 | 10 ± 3 | CCGAACAG | 28 |
| | | | | | CTCGTTGGG | |
| | | | | | CGT | |
| 28 | 2182 | E | 212 ± 24 | 40 ± 13 | GCCGAACA | 29 |
| | | | | | GCTCGTTGG | |
| | | | | | GCG | |
| 29 | 2183 | E | 107 ± 20 | 34 ± 10 | GGCCGAAC | 30 |
| | | | | | AGCTCGTTG | |
| | | | | | GGC | |
| 30 | 2266 | E | 91 ± 18 | 75 ± 19 | ACTGCGTCT | 31 |
| | | | | | CGCGGT | |
| 31 | 2266 | E | 32 ± 11 | 9 ± 4 | GTGCGACT | 32 |
| | | | | | GCGTCTCGC | |
| | | | | | GGT | |
| 32 | 2275 | E | 52 ± 9 | 13 ± 5 | GAAGCTGT | 33 |
| | | | | | AGTGCGACT | |
| | | | | | GCG | |
| 33 | 2294 | E | 25 ± 4 | 8 ± 3 | TAGCGCAGT | 34 |
| | | | | | GATTCGG | |
| 34 | 2294 | E | 38 ± 15 | 11 ± 5 | CGTCTAGCG | 35 |
| | | | | | CAGTGATTC | |
| | | | | | GG | |
| 35 | 2303 | E | 51 ± 12 | 28 ± 3 | CGCCACCGT | 36 |
| | | | | | CTAGCGCA | |
| 36 | 2305 | E | 65 ± 12 | 21 ± 4 | TCGCCACCG | 37 |
| | | | | | TCTAGCG | |
| 37 | 2305 | E | 19 ± 7 | 8 ± 1 | GTCGCCACC | 38 |
| | | | | | GTCTAGCG | |
| 38 | 2306 | E | 24 ± 5 | 12 ± 3 | GGTCGCCAC | 39 |
| | | | | | CGTCTAGC | |
| 39 | 2307 | E | 20 ± 2 | 10 ± 2 | GTCGCCACC | 40 |
| | | | | | GTCTAG | |
| 40 | 2308 | E | 53 ± 20 | 12 ± 10 | CGGCGGGT | 41 |
| | | | | | CGCCACCGT | |
| | | | | | CTA | |
| 41 | 2375 | E | 46 ± 14 | 12 ± 4 | CGCTAAACA | 42 |
| | | | | | CCGGGTTGT | |
| | | | | | TG | |
| 42 | 2382 | E | 31 ± 7 | 26 ± 1 | CTCGCTAAA | 43 |
| | | | | | CACCGG | |
| 43 | 2382 | E | 42 ± 9 | 11 ± 4 | GTGGACTC | 44 |
| | | | | | GCTAAACAC | |
| | | | | | CGG | |
| 44 | 2383 | E | 102 ± 39 | 26 ± 13 | GGTGGACT | 45 |
| | | | | | CGCTAAACA | |
| | | | | | CCG | |
| 45 | 2441 | E | 37 ± 12 | 11 ± 3 | CGTTGAGG | 46 |
| | | | | | CGGATGAC | |
| | | | | | GGGT | |
| 46 | 2507 | E | 27 ± 11 | 9 ± 2 | GGTCGTTGA | 47 |
| | | | | | CGTAGCCAT | |
| | | | | | AG | |
| 47 | 2508 | E | 24 ± 9 | 11 ± 0 | CGGTCGTTG | 48 |
| | | | | | ACGTAGCCA | |
| | | | | | TA | |
| 48 | 2509 | E | 13 ± 3 | 7 ± 0 | GTCGTTGAC | 49 |
| | | | | | GTAGCCAT | |
| 49 | 2509 | E | 21 ± 5 | 12 ± 1 | GCGGTCGTT | 50 |
| | | | | | GACGTAGC | |
| | | | | | CAT | |
| 50 | 2510 | E | 17 ± 5 | 12 ± 2 | GGTCGTTGA | 51 |
| | | | | | CGTAGCCA | |
| 51 | 2510 | E | 16 ± 3 | 14 ± 10 | TGCGGTCGT | 52 |
| | | | | | TGACGTAGC | |
| | | | | | CA | |
| 52 | 2511 | E | 19 ± 2 | 8 ± 3 | GGTCGTTGA | 53 |
| | | | | | CGTAGCC | |
| 53 | 2511 | E | 18 ± 10 | 11 ± 5 | CGGTCGTTG | 54 |
| | | | | | ACGTAGCC | |
| 54 | 2511 | E | 18 ± 3 | 11 ± 2 | GTGCGGTC | 55 |
| | | | | | GTTGACGTA | |
| | | | | | GCC | |
| 55 | 2512 | E | 19 ± 6 | 10 ± 1 | CGGTCGTTG | 56 |
| | | | | | ACGTAGC | |
| 56 | 2512 | E | 22 ± 8 | 12 ± 2 | CGTGCGGTC | 57 |
| | | | | | GTTGACGTA | |
| | | | | | GC | |
| 57 | 2513 | E | 21 ± 8 | 10 ± 1 | GCGTGCGG | 58 |
| | | | | | TCGTTGACG | |
| | | | | | TAG | |
| 58 | 2514 | E | 21 ± 8 | 11 ± 2 | GTGCGGTC | 59 |
| | | | | | GTTGACGTA | |
| 59 | 2514 | E | 19 ± 3 | 8 ± 2 | CGCGTGCG | 60 |
| | | | | | GTCGTTGAC | |
| | | | | | GTA | |
| 60 | 2515 | E | 17 ± 4 | 8 ± 3 | GTGCGGTC | 61 |
| | | | | | GTTGACGT | |
| 61 | 2515 | E | 23 ± 10 | 13 ± 4 | CGTGCGGTC | 62 |
| | | | | | GTTGACGT | |
| 62 | 2516 | E | 43 ± 4 | 17 ± 3 | CGTGCGGTC | 63 |
| | | | | | GTTGACG | |
| 63 | 2516 | E | 22 ± 6 | 12 ± 2 | GCGTGCGG | 64 |
| | | | | | TCGTTGACG | |
| 64 | 2517 | E | 46 ± 17 | 30 ± 3 | CGTGCGGTC | 65 |
| | | | | | GTTGAC | |
| 65 | 2517 | E | 20 ± 6 | 13 ± 3 | CGCGTGCG | 66 |
| | | | | | GTCGTTGAC | |
| 66 | 2518 | E | 24 ± 7 | 21 ± 4 | GCGTGCGG | 67 |
| | | | | | TCGTTGA | |
| 67 | 2518 | E | 19 ± 2 | 10 ± 2 | CGCGTGCG | 68 |
| | | | | | GTCGTTGA | |
| 68 | 2540 | E | 40 ± 17 | 17 ± 2 | TGTGCGGG | 69 |
| | | | | | TCGATCTGA | |
| | | | | | AAG | |
| 69 | 2543 | E | 17 ± 5 | 9 ± 4 | GTGCGGGT | 70 |
| | | | | | CGATCTGA | |
| 70 | 2577 | E | 27 ± 5 | 13 ± 2 | TCGTAGTCT | 71 |
| | | | | | AAAGCGCC | |
| | | | | | AGT | |
| 71 | 2578 | E | 21 ± 8 | 11 ± 2 | TTCGTAGTC | 72 |
| | | | | | TAAAGCGCC | |
| | | | | | AG | |
| 72 | 2579 | E | 16 ± 4 | 7 ± 2 | CGTAGTCTA | 73 |
| | | | | | AAGCGCCA | |
| 73 | 2579 | E | 32 ± 3 | 8 ± 2 | CTTCGTAGT | 74 |
| | | | | | CTAAAGCGC | |
| | | | | | CA | |
| 74 | 2580 | E | 18 ±2 | 15 ± 4 | GTAGTCTAA | 75 |
| | | | | | AGCGCC | |
| 75 | 2580 | E | 21 ± 7 | 14 ± 3 | TCGTAGTCT | 76 |
| | | | | | AAAGCGCC | |
| 76 | 2580 | E | 33 ± 11 | 13 ± 6 | TCTTCGTAG | 77 |
| | | | | | TCTAAAGCG | |
| | | | | | CC | |
| 77 | 2581 | E | 22 ± 5 | 17 ± 9 | CGTAGTCTA | 78 |
| | | | | | AAGCGC | |
| 78 | 2581 | E | 26 ± 5 | 15 ± 4 | TCGTAGTCT | 79 |
| | | | | | AAAGCGC | |
| 79 | 2581 | E | 28 ± 8 | 16 ± 2 | TTCGTAGTC | 80 |
| | | | | | TAAAGCGC | |
| 80 | 2581 | E | 30 ± 13 | 15 ± 6 | CTCTTCGTA | 81 |
| | | | | | GTCTAAAGC | |
| | | | | | GC | |
| 81 | 2582 | E | 38 ± 16 | 11 ± 2 | CTTCGTAGT | 82 |
| | | | | | CTAAAGCG | |
| 82 | 2587 | E | 29 ± 9 | 10 ± 1 | GTGCCCCTC | 83 |
| | | | | | TTCGTAGTC | |
| | | | | | TA | |
| 83 | 2696 | E | 39 ± 8 | 24 ± 4 | ACCGGCGG | 84 |
| | | | | | ATTGTCA | |
| 84 | 2696 | E | 62 ± 10 | 34 ± 7 | GACCGGCG | 85 |
| | | | | | GATTGTCA | |
| 85 | 2696 | E | 82 ± 25 | 39 ± 14 | TGACCGGC | 86 |
| | | | | | GGATTGTCA | |
| 86 | 2700 | E | 37 ± 7 | 10 ± 3 | TTGATGACC | 87 |
| | | | | | GGCGGATT | |
| 87 | 2700 | E | 27 ± 9 | 10 ± 1 | AGGTTGAT | 88 |
| | | | | | GACCGGCG | |
| | | | | | GATT | |
| 88 | 2701 | E | 24 ± 5 | 17 ± 6 | TGATGACCG | 89 |
| | | | | | GCGGAT | |
| 89 | 2701 | E | 37 ± 4 | 11 ± 1 | TTGATGACC | 90 |
| | | | | | GGCGGAT | |
| 90 | 2767 | E | 57 ± 15 | 125 ± 17 | CAAGGCGA | 91 |
| | | | | | TCACGTAGC | |
| | | | | | CCG | |
| 91 | 2772 | E | 62 ± 16 | 22 ± 6 | CGCACCAA | 92 |
| | | | | | GGCGATCA | |
| | | | | | CGTA | |
| 92 | 2773 | E | 50 ± 12 | 20 ± 6 | CCGCACCAA | 93 |
| | | | | | GGCGATCA | |
| | | | | | CGT | |
| 93 | 2790 | E | 45 ± 9 | 26 ± 6 | TCGCGATCA | 94 |
| | | | | | GACACCCG | |
| 94 | 2790 | E | 34 ± 5 | 11 ± 4 | GAGTCGCG | 95 |
| | | | | | ATCAGACAC | |
| | | | | | CCG | |
| 95 | 2911 | E | 19 ± 4 | 11 ± 4 | TTGGTCGTG | 96 |
| | | | | | CTGCTCGCG | |
| | | | | | GT | |
| 96 | 2912 | E | 17 ± 3 | 9 ± 2 | ATTGGTCGT | 97 |
| | | | | | GCTGCTCGC | |
| | | | | | GG | |
| 97 | 2915 | E | 10 ± 3 | 10 ± 1 | ATTGGTCGT | 98 |
| | | | | | GCTGCTCG | |
| 98 | 2921 | E | 39 ± 8 | 11 ± 2 | TGAGGTTGT | 99 |
| | | | | | ATTGGTCGT | |
| | | | | | GC | |
| 99 | 2941 | E | 63 ± 3 | 19 ± 8 | TCGCGTGCC | 100 |
| | | | | | TGAATTG | |
| 100 | 2941 | E | 30 ± 8 | 10 ± 3 | GTCGCGTGC | 101 |
| | | | | | CTGAATTG | |
| 101 | 2941 | E | 74 ± 20 | 16 ± 2 | GCCGTCGC | 102 |
| | | | | | GTGCCTGAA | |
| | | | | | TTG | |
| 102 | 2942 | E | 63 ± 11 | 33 ± 10 | TCGCGTGCC | 103 |
| | | | | | TGAATT | |
| 103 | 2942 | E | 77 ± 5 | 15 ± 2 | CGCCGTCGC | 104 |
| | | | | | GTGCCTGAA | |
| | | | | | TT | |
| 104 | 2943 | E | 47 ± 12 | 14 ± 5 | CCGTCGCGT | 105 |
| | | | | | GCCTGAAT | |
| 105 | 2943 | E | 82 ± 16 | 19 ± 2 | CCGCCGTCG | 106 |
| | | | | | CGTGCCTGA | |
| | | | | | AT | |
| 106 | 2944 | E | 15 ± 2 | 8 ± 2 | GCCGTCGC | 107 |
| | | | | | GTGCCTGAA | |
| 107 | 2945 | E | 17 ± 0 | 6 ±1 | GCCGTCGC | 108 |
| | | | | | GTGCCTGA | |
| 108 | 2945 | E | 15 ± 3 | 8 ± 1 | CGCCGTCGC | 109 |
| | | | | | GTGCCTGA | |
| 109 | 2947 | E | 64 ± 27 | 21 ± 5 | CGCCGTCGC | 110 |
| | | | | | GTGCCT | |
| 110 | 3068 | E | 56 ± 5 | 8 ± 1 | CGCCAGGC | 111 |
| | | | | | GTGTTGTTC | |
| 111 | 3069 | E | 43 ± 15 | 8 ± 1 | GCGCCAGG | 112 |
| | | | | | CGTGTTGTT | |
| 112 | 3070 | E | 40 ± 5 | 15 ± 4 | GCGCCAGG | 113 |
| | | | | | CGTGTTGT | |
| 113 | 3101 | E | 53 ± 7 | 37 ± 4 | GGTCGGGG | 114 |
| | | | | | TCGCGAGC | |
| | | | | | AGAC | |
| 114 | 3107 | E | 64 ± 17 | 28 ± 6 | GACCCAGG | 115 |
| | | | | | TCGGGGTC | |
| | | | | | GCGA | |
| 115 | 3142 | E | 37 ± 11 | 10 ± 1 | CGACGGCA | 116 |
| | | | | | CGATCTGGT | |
| | | | | | AGG | |
| 116 | 3149 | E | 60 ± 13 | 14 ± 5 | GCACCTGCG | 117 |
| | | | | | ACGGCACG | |
| | | | | | ATC | |
| 117 | 3150 | E | 51 ± 5 | 29 ± 3 | CTGCGACG | 118 |
| | | | | | GCACGAT | |
| 118 | 3150 | E | 29 ± 10 | 14 ± 3 | CCTGCGACG | 119 |
| | | | | | GCACGAT | |
| 119 | 3150 | E | 36 ± 7 | 13 ± 4 | ACCTGCGAC | 120 |
| | | | | | GGCACGAT | |
| 120 | 3150 | E | 75 ± 9 | 14 ± 1 | CGCACCTGC | 121 |
| | | | | | GACGGCAC | |
| | | | | | GAT | |
| 121 | 3151 | E | 17 ± 4 | 14 ± 3 | CCTGCGACG | 122 |
| | | | | | GCACGA | |
| 122 | 3151 | E | 41 ± 7 | 11 ± 2 | ACCTGCGAC | 123 |
| | | | | | GGCACGA | |
| 123 | 3157 | E | 88 ± 5 | 16 ± 4 | CATGTCCCG | 124 |
| | | | | | CACCTGCGA | |
| | | | | | CG | |
| 124 | 3212 | E | 34 ± 12 | 19 ± 3 | CGCGTAGAT | 125 |
| | | | | | GTCGCCT | |
| 125 | 3213 | E | 62 ± 12 | 31 ± 9 | GCGCGTAG | 126 |
| | | | | | ATGTCGCC | |
| 126 | 3215 | E | 36 ± 13 | 13 ± 4 | ATCGCAGC | 127 |
| | | | | | GCGTAGAT | |
| | | | | | GTCG | |
| 127 | 3216 | E | 63 ± 9 | 15 ± 5 | GATCGCAG | 128 |
| | | | | | CGCGTAGAT | |
| | | | | | GTC | |
| 128 | 3217 | E | 26 ± 7 | 10 ± 2 | TCGCAGCGC | 129 |
| | | | | | GTAGATGT | |
| 129 | 3217 | E | 37 ± 23 | 12 ± 3 | GGATCGCA | 130 |
| | | | | | GCGCGTAG | |
| | | | | | ATGT | |
| 130 | 3218 | E | 35 ± 8 | 17 ± 4 | ATCGCAGC | 131 |
| | | | | | GCGTAGAT | |
| | | | | | G | |
| 131 | 3219 | E | 60 ± 14 | 30 ± 7 | ATCGCAGC | 132 |
| | | | | | GCGTAGAT | |
| 132 | 3219 | E | 43 ± 8 | 18 ± 2 | GATCGCAG | 133 |
| | | | | | CGCGTAGAT | |
| 133 | 3220 | E | 57 ± 9 | 37 ± 7 | ATCGCAGC | 134 |
| | | | | | GCGTAGA | |
| 134 | 3220 | E | 35 ± 9 | 34 ± 8 | GATCGCAG | 135 |
| | | | | | CGCGTAGA | |
| 135 | 3224 | E | 27 ± 10 | 8 ± 2 | GGTTAAAG | 136 |
| | | | | | GATCGCAG | |
| | | | | | CGCG | |
| 136 | 3225 | E | 37 ± 13 | 8 ± 4 | TGGTTAAAG | 137 |
| | | | | | GATCGCAG | |
| | | | | | CGC | |
| 137 | 3226 | E | 36 ± 14 | 10 ± 4 | GTGGTTAAA | 138 |
| | | | | | GGATCGCA | |
| | | | | | GCG | |
| 138 | 3252 | E | 107 ± 10 | 19 ± 9 | AATTCGAAC | 139 |
| | | | | | GCCTTGGT | |
| 139 | 3253 | E | 27 ± 11 | 15 ± 5 | ATTCGAACG | 140 |
| | | | | | CCTTGG | |
| 140 | 3253 | E | 56 ± 12 | 19 ± 5 | AATTCGAAC | 141 |
| | | | | | GCCTTGG | |
| 141 | 3255 | E | 28 ± 3 | 19 ± 9 | GAATTCGAA | 142 |
| | | | | | CGCCTT | |
| 142 | 3257 | E | 20 ± 3 | 19 ± 4 | TTGAATTCG | 143 |
| | | | | | AACGCC | |
| 143 | 3305 | E | 20 ± 3 | 10 ± 2 | CGTAGCGTT | 144 |
| | | | | | GCTTTGC | |
| 144 | 3305 | E | 23 ± 1 | 12 ± 5 | CCGTAGCGT | 145 |
| | | | | | TGCTTTGC | |
| 145 | 3306 | E | 29 ± 9 | 23 ± 10 | CGTAGCGTT | 146 |
| | | | | | GCTTTG | |
| 146 | 3306 | E | 39 ± 13 | 22 ± 3 | CCGTAGCGT | 147 |
| | | | | | TGCTTTG | |
| 147 | 3335 | E | 20 ± 10 | 11 ± 3 | GTCGTTGAC | 148 |
| | | | | | GTCGAGG | |
| 148 | 3377 | E | 73 ± 14 | 24 ± 9 | CGGCAGTG | 149 |
| | | | | | CCGTTAATC | |
| | | | | | AGA | |
| 149 | 3378 | E | 55 ± 16 | 13 ± 6 | TCGGCAGT | 150 |
| | | | | | GCCGTTAAT | |
| | | | | | CAG | |
| 150 | 3456 | E | 102 ± 12 | 17 ± 11 | TTTTCGCCC | 151 |
| | | | | | TCATCGTAG | |
| | | | | | TC | |
| 151 | 3457 | E | 54 ± 9 | 34 ± 7 | TCGCCCTCA | 152 |
| | | | | | TCGTAGT | |
| 152 | 3458 | E | 39 ± 11 | 14 ± 4 | TTCGCCCTC | 153 |
| | | | | | ATCGTAG | |
| 153 | 3469 | E | 43 ± 6 | 14 ± 2 | GGTGACTC | 154 |
| | | | | | GGCCATTTT | |
| | | | | | CGC | |
| 154 | 3475 | E | 36 ± 8 | 14 ± 1 | GTCGTAGGT | 155 |
| | | | | | GACTCGGCC | |
| | | | | | AT | |
| 155 | 3502 | E | 30 ± 7 | 18 ± 9 | AAAGAAGC | 156 |
| | | | | | CGCGGTCG | |
| | | | | | CCCT | |
| 156 | 3504 | E | 28 ± 10 | 11 ± 3 | TCAAAGAA | 157 |
| | | | | | GCCGCGGT | |
| | | | | | CGCC | |
| 157 | 3505 | E | 89 ± 25 | 9 ± 2 | AAAGAAGC | 158 |
| | | | | | CGCGGTCG | |
| | | | | | C | |
| 158 | 3505 | E | 28 ± 8 | 14 ± 3 | TTCAAAGAA | 159 |
| | | | | | GCCGCGGT | |
| | | | | | CGC | |
| 159 | 3506 | E | 38 ± 13 | 21 ± 8 | TTTCAAAGA | 160 |
| | | | | | AGCCGCGG | |
| | | | | | TCG | |
| 160 | 3507 | E | 37 ± 5 | 20 ± 9 | ATTTCAAAG | 161 |
| | | | | | AAGCCGCG | |
| | | | | | GTC | |
| 161 | 3508 | E | 29 ± 3 | 19 ± 5 | TTCAAAGAA | 162 |
| | | | | | GCCGCGGT | |
| 162 | 3509 | E | 29 ± 1 | 16 ± 3 | TTCAAAGAA | 163 |
| | | | | | GCCGCGG | |
| 163 | 3509 | E | 27 ± 4 | 23 ± 6 | CTATTTCAA | 164 |
| | | | | | AGAAGCCG | |
| | | | | | CGG | |
| 164 | 3510 | E | 34 ± 9 | 16 ± 5 | ATTTCAAAG | 165 |
| | | | | | AAGCCGCG | |
| 165 | 3606 | E | 61 ± 19 | 13 ± 1 | GTCTTGCCG | 166 |
| | | | | | TGGTCGTGA | |
| | | | | | GC | |
| 166 | 3607 | E | 64 ± 7 | 18 ± 4 | TGTCTTGCC | 167 |
| | | | | | GTGGTCGT | |
| | | | | | GAG | |
| 167 | 3608 | E | 53 ± 8 | 20 ± 5 | ATGTCTTGC | 168 |
| | | | | | CGTGGTCGT | |
| | | | | | GA | |
| 168 | 3610 | E | 17 ± 2 | 32 ± 7 | TGTCTTGCC | 169 |
| | | | | | GTGGTCGT | |
| 169 | 3761 | E | 33 ± 6 | 15 ± 2 | TTGCCACGA | 170 |
| | | | | | AGATCATA | |
| 170 | 3775 | E | 32 ± 4 | 9 ± 1 | CGCTTGCAC | 171 |
| | | | | | TTGATTG | |
| 171 | 3803 | E | 25 ± 2 | 11 ± 2 | AGTTGTAG | 172 |
| | | | | | GTCCGGATC | |
| 172 | 3804 | E | 37 ± 11 | 10 ± 2 | AGTTGTAG | 173 |
| | | | | | GTCCGGAT | |
| 173 | 5902 | I | 112 ± 24 | 30 ± 13 | ACCCTAGTA | 174 |
| | | | | | CTAAGGTTG | |
| | | | | | AT | |
| 174 | 6199 | I | 80 ± 15 | 12 ± 4 | GATCATCTC | 175 |
| | | | | | GCAAGTCTC | |
| | | | | | AT | |
| 175 | 6203 | I | 30 ± 7 | 15 ± 3 | GATCATCTC | 176 |
| | | | | | GCAAGTC | |
| 176 | 6584 | I | 64 ± 33 | 40 ± 14 | AGGAATTTA | 177 |
| | | | | | AAGGTTAGT | |
| | | | | | GC | |
| 177 | 6620 | I | 108 ± 21 | 40 ± 17 | CCGAGGAG | 178 |
| | | | | | ACAAGTGAT | |
| | | | | | GGT | |
| 178 | 6691 | I | 75 ± 8 | 20 ± 5 | CTTGCTCCT | 179 |
| | | | | | CGCTAATGG | |
| | | | | | GA | |
| 179 | 7523 | E | 28 ± 4 | 8 ± 3 | CTGACAACG | 180 |
| | | | | | TTCATCTTG | |
| | | | | | TC | |
| 180 | 7609 | E | 12 ± 3 | 6 ± 2 | AAGTGCTCT | 181 |
| | | | | | CAGAGCGG | |
| | | | | | CGG | |
| 181 | 7610 | E | 18 ± 2 | 9 ± 4 | AAAGTGCTC | 182 |
| | | | | | TCAGAGCG | |
| | | | | | GCG | |
| 182 | 7639 | E | 16 ± 3 | 5 ± 2 | TGCGGGTAT | 183 |
| | | | | | TCTGGTTCT | |
| | | | | | CC | |
| 183 | 8495 | I | 58 ± 19 | 42 ± 6 | CGTTGTTAG | 184 |
| | | | | | AGACGA | |
| 184 | 8495 | I | 69 ± 19 | 62 ± 3 | TTCGTTGTT | 185 |
| | | | | | AGAGACGA | |
| 185 | 9724 | I | 33 ± 10 | 6 ± 1 | GAGCTATTG | 186 |
| | | | | | GTGATCCAC | |
| | | | | | AA | |
| 186 | 11453 | I | 81 ± 9 | 8 ± 3 | GAGTGGAC | 187 |
| | | | | | TCGTCTATA | |
| | | | | | ATC | |
| 187 | 11456 | I | 37 ± 12 | 9 ± 2 | AGTGGACTC | 188 |
| | | | | | GTCTATA | |
| 188 | 11457 | I | 46 ± 17 | 55 ± 30 | GAGTGGAC | 189 |
| | | | | | TCGTCTAT | |
| 189 | 11458 | I | 30 ± 8 | 24 ± 8 | TGAGTGGA | 190 |
| | | | | | CTCGTCTA | |
| 190 | 11460 | I | 14 ± 1 | 7 ± 2 | GTCTGAGTG | 191 |
| | | | | | GACTCGTC | |
| 191 | 17511 | I | 22 ± 3 | 5 ± 1 | AGCCATATG | 192 |
| | | | | | CCTCTGTTC | |
| | | | | | GT | |
| 192 | 17512 | I | 32 ± 12 | 10 ± 5 | TAGCCATAT | 193 |
| | | | | | GCCTCTGTT | |
| | | | | | CG | |
| 193 | 18902 | I | 41 ± 12 | 10 ± 3 | GCCTTTATG | 194 |
| | | | | | GCAGAACTC | |
| | | | | | GC | |
| 194 | 19092 | I | 43 ± 4 | 16 ± 10 | ACTTAAAAG | 195 |
| | | | | | GAGATCTCG | |
| | | | | | GT | |
| 195 | 19985 | I | 36 ± 5 | 12 ± 1 | GAGCCTAAC | 196 |
| | | | | | TACTAAACA | |
| | | | | | GT | |
| 196 | 23931 | I | 15 ± 1 | 8 ± 2 | TCGTGTGCT | 197 |
| | | | | | TAGTAGGG | |
| | | | | | CTC | |
| 197 | 23933 | I | 14 ± 4 | 13 ± 4 | CATCGTGTG | 198 |
| | | | | | CTTAGTAGG | |
| | | | | | GC | |
| 198 | 23935 | I | 20 ± 2 | 17 ± 2 | AGCATCGTG | 199 |
| | | | | | TGCTTAGTA | |
| | | | | | GG | |
| 199 | 24118 | I | 45 ± 4 | 16 ± 3 | CCCTCATCG | 200 |
| | | | | | GCACTGGTA | |
| | | | | | AA | |
| 200 | 24120 | I | 31 ± 9 | 16 ± 6 | CTCCCTCAT | 201 |
| | | | | | CGGCACTG | |
| | | | | | GTA | |
| 201 | 28847 | I | 35 ± 7 | 11 ± 4 | GCCGTTGAA | 202 |
| | | | | | GATAGC | |
| 202 | 31641 | I | 26 ± 4 | 14 ± 2 | ACGTGCTAG | 203 |
| | | | | | CCAGTATAA | |
| | | | | | CC | |
| 203 | 31651 | I | 57 ± 14 | 33 ± 9 | GTATTAACG | 204 |
| | | | | | TGCTAGC | |
| 204 | 31860 | I | 33 ± 12 | 11 ± 5 | ACCTGTTCG | 205 |
| | | | | | TCAATAC | |
| 205 | 32315 | I | 38 ± 4 | 17 ± 3 | CGTTGGTGA | 206 |
| | | | | | TTAACCAGA | |
| | | | | | CT | |
| 206 | 32396 | I | 21 ± 7 | 9 ± 5 | GGTTCGATG | 207 |
| | | | | | TGATTGG | |
| 207 | 32396 | I | 27 ± 5 | 15 ± 3 | ATAGGGTTC | 208 |
| | | | | | GATGTGATT | |
| | | | | | GG | |
| 208 | 35911 | I | 37 ± 5 | 16 ± 9 | AGGTCGCA | 209 |
| | | | | | CTAAACAAT | |
| | | | | | TTA | |
| 209 | 36014 | I | 41 ± 10 | 14 ± 2 | CTTGCCAAG | 210 |
| | | | | | AATAGCTTA | |
| | | | | | GC | |
| 210 | 38436 | I | 51 ± 21 | 23 ± 11 | AAGGTCCTT | 211 |
| | | | | | CATGGACG | |
| | | | | | AGA | |
| 211 | 39063 | I | 62 ± 31 | 22 ± 10 | TCGTGGTCC | 212 |
| | | | | | TTCCTACTA | |
| | | | | | GA | |
| 212 | 40213 | I | 98 ± 35 | 33 ± 14 | CGACCACCA | 213 |
| | | | | | GATAGGCA | |
| | | | | | AGC | |
| 213 | 40814 | I | 78 ± 19 | 22 ± 6 | GGCCTTATA | 214 |
| | | | | | CCTAAGAAC | |
| | | | | | GA | |
| 214 | 40815 | I | 120 ± 30 | 47 ± 9 | TGGCCTTAT | 215 |
| | | | | | ACCTAAGAA | |
| | | | | | CG | |
| 215 | 42131 | I | 47 ± 13 | 29 ± 3 | CCGAAGGG | 216 |
| | | | | | GTAGCTA | |
| 216 | 43211 | I | 143 ± 43 | 61 ± 10 | TGGAATCG | 217 |
| | | | | | ATGAGAAG | |
| | | | | | GGTA | |
| 217 | 43743 | I | 61 ± 15 | 22 ± 5 | CCCGTGTTG | 218 |
| | | | | | GAGTCATAC | |
| | | | | | CT | |
| 218 | 43902 | I | 133 ± 31 | 99 ± 25 | GACAAGTAT | 219 |
| | | | | | TGCTCCCTG | |
| | | | | | GT | |
| 219 | 44763 | I | 85 ± 18 | 40 ± 17 | GCCTACAGT | 220 |
| | | | | | AGCTTTATT | |
| | | | | | GC | |
| 220 | 44892 | I | 80 ± 23 | 40 ± 18 | AATGGAAG | 221 |
| | | | | | CCCAATTGG | |
| | | | | | CTC | |
| 221 | 46416 | I | 52 ± 8 | 48 ± 16 | TAGGTAGA | 222 |
| | | | | | ATGTGCGTG | |
| | | | | | CGT | |
| 222 | 46417 | I | 39 ± 15 | 15 ± 5 | GGTAGAAT | 223 |
| | | | | | GTGCGTGC | |
| | | | | | G | |
| 223 | 48364 | I | 44 ± 5 | 31 ± 12 | CTGCTTAAA | 224 |
| | | | | | CACGACAG | |
| | | | | | GTT | |
| 224 | 48370 | I | 42 ± 3 | 22 ± 7 | GCCTGCTTA | 225 |
| | | | | | AACACGA | |
| 225 | 53050 | I | 25 ± 2 | 20 ± 8 | ACTTGTACG | 226 |
| | | | | | CAGGCTACT | |
| | | | | | GG | |
| 226 | 53051 | I | 46 ± 5 | 24 ± 10 | TACTTGTAC | 227 |
| | | | | | GCAGGCTA | |
| | | | | | CTG | |
| 227 | 53052 | I | 45 ± 12 | 34 ± 14 | ATACTTGTA | 228 |
| | | | | | CGCAGGCT | |
| | | | | | ACT | |
| 228 | 53053 | I | 62 ± 10 | 17 ± 6 | TTGTACGCA | 229 |
| | | | | | GGCTAC | |
| 229 | 53053 | I | 58 ± 3 | 63 ± 13 | TATACTTGT | 230 |
| | | | | | ACGCAGGC | |
| | | | | | TAC | |
| 230 | 53054 | I | 28 ± 15 | 7 ± 2 | CTTGTACGC | 231 |
| | | | | | AGGCTA | |
| 231 | 54334 | I | 31 ± 4 | 21 ± 11 | CGTGCCCTA | 232 |
| | | | | | GTCTCTAGA | |
| | | | | | AG | |
| 232 | 54338 | I | 42 ± 4 | 20 ± 6 | GAATCGTGC | 233 |
| | | | | | CCTAGTCTC | |
| | | | | | TA | |
| 233 | 54341 | I | 40 ± 3 | 19 ± 6 | ACTGAATCG | 234 |
| | | | | | TGCCCTAGT | |
| | | | | | CT | |
| 234 | 54496 | I | 24 ± 3 | 18 ± 3 | GATTCTAGT | 235 |
| | | | | | AAGACGTTT | |
| | | | | | GG | |
| 235 | 54506 | I | 84 ± 25 | 58 ± 18 | GATATAGG | 236 |
| | | | | | AGGATTCTA | |
| | | | | | GTA | |
| 236 | 54840 | I | 36 ± 11 | 17 ± 12 | TAGGATCGT | 237 |
| | | | | | CACCAACCT | |
| | | | | | TC | |
| 237 | 54843 | I | 17 ± 10 | 6 ± 2 | TAGGATCGT | 238 |
| | | | | | CACCAACC | |
| 238 | 54843 | I | 65 ± 30 | 17 ± 10 | ACATAGGAT | 239 |
| | | | | | CGTCACCAA | |
| | | | | | CC | |
| 239 | 54850 | I | 54 ± 18 | 20 ± 3 | TAACATAGG | 240 |
| | | | | | ATCGTC | |
| 240 | 54850 | I | 34 ± 4 | 25 ± 13 | TTAACATAG | 241 |
| | | | | | GATCGTC | |
| 241 | 56292 | I | 74 ± 20 | 49 ± 16 | TAATCCGTC | 242 |
| | | | | | TCAGGTC | |
| 242 | 56292 | I | 32 ± 12 | 16 ± 2 | GTAATCCGT | 243 |
| | | | | | CTCAGGTC | |
| 243 | 56293 | I | 60 ± 16 | 19 ± 5 | GTAATCCGT | 244 |
| | | | | | CTCAGGT | |
| 244 | 56376 | I | 78 ± 25 | 47 ± 10 | TAGCGCTAG | 245 |
| | | | | | TCAATGTGG | |
| | | | | | CA | |
| 245 | 56377 | I | 44 ± 21 | 41 ± 15 | GTAGCGCTA | 246 |
| | | | | | GTCAATGTG | |
| | | | | | GC | |
| 246 | 56378 | I | 46 ± 18 | 42 ± 15 | AGTAGCGCT | 247 |
| | | | | | AGTCAATGT | |
| | | | | | GG | |
| 247 | 56379 | I | 61 ± 25 | 34 ± 7 | TAGTAGCGC | 248 |
| | | | | | TAGTCAATG | |
| | | | | | TG | |
| 248 | 56380 | I | 41 ± 15 | 17 ± 4 | GTAGCGCTA | 249 |
| | | | | | GTCAATGT | |
| 249 | 56380 | I | 97 ± 19 | 37 ± 9 | TTAGTAGCG | 250 |
| | | | | | CTAGTCAAT | |
| | | | | | GT | |
| 250 | 56382 | I | 32 ± 8 | 11 ± 5 | GTAGCGCTA | 251 |
| | | | | | GTCAAT | |
| 251 | 56382 | I | 75 ± 10 | 23 ± 17 | AGTAGCGCT | 252 |
| | | | | | AGTCAAT | |
| 252 | 56383 | I | 47 ± 19 | 21 ± 9 | AGTAGCGCT | 253 |
| | | | | | AGTCAA | |
| 253 | 56383 | I | 71 ± 11 | 29 ± 9 | TAGTAGCGC | 254 |
| | | | | | TAGTCAA | |
| 254 | 56383 | I | 29 ± 3 | 22 ± 4 | TTAGTAGCG | 255 |
| | | | | | CTAGTCAA | |
| 255 | 56384 | I | 62 ± 11 | 29 ± 4 | TTAGTAGCG | 256 |
| | | | | | CTAGTCA | |
| 256 | 56385 | I | 74 ± 7 | 40 ± 13 | TTAGTAGCG | 257 |
| | | | | | CTAGTC | |
| 257 | 56385 | I | 73 ± 19 | 41 ± 15 | ATTAGTAGC | 258 |
| | | | | | GCTAGTC | |
| 258 | 56385 | I | 71 ± 20 | 52 ± 32 | AATTAGTAG | 259 |
| | | | | | CGCTAGTC | |
| 259 | 56385 | I | 52 ± 9 | 21 ± 10 | TGCAATTAG | 260 |
| | | | | | TAGCGCTAG | |
| | | | | | TC | |
| 260 | 56386 | I | 45 ± 15 | 52 ± 8 | ATTAGTAGC | 261 |
| | | | | | GCTAGT | |
| 261 | 56386 | I | 68 ± 7 | 34 ± 1 | AATTAGTAG | 262 |
| | | | | | CGCTAGT | |
| 262 | 56386 | I | 56 ± 8 | 25 ± 9 | CAATTAGTA | 263 |
| | | | | | GCGCTAGT | |
| 263 | 56386 | I | 66 ± 24 | 43 ± 21 | ATGCAATTA | 264 |
| | | | | | GTAGCGCTA | |
| | | | | | GT | |
| 264 | 56387 | I | 40 ± 5 | 24 ± 7 | CAATTAGTA | 265 |
| | | | | | GCGCTAG | |
| 265 | 56387 | I | 43 ± 6 | 18 ± 2 | GCAATTAGT | 266 |
| | | | | | AGCGCTAG | |
| 266 | 56388 | I | 47 ± 7 | 42 ± 8 | CAATTAGTA | 267 |
| | | | | | GCGCTA | |
| 267 | 56390 | I | 51 ± 14 | 34 ± 8 | TGCAATTAG | 268 |
| | | | | | TAGCGC | |
| 268 | 57336 | I | 74 ± 6 | 35 ± 5 | ACCCGGAAT | 269 |
| | | | | | TGATTAAGC | |
| | | | | | AC | |
| 269 | 59591 | E | 47 ± 4 | 11 ± 1 | CGGCTATTC | 270 |
| | | | | | ACGTAGTTG | |
| | | | | | GA | |
| 270 | 59592 | E | 53 ± 12 | 11 ± 3 | TCGGCTATT | 271 |
| | | | | | CACGTAGTT | |
| | | | | | GG | |
| 271 | 59594 | E | 68 ± 10 | 15 ± 1 | GCTCGGCTA | 272 |
| | | | | | TTCACGTAG | |
| | | | | | TT | |
| 272 | 59595 | E | 55 ± 9 | 14 ± 5 | CGGCTATTC | 273 |
| | | | | | ACGTAGT | |
| 273 | 59596 | E | 79 ± 7 | 51 ± 6 | CGGCTATTC | 274 |
| | | | | | ACGTAG | |
| 274 | 59596 | E | 52 ± 13 | 21 ± 4 | TCGGCTATT | 275 |
| | | | | | CACGTAG | |
| 275 | 59596 | E | 60 ± 8 | 17 ± 3 | GGGCTCGG | 276 |
| | | | | | CTATTCACG | |
| | | | | | TAG | |
| 276 | 59597 | E | 115 ± 43 | 85 ± 37 | TCGGCTATT | 277 |
| | | | | | CACGTA | |
| 277 | 59598 | E | 66 ± 6 | 29 ± 9 | CTCGGCTAT | 278 |
| | | | | | TCACGT | |
| 278 | 59599 | E | 62 ± 6 | 23 ± 6 | GCTCGGCTA | 279 |
| | | | | | TTCACG | |
| 279 | 59599 | E | 49 ± 3 | 20 ± 4 | GGGCTCGG | 280 |
| | | | | | CTATTCACG | |
| 280 | 59606 | E | 31 ± 6 | 19 ± 11 | TTGATTAAA | 281 |
| | | | | | TGGGCTCG | |
| | | | | | GCT | |
| 281 | 59608 | E | 23 ± 4 | 12 ± 1 | TGATTAAAT | 282 |
| | | | | | GGGCTCGG | |
| 282 | 60855 | I | 57 ± 3 | 20 ± 4 | CTGATAATA | 283 |
| | | | | | CAAGGGCT | |
| | | | | | ACC | |
| 283 | 62557 | I | 97 ± 26 | 73 ± 8 | CCGAAAAG | 284 |
| | | | | | AAGGGTCC | |
| | | | | | AAGA | |
| 284 | 64064 | I | 70 ± 2 | 28 ± 1 | GCATGACG | 285 |
| | | | | | AATGAACAT | |
| | | | | | CAC | |
| 285 | 66561 | I | 38 ± 5 | 18 ± 8 | TGAATCCAT | 286 |
| | | | | | TCCCTAGTC | |
| | | | | | GG | |
| 286 | 68152 | I | 103 ± 23 | 108 ± 20 | CGACCGTG | 287 |
| | | | | | ACAGATCTG | |
| | | | | | ATT | |
| 287 | 68153 | I | 103 ± 28 | 92 ± 7 | ACGACCGT | 288 |
| | | | | | GACAGATCT | |
| | | | | | GAT | |
| 288 | 68155 | I | 41 ± 7 | 63 ± 6 | CGACCGTG | 289 |
| | | | | | ACAGATCTG | |
| 289 | 68156 | I | 80 ± 12 | 43 ± 3 | ACGACCGT | 290 |
| | | | | | GACAGATCT | |
| 290 | 68157 | I | 68 ± 20 | 75 ± 14 | TACGACCGT | 291 |
| | | | | | GACAGATC | |
| 291 | 68157 | I | 82 ± 23 | 81 ± 4 | ATTTACGAC | 292 |
| | | | | | CGTGACAG | |
| | | | | | ATC | |
| 292 | 68158 | I | 67 ± 7 | 101 ± 31 | ACGACCGT | 293 |
| | | | | | GACAGAT | |
| 293 | 68158 | I | 57 ± 8 | 58 ± 7 | TACGACCGT | 294 |
| | | | | | GACAGAT | |
| 294 | 68158 | I | 76 ± 32 | 76 ± 8 | TTACGACCG | 295 |
| | | | | | TGACAGAT | |
| 295 | 68159 | I | 60 ± 9 | 78 ± 13 | TACGACCGT | 296 |
| | | | | | GACAGA | |
| 296 | 68160 | I | 72 ± 30 | 32 ± 18 | TTACGACCG | 297 |
| | | | | | TGACAG | |
| 297 | 68160 | I | 57 ± 9 | 58 ± 7 | TTTACGACC | 298 |
| | | | | | GTGACAG | |
| 298 | 68162 | I | 64 ± 13 | 63 ± 10 | AATTTACGA | 299 |
| | | | | | CCGTGAC | |
| 299 | 68162 | I | 65 ± 15 | 66 ± 17 | TAATTTACG | 300 |
| | | | | | ACCGTGAC | |
| 300 | 68163 | I | 57 ± 11 | 31 ± 4 | AATTTACGA | 301 |
| | | | | | CCGTGA | |
| 301 | 68163 | I | 70 ± 17 | 57 ± 13 | TAATTTACG | 302 |
| | | | | | ACCGTGA | |
| 302 | 68163 | I | 47 ± 11 | 72 ± 15 | ATAATTTAC | 303 |
| | | | | | GACCGTGA | |
| 303 | 68164 | I | 80 ± 9 | 58 ± 16 | TAATTTACG | 304 |
| | | | | | ACCGTG | |
| 304 | 68164 | I | 63 ± 13 | 44 ± 12 | ATAATTTAC | 305 |
| | | | | | GACCGTG | |
| 305 | 68164 | I | 55 ± 6 | 62 ± 6 | GATAATTTA | 306 |
| | | | | | CGACCGTG | |
| 306 | 68165 | I | 39 ± 3 | 30 ± 10 | ATAATTTAC | 307 |
| | | | | | GACCGT | |
| 307 | 68165 | I | 91 ± 23 | 52 ± 9 | TGATAATTT | 308 |
| | | | | | ACGACCGT | |
| 308 | 68166 | I | 56 ± 9 | 39 ± 10 | GATAATTTA | 309 |
| | | | | | CGACCG | |
| 309 | 68166 | I | 70 ± 13 | 35 ± 2 | TGATAATTT | 310 |
| | | | | | ACGACCG | |
| 310 | 68274 | E | 35 ± 14 | 11 ± 5 | TCCGCTGGA | 311 |
| | | | | | CATCATGCT | |
| | | | | | CA | |
| 311 | 68276 | E | 46 ± 11 | 11 ± 6 | GCTCCGCTG | 312 |
| | | | | | GACATCATG | |
| | | | | | CT | |
| 312 | 68277 | E | 62 ± 19 | 34 ± 2 | GGCTCCGCT | 313 |
| | | | | | GGACATCAT | |
| | | | | | GC | |
| 313 | 68279 | E | 42 ± 9 | 17 ± 4 | GCTCCGCTG | 314 |
| | | | | | GACATCAT | |
| 314 | 68280 | E | 26 ± 6 | 12 ± 1 | GGCTCCGCT | 315 |
| | | | | | GGACATCA | |
| 315 | 68284 | E | 53 ± 17 | 17 ± 7 | CAATACAG | 316 |
| | | | | | GCTCCGCTG | |
| | | | | | GAC | |
| 316 | 68285 | E | 30 ± 8 | 12 ± 3 | ACAATACAG | 317 |
| | | | | | GCTCCGCTG | |
| | | | | | GA | |
| 317 | 68288 | E | 74 ± 12 | 22 ± 4 | CAATACAG | 318 |
| | | | | | GCTCCGCT | |
| 318 | 72766 | I | 26 ± 4 | 12 ± 1 | CGTATTAAT | 319 |
| | | | | | TCATGGGCA | |
| | | | | | CT | |
| 319 | 73373 | I | 21 ± 6 | 12 ± 6 | GCATGATCC | 320 |
| | | | | | CTATAAACC | |
| | | | | | CC | |
| 320 | 74211 | I | 56 ± 26 | 18 ± 9 | GATCTAATC | 321 |
| | | | | | AACCGCTTT | |
| | | | | | GT | |
| 321 | 74212 | I | 27 ± 14 | 13 ± 6 | GGATCTAAT | 322 |
| | | | | | CAACCGCTT | |
| | | | | | TG | |
| 322 | 74214 | I | 28 ± 11 | 20 ± 6 | GGGGATCT | 323 |
| | | | | | AATCAACCG | |
| | | | | | CTT | |
| 323 | 74216 | I | 50 ± 13 | 33 ± 12 | GGATCTAAT | 324 |
| | | | | | CAACCGC | |
| 324 | 74928 | I | 32 ± 9 | 28 ± 21 | GCCTCTAAA | 325 |
| | | | | | CCAGCAAG | |
| | | | | | GTA | |
| 325 | 76740 | I | 22 ± 15 | 8 ± 1 | CGATCCTAT | 326 |
| | | | | | GTAGTCTA | |
| 326 | 76741 | I | 23 ± 2 | 10 ± 6 | CGATCCTAT | 327 |
| | | | | | GTAGTCT | |
| 327 | 76741 | I | 14 ± 8 | 9 ± 1 | CCGATCCTA | 328 |
| | | | | | TGTAGTCT | |
| 328 | 76741 | I | 18 ± 7 | 8 ±1 | TCACCGATC | 329 |
| | | | | | CTATGTAGT | |
| | | | | | CT | |
| 329 | 76742 | I | 19 ± 6 | 10 ± 1 | CGATCCTAT | 330 |
| | | | | | GTAGTC | |
| 330 | 76742 | I | 18 ± 2 | 12 ± 5 | CCGATCCTA | 331 |
| | | | | | TGTAGTC | |
| 331 | 76742 | I | 12 ± 5 | 8 ± 0 | ACCGATCCT | 332 |
| | | | | | ATGTAGTC | |
| 332 | 76742 | I | 20 ± 8 | 9 ± 2 | TTCACCGAT | 333 |
| | | | | | CCTATGTAG | |
| | | | | | TC | |
| 333 | 76743 | I | 17 ± 2 | 14 ± 2 | CCGATCCTA | 334 |
| | | | | | TGTAGT | |
| 334 | 76743 | I | 22 ± 8 | 11 ± 9 | ACCGATCCT | 335 |
| | | | | | ATGTAGT | |
| 335 | 76743 | I | 32 ± 13 | 16 ± 5 | CTTCACCGA | 336 |
| | | | | | TCCTATGTA | |
| | | | | | GT | |
| 336 | 76744 | I | 22 ± 4 | 14 ± 4 | ACCGATCCT | 337 |
| | | | | | ATGTAG | |
| 337 | 76744 | I | 33 ± 17 | 15 ± 3 | TCACCGATC | 338 |
| | | | | | CTATGTAG | |
| 338 | 76745 | I | 35 ± 4 | 13 ± 3 | TCACCGATC | 339 |
| | | | | | CTATGTA | |
| 339 | 81342 | I | 19 ± 2 | 14 ± 7 | CTCTAATTA | 340 |
| | | | | | GCGTCAC | |
| 340 | 81582 | I | 71 ± 26 | 52 ± 15 | TACTTCAGT | 341 |
| | | | | | ACCACCGG | |
| | | | | | AAT | |
| 341 | 90521 | I | 57 ± 10 | 43 ± 6 | CTAGTAGTC | 342 |
| | | | | | CTGAACTAA | |
| | | | | | TG | |
| 342 | 90553 | I | 20 ± 7 | 15 ± 4 | GGATTTGGT | 343 |
| | | | | | AGTTCCATC | |
| | | | | | AC | |
| 343 | 91819 | I | 46 ± 17 | 27 ± 11 | GCTAGCCTT | 344 |
| | | | | | ATCATTTGA | |
| | | | | | CC | |
| 344 | 91825 | I | 20 ± 5 | 10 ± 3 | GTCCAAGCT | 345 |
| | | | | | AGCCTTATC | |
| | | | | | AT | |
| 345 | 96127 | I | 62 ± 18 | 27 ± 14 | TAACGATTT | 346 |
| | | | | | TACGGAGA | |
| | | | | | CAA | |
| 346 | 96128 | I | 26 ± 4 | 13 ± 10 | CGATTTTAC | 347 |
| | | | | | GGAGACA | |
| 347 | 96128 | I | 17 ± 6 | 10 ± 1 | ACGATTTTA | 348 |
| | | | | | CGGAGACA | |
| 348 | 96128 | I | 39 ± 9 | 20 ± 11 | GTAACGATT | 349 |
| | | | | | TTACGGAG | |
| | | | | | ACA | |
| 349 | 96129 | I | 14 ± 2 | 8 ± 2 | CGATTTTAC | 350 |
| | | | | | GGAGAC | |
| 350 | 96129 | I | 28 ± 4 | 12 ± 10 | ACGATTTTA | 351 |
| | | | | | CGGAGAC | |
| 351 | 96129 | I | 26 ± 6 | 11 ± 1 | AACGATTTT | 352 |
| | | | | | ACGGAGAC | |
| 352 | 96129 | I | 34 ± 3 | 16 ± 5 | AGTAACGAT | 353 |
| | | | | | TTTACGGAG | |
| | | | | | AC | |
| 353 | 96130 | I | 41 ± 10 | 11 ± 2 | AACGATTTT | 354 |
| | | | | | ACGGAGA | |
| 354 | 96131 | I | 26 ± 3 | 13 ± 2 | AACGATTTT | 355 |
| | | | | | ACGGAG | |
| 355 | 96131 | I | 31 ± 16 | 11 ± 3 | GTAACGATT | 356 |
| | | | | | TTACGGAG | |
| 356 | 96132 | I | 50 ± 15 | 19 ± 7 | GTAACGATT | 357 |
| | | | | | TTACGGA | |
| 357 | 96133 | I | 34 ± 7 | 26 ± 7 | GTAACGATT | 358 |
| | | | | | TTACGG | |
| 358 | 96133 | I | 71 ± 14 | 36 ± 12 | AGTAACGAT | 359 |
| | | | | | TTTACGG | |
| 359 | 96133 | I | 34 ± 5 | 15 ± 3 | CATGAGTAA | 360 |
| | | | | | CGATTTTAC | |
| | | | | | GG | |
| 360 | 96134 | I | 55 ± 14 | 20 ± 6 | GAGTAACG | 361 |
| | | | | | ATTTTACG | |
| 361 | 96134 | I | 34 ± 18 | 13 ± 2 | TGAGTAAC | 362 |
| | | | | | GATTTTACG | |
| 362 | 96134 | I | 54 ± 10 | 21 ± 4 | TCATGAGTA | 363 |
| | | | | | ACGATTTTA | |
| | | | | | CG | |
| 363 | 96890 | I | 24 ± 7 | 17 ± 11 | TCCGTTCAA | 364 |
| | | | | | GCCTGCATG | |
| | | | | | AT | |
| 364 | 97386 | I | 75 ± 6 | 40 ± 8 | TGCACGTAG | 365 |
| | | | | | GAGTAGC | |
| 365 | 97386 | I | 69 ± 10 | 46 ± 6 | TTGCACGTA | 366 |
| | | | | | GGAGTAGC | |
| 366 | 97390 | I | 32 ± 6 | 15 ± 4 | CGAGCCTTT | 367 |
| | | | | | GCACGTAG | |
| | | | | | GAG | |
| 367 | 97391 | I | 56 ± 8 | 43 ± 6 | TCGAGCCTT | 368 |
| | | | | | TGCACGTAG | |
| | | | | | GA | |
| 368 | 97393 | I | 62 ± 25 | 33 ± 7 | ACTCGAGCC | 369 |
| | | | | | TTTGCACGT | |
| | | | | | AG | |
| 369 | 113516 | E | 88 ± 6 | 39 ± 4 | AATAGACA | 370 |
| | | | | | GCGCGATG | |
| | | | | | ATGT | |
| 370 | 113518 | E | 85 ± 22 | 37 ± 8 | TCAATAGAC | 371 |
| | | | | | AGCGCGAT | |
| | | | | | GAT | |
| 371 | 113519 | E | 57 ± 3 | 28 ± 9 | TAGACAGC | 372 |
| | | | | | GCGATGA | |
| 372 | 113519 | E | 52 ± 5 | 33 ± 16 | ATAGACAG | 373 |
| | | | | | CGCGATGA | |
| 373 | 113520 | E | 58 ± 5 | 27 ± 7 | ATAGACAG | 374 |
| | | | | | CGCGATG | |
| 374 | 113520 | E | 57 ± 11 | 31 ± 6 | AATAGACA | 375 |
| | | | | | GCGCGATG | |
| 375 | 113520 | E | 88 ± 13 | 35 ± 7 | CTTCAATAG | 376 |
| | | | | | ACAGCGCG | |
| | | | | | ATG | |
| 376 | 113521 | E | 61 ± 6 | 25 ± 8 | AATAGACA | 377 |
| | | | | | GCGCGAT | |
| 377 | 113521 | E | 63 ± 17 | 35 ± 12 | CAATAGACA | 378 |
| | | | | | GCGCGAT | |
| 378 | 113521 | E | 59 ± 7 | 33 ± 15 | TCAATAGAC | 379 |
| | | | | | AGCGCGAT | |
| 379 | 113521 | E | 56 ± 19 | 25 ± 3 | GCTTCAATA | 380 |
| | | | | | GACAGCGC | |
| | | | | | GAT | |
| 380 | 113523 | E | 42 ± 2 | 16 ± 4 | TCAATAGAC | 381 |
| | | | | | AGCGCG | |
| 381 | 113582 | E | 45 ± 8 | 17 ± 5 | GGTTGCGA | 382 |
| | | | | | AAGTCCGTT | |
| | | | | | TGG | |
| 382 | 113970 | E | 85 ± 17 | 20 ± 3 | CGATATCCT | 383 |
| | | | | | TCAGACGCT | |
| | | | | | TC | |
| 383 | 113973 | E | 59 ± 18 | 17 ± 6 | GAACGATAT | 384 |
| | | | | | CCTTCAGAC | |
| | | | | | GC | |
| 384 | 113974 | E | 91 ± 13 | 50 ± 18 | GATATCCTT | 385 |
| | | | | | CAGACG | |

¹Specificity of ASOs is human/non-human primate, with the exception of ASO numbers 13, 109-112, 138-140, 155-164, 169-172, 176, 179-182, 209, 224, 280, 281, 310-317, 324, 341, and 384, for which specificity is human/non-human primate/canine

1. A single-stranded oligonucleotide that is 10-80 nucleosides in length comprising a nucleobase sequence that comprises a portion of 10 contiguous nucleobases that is at least 80% complementary to an equal length portion of a target region of a human protocadherin 19 (PCDH19) gene, a pre-mRNA transcript thereof and/or an mRNA transcript thereof.

2. The oligonucleotide of claim 1, wherein the target region is within the nucleotide sequence set forth in SEQ ID NO: 1 or a variant thereof having at least or about 80% sequence identity to SEQ ID NO: 1.

3. The oligonucleotide of claim 1, wherein the mRNA transcript or the pre-mRNA transcript includes at least one PCDH19 mutation.

4. The oligonucleotide of claim 3, wherein the at least one PCDH19 mutation is selected from mutations that encode N340S, E307K, V441E, Q85X, N557K, D594H, S671X, L677fsX717, 1119fsX122, P364fsX375, or combinations.

5. The oligonucleotide of claim 3, wherein the oligonucleotide selectively hybridizes to the mRNA transcript or pre-mRNA transcript that includes the at least one PCDH19 mutation over a wild-type mRNA transcript or pre-mRNA transcript.

6. The oligonucleotide of claim 1 wherein the oligonucleotide consists of 12 to 40 nucleobases, 16 to 30 nucleobases, or 18 to 22 nucleobases.

7. The oligonucleotide of claim 1, wherein the oligonucleotide further comprises:
(a) a gap segment comprising linked deoxyribonucleosides;
(b) a 5′ wing segment comprising linked nucleosides; and
(c) a 3′ wing segment comprising linked nucleosides;
wherein the gap segment comprises a region of at least 10 contiguous nucleobases that is at least 80% complementary to an equal length portion of the target region of the pre-mRNA transcript or the mRNA transcript of the human PCDH19 gene positioned between the 5′ wing segment and the 3′ wing segment,
wherein the 5′ wing segment and the 3′ wing segment each comprise at least two linked nucleosides, and
wherein at least one nucleoside of each wing segment comprises an alternative nucleoside.

8. The oligonucleotide of claim 1, wherein the oligonucleotide comprises at least one alternative internucleoside linkage, at least one alternative nucleobase, and/or at least one alternative sugar moiety.

9. The oligonucleotide of claim 8, wherein the at least one alternative internucleoside linkage is selected from a phosphorothioate internucleoside linkage, a 2′-alkoxy internucleoside linkage, or an alkyl phosphate internucleoside linkage.

10. The oligonucleotide of claim 8, wherein the alternative nucleobase is a 5′-methylcytosine, a pseudouridine, or a 5-methoxyuridine.

11. The oligonucleotide of claim 8, wherein the alternative sugar moiety is a 2′-OMe modified sugar moiety or a bicyclic sugar moiety.

12. The oligonucleotide of claim 1, wherein the oligonucleotide further comprises a ligand conjugated to the 5′ end or the 3′ end of the oligonucleotide through a monovalent, branched bivalent, or trivalent linker.

13. The oligonucleotide of claim 1, wherein the oligonucleotide comprises a region complementary to at least 17 or to at least 19 contiguous nucleotides of the PCDH19 gene.

14. The oligonucleotide of claim 1, wherein the oligonucleotide comprises a nucleobase sequence as set forth in any one of SEQ ID NOs: 2 to 385.

15. A pharmaceutical composition comprising the oligonucleotide of claim 1 and a pharmaceutically acceptable carrier or excipient.

16. The composition of claim 15, comprising a lipid nanoparticle, a polyplex nanoparticle, a lipoplex nanoparticle, or a liposome.

17. A method of treating a PCDH19 related disorder in a subject in need thereof, the method comprising administering a therapeutically effective amount of the oligonucleotide of claim 1 to the subject.

18. (canceled)

19. The method of claim 17, wherein the PCDH19 related disorder is selected from the group consisting of epilepsy, schizophrenia, and autism.

20. The method of claim 17, wherein the subject has a mutation in at least one allele of the PCDH19 gene.

21. (canceled)

22. (canceled)

23. The method of claim 17, wherein the treating reduces one or more symptoms of the PCDH19 related disorder, wherein the one or more symptoms of the PCDH19 related disorder is selected from the group consisting of: prolonged seizures, frequent seizures, behavioral and developmental delays, movement and balance issues, orthopedic conditions, delayed language and speech issues, growth and nutrition issues, sleeping difficulties, chronic infection, sensory integration disorder, disruption of the autonomic nervous system, and sweating.

24. (canceled)
